(19) United States
(12) Patent Application Publication
(10) Pub. No.: US 2007/0213293 A1
(43) Pub. Date: Sep. 13, 2007

(54) RNAI THERAPEUTIC FOR RESPIRATORY VIRUS INFECTION

(75) Inventors: James Anthony McSwiggen, Bothell, WA (US); Qing Ge, Rolla, MO (US)

Correspondence Address: NASTECH PHARMACEUTICAL COMPANY INC, 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/687,564

(22) Filed: Mar. 16, 2007

Related U.S. Application Data
(63) Continuation-in-part of application No. PCT/US06/13374, filed on Apr. 7, 2006.
(60) Provisional application No. 60/669,942, filed on Apr. 8, 2005.

(51) Int. Cl. A61K 48/00; C07H 21/02

(52) U.S. Cl. 514/44; 536/23.1

(57) ABSTRACT

Double stranded siRNA molecules for combating a respiratory virus, wherein the strands of an siRNA molecule may be from about 15 to about 60 nucleotides, and uses thereof. One strand of an siRNA molecule can be a nucleic acid sequence identical to a conserved site, or a variant thereof, within the nucleic acid sequence of the respiratory virus.

### CROSS REFERENCE TO RELATED APPLICATIONS

This application is a continuation-in-part application under 35 U.S.C. §120 of copending PCT International Application No. PCT/2006/013374, filed Apr. 7, 2006, which claims the benefit of U.S. Provisional Application No. 60/669,942, filed Apr. 8, 2005, the disclosures of each of which are hereby incorporated by reference in their entirety.

### BACKGROUND OF THE INVENTION

Pathogenic viral infections are some of the most widely spread infections worldwide. For example, a family of such viruses is the influenza family. An estimated 20 to 40 million people died during the 1918 influenza A virus pandemic. In the United States, about 20 to 40 thousand people die from influenza A virus infection or its complications each year. During epidemics, the number of influenza related hospitalizations may reach over 300,000 in a single winter season. There is no superior therapy for influenza virus infection, and existing vaccines are limited in value in part because of the properties of antigenic shift and drift. Treatment or prevention of infections by a number of other viruses that are pathogenic to humans and other animals face similar difficulties. There is a great need for new means to combat such pathogenic viral infections.

RNA Interference (RNAi) refers to methods of sequence-specific post-transcriptional gene silencing which is mediated by a double-stranded RNA (dsRNA) called a short interfering RNA (siRNA). See Fire, et al., Nature 391:806, 1998, and Hamilton, et al., Science 286:950-951, 1999. RNAi is shared by diverse flora and phyla and is believed to be an evolutionarily-conserved cellular defense mechanism against the expression of foreign genes. See Fire, et al., Trends Genet. 15:358, 1999.

RNAi is therefore a ubiquitous, endogenous mechanism that uses small noncoding RNAs to silence gene expression. See Dykxhoorn, D. M. and J. Lieberman, Annu. Rev. Biomed. Eng. 8:377-402, 2006. RNAi can regulate important genes involved in cell death, differentiation, and development. RNAi may also protect the genome from invading genetic elements, encoded by transposons and viruses. When a siRNA is introduced into a cell, it binds to the endogenous RNAi machinery to disrupt the expression of mRNA containing complementary sequences with high specificity. Any disease-causing gene and any cell type or tissue can potentially be targeted. This technique has been rapidly utilized for gene-function analysis and drug-target discovery and validation. Harnessing RNAi also holds great promise for therapy, although introducing siRNAs into cells in vivo remains an important obstacle.

The mechanism of RNAi, although not yet fully characterized, is through cleavage of a target mRNA. The RNAi response involves an endonuclease complex known as the RNA-induced silencing complex (RISC), which mediates cleavage of a single-stranded RNA complementary to the antisense strand of the siRNA duplex. Cleavage of the target RNA takes place in the middle of the region complementary to the antisense strand of the siRNA duplex (Elbashir, et al., Genes Dev. 15:188, 2001).

One way to carry out RNAi is to introduce or express a siRNA in cells. Another way is to make use of an endogenous ribonuclease III enzyme called dicer. One activity of dicer is to process a long dsRNA into siRNAs. See Hamilton, et al., Science 286:950-951, 1999; Berstein, et al., Nature 409:363, 2001. A siRNA derived from dicer is typically about 21-23 nucleotides in overall length with about 19 base pairs duplexed. See Hamilton, et al., supra; Elbashir, et al., Genes Dev. 15:188, 2001. In essence, a long dsRNA can be introduced in a cell as a precursor of a siRNA.

An unmet need exists for molecules, compositions, and modalities for effective therapeutics in treating, ameliorating, or preventing pathogenic viral infections, diseases and disorders. This invention meets such needs and provides related advantages.

### BRIEF DESCRIPTION OF THE FIGURES

FIG. 1. Therapeutic administration of siRNA inhibits influenza virus production in mice. Mice were injected with NP-siRNA or PA-siRNA complexed with jetPEI 5 hours following influenza infection. Virus titers were measured in lung homogenates 28 hours post-infection by MDCK-HA assay. Each data point represents one mouse. P values between groups indicate statistical significance.

FIG. 2. Influenza-specific siRNA treatment provides broad cross-protection against lethal challenge with highly pathogenic H5 and H7 avian influenza A viruses. BALB/c mice (8 per group) were given 50 μg siRNA intravenously one day before virus challenge and another 20 μg of siRNA intranasally on the day of virus challenge. Body weights and survival of mice were monitored for 16 days after 10 LD50 dose of intranasal virus challenge. Filled circles, GFP-specific siRNA; open circles, NP plus PA-specific siRNAs. P values are indicated.

FIG. 3. BALB/c mice were treated intranasally with indicated amounts of NP specific siRNA in PBS or PBS control. Two hours later, all mice were infected intranasally (1000 pfu/mouse) with the PR8 serotype. The lungs were harvested 24 hours post-infection, and viral titer was measured from lung homogenates by MDCK-HA assay. P values between PBS and siRNA groups indicate statistical significance with 0.5, 1 and 2 mg/kg siRNA treated groups.

FIG. 4. BALB/c mice were administered control and NP-targeting siRNA intranasally (10 mg/kg, in PBS). Three hours later all the mice were infected i.n with PR8 virus (50 pfu/mouse). The lungs were harvested at 24 and 48 hours post-infection and total RNA was isolated from the left lung. Total mRNA was reverse transcribed to cDNA using dT18 primers. Real time PCR was carried out using PB1 specific primers to quantify viral mRNA levels. GAPDH was used as an internal control. The right and middle lungs were homogenized and the viral titer was measured by MDCK-HA assay. The virus titer in the samples at 48 hour post-infection is shown in the figure (statistic significance was found between PBS and NP siRNA treated group using student t test (p=0.01); the titer in the samples 24 hours post-infection was too low to detect, possibly due to siRNA directed suppression.

FIG. 5. Balb/c mice were treated intranasally with 10 mg/kg cyclophilin B specific siRNA or GFP siRNA in PBS or PBS control. There were five mice per group. The mouse lungs were harvested 24 later. Total RNA was purified from the lung samples and reverse transcription was conducted using dT18 primer. Cyclophilin B-specific primers were used in real-time PCR to quantify the target mRNA level. GAPDH-specific primers were also used in the PCR reaction as control.

FIG. 6. Influenza virus suppression in vivo by intranasal administration of cochleate siRNA formulations is shown.

FIG. 7. Influenza virus suppression in vivo by intravenous administration of cochleate siRNA formulations is shown.

FIG. 8A. Dose-response profile of intravenously administered siRNA delivered in cochleate formulations for influenza virus suppression.

FIG. 8B. Influenza virus suppression in vivo by oral gavage administration of cochleate siRNA formulations is shown.

### DETAILED DESCRIPTION OF THE INVENTION

This invention is based in part on the phenomenon of RNA Interference (RNAi). Therein, the presence in a cell of double-stranded RNA containing a portion that is complementary to a target RNA inhibits expression of the target RNA in a sequence-specific manner. Generally, inhibition is caused by cleavage of the target or inhibition of its translation. While RNAi is a normal cellular response to insults such as pathogen infection, it is also an effective mechanism to return to stasis the system perturbed by such an infection. Further, RNAi can be used to specifically disrupt cellular signaling pathways.

The double-stranded RNA structures that drive RNAi activity are siRNAs, shRNAs, and other double-stranded structures (dsRNAs) that can be processed to yield an siRNA or shRNA (or any other small RNA species that inhibits expression of a target transcript by RNA interference). RNAi-inducing entities such as siRNAs and shRNAs can be introduced into a subject, or an isolated cell thereof, and modulate specific signaling pathways. Further, these dsRNAs are useful therapeutics to prevent and treat diseases or disorders characterized by aberrant cell signaling. For instance, virus that infect mammals replicate by taking control of cellular machinery of the host cell. It is therefore useful to use RNAi technology to disrupt the viral signaling pathway that controls virus production.

It is known that certain viral genes control critical in two stages of the viral life cycle. Prior work in the art has focused on the viral polymerases are effective targets for siRNA as they are required for viral replication. For example, RNA-dependent RNA polymerase (RdRP) is an essential polypeptide in both transcription and replication. This has led to the speculation that silencing of RdRP subunits would lead to a nearly total loss of all RNA synthesis and thus result in a drastic inhibition of virus production. Indeed, this result has been observed in RSV, vesicular stomatitis and parainfluenza virus. (See, Barik S., Control of nonsegmented negative-strand RNA virus replication by siRNA, Virus Res. Jun. 1, 2001;102(1):27-35; and Bitko, et al., Phenotypic silencing of cytoplasmic genes using sequence-specific double-stranded short interfering RNA and its application in the reverse genetics of wild type negative-strand RNA viruses, BMC Microbiol. 2001;1(1):34. Epub, Dec. 20, 2001.) Another viral protein, variously termed nucleoprotein, capsid, or nucleocapsid, is recognized to be involved in, e.g., viral transcription and replication. However, antisense studies performed on nucleoprotein did not indicate that the transcript encoding this polypeptide is as suitable a target as polymerase. (See, Hatta, et al., Inhibition of influenza virus RNA polymerase and nucleoprotein genes expression by unmodified, phosphorothioated, and liposomally encapsulated oligonucleotides, Biochem. Biophys. Res. Commun. Jun. 14, 1996; 223(2):341-6; and Mizuta, et al., Antisense oligonucleotides directed against the viral RNA polymerase gene enhance survival of mice infected with influenza A, Nat. Biotechnol. June 1999;17(6):583-7). Recent studies with siRNA have suggested that nucleoprotein be investigated as a possible target of siRNA. Gitlin, et al., Short interfering RNA confers intracellular antiviral immunity in human cells, Nature Jul. 25, 2002; 418(6896):430-4; Fowler, et al., Inhibition of Marburg virus protein expression and viral release by RNA interference, J. Gen. Virol. April 2005; 86(Pt 4):1181-8; and Yuan, et al., Inhibition of coxsackievirus B3 replication by small interfering RNAs requires perfect sequence match in the central region of the viral positive strand, J. Virol. February 2005;79(4):2151-9). The present invention demonstrates the use of siRNAs directed to viral nucleoprotein sequences to disrupt viral signaling pathways and inhibit viral replication. Further, the present inventors determined that inhibition or silencing of another viral protein, nucleoprotein or nucleocapsid protein, has similar effects to inhibiting polymerase activity. Thus, a nucleoprotein transcript is preferred target for siRNA.

While not wishing to be bound by any particular theory, the broad effect of NP siRNA is likely a result of the importance of NP in binding and stabilizing vRNA and cRNA, instead of NP-specific siRNA non-specifically targeting RNA degradation. For example, the NP gene segment in influenza virus encodes a single-stranded RNA-binding nucleoprotein, which can bind to both vRNA and cRNA. During the viral life cycle, NP mRNA is first transcribed and translated. The primary function of the NP protein is to encapsidate the virus genome for the purpose of RNA transcription, replication and packaging. In the absence of NP protein, the full-length synthesis of both vRNA and cRNA is strongly impaired. When NP siRNA induces the degradation of NP RNA, NP protein synthesis is impaired and the resulting lack of sufficient NP protein subsequently affects the replication of other viral gene segments. In this way, NP siRNA is able to potently inhibit virus production at a very early stage. Thus, the multifunctional properties of viral nucleoproteins make them useful targets for RNAi-based therapy, offering the opportunity to intervene at multiple different stages of the viral life cycle by inhibiting a single gene.

It has been hypothesized that the number of NP protein molecules in infected host cells regulates mRNA synthesis, as opposed to replication of genome RNA (vRNA and cRNA). Using a temperature-sensitive mutation in the NP protein, previous studies have shown that cRNA, but not mRNA, synthesis was temperature sensitive both in vitro and in vivo. NP protein has been shown to be required for elongation and anti-termination of the nascent cRNA and vRNA transcripts. The results presented herein demonstrate that viral NP-specific siRNA inhibits the accumulation of all viral RNAs in infected cells. While not wishing to be bound by any particular theory, it appears probable that in the presence of NP-specific siRNA, the newly transcribed NP mRNA is degraded, resulting in the inhibition of NP protein synthesis following virus infection. Without newly synthesized NP, further viral transcription and replication, and therefore new virion production is inhibited.

### DEFINITIONS

For convenience, certain terms used in the specification, examples, and appended claims are collected here. Unless otherwise defined, all technical and scientific terms used herein have the same meaning as commonly understood by one of ordinary skill in the art to which this invention pertains. However, to the extent that these definitions vary from meanings circulating within the art, the definitions below are to control.

A “nucleotide” comprises a nitrogenous base, a sugar molecule, and a phosphate group. A “nucleoside” comprises a nitrogenous base (nucleobase) linked to a sugar molecule. In a naturally occurring nucleic acid, phosphate groups covalently link adjacent nucleosides to form a polymer. A nucleic acid may include naturally occurring nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose).

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g, by DNA replication or transcription of DNA or RNA, respectively). RNA can be post-transcriptionally modified. DNA and RNA can also be chemically synthesized. The terms “target mRNA” and “target transcript” are synonymous as used herein.

The term “RNA interference” (“RNAi”) refers to selective intracellular degradation of RNA (also referred to as gene silencing). RNAi also includes translational repression by microRNAs or siRNAs acting like microRNAs. RNAi can be initiated by introduction of small interfering RNAs (siRNAs) or production of siRNAs intracellularly (e.g., from a plasmid or transgene), to silence the expression of one or more target genes. Alternatively, RNAi occurs in cells naturally to remove foreign RNAs (e.g., viral RNAs). Natural RNAi proceeds via dicer-directed fragmentation of precursor dsRNA which direct the degradation mechanism to other cognate RNA sequences.

The term “small interfering RNA” (“siRNA”), also referred to in the art as “short interfering RNAs,” refers to an RNA (or RNA analog) comprising between about 10-60 nucleotides (or nucleotide analogs) that is capable of directing or mediating RNA interference. The term “siRNA” includes both double stranded siRNA and single stranded siRNA. Generally, as used herein the term “siRNA” refers to double stranded siRNA (as compared to single stranded or antisense RNA). The term “short hairpin RNA” (“shRNA”) refers to an siRNA (or siRNA analog) precursor that is folded into a hairpin structure and contains a single stranded portion of at least one nucleotide (a “loop”), e.g., an RNA molecule that contains at least two complementary portions hybridized or capable of hybridizing to form a double-stranded (duplex) structure sufficiently long to mediate RNAi (as described for siRNA duplexes), and at least one single-stranded portion, typically between approximately 1 and 10 nucleotides in length that forms a loop connecting the regions of the shRNA that form the duplex portion. The duplex portion may, but typically does not, contain one or more mismatches and/or one or more bulges consisting of one or more unpaired nucleotides in either or both strands. Without wishing to be bound by theory, shRNAs are thought to be processed into siRNAs by the conserved cellular RNAi machinery. shRNAs are capable of inhibiting expression of a target transcript that is complementary to a portion of the shRNA (referred to as the antisense or guide strand of the shRNA). In general, the features of the duplex formed between the guide strand of the shRNA and a target transcript are similar to those of the duplex formed between the guide strand of an siRNA and a target transcript. In certain embodiments of the invention the 5′ end of an shRNA has a phosphate group while in other embodiments it does not. In certain embodiments of the invention the 3′ end of an shRNA has a hydroxyl group.

The term “RNAi-inducing entity” or “RNAi agent” refers to an RNA species (other than a naturally occurring molecule not modified by the hand of man or transported into its location by the hand of man) whose presence within a cell results in RNAi and leads to reduced expression of an RNA to which the RNAi agent is targeted. The RNAi agent may be, for example, an siRNA or shRNA. In certain embodiments of the invention an siRNA may contain a strand that inhibits expression of a target RNA via a translational repression pathway utilized by endogenous small RNAs referred to as microRNAs. In certain embodiments of the invention an shRNA may be processed intracellularly to generate an siRNA that inhibits expression of a target RNA via this microRNA translational repression pathway. Any “target RNA” may be referred to as a “target transcript” regardless of whether the target RNA is a messenger RNA. The terms “target RNA” and “target transcript” are used interchangeably herein. The term RNAi-inducing agent encompasses RNAi agents and vectors (other than naturally occurring molecules not modified by the hand of man as described above) whose presence within a cell results in RNAi and leads to reduced expression of a transcript to which the RNAi agent is targeted.

An “RNAi-inducing vector” includes a vector whose presence within a cell results in transcription of one or more RNAs that self-hybridize or hybridize to each other to form an RNAi agent. In various embodiments of the invention this term encompasses plasmids, e.g., DNA vectors (whose sequence may comprise sequence elements derived from a virus), or viruses, (other than naturally occurring viruses or plasmids that have not been modified by the hand of man), whose presence within a cell results in production of one or more RNAs that self-hybridize or hybridize to each other to form an RNAi agent. In general, the vector comprises a nucleic acid operably linked to expression signal(s) so that one or more RNA molecules that hybridize or self-hybridize to form an RNAi agent is transcribed when the vector is present within a cell. Thus the vector provides a template for intracellular synthesis of the RNAi agent. For purposes of inducing RNAi, presence of a viral genome into a cell (e.g., following fusion of the viral envelope with the cell membrane) is considered sufficient to constitute presence of the virus within the cell. In addition, for purposes of inducing RNAi, a vector is considered to be present within a cell if it is introduced into the cell, enters the cell, or is inherited from a parental cell, regardless of whether it is subsequently modified or processed within the cell. An RNAi-inducing vector is considered to be targeted to a transcript if presence of the vector within a cell results in production of one or more RNAs that hybridize to each other or self-hybridize to form an RNAi agent that is targeted to the transcript, i.e., if presence of the vector within a cell results in production of one or more RNAi agent targeted to the transcript. Use of the term “induce” is not intended to indicate that the RNAi agent necessarily activates or upregulates RNAi in general but simply indicates that presence of the vector within a cell results in production of an RNAi agent within the cell, leading to an RNAi-mediated reduction in expression of an RNA to which the agent is targeted.

An RNAi-inducing entity is considered to be targeted to a target transcript for the purposes described herein if (1) the agent comprises a strand that is substantially complementary to the target transcript over a window of evaluation between 15-29 nucleotides in length, e.g., 15, more preferably at least about 17, yet more preferably at least about 18 or 19 to about 21-23 or 24-29 nucleotides in length. For example, in various embodiments of the invention the agent comprises a strand that has at least about 70%, preferably at least about 80%, 84%, 89%, 90%, 91%, 92%, 93%, 94%, 95%, 96%, 97%, 98%, 99%, or 100% precise sequence complementarity with the target transcript over a window of evaluation between 15-29 nucleotides in length, e.g., over a window of evaluation of at least 15, more preferably at least about 17, yet more preferably at least about 18 or 19 to about 21-23 or 24-29 nucleotides in length; or (2) one strand of the RNAi agent hybridizes to the target transcript under stringent conditions for hybridization of small (<50 nucleotide) RNA molecules in vitro and/or under conditions typically found within the cytoplasm or nucleus of mammalian cells. In addition, in the case of agents that act via the microRNA translational repression pathway, the duplex formed by the agent and the target contains at least one bulge and/or mismatch. In certain embodiments of the invention a GU or UG base pair in a duplex formed by a guide strand and a target transcript is not considered a mismatch for purposes of determining whether an RNAi agent is targeted to a transcript.

An RNA-inducing vector whose presence within a cell results in production of an RNAi agent that is targeted to a transcript is also considered to be targeted to the transcript. Since the effect of targeting a transcript is to reduce or inhibit expression of the gene that directs synthesis of the transcript, an RNAi agent targeted to a transcript is also considered to target the gene that directs synthesis of the transcript even though the gene itself (e.g., genomic DNA in the case of a cell) is not thought to interact with the agent or components of the cellular silencing machinery. Thus an RNAi agent or vector that targets a transcript is understood to target the gene that provides a template for synthesis of the transcript.

A viral “nucleoprotein” (also termed a “capsid protein” or a “nucleocapsid protein”) is a viral polypeptide that sequesters viral RNA and affects viral transcription. The viral nucleoprotein is capable of forming a nucleic acid/protein complex (i.e., a ribonucleoprotein (RNP) complex). Nucleoproteins are also termed “NS” in double stranded viruses (e.g., NS-6). A nucleoprotein is distinguished from an outer capsid protein, which generally does not contact and sequester the viral genome. The terms “nucleoprotein mRNA,” “NP mRNA”, “nucleoprotein transcript,” and “NP transcript” are understood to include any mRNA that encodes a viral nucleoprotein or its functional equivalent as described herein.

As will be appreciated by one of ordinary skill in the art, proteins fulfilling one or more functions of a viral nucleoprotein are referred to by a number of different names, depending on the particular virus of interest. For example, in the case of certain viruses such as influenza the protein is known as nucleoprotein (NP) while in the case of a number of other single-stranded RNA viruses, proteins that fulfill a similar role are referred to as nucleocapsid (NC or N) proteins. In yet other viruses, analogous proteins that both interact with genomic nucleic acid and play a structural role in the viral particle are considered to be capsid (C) proteins.

As used herein, the terms “nucleoprotein mRNA,” “NP mRNA”, “nucleoprotein transcript,” and “NP transcript” are understood to include any mRNA that encodes a viral nucleoprotein or its functional equivalent as described herein. Any virus containing a nucleoprotein gene or the functional equivalent thereof is suitable as an siRNA target. By way of non-limiting example, several groups of target viruses are described herein in greater detail.

“Subject” includes living organisms such as humans, monkeys, cows, sheep, horses, pigs, cattle, goats, dogs, cats, mice, rats, cultured cells therefrom, and transgenic species thereof In a preferred embodiment, the subject is a human. A subject is synonymous with a “patient.” Administration of the compositions of the present invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The term “complementary” is used herein in accordance with its art-accepted meaning to refer to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation). A degree of complementarity of two nucleic acids or portions thereof may be evaluated by determining the total number of nucleotides in both strands that form complementary base pairs as a percentage of the total number of nucleotides over a window of evaluation when the two nucleic acids or portions thereof are aligned in antiparallel orientation for maximum complementarity. For example, AAAAAAAA (SEQ ID NO: 17260) and TTTGTTAT (SEQ ID NO: 17261) are 75% complementary since there are 12 nucleotides in complementary base pairs out of a total of 16. Nucleic acids that are at least 70% complementary over a window of evaluation are considered substantially complementary over that window. Specifically, if the window of evaluation is 15-16 nucleotides long, substantially complementary nucleic acids may have 0-3 mismatches within the window; if the window is 17 nucleotides long, substantially complementary nucleic acids may have 0-4 mismatches within the window; if the window is 18 nucleotides long, substantially complementary nucleic acids may have may contain 0-5 mismatches within the window; if the window is 19 nucleotides long, substantially complementary nucleic acids may contain 0-6 mismatches within the window. In certain embodiments the mismatches are not at continuous positions. In certain embodiments the window contains no stretch of mismatches longer than two nucleotides in length. In preferred embodiments a window of evaluation of 15-19 nucleotides contains 0-1 mismatch (preferably 0), and a window of evaluation of 20-29 nucleotides contains 0-2 mismatches (preferably 0-1, more preferably 0).

“Substantially pure” includes compounds, e.g., drugs, proteins or polypeptides that have been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” are compounds, such as proteins or polypeptides, which are homogeneously pure, for example, where at least 95% of the total protein (by volume, by wet or dry weight, or by mole percent or mole fraction) in a sample is the protein or polypeptide of interest.

“Administering” includes routes of administration which allow the compositions of the invention to perform their intended function, e.g., treating or preventing viral disease. A variety of routes of administration are possible including, but not necessarily limited to parenteral (e.g., intravenous, intraarterial, intramuscular, subcutaneous injection), oral (e.g., dietary), inhalation (e.g., aerosol to lung), topical, nasal, rectal, or via slow releasing microcarriers depending on the disease or condition to be treated. Inhalation and parenteral administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of the composition of the invention which allow it to perform its intended function, e.g., treating or preventing, partially or totally, viral infection as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 100 mg/kg/day, advantageously from about 0.1 mg/kg/day to about 10 mg/kg/day. For example, an siRNA is delivered to a subject in need thereof at a dosage of from about 0.1 mg/kg/day to about 5 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, e.g., in Remington's Pharmaceutical Sciences.

“Conserved sites” of a virus are those sites or sequences that are found to be present in more than about 70% of all known sequences for a given region. The set of siRNA having sequence identity to conserved sites are determined by deriving all 19-mer sequence fragments from each of the known viral sequences, and evaluating the frequency in which each sequence fragment is present as an exact match within each of the set of viral sequences. A first viral sequence contains a 19-mer sequence fragment that extends from position 1 through 19, another from position 2 through 20, another from position 3 through 21, and so on until the 19 nucleotide site at the end of the strand.

Likewise the second, third, and fourth viral sequences are extracted in the same way, all the way down to the last viral sequence in the list. The sequence fragments are then added to a growing table of sequence fragments and a count is maintained of the number of viral sequences that contain each 19-mer fragment. The fragment frequency is expressed as the percent of the viral sequences that contain each specific 19-mer fragment. The set of siRNA of the invention are those having sequence identity with greater than a majority of the known sequences, preferably greater than about 70% of the known sequences.

“Conserved sites for influenza virus” do not include sequences disclosed in U.S. patent application Ser. No. 10/674,159 filed Sep. 29, 2003, Publication No. US-2004-0242518 A1 (J. Chen, Q. Ge and M. Eisen, “Influenza Therapeutic”). Conserved sites for influenza virus may exclude some embodiments disclosed in copending U.S. patent application Ser. No. 11/102097 filed Apr. 8, 2005 (a CIP of the above identified application), hereby incorporated by reference in its entirety. Conserved sites for influenza virus include embodiments disclosed in copending PCT Patent Application No. PCT/US06/013374 filed Apr. 7, 2006, hereby incorporated by reference in its entirety.

“Variants of a conserved site” include a small number of mismatches that are tolerated between the target RNA and the antisense guide sequence of the siRNA duplex. Thus, a single siRNA duplex targeting a highly conserved site in a virus will often still be active against minor variant species having only one or a few mismatches relative to the conserved site. We used the viral mismatch data in an algorithm to expand the list of potential influenza A viral sequence variants that are targetable by a given siRNA duplex, described below in Example 15.

### Nucleoproteins as RNAi Targets

The present invention provides compositions and methods using RNAi for treating or preventing virus replication or infection in a subject, such as a human or non-human mammal. Preferably, the virus is an RNA virus. For example, the RNA virus is a negative strand virus. Alternatively, the virus is a positive strand virus or a double stranded (ds) virus. A preferred target RNA is the nucleoprotein (also termed nucleocapsid) transcript, or a transcript of a viral gene that accomplishes the function of the viral nucleoprotein. Any virus containing a nucleoprotein gene or the functional equivalent thereof is suitable as an siRNA target. By way of non-limiting example, several groups of target viruses are described herein in greater detail.

### Negative Strand RNA Viruses

Negative strand RNA viruses have a viral genome that is in the complementary sense of mRNA. Therefore, one of the first activities of negative strand RNA viruses following entry into a host cell is transcription and production of viral mRNAs. For this purpose, the virions carry an N-RNA structure that consists of the viral RNA (vRNA) that is tightly associated with the viral nucleoprotein (N or NP, sometimes called nucleocapsid protein). The RNA-dependent RNA polymerase binds either directly to the N-RNA, as is the case for influenza virus, or it binds with the help of a co-factor, like the phosphoprotein of the paramyxoviruses and the rhabdoviruses. The intact N-RNA is the actual template for transcription rather than the naked vRNA and nucleoprotein contributes to exposure of the nucleotide bases of the N-RNA for efficient reading by the polymerase.

Commonalties in expression and replication of ssRNA(−) viruses appear to include distinct transcription and replication functions for the RdRp, probably triggered by binding of the virion nucleoprotein (N or NP) subunits. Thus, both RNA(−) and RNA(+) may be found complexed with N proteins in replication complexes.

### Influenza

Influenza viruses are enveloped, negative-stranded RNA viruses of the Orthomyxoviridae family. They are classified as influenza types A, B, and C, of which influenza A is the most pathogenic and is believed to be the only type able to undergo reassortment with animal strains. Current vaccines based upon inactivated virus are able to prevent illness in approximately 70-80% of healthy individuals under age 65; however, this percentage is far lower in the elderly or immunocompromised. In addition, the expense and potential side effects associated with vaccine administration make this approach less than optimal. There are four antiviral drugs currently approved in the United States for treatment and/or prophylaxis of influenza, amantadine, rimanadine, zanamivir, and oseltamivir, but their use is limited due to concerns about side effects, compliance, and possible emergence of resistant strains. Therefore, there remains a need for the development of effective therapies for the treatment and prevention of influenza infection.

Influenza nucleocapsid protein or nucleoprotein (NP) is the major structural protein that interacts with the RNA segments to form RNP. It is encoded by RNA segment 5 of influenza A virus and is 1,565 nucleotides in length. NP contains 498 amino acids. NP protein is critical in virus replication. The number of NP protein molecules in infected cells has been hypothesized to regulate the levels of mRNA synthesis versus genome RNA (vRNA and cRNA) replication (1) Using a temperature-sensitive mutation in the NP protein, previous studies have shown that cRNA, but not mRNA, synthesis was temperature-sensitive both in vitro and in vivo (28, 29). NP protein was also shown to be required for elongation and antitermination of nascent cRNA and vRNA transcripts (29, 30). The present inventors have found that NP-specific siRNA inhibited the accumulation of all viral RNAs in infected cells. Probably, in the presence of NP-specific siRNA, the newly transcribed NP mRNA is degraded, resulting in inhibition of NP protein synthesis. Without newly synthesized NP, further viral transcription and replication are blocked, as is new virion production.

### RNA-Inducing Entities—siRNA and shRNA Molecules

The present invention features siRNA molecules, methods of making siRNA molecules and methods (e.g., prophylactic and/or therapeutic methods and methods for research) for using siRNA molecules. The siRNA molecule can have a length from about 10-60 or more nucleotides (or nucleotide analogs), about 15-25 nucleotides (or nucleotide analogs), or about 19-23 nucleotides (or nucleotide analogs). The siRNA molecule can have nucleotide (or nucleotide analog) lengths of about 10-20, 20-30, 30-40, 40-50, 15, 16, 17, 18, 19, 20, 21, 22, 23, 24, 25, 26, 27, 28, or 29. In a preferred embodiment, the siRNA molecule has a length of 19 nucleotides. It is to be understood that all ranges and values encompassed in the above ranges are within the scope of the present invention. Generally, long dsRNAs (over 60 nucleotides) are less preferable, as they have been found to induce cell death (termed the “interferon response”) in mammalian cells, such as human cells. siRNAs can preferably include 5′ terminal phosphate and a 3′ short overhang of about 1 or 2 nucleotides. In a preferred embodiment, the RNAi-inducing entity can be a short hairpin siRNA (shRNA) or an expressed shRNA. Examples of such shRNAs and methods of manufacturing the same are discussed in the examples. In another embodiment, the siRNA can be associated with one or more proteins in an siRNA complex.

The siRNA molecules of the invention are provided to reduce viral gene expression in a host cell by, at least in part, binding to target viral transcripts in a manner that results in destruction of the target viral transcript by the host cell machinery. Thus, the siRNA molecules of the invention include a sequence that is sequence sufficiently complementary to a portion of the viral nucleoprotein gene to mediate RNA interference (RNAi), as defined herein, i e., the siRNA has a sequence sufficiently specific to trigger the degradation of the target RNA by the RNAi machinery or process. The siRNA molecule can be designed such that every residue of the antisense strand is complementary to a residue in the target molecule. Alternatively, substitutions can be made within the molecule to increase stability and/or enhance processing activity of said molecule. Substitutions can be made within the strand or can be made to residues at the ends of the strand.

The target RNA cleavage reaction guided by siRNAs is highly sequence specific. In general, siRNAs containing a nucleotide sequence identical to a portion of the target gene are preferred for inhibition. As the siRNAs of the invention are generally provided as double stranded molecules, identity and complimentarily of the antisense strand of the siRNA can be determined relative to the target transcript. Thus, as used herein, disclosure of a nucleic acid sequence that is identical to a portion of a nucleic acid encoding a viral nucleoprotein includes both strands of a double stranded siRNA. However, it is recognized that 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence are effective for inhibition. Alternatively, siRNA sequences with nucleotide analog substitutions or insertions are effective for inhibition. Moreover, not all positions of a siRNA contribute equally to target recognition. Mismatches in the center of the siRNA are most critical and can essentially abolish target RNA cleavage. In contrast, the 3′ nucleotides of the siRNA (e.g., the 3′ nucleotides of the siRNA antisense strand) typically do not contribute significantly to specificity of the target recognition. In particular, 3′ residues of the siRNA sequence which are complementary to the target RNA (e.g., the guide sequence) generally are not as critical for target RNA cleavage.

It is known in the art that not all siRNAs are equally effective in reducing or inhibiting expression of any particular target gene. (See, e.g., Holen, T., et al., Nucleic Acids Res. 30(8): 1757-1766, reporting variability in the efficacy of different siRNAs), and a variety of considerations may be employed to increase the likelihood that a selected siRNA may be effective. For example, it may be preferable to select target portions within exons rather than introns. siRNAs may generally be designed in accordance with principles described in Technical Bulletin #003-Revision B, “siRNA Oligonucleotides for RNAi Applications” and Technical Bulletin #4, Dharmacon Research, Inc., Lafayette, CO 80026, a commercial supplier of RNA reagents. The RNAi Technical Reference & Application Guide, from Dharmacon, contains a variety of information regarding siRNA design parameters, synthesis, etc., and is incorporated herein by reference. Additional design considerations that may also be employed are described in Semizarov, D., et al., Proc. Natl. Acad. Sci. 100(11):6347-6352.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology equals the number of identical positions divided by the total number of positions multiplied by 100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin & Altschul, Proc. Natl. Acad. Sci. USA 87:2264-68, 1990, modified as in Karlin & Altschul, Proc. Natl. Acad. Sci. USA 90:5873-77, 1993. Such an algorithm is incorporated into the BLAST programs (version 2.0) of Altschul, et al., J. Mol. Biol. 215:403-10, 1990. In another embodiment, the alignment is optimized by introducing appropriate gaps and percent identity is determined over the length of the aligned sequences (i.e., a gaped alignment). To obtain gaped alignments for comparison purposes, Gaped BLAST can be utilized as described in Altschul, et al., Nucleic Acids Res. 25(17):3389-3402, 1997. In another embodiment, the alignment is optimized by introducing appropriate gaps and percent identity is determined over the entire length of the sequences aligned (i.e., a global alignment). A so preferred, non-limiting example of a mathematical algorithm utilized for the global comparison of sequences is the algorithm of Myers and Miller, CABIOS, 1989. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In some embodiments, equal to or more than 70% sequence identity between the siRNA (e.g., the antisense strand of the siRNA) and the portion of the target gene is preferred, which means that the antisense strand of the siRNA is equal to or more than 70% complementary to the target gene.

In some embodiments, greater than 80% sequence identity, e.g., 84%, 89%, 91%, 92%, 93%, 94%, 95%, 96%, 97%, 98%, 99% or even 100% sequence identity, between the siRNA (e.g., the antisense strand of the siRNA) and the portion of the target gene is preferred. In the context of an siRNA of about 19-25 nucleotides, e.g., at least 16-21 identical nucleotides are preferred, more preferably at least 17-22 identical nucleotides, and even more preferably at least 18-23 or 19-24 identical nucleotides. Alternatively worded, in an siRNA of about 19-25 nucleotides in length, siRNAs having no greater than about 4 mismatches are preferred, preferably no greater than 3 mismatches, more preferably no greater than 2 mismatches, and even more preferably no greater than 1 mismatch. For example, the siRNA contains an antisense strand having 1, 2, 3 or 4 mismatches with the target sequence.

Alternatively, the siRNA may be defined functionally as including a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41(% G+C) (600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., et al., 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel, et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

In one embodiment, the RNA molecules of the present invention are modified, such as to improve stability in serum or in growth medium for cell cultures. In order to enhance the stability, the 3′-residues may be stabilized against degradation, e.g., they may be selected such that they consist of purine nucleotides, e.g., adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

In a preferred embodiment of the present invention the RNA molecule may contain at least one modified nucleotide analogue (or analog). The nucleotide analogues may be located at positions where the target-specific activity, e.g., the RNAi mediating activity is not substantially affected, e.g., in a region at the 5′-end and/or the 3′-end of the RNA molecule. Particularly, the ends may be stabilized by incorporating modified nucleotide analogues. Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphorothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Also preferred are nucleobase-modified ribonucleotides, i.e., ribonucleotides containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O— and N-alkylated nucleotides, e.g., N6-methyl adenosine are suitable. It should be noted that the above modifications may be combined.

In some embodiments, the siRNA can be modified by the substitution of at least one nucleotide with a modified nucleotide. The siRNA can have one or more mismatches when compared to the target sequence of the nucleoprotein transcript and still mediate RNAi as demonstrated in the examples below.

The ability of the nucleoprotein-directed siRNAs of the present invention to mediate RNAi is particularly advantageous considering the rapid mutation rate of some of the genes of the viruses provided herein, such as genes of an influenza virus. The inventors provide for the use of the nucleoprotein gene as an RNAi target as the inventors have recognized that the nucleoprotein gene generally has a lower rate of mutations as compared to other viral genes. Moreover, in embodiments of the invention, siRNAs are targeted towards conserved regions of the viral nucleoprotein gene. The invention contemplates several embodiments which further leverage this ability by, e.g., synthesizing patient-specific siRNAs or plasmids, and/or introducing several siRNAs staggered along the nucleoprotein gene. In one embodiment, highly and/or moderately conserved regions of the nucleoprotein gene are targeted as discussed in greater detail below. In other embodiments, a biological sample is obtained from a subject. As used herein, a biological sample is any material obtained from the subject containing a viral nucleic acid. For example, one or more of a host subject's infected cells are procured and the genome of the viral nucleoprotein gene within it sequenced or otherwise analyzed to select or synthesize one or more corresponding siRNAs, plasmids or transgenes.

### Manufacture of siRNA

In one embodiment, siRNAs are synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, or splice donor and acceptor) may be used to transcribe the siRNA. Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses siRNA from a recombinant construct may be produced by introducing the construct into a zygote, an embryonic stem cell, or another multipotent cell derived from the appropriate organism.

In addition, not only can an siRNA be used to cleave multiple RNAs within the cell, but the siRNAs can be replicated and amplified within a cell by the host cell enzymes. Alberts, et al., The Cell 452 (4th ed. 2002).

RNA may be produced enzymatically or by partial/total organic synthesis, any modified ribonucleotide can be introduced by in vitro enzymatic or organic synthesis. In one embodiment, a siRNA is prepared chemically. Methods of synthesizing RNA molecules are known in the art, in particular, the chemical synthesis methods as described in Verma and Eckstein, Annul Rev. Biochem. 67:99-134 (1998). In another embodiment, a siRNA is prepared enzymatically. For example, a siRNA can be prepared by enzymatic processing of a long dsRNA having sufficient complementarity to the desired target RNA. Processing of long dsRNA can be accomplished in vitro, for example, using appropriate cellular lysates and ds-siRNAs can be subsequently purified by gel electrophoresis or gel filtration. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing.

The siRNAs can also be prepared by enzymatic transcription from synthetic DNA templates or from DNA plasmids isolated from recombinant bacteria. Typically, phage RNA polymerases are used such as T7, T3 or SP6 RNA polymerase (Milligan & Uhlenbeck, Methods Enzymol. 180:51-62 (1989)). The RNA may be dried for storage or dissolved in an aqueous solution. The solution may contain buffers or salts to inhibit annealing, and/or promote stabilization of the single strands.

siRNA Vectors

Another aspect of the present invention includes a vector that expresses one or more siRNAs that include sequences sufficiently complementary to a portion of the nucleoprotein gene genome to mediate RNAi. The vector can be administered in vivo to thereby initiate RNAi therapeutically or prophylactically by expression of one or more copies of the siRNAs. In one embodiment, synthetic shRNA is expressed in a plasmid vector. In another, the plasmid is replicated in vivo. In another embodiment, the vector can be a viral vector, e.g., a retroviral vector. Examples of such plasmids and methods of making the same are illustrated in the examples. Use of vectors and plasmids are advantageous because the vectors can be more stable than synthetic siRNAs and thus effect long-term expression of the siRNAs.

Some target viruses mutate rapidly and may result in a mismatch of even one nucleotide that can, in some instances, impede RNAi. Accordingly, in one embodiment, a vector is contemplated that expresses a plurality of siRNAs to increase the probability of sufficient homology to mediate RNAi. Preferably, these siRNAs are staggered along the nucleoprotein gene, or are clustered in one region of the nucleoprotein gene. For example, a plurality of siRNAs is directed towards a region of the nucleoprotein gene that is about 200 nucleotides in length and contains the 3′ end of the nucleoprotein gene. In one embodiment, one or more of the siRNAs expressed by the vector is a shRNA. The siRNAs can be staggered along one portion of the nucleoprotein gene or target different portions of the nucleoprotein gene. In one embodiment, the vector encodes about 3 siRNAs, more preferably about 5 siRNAs. The siRNAs can be targeted to conserved regions of the nucleoprotein gene.

### Methods of Introducing RNAs, Vectors, and Host Cells

Physical methods of introducing the agents of the present invention (e.g., siRNAs, vectors, or transgenes) include injection of a solution containing the agent, bombardment by particles covered by the agent, soaking the cell or organism in a solution of the agent, or electroporation of cell membranes in the presence of the agent. A viral construct packaged into a viral particle would accomplish both efficient introduction of an expression construct into the cell and transcription of RNA, including siRNAs, encoded by the expression construct. Other methods known in the art for introducing nucleic acids to cells may be used, such as lipid-mediated carrier transport, chemical-mediated transport, such as calcium phosphate, and the like. Thus the siRNA may be introduced along with components that perform one or more of activities, e.g., enhance siRNA uptake by the cell, inhibit annealing of the two siRNA strands to each other, stabilize the single strands, or otherwise increase inhibition of the target gene.

The agents may be directly introduced into the cell (i.e., intracellularly); or introduced extracellularly into a cavity, interstitial space, into the circulation of an organism, introduced orally, by inhalation, or may be introduced by bathing a cell or organism in a solution containing the RNA. Vascular or extravascular circulation, the blood or lymph system, and the cerebrospinal fluid are sites where the agent may be introduced.

Cells may be infected with a target virus upon delivery of the agent or exposed to the target virus after delivery of agent. The cells may be derived from or contained in any organism. The cell may be from the germ line, somatic, totipotent or pluripotent, dividing or non-dividing, parenchyma or epithelium, immortalized or transformed, or the like. The cell may be a stem cell, or a differentiated cell.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of viral protein, RNA, and/or DNA. Specificity refers to the ability to inhibit the target gene without manifesting effects on other genes, particularly those of the host cell. The consequences of inhibition can be confirmed by examination of the outward properties of the cell or organism or by biochemical techniques such as RNA solution hybridization, nuclease protection, Northern hybridization, reverse transcription gene expression monitoring with a microarray, antibody binding, enzyme linked immunosorbent assay (ELISA), integration assay, Western blotting, radioimmunoassay (RIA), other immunoassays, and fluorescence activated cell analysis (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of siRNA may result in inhibition in a smaller fraction of cells (e.g., at least 10%, 20%, 50%, 75%, 90%, or 95% of targeted cells).

Quantitation of gene expression in a cell may show similar amounts of inhibition at the level of accumulation of target RNA or translation of target protein. As an example, the efficiency of inhibition may be determined by assessing the amount of gene product in the cell; RNA may be detected with a hybridization probe having a nucleotide sequence outside the region used for the inhibitory double-stranded RNA, or translated polypeptide may be detected with an antibody raised against the polypeptide sequence of that region.

The siRNA may be introduced in an amount that allows delivery of at least one copy per cell. Higher doses (e.g., at least 5, 10, 100, 500 or 1000 copies per cell) of material may yield more effective inhibition; lower doses may also be useful for specific applications.

### Diagnostic Methods and Kits

The invention encompasses the recognition that RNAi-based therapy of infectious diseases, e.g., infections caused by a virus, can desirably incorporate a diagnostic step that determines whether a subject in need of treatment is infected with an infectious agent that is susceptible to inhibition by one or more RNAi-inducing entities. By “susceptible to inhibition” is meant that one or more biological activities of the infectious agent can be effectively inhibited by administration of the RNAi-inducing entity to a subject. Preferably replication, pathogenicity, spread, and/or production of the infectious agent are inhibited. For example, preferably replication, pathogenicity, spread, or production of the agent is inhibited by at least 25% when the RNAi-inducing entity is administered to a subject at a tolerated dose. Preferably the inhibition is sufficient to produce a therapeutically useful effect.

Influenza virus is used as a non-limiting example to illustrate the diagnostic methods of the invention, which are tailored to allow the selection of an RNAi-inducing entity that is suitable for a subject suffering from an infection. However, it is understood that the methods disclosed herein are appropriate to any virus described herein or any virus that would be recognized by one skilled in the art. The selected RNAi-inducing entity may, of course, also be administered for prophylaxis, e.g., to individuals who have come in contact with the infected individual, regardless of whether those individuals have developed symptoms of infection.

The invention therefore provides methods for diagnosing virus infection and for determining whether a subject is infected with a virus. In certain embodiments the method comprises determining whether a subject is infected with a virus that is inhibited by one or more of the RNAi-inducing entities of the invention that target a viral nucleoprotein transcript. For example, a sample (e.g., sputum, saliva, nasal washings, nasal swab, throat swab, bronchial washings, broncheal alveolar lavage (BAL) fluid, biopsy specimens, etc.) is obtained from a subject who may be suspected of having a viral infection, e.g., influenza. The sample can be subjected to one or more processing steps. Any such processed sample is considered to be obtained from the subject. The sample is analyzed to determine whether it contains a virus-specific nucleic acid, particularly a nucleoprotein transcript. A “virus-specific nucleic acid” is any nucleic acid, or its complement, that originates from or is derived from a virus and can serve as an indication of the presence of a virus in a sample and, optionally, be used to identify the strain and/or the sequence of a viral gene. The nucleic acid may have been subjected to processing steps following its isolation. For example, it may be reverse transcribed, amplified, cleaved, etc. In certain embodiments the sequence of a virus-specific nucleic acid present in the sample, or its complement, is compared with the sequence of the antisense or sense strand of an RNAi-inducing agent such as an siRNA or shRNA. The word “comparison” is used in a broad sense to refer to any method by which a sequence can be evaluated, e.g., which it can be determined whether the sequence is the same as or different to a reference sequence at one or more positions, or by which the extent of difference can be assessed.

Any of a wide variety of nucleic acid-based assays can be used. In certain embodiments the diagnostic assay utilizes a nucleic acid comprising a favorably and/or highly conserved target portion or its complement, or a fragment of the favorably and/or highly conserved portion or its complement. In certain embodiments the nucleic acid serves as an amplification primer or a hybridization probe, e.g., in an assay such as those described below.

In certain embodiments an influenza-specific nucleic acid in the sample is amplified. Isothermal target amplification methods include transcription mediated amplification (TMA), self-sustained sequence replication (3SR), Nucleic Acid Sequence Based Amplification (NASBA), and variations thereof. Detection or comparison can be performed using any of a variety of methods known in the art, e.g., amplification-based assays, hybridization assays, primer extension assays (e.g., allele-specific primer extension in which the corresponding target portions of different influenza virus strains are analogous to different alleles of a gene), oligonucleotide ligation assays (U.S. Pat. Nos. 5,185,243, 5,679,524 and 5,573,907), cleavage assays, heteroduplex tracking analysis (HTA) assays, etc. Examples include the Taqman® assay, Applied Biosystems (U.S. Pat. No. 5,723,591). Cycling probe technology (CPT), which is a nucleic acid detection system based on signal or probe amplification rather than target amplification (U.S. Pat. Nos. 5,011,769, 5,403,711, 5,660,988, and 4,876,187), could also be employed. Invasive cleavage assays, e.g., Invader® assays (Third Wave Technologies), described in Eis, P. S. et al., Nat. Biotechnol. 19:673, 2001, can also be used to detect influenza-specific nucleic acids. Assays based on molecular beacons (U.S. Pat. Nos. 6,277,607; 6,150,097; 6,037,130) or fluorescence energy transfer (FRET) may be used. Molecular beacons are oligonucleotide hairpins which undergo a conformational change upon binding to a perfectly matched template.

In certain embodiments the assay determines whether an influenza-specific nucleic acid in the sample comprises a portion that is identical to or different from a sense or antisense strand of an RNAi-inducing entity. Optionally the exact differences, if any, are identified. This information is used to determine whether the influenza virus is susceptible to inhibition by the RNAi-inducing entity. In addition to those discussed above, suitable assays for detection and/or genotyping of infectious agents are described in Molecular Microbiology: Diagnostic Principles and Practice, Persing, D. H., et al., (eds.) Washington, D.C.: ASM Press, 2004.

The diagnostic assays may employ any of the nucleic acids described herein. In certain embodiments of the invention the nucleic acid comprises a nucleic acid portion that is not substantially complementary or substantially identical to a nucleoprotein transcript. For example, the nucleic acid may comprise a primer binding site (e.g., a binding site for a universal sequencing primer or amplification primer), a hybridization tag (which may, for example, be used to isolate the nucleic acid from a sample comprising other nucleic acids), etc. In certain embodiments of the invention the nucleic acid comprises a non-nucleotide moiety. The non-nucleotide moiety may be attached to a terminal nucleotide of the nucleic acid, e.g., at the 3′ end. The moiety may protect the nucleic acid from degradation. In certain embodiments the non-nucleotide moiety is a detectable moiety such as a fluorescent dye, radioactive atom, member of a fluorescence energy transfer (FRET) pair, quencher, etc. In certain embodiments the non-nucleotide moiety is a binding moiety, e.g. biotin or avidin. In certain embodiments the non-nucleotide moiety is a hapten such as digoxygenin, 2,4-Dinitrophenyl (TEG), etc. In certain embodiments the non-nucleotide moiety is a tag usable for isolation of the nucleic acid.

In certain embodiments of the invention a nucleic acid is attached to a support, e.g., a microparticle such as a bead, which is optionally magnetic. The invention further provides an array comprising a multiplicity of nucleic acids of the invention, e.g., at least 10, 20, 50, etc. The nucleic acids are covalently or noncovalently attached to a support, e.g., a substantially planar support such as a glass slide. See, e.g., U.S. Pat. Nos. 5,744,305; 5,800,992; 6,646,243.

Information obtained from experiments or from previous experience in treating a virus having a particular sequence within the nucleoprotein gene can also be used to decide whether the virus is susceptible to inhibition by a given RNAi-inducing entity or combination thereof. Susceptibility information can also include theoretical predictions based, for example, on the expected effect of any mismatches that exist between the nucleoprotein virus sequence and the antisense strand of an inhibitory agent.

The invention provides diagnostic kits for detecting virus infection. Certain of the kits comprise one or more nucleic acids of the invention. Certain of the kits comprise one or more nucleic acids that can be used to detect a portion of an nucleoprotein virus transcript that comprises a preferred target portion for RNAi. The kits may comprise one or more items selected from the group consisting of: a probe, a primer, a sequence-specific oligonucleotide, an enzyme, a substrate, an antibody, a population of nucleotides, a buffer, a positive control, and a negative control. The nucleotides may be labeled. For example, one or more populations of fluorescently labeled nucleotides such as dNTPs, ddNTPs, etc. may be provided.

The probe can be a nucleic acid that includes all or part of a target portion, e.g., a highly or favorably conserved nucleoprotein target portion, or its complement, or is at least 80% identical or complementary to a target portion, e.g., 100% identical or complementary. In certain embodiments a plurality of probes are provided. The probes differ at one or more positions and can be used for determining the exact sequence of a nucleoprotein virus transcript at such positions. For example, the probes may differentially hybridize to the transcript (e.g., hybridization occurs only if the probe is 100% complementary to a target portion of the transcript). Kits of the invention can comprise specimen collection materials, e.g., a swab, a tube, etc. The components of the kit may be packaged in individual vessels or tubes which will generally be provided in a container, e.g., a plastic or styrofoam container suitable for commercial sale, together with instructions for use of the kit.

### Methods of Treatment

The present invention provides for both prophylactic and therapeutic methods for treating a subject at risk of (or susceptible to) or a subject having a virus. “Treatment”, or “treating” as used herein, is defined as the application or administration of a therapeutic agent (e.g., a siRNA or vector or transgene encoding same) to a patient, or application or administration of a therapeutic agent to an isolated tissue or cell line from a patient, who has a virus with the purpose to cure, heal, alleviate, relieve, alter, remedy, ameliorate, improve or affect the virus, or symptoms of the virus. The term “treatment” or “treating” is also used herein in the context of administering agents prophylactically, e.g., to inoculate against a virus.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype.

In related embodiments, a population of two or more different RNAi-inducing agents are administered to a subject, who may be a host to a virus. In one embodiment, the population of two or more RNAi-inducing agents include agents that contain guide strands whose sequences are substantially complementary (preferably 100% complementary) to the same highly conserved region from a variety of strains of a particular virus. In another embodiment, the population of two or more RNAi-inducing agents includes agents that contain guide strands whose sequences are substantially complementary (preferably 100% complementary) to different highly conserved regions from the same virus strain. In yet another embodiment, the population of two or more RNAi-inducing agents include agents that contain guide strands whose sequences are substantially complementary (preferably 100% complementary) to the same highly conserved region from a variety of strains of a particular virus, e.g., an influenza virus and RNAi-inducing agents includes agents that contain guide strands whose sequences are substantially complementary (preferably 100% complementary) to different highly conserved regions from the same virus strain.

### Prophylactic Methods

In one aspect, the invention provides a method for preventing in a subject, infection with a virus or a condition associated with a viral infection, by administering to the subject a prophylactically effective agent that includes any of the siRNAs or vectors or transgenes discussed herein. Administration of a prophylactic agent can occur prior to the manifestation of symptoms characteristic of a viral infection, such that the viral infection is prevented.

In a preferred embodiment, the prophylactically effective agent is administered to the subject prior to exposure to the target virus. In another embodiment, the agent is administered to the subject after exposure to the target virus to delay or inhibit its progression, or prevent its integration into the DNA of healthy cells or cells that do not contain a provirus. Preferably, target virus formation is inhibited or prevented. Additionally or alternatively, it is preferable that target virus replication is inhibited or prevented. In one embodiment, the siRNA degrades the target virus RNA in the early stages of its replication, for example, immediately upon entry into the cell. In this manner, the agent can prevent healthy cells in a subject from becoming infected. In another embodiment, the siRNA degrades the viral MRNA in the late stages of replication. Any of the strategies discussed herein can be employed in these methods, such as administration of a vector that expresses a plurality of siRNAs sufficiently complementary to the viral nucleoprotein gene to mediate RNAi.

### Therapeutic Methods

Another aspect of the invention pertains to methods of modulating target gene expression, protein expression or activity for therapeutic purposes. Accordingly, in an exemplary embodiment, the modulatory method of the invention involves contacting a cell infected with the virus with a therapeutic agent (e.g., a siRNA or vector or transgene encoding same) that is specific for a portion of the viral genome such that RNAi is mediated. These modulatory methods can be performed ex vivo (e.g., by culturing the cell with the agent) or, alternatively, in vivo (e.g., by administering the agent to a subject). The methods can be performed ex vivo and then the products introduced to a subject (e.g., gene therapy).

The therapeutic methods of the invention generally include initiating RNAi by administering the agent to a subject infected with the virus (e.g., influenza). The agent can include one or more siRNAs, one or more siRNA complexes, vectors that express one or more siRNAs (including shRNAs), or transgenes that encode one or more siRNAs. The therapeutic methods of the invention are capable of reducing viral production (e.g., viral titer or provirus titer), by about 30-50-fold, preferably by about 60-80-fold, and more preferably about (or at least) 90-fold, 100-fold, 200-fold, 300-fold, 400-fold, 500-fold or 1000-fold.

Additionally, the therapeutic agents and methods of the present invention can be used in co-therapy with post-transcriptional approaches (e.g., with ribozymes and/or antisense siRNAs).

### Dual Prophylactic and Therapeutic Methods

In a preferred method, a two-pronged attack on the target virus is effected in a subject that has been exposed to the target virus. An infected subject can thus be treated both prophylactically and therapeutically by degrading the virus during early stages of replication and prior to integration into the host cell genome, and also retards replication of the virus in cells in which the target virus has already begun to replicate.

One skilled in the art can readily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect (e.g., with surrogate indicators of viral infection) analysis of appropriate patient samples (e.g., blood and/or tissues).

The prophylactic or therapeutic pharmaceutical compositions of the present invention can contain other pharmaceuticals, in conjunction with a vector according to the invention, when used to therapeutically treat viral infections. Further representative examples of these additional pharmaceuticals that can be used in addition to those previously described, include antiviral compounds, immunomodulators, immunostimulants, antibiotics, and other agents and treatment regimes (including those recognized as alternative medicine) that can be employed to treat viral infections. Immunomodulators and immunostimulants include, but are not limited to, various interleukins, CD4, cytokines, antibody preparations, blood transfusions, and cell transfusions.

### Pharmaceutical Compositions

The invention pertains to uses of the above-described RNAi-inducing entities for the prophylactic and therapeutic treatments of viral infection, as described infra. Accordingly, the agents of the present invention can be incorporated into pharmaceutical compositions suitable for administration. Such compositions typically comprise the agent and a pharmaceutically acceptable carrier.

A pharmaceutical composition of the invention is formulated to be compatible with its intended route of administration. Examples of routes of administration include oral, by inhalation, intranasal, parenteral (e.g., intravenous, intradermal, subcutaneous, intraperitoneal, and intramuscular), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol, and liquid polyetheylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal agents (e.g., parabens, chlorobutanol, phenol, ascorbic acid, thimerosal, and the like). In many cases, it will be preferable to include isotonic agents (e.g., sugars, polyalcohols such as manitol, sorbitol, and sodium chloride) in the composition. Prolonged absorption of the injectable compositions can be brought about by including in the composition an agent that delays absorption (e.g., aluminum monostearate and gelatin).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Inhalational administration means the RNAi-inducing entity is introduced directly to the respiratory system by inhalation through the nose or mouth and into the lungs. The entity is in naked form or with a delivery agent In certain embodiments the RNAi-inducing agent is administered in an amount effective to treat or prevent a condition that affects the respiratory system, such as a respiratory virus infection, while resulting in minimal absorption into the blood and thus minimal systemic delivery of the RNAi-inducing agent. In particular, the invention provides dry powder compositions containing RNAi-inducing entities that are preferably delivered in the form of an aerosol spray from a pressured container or dispenser which contains a suitable propellant, e.g., a gas such as carbon dioxide, or a nebulizer. In certain embodiments the delivery system is suitable for delivering the composition into major airways (trachea and bronchi) of a subject (e.g., an animal or human) and/or deeper into the lung (bronchioles and/or alveoli). The present invention also includes delivery of compositions comprising an RNAi-inducing entity using a nasal spray. According to certain embodiments of the invention delivery agents to facilitate nucleic acid uptake by cells in the respiratory system are included in the pharmaceutical composition. However, the inventors have also discovered that RNAi-inducing agents can effectively inhibit influenza virus when delivered to the respiratory system via the respiratory passages in the absence of specific delivery agents. For example, RNAi-inducing agents can be delivered to the lungs as a composition that consists essentially of the RNAi-inducing agent in dry form (e.g., dry powder) or in an aqueous medium that consists essentially of water, optionally also including a salt (e.g., NaCl, a phosphate salt), buffer, and/or an alcohol, e.g., as naked siRNA or shRNA.

The invention also provides means of systemic circulatory delivery of an RNAi-inducing entity by the pulmonary circulation. For a respiratory disease it is preferable to have minimal transfer to the circulation.

Oral compositions generally include an inert diluent or an edible carrier. They can be enclosed in gelatin capsules or compressed into tablets. For the purpose of oral therapeutic administration, the active compound can be incorporated with excipients and used in the form of so tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate inhalational, oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture or non-human animal assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

All publications, references, patents, patent publications and patent applications cited herein are each hereby specifically incorporated by reference in their entirety.

While this invention has been described in relation to certain embodiments, and many details have been set forth for purposes of illustration, it will be apparent to those skilled in the art that this invention includes additional embodiments, and that some of the details described herein may be varied considerably without departing from this invention. This invention includes such additional embodiments, modifications and equivalents. In particular, this invention includes any combination of the features, terms, or elements of the various illustrative components and examples.

The use herein of the terms “a,” “an,” “the,” and similar terms in describing the invention, and in the claims, are to be construed to include both the singular and the plural. The terms “comprising,” “having,” “including,” and “containing” are to be construed as open-ended terms which mean, for example, “including, but not limited to.” Recitation of a range of values herein refers individually to each and any separate value falling within the range as if it were individually recited herein, whether or not some of the values within the range are expressly recited. Specific values employed herein will be understood as exemplary and not to limit the scope of the invention.

Definitions of technical terms provided herein should be construed to include without recitation those meanings associated with these terms known to those skilled in the art, and are not intended to limit the scope of the invention.

The examples given herein, and the exemplary language used herein are solely for the purpose of illustration, and are not intended to limit the scope of the invention.

When a list of examples is given herein, such as a list of compounds or molecules suitable for this invention, it will be apparent to those skilled in the art that mixtures of the listed compounds or molecules are also suitable.

### EXAMPLE 1

### Identification of Viral Nucleoproteins and siRNAs

Highly conserved sites are considered to be those sites or sequences that are found to be present in a high proportion of all the published human influenza sequences. A subsidiary goal was to identify 19-mer and 25-mer sequences in human influenza isolates that are similar to the highly conserved 19-mer and 25-mer sequences, but that differ by only one or a few nucleotide changes.

There are eight separate RNA segments that compose the influenza viral genome. All analyses were done separately for each of the viral segments. Thus, for example, a search for conserved sites was performed for viral segment #1 using only sequences obtained from segment #1.

Influenza A viral sequences from each of the eight viral segments was obtained from the Influenza Sequence Database (Macken, C., Lu, H., Goodman, J., & Boykin, L., “The value of a database in surveillance and vaccine selection.” in Options for the Control of Influenza IV. A. D. M. E. Osterhaus, N. Cox & A. W. Hampson (Eds.) Amsterdam: Elsevier Science, 2001, 103-106), abbreviated in this document as ISD. The list was screened to remove all but nearly full-length sequences (those with a length that is at least 90% of the longest observed length for a given segment), so that a failure to find a 19-mer or 25-mer fragment match within a given target sequence could not be ascribed to sequence truncation. Table 1 lists the accession numbers of the human influenza sequences that met the preceding criteria and that were used in the subsequent analyses. Accession numbers starting with ISDN are ISD accession numbers; all others are GenBank accession numbers, although the sequences were obtained from the ISD.

TABLE 1
1: GenBank accession numbers for PB2 sequences (segment 1) used in this
analysis.
AB036782, AB126626, AB126635, AB212050, AB212051, AB262460, AF036363, AF037412,
AF037413, AF037414, AF037415, AF037416, AF037417, AF046093, AF084261, AF084262,
AF084263, AF115290, AF115291, AF258524, AF258525, AF258835, AF258836, AF258837,
AF258838, AF258839, AF258840, AF258841, AF258842, AF258843, AF258844, AF258845,
AF258846, AF348170, AF348171, AF389115, AF398866, AF483602, AJ293920, AJ404630,
AJ404631, AJ404632, AJ564804, AJ564805, AY043030, AY209934, AY209935, AY209936,
AY209937, AY209938, AY209939, AY209940, AY209941, AY209942, AY209943, AY209944,
AY209945, AY209946, AY209947, AY209948, AY209949, AY209950, AY209951, AY210137,
AY210138, AY210139, AY210140, AY210141, AY210142, AY210143, AY210144, AY210145,
AY210146, AY210147, AY210148, AY210149, AY210150, AY576380, AY576381, AY651718,
AY651719, AY651721, AY818126, CY000008, CY000016, CY000024, CY000032, CY000040,
CY000048, CY000056, CY000064, CY000072, CY000080, CY000088, CY000096, CY000104,
CY000112, CY000120, CY000128, CY000136, CY000144, CY000152, CY000160, CY000168,
CY000176, CY000184, CY000192, CY000200, CY000208, CY000216, CY000224, CY000232,
CY000240, CY000248, CY000255, CY000264, CY000272, CY000280, CY000288, CY000296,
CY000304, CY000312, CY000320, CY000328, CY000336, CY000344, CY000352, CY000360,
CY000368, CY000376, CY000384, CY000392, CY000400, CY000408, CY000416, CY000424,
CY000432, CY000440, CY000448, CY000455, CY000464, CY000472, CY000480, CY000488,
CY000496, CY000504, CY000512, CY000519, CY000528, CY000536, CY000544, CY000552,
CY000560, CY000568, CY000576, CY000583, CY000592, CY000600, CY000608, CY000616,
CY000624, CY000632, CY000640, CY000648, CY000656, CY000664, CY000672, CY000680,
CY000688, CY000696, CY000704, CY000712, CY000720, CY000728, CY000736, CY000744,
CY000752, CY000760, CY000768, CY000776, CY000784, CY000792, CY000800, CY000808,
CY000816, CY000824, CY000832, CY000840, CY000848, CY000856, CY000864, CY000872,
CY000880, CY000888, CY000896, CY000908, CY000916, CY000924, CY000932, CY000940,
CY000948, CY000956, CY000964, CY000972, CY000980, CY000988, CY000996, CY001004,
CY001012, CY001020, CY001028, CY001036, CY001044, CY001052, CY001060, CY001071,
CY001079, CY001087, CY001095, CY001103, CY001111, CY001119, CY001127, CY001135,
CY001143, CY001151, CY001159, CY001167, CY001175, CY001183, CY001191, CY001196,
CY001204, CY001212, CY001220, CY001228, CY001236, CY001244, CY001252, CY001260,
CY001268, CY001276, CY001284, CY001292, CY001300, CY001308, CY001316, CY001324,
CY001332, CY001340, CY001348, CY001356, CY001364, CY001372, CY001380, CY001388,
CY001404, CY001412, CY001420, CY001428, CY001436, CY001444, CY001452, CY001460,
CY001468, CY001476, CY001484, CY001492, CY001500, CY001511, CY001519, CY001527,
CY001535, CY001543, CY001551, CY001559, CY001567, CY001575, CY001583, CY001591,
CY001599, CY001607, CY001615, CY001623, CY001631, CY001639, CY001647, CY001655,
CY001663, CY001671, CY001679, CY001687, CY001695, CY001703, CY001711, CY001719,
CY001727, CY001735, CY001743, CY001751, CY001759, CY001767, CY001775, CY001783,
CY001791, CY001799, CY001807, CY001815, CY001823, CY001831, CY001839, CY001847,
CY001855, CY001863, CY001871, CY001879, CY001887, CY001895, CY001903, CY001911,
CY001919, CY001927, CY001935, CY001943, CY001951, CY001959, CY001967, CY001975,
CY001983, CY001991, CY001999, CY002007, CY002015, CY002023, CY002031, CY002039,
CY002047, CY002055, CY002063, CY002071, CY002079, CY002087, CY002095, CY002103,
CY002111, CY002119, CY002127, CY002135, CY002143, CY002151, CY002159, CY002167,
CY002175, CY002183, CY002191, CY002199, CY002207, CY002215, CY002223, CY002231,
CY002239, CY002247, CY002255, CY002263, CY002271, CY002279, CY002287, CY002295,
CY002303, CY002311, CY002319, CY002335, CY002343, CY002351, CY002359, CY002367,
CY002375, CY002383, CY002391, CY002399, CY002407, CY002415, CY002423, CY002431,
CY002439, CY002447, CY002455, CY002463, CY002471, CY002479, CY002487, CY002495,
CY002503, CY002511, CY002519, CY002527, CY002535, CY002543, CY002551, CY002559,
CY002567, CY002575, CY002583, CY002591, CY002599, CY002607, CY002615, CY002623,
CY002631, CY002639, CY002647, CY002655, CY002663, CY002671, CY002679, CY002687,
CY002695, CY002703, CY002711, CY002719, CY002727, CY002735, CY002743, CY002751,
CY002759, CY002767, CY002775, CY002783, CY002791, CY002799, CY002807, CY002815,
CY002823, CY002913, CY002921, CY002929, CY002937, CY002945, CY002953, CY002961,
CY002969, CY002976, CY002983, CY002991, CY002999, CY003007, CY003015, CY003023,
CY003031, CY003039, CY003047, CY003055, CY003063, CY003071, CY003079, CY003087,
CY003095, CY003103, CY003111, CY003119, CY003132, CY003135, CY003143, CY003151,
CY003159, CY003167, CY003175, CY003183, CY003191, CY003199, CY003207, CY003215,
CY003223, CY003231, CY003239, CY003247, CY003255, CY003263, CY003271, CY003279,
CY003287, CY003295, CY003303, CY003311, CY003319, CY003327, CY003335, CY003343,
CY003351, CY003359, CY003375, CY003383, CY003391, CY003399, CY003407, CY003415,
CY003423, CY003431, CY003439, CY003447, CY003455, CY003463, CY003471, CY003479,
CY003487, CY003495, CY003503, CY003511, CY003519, CY003527, CY003535, CY003543,
CY003551, CY003559, CY003567, CY003575, CY003583, CY003591, CY003599, CY003607,
CY003615, CY003623, CY003631, CY003639, CY003647, CY003655, CY003663, CY003671,
CY003679, CY003687, CY003695, CY003703, CY003711, CY003719, CY003727, CY003735,
CY003743, CY003751, CY003759, CY003768, CY003776, CY003784, CY003792, CY003800,
CY003808, CY003816, CY003824, CY003832, CY003840, CY006051, CY006059, CY006067,
CY006075, CY006083, CY006091, CY006099, CY006106, CY006114, CY006122, CY006130,
CY006138, CY006146, CY006154, CY006162, CY006170, CY006178, CY006186, CY006194,
CY006202, CY006210, CY006218, CY006226, CY006234, CY006242, CY006250, CY006258,
CY006266, CY006274, CY006282, CY006290, CY006298, CY006306, CY006314, CY006322,
CY006330, CY006338, CY006346, CY006354, CY006362, CY006370, CY006378, CY006386,
CY006394, CY006402, CY006410, CY006418, CY006426, CY006434, CY006442, CY006450,
CY006458, CY006466, CY006474, CY006482, CY006490, CY006498, CY006506, CY006514,
CY006522, CY006530, CY006538, CY006546, CY006554, CY006562, CY006570, CY006578,
CY006586, CY006594, CY006602, CY006610, CY006618, CY006626, CY006634, CY006642,
CY006666, CY006674, CY006682, CY006690, CY006698, CY006706, CY006714, CY006722,
CY006730, CY006738, CY006746, CY006754, CY006762, CY006770, CY006778, CY006786,
CY006794, CY006802, CY006810, CY006818, CY006826, CY006834, CY006842, CY006850,
CY006858, CY006866, CY006874, CY006882, CY006890, CY006898, CY006906, CY006914,
CY006922, CY006930, CY006938, CY006946, CY006954, CY006962, CY006970, CY006978,
CY006986, CY006994, CY007002, CY007010, CY007018, CY007026, CY007034, CY007042,
CY007050, CY007058, CY007066, CY007074, CY007082, CY007090, CY007098, CY007106,
CY007114, CY007122, CY007130, CY007138, CY007146, CY007154, CY007162, CY007170,
CY007178, CY007186, CY007194, CY007202, CY007210, CY007218, CY007226, CY007234,
CY007242, CY007250, CY007258, CY007266, CY007274, CY007282, CY007290, CY007298,
CY007306, CY007314, CY007322, CY007330, CY007338, CY007346, CY007354, CY007362,
CY007370, CY007378, CY007386, CY007394, CY007402, CY007410, CY007418, CY007426,
CY007434, CY007442, CY007450, CY007458, CY007466, CY007474, CY007482, CY007490,
CY007498, CY007506, CY007514, CY007522, CY007530, CY007538, CY007546, CY007554,
CY007562, CY007570, CY007578, CY007586, CY007594, CY007602, CY007610, CY007618,
CY007626, CY007634, CY007642, CY007650, CY007658, CY007666, CY007674, CY007682,
CY007690, CY007698, CY007706, CY007714, CY007722, CY007730, CY007738, CY007746,
CY007754, CY007762, CY007770, CY007778, CY007786, CY007794, CY007802, CY007810,
CY007818, CY007826, CY007834, CY007842, CY007850, CY007858, CY007866, CY007874,
CY007882, CY007890, CY007898, CY007906, CY007914, CY007922, CY007930, CY007938,
CY007946, CY007954, CY007962, CY007970, CY007978, CY007986, CY007994, CY008002,
CY008010, CY008018, CY008026, CY008034, CY008042, CY008050, CY008058, CY008066,
CY008074, CY008082, CY008090, CY008098, CY008106, CY008114, CY008122, CY008130,
CY008138, CY008146, CY008155, CY008163, CY008171, CY008179, CY008187, CY008195,
CY008203, CY008211, CY008219, CY008227, CY008235, CY008243, CY008251, CY008259,
CY008267, CY008275, CY008283, CY008291, CY008299, CY008307, CY008315, CY008323,
CY008331, CY008339, CY008347, CY008355, CY008363, CY008371, CY008379, CY008387,
CY008395, CY008403, CY008411, CY008419, CY008427, CY008435, CY008443, CY008451,
CY008459, CY008467, CY008475, CY008483, CY008491, CY008499, CY008507, CY008515,
CY008523, CY008531, CY008539, CY008547, CY008555, CY008563, CY008571, CY008579,
CY008587, CY008595, CY008603, CY008611, CY008619, CY008627, CY008635, CY008643,
CY008651, CY008659, CY008667, CY008675, CY008683, CY008691, CY008699, CY008707,
CY008715, CY008723, CY008731, CY008739, CY008747, CY008755, CY008763, CY008771,
CY008779, CY008787, CY008795, CY008803, CY008811, CY008819, CY008827, CY008835,
CY008843, CY008851, CY008859, CY008867, CY008875, CY008883, CY008891, CY008899,
CY008907, CY008915, CY008923, CY008931, CY008939, CY008947, CY008955, CY008963,
CY008971, CY008979, CY008987, CY008995, CY009003, CY009011, CY009019, CY009027,
CY009035, CY009043, CY009051, CY009059, CY009067, CY009075, CY009083, CY009091,
CY009099, CY009107, CY009115, CY009123, CY009131, CY009139, CY009147, CY009155,
CY009163, CY009171, CY009179, CY009187, CY009195, CY009203, CY009211, CY009219,
CY009227, CY009235, CY009243, CY009251, CY009259, CY009267, CY009275, CY009283,
CY009291, CY009299, CY009323, CY009331, CY009339, CY009347, CY009355, CY009363,
CY009371, CY009395, CY009403, CY009411, CY009419, CY009427, CY009435, CY009443,
CY009451, CY009459, CY009467, CY009475, CY009483, CY009491, CY009499, CY009507,
CY009515, CY009523, CY009531, CY009539, CY009547, CY009555, CY009563, CY009571,
CY009579, CY009587, CY009595, CY009603, CY009611, CY009619, CY009627, CY009643,
CY009651, CY009659, CY009667, CY009675, CY009683, CY009691, CY009699, CY009707,
CY009715, CY009723, CY009731, CY009739, CY009747, CY009755, CY009763, CY009771,
CY009779, CY009787, CY009795, CY009803, CY009811, CY009819, CY009827, CY009835,
CY009843, CY009851, CY009859, CY009867, CY009875, CY009883, CY009891, CY009907,
CY009915, CY009931, CY009939, CY009947, CY009955, CY009963, CY009971, CY009979,
CY009987, CY009995, CY010003, CY010011, CY010019, CY010027, CY010035, CY010043,
CY010051, CY010059, CY010067, CY010075, CY010083, CY010091, CY010099, CY010107,
CY010115, CY010123, CY010131, CY010139, CY010147, CY010155, CY010163, CY010171,
CY010179, CY010187, CY010195, CY010203, CY010211, CY010219, CY010227, CY010235,
CY010243, CY010251, CY010259, CY010267, CY010275, CY010283, CY010291, CY010299,
CY010307, CY010315, CY010323, CY010331, CY010339, CY010347, CY010355, CY010363,
CY010371, CY010379, CY010387, CY010395, CY010403, CY010411, CY010419, CY010427,
CY010435, CY010443, CY010451, CY010459, CY010467, CY010475, CY010483, CY010491,
CY010499, CY010507, CY010515, CY010523, CY010531, CY010539, CY010547, CY010555,
CY010563, CY010595, CY010603, CY010611, CY010619, CY010627, CY010635, CY010643,
CY010651, CY010659, CY010667, CY010675, CY010683, CY010691, CY010699, CY010707,
CY010715, CY010723, CY010731, CY010739, CY010747, CY010755, CY010763, CY010771,
CY010779, CY010787, CY010795, CY010803, CY010811, CY010819, CY010827, CY010835,
CY010843, CY010851, CY010859, CY010867, CY010875, CY010883, CY010891, CY010899,
CY010907, CY010915, CY010923, CY010931, CY010939, CY010947, CY010955, CY010963,
CY010971, CY010979, CY010987, CY010995, CY011003, CY011011, CY011019, CY011027,
CY011071, CY011079, CY011087, CY011095, CY011127, CY011135, CY011143, CY011151,
CY011159, CY011167, CY011175, CY011183, CY011191, CY011199, CY011207, CY011215,
CY011223, CY011231, CY011239, CY011247, CY011263, CY011271, CY011279, CY011287,
CY011295, CY011303, CY011311, CY011319, CY011327, CY011335, CY011343, CY011351,
CY011359, CY011367, CY011375, CY011383, CY011391, CY011399, CY011407, CY011415,
CY011423, CY011431, CY011439, CY011447, CY011455, CY011463, CY011471, CY011479,
CY011487, CY011495, CY011503, CY011511, CY011519, CY011527, CY011535, CY011543,
CY011551, CY011559, CY011567, CY011575, CY011583, CY011591, CY011599, CY011607,
CY011615, CY011623, CY011631, CY011639, CY011647, CY011655, CY011663, CY011671,
CY011679, CY011687, CY011695, CY011703, CY011711, CY011719, CY011727, CY011735,
CY011743, CY011751, CY011759, CY011767, CY011775, CY011783, CY011791, CY011799,
CY011807, CY011815, CY011823, CY011831, CY011839, CY011847, CY011855, CY011863,
CY011871, CY011879, CY011887, CY011895, CY011903, CY011911, CY011919, CY011927,
CY011935, CY011943, CY011951, CY011959, CY011967, CY011975, CY011983, CY011991,
CY011999, CY012007, CY012015, CY012023, CY012031, CY012039, CY012047, CY012055,
CY012063, CY012071, CY012079, CY012087, CY012095, CY012103, CY012111, CY012119,
CY012127, CY012135, CY012143, CY012151, CY012159, CY012167, CY012175, CY012183,
CY012191, CY012199, CY012207, CY012215, CY012223, CY012231, CY012239, CY012247,
CY012255, CY012263, CY012271, CY012279, CY012287, CY012295, CY012303, CY012311,
CY012319, CY012327, CY012335, CY012343, CY012351, CY012359, CY012367, CY012375,
CY012383, CY012391, CY012399, CY012407, CY012415, CY012423, CY012431, CY012439,
CY012447, CY012455, CY012463, CY012471, CY012479, CY012487, CY012495, CY012503,
CY012511, CY012519, CY012527, CY012535, CY012543, CY012551, CY012559, CY012567,
CY012575, CY012583, CY012591, CY012599, CY012607, CY012615, CY012623, CY012631,
CY012639, CY012647, CY012655, CY012663, CY012671, CY012679, CY012687, CY012695,
CY012703, CY012711, CY012719, CY012727, CY012735, CY012743, CY012751, CY012759,
CY012767, CY012775, CY012783, CY012791, CY012799, CY012855, CY012863, CY012871,
CY012879, CY012887, CY012895, CY012903, CY012911, CY012919, CY012927, CY012935,
CY012943, CY012951, CY012959, CY012967, CY012975, CY012983, CY012991, CY012999,
CY013007, CY013015, CY013023, CY013031, CY013039, CY013047, CY013055, CY013063,
CY013071, CY013079, CY013087, CY013095, CY013103, CY013111, CY013119, CY013127,
CY013135, CY013143, CY013151, CY013159, CY013167, CY013175, CY013183, CY013191,
CY013199, CY013207, CY013215, CY013223, CY013231, CY013239, CY013247, CY013278,
CY013286, CY013294, CY013302, CY013310, CY013318, CY013326, CY013334, CY013342,
CY013350, CY013358, CY013366, CY013374, CY013382, CY013396, CY013404, CY013412,
CY013420, CY013428, CY013436, CY013444, CY013452, CY013460, CY013468, CY013476,
CY013484, CY013492, CY013500, CY013508, CY013516, CY013524, CY013532, CY013540,
CY013548, CY013556, CY013564, CY013572, CY013580, CY013588, CY013596, CY013604,
CY013612, CY013620, CY013628, CY013636, CY013644, CY013652, CY013660, CY013668,
CY013676, CY013684, CY013692, CY013700, CY013708, CY013716, CY013724, CY013732,
CY013740, CY013748, CY013756, CY013764, CY013772, CY013780, CY013788, CY013796,
CY013804, CY013812, CY013820, CY013828, CY013836, CY013844, CY013852, CY013860,
CY013862, CY013878, CY013886, CY013894, CY013902, CY013910, CY013918, CY013926,
CY013934, CY013942, CY013950, CY013958, CY013966, CY013974, CY013982, CY013990,
CY013998, CY014006, CY014014, CY014022, CY014030, CY014038, CY014046, CY014054,
CY014062, CY014070, CY014078, CY014086, CY014094, CY014102, CY014110, CY014118,
CY014126, CY014134, CY014142, CY014150, CY014158, CY014166, CY014167, CY014175,
CY014269, CY014277, CY014285, CY014293, CY014308, CY014320, CY014322, CY014325,
CY014329, CY014332, CY014333, CY014337, CY014341, CY014344, CY014346, CY014348,
CY014352, CY014356, CY014359, CY014360, CY014364, CY014367, CY014375, CY014383,
CY014391, CY014392, CY014396, CY014404, CY014412, CY014420, CY014428, CY014436,
CY014444, CY014452, CY014460, CY014468, CY014476, CY014484, CY014492, CY014500,
CY014512, CY014520, CY014526, CY014534, CY014983, CY015013, CY015515, CY015523,
CY015531, CY015539, CY015547, CY015555, CY015563, CY015571, CY015579, CY015587,
CY015595, CY015603, CY015611, CY015619, CY015627, CY015635, CY015643, CY015651,
CY015659, CY015667, CY015675, CY015683, CY015691, CY015699, CY015707, CY015715,
CY015723, CY015731, CY015739, CY015747, CY015755, CY015763, CY015771, CY015779,
CY015787, CY015795, CY015803, CY015811, CY015819, CY015827, CY015835, CY015843,
CY015851, CY015859, CY015867, CY015875, CY015883, CY015891, CY015899, CY015907,
CY015915, CY015923, CY015931, CY015939, CY015947, CY015955, CY015963, CY015971,
CY015979, CY015987, CY015995, CY016003, CY016011, CY016019, CY016027, CY016035,
CY016043, CY016051, CY016059, CY016067, CY016075, CY016083, CY016091, CY016099,
CY016107, CY016115, CY016123, CY016203, CY016211, CY016219, CY016235, CY016243,
CY016251, CY016259, CY016267, CY016275, CY016410, CY016434, CY016442, CY016450,
CY016458, CY016466, CY016474, CY016482, CY016490, CY016498, CY016506, CY016514,
CY016522, CY016530, CY016538, CY016546, CY016554, CY016562, CY016570, CY016578,
CY016586, CY016594, CY016602, CY016610, CY016634, CY016642, CY016650, CY016658,
CY016666, CY016674, CY016682, CY016690, CY016698, CY016706, CY016714, CY016722,
CY016730, CY016738, CY016746, CY016754, CY016762, CY016770, CY016778, CY016970,
CY016978, CY016986, CY016994, CY017002, CY017010, CY017018, CY017026, CY017090,
CY017098, CY017106, CY017114, CY017122, CY017130, CY017138, CY017146, CY017154,
CY017162, CY017170, CY017178, CY017202, CY017210, CY017218, CY017226, CY017234,
CY017242, CY017250, CY017258, CY017266, CY017274, CY017290, CY017298, CY017306,
CY017314, CY017322, CY017330, CY017338, CY017346, CY017354, CY017362, CY017370,
CY017378, CY017386, CY017394, CY017402, CY017426, CY017434, CY017442, CY017450,
CY017458, CY017466, CY017474, CY017482, CY017490, CY017498, CY017506, CY017514,
CY017522, CY017530, CY017538, CY017546, CY017554, CY017562, CY017570, CY017578,
CY017586, CY017594, CY017602, CY017610, CY017618, CY017626, CY017634, CY017635,
CY017643, CY017651, CY017659, CY017667, CY017675, CY017683, CY017685, DQ138181,
DQ208309, DQ226172, DQ249263, DQ360837, DQ372598, DQ415283, DQ415284, DQ415285,
DQ415286, DQ415287, DQ415288, DQ415289, DQ415290, DQ415291, DQ415292, DQ415293,
DQ469955, DQ486029, DQ487334, DQ492894, DQ492896, DQ492900, DQ492902, DQ508822,
DQ508830, DQ508838, DQ508846, DQ508854, DQ508862, DQ508870, DQ508878, DQ508886,
DQ508894, DQ508902, DQ508926, DQ535731, DQ643811, EF101747, EF101754,
ISDN125790, ISDN125870, ISDN128033, ISDN129529, ISDN129579, ISDN130372,
ISDN130380, ISDN132413, ISDN132421, ISDN133103, ISDN133322, ISDN133329,
ISDN133363, ISDN133371, ISDN133377, ISDN13419, ISDN136848, ISDN136856,
ISDN138755, ISDN138771, ISDN138779, ISDN140026, ISDN140027, ISDN140028,
ISDN140029, ISDN140030, ISDN140031, ISDN140817, ISDN140825, ISDN140832,
ISDN140840, ISDN140848, ISDN140856, ISDN140915, ISDN140916, ISDN140917,
ISDN140933, ISDN181362, ISDN183249, ISDN183251, ISDN183254, ISDN183258,
ISDN183261, ISDN183262, ISDN183266, ISDN183270, ISDN183273, ISDN183275,
ISDN183277, ISDN183281, ISDN183285, ISDN183288, ISDN183289, ISDN183293,
ISDN183296, ISDN183304, ISDN183312, ISDN183320, ISDN183321, ISDN183701,
ISDN183709, ISDN183717, ISDN183725, ISDN183733, ISDN183741, ISDN183749,
ISDN183757, ISDN183765, ISDN183773, ISDN183781, ISDN185505, ISDN187428,
ISDN187436, ISDN187444, ISDN187452, ISDN187460, ISDN204136, ISDN204144,
ISDN204152, ISDN207105, ISDN207113, ISDN40379, ISDN40380, ISDN40383, ISDN49457,
J02140, J02179, M23970, M38277, M73517, M73521, M73524, M81575, M81581, M81587,
M91712, M91713, NC_002023, U62543, U71132, U71133, U71134, U71135, X15283, X99035,
X99036
2: GenBank and LANL accession numbers for PB1 sequences (segment 2) used in this
analysis.
AB036782, AB126626, AB126635, AB212050, AB212051, AB262460, AF036363, AF037412,
AF037413, AF037414, AF037415, AF037416, AF037417, AF046093, AF084261, AF084262,
AF084263, AF115290, AF115291, AF258524, AF258525, AF258835, AF258836, AF258837,
AF258838, AF258839, AF258840, AF258841, AF258842, AF258843, AF258844, AF258845,
AF258846, AF348170, AF348171, AF389115, AF398866, AF483602, AJ293920, AJ404630,
AJ404631, AJ404632, AJ564804, AJ564805, AY043030, AY209934, AY209935, AY209936,
AY209937, AY209938, AY209939, AY209940, AY209941, AY209942, AY209943, AY209944,
AY209945, AY209946, AY209947, AY209948, AY209949, AY209950, AY209951, AY210137,
AY210138, AY210139, AY210140, AY210141, AY210142, AY210143, AY210144, AY210145,
AY210146, AY210147, AY210148, AY210149, AY210150, AY576380, AY576381, AY651718,
AY651719, AY651721, AY818126, CY000008, CY000016, CY000024, CY000032, CY000040,
CY000048, CY000056, CY000064, CY000072, CY000080, CY000088, CY000096, CY000104,
CY000112, CY000120, CY000128, CY000136, CY000144, CY000152, CY000160, CY000168,
CY000176, CY000184, CY000192, CY000200, CY000208, CY000216, CY000224, CY000232,
CY000240, CY000248, CY000255, CY000264, CY000272, CY000280, CY000288, CY000296,
CY000304, CY000312, CY000320, CY000328, CY000336, CY000344, CY000352, CY000360,
CY000368, CY000376, CY000384, CY000392, CY000400, CY000408, CY000416, CY000424,
CY000432, CY000440, CY000448, CY000455, CY000464, CY000472, CY000480, CY000488,
CY000496, CY000504, CY000512, CY000519, CY000528, CY000536, CY000544, CY000552,
CY000560, CY000568, CY000576, CY000583, CY000592, CY000600, CY000608, CY000616,
CY000624, CY000632, CY000640, CY000648, CY000656, CY000664, CY000672, CY000680,
CY000688, CY000696, CY000704, CY000712, CY000720, CY000728, CY000736, CY000744,
CY000752, CY000760, CY000768, CY000776, CY000784, CY000792, CY000800, CY000808,
CY000816, CY000824, CY000832, CY000840, CY000848, CY000856, CY000864, CY000872,
CY000880, CY000888, CY000896, CY000908, CY000916, CY000924, CY000932, CY000940,
CY000948, CY000956, CY000964, CY000972, CY000980, CY000988, CY000996, CY001004,
CY001012, CY001020, CY001028, CY001036, CY001044, CY001052, CY001060, CY001071,
CY001079, CY001087, CY001095, CY001103, CY001111, CY001119, CY001127, CY001135,
CY001143, CY001151, CY001159, CY001167, CY001175, CY001183, CY001191, CY001196,
CY001204, CY001212, CY001220, CY001228, CY001236, CY001244, CY001252, CY001260,
CY001268, CY001276, CY001284, CY001292, CY001300, CY001308, CY001316, CY001324,
CY001332, CY001340, CY001348, CY001356, CY001364, CY001372, CY001380, CY001388,
CY001404, CY001412, CY001420, CY001428, CY001436, CY001444, CY001452, CY001460,
CY001468, CY001476, CY001484, CY001492, CY001500, CY001511, CY001519, CY001527,
CY001535, CY001543, CY001551, CY001559, CY001567, CY001575, CY001583, CY001591,
CY001599, CY001607, CY001615, CY001623, CY001631, CY001639, CY001647, CY001655,
CY001663, CY001671, CY001679, CY001687, CY001695, CY001703, CY001711, CY001719,
CY001727, CY001735, CY001743, CY001751, CY001759, CY001767, CY001775, CY001783,
CY001791, CY001799, CY001807, CY001815, CY001823, CY001831, CY001839, CY001847,
CY001855, CY001863, CY001871, CY001879, CY001887, CY001895, CY001903, CY001911,
CY001919, CY001927, CY001935, CY001943, CY001951, CY001959, CY001967, CY001975,
CY001983, CY001991, CY001999, CY002007, CY002015, CY002023, CY002031, CY002039,
CY002047, CY002055, CY002063, CY002071, CY002079, CY002087, CY002095, CY002103,
CY002111, CY002119, CY002127, CY002135, CY002143, CY002151, CY002159, CY002167,
CY002175, CY002183, CY002191, CY002199, CY002207, CY002215, CY002223, CY002231,
CY002239, CY002247, CY002255, CY002263, CY002271, CY002279, CY002287, CY002295,
CY002303, CY002311, CY002319, CY002335, CY002343, CY002351, CY002359, CY002367,
CY002375, CY002383, CY002391, CY002399, CY002407, CY002415, CY002423, CY002431,
CY002439, CY002447, CY002455, CY002463, CY002471, CY002479, CY002487, CY002495,
CY002503, CY002511, CY002519, CY002527, CY002535, CY002543, CY002551, CY002559,
CY002567, CY002575, CY002583, CY002591, CY002599, CY002607, CY002615, CY002623,
CY002631, CY002639, CY002647, CY002655, CY002663, CY002671, CY002679, CY002687,
CY002695, CY002703, CY002711, CY002719, CY002727, CY002735, CY002743, CY002751,
CY002759, CY002767, CY002775, CY002783, CY002791, CY002799, CY002807, CY002815,
CY002823, CY002913, CY002921, CY002929, CY002937, CY002945, CY002953, CY002961,
CY002969, CY002976, CY002983, CY002991, CY002999, CY003007, CY003015, CY003023,
CY003031, CY003039, CY003047, CY003055, CY003063, CY003071, CY003079, CY003087,
CY003095, CY003103, CY003111, CY003119, CY003132, CY003135, CY003143, CY003151,
CY003159, CY003167, CY003175, CY003183, CY003191, CY003199, CY003207, CY003215,
CY003223, CY003231, CY003239, CY003247, CY003255, CY003263, CY003271, CY003279,
CY003287, CY003295, CY003303, CY003311, CY003319, CY003327, CY003335, CY003343,
CY003351, CY003359, CY003375, CY003383, CY003391, CY003399, CY003407, CY003415,
CY003423, CY003431, CY003439, CY003447, CY003455, CY003463, CY003471, CY003479,
CY003487, CY003495, CY003503, CY003511, CY003519, CY003527, CY003535, CY003543,
CY003551, CY003559, CY003567, CY003575, CY003583, CY003591, CY003599, CY003607,
CY003615, CY003623, CY003631, CY003639, CY003647, CY003655, CY003663, CY003671,
CY003679, CY003687, CY003695, CY003703, CY003711, CY003719, CY003727, CY003735,
CY003743, CY003751, CY003759, CY003768, CY003776, CY003784, CY003792, CY003800,
CY003808, CY003816, CY003824, CY003832, CY003840, CY006051, CY006059, CY006067,
CY006075, CY006083, CY006091, CY006099, CY006106, CY006114, CY006122, CY006130,
CY006138, CY006146, CY006154, CY006162, CY006170, CY006178, CY006186, CY006194,
CY006202, CY006210, CY006218, CY006226, CY006234, CY006242, CY006250, CY006258,
CY006266, CY006274, CY006282, CY006290, CY006298, CY006306, CY006314, CY006322,
CY006330, CY006338, CY006346, CY006354, CY006362, CY006370, CY006378, CY006386,
CY006394, CY006402, CY006410, CY006418, CY006426, CY006434, CY006442, CY006450,
CY006458, CY006466, CY006474, CY006482, CY006490, CY006498, CY006506, CY006514,
CY006522, CY006530, CY006538, CY006546, CY006554, CY006562, CY006570, CY006578,
CY006586, CY006594, CY006602, CY006610, CY006618, CY006626, CY006634, CY006642,
CY006666, CY006674, CY006682, CY006690, CY006698, CY006706, CY006714, CY006722,
CY006730, CY006738, CY006746, CY006754, CY006762, CY006770, CY006778, CY006786,
CY006794, CY006802, CY006810, CY006818, CY006826, CY006834, CY006842, CY006850,
CY006858, CY006866, CY006874, CY006882, CY006890, CY006898, CY006906, CY006914,
CY006922, CY006930, CY006938, CY006946, CY006954, CY006962, CY006970, CY006978,
CY006986, CY006994, CY007002, CY007010, CY007018, CY007026, CY007034, CY007042,
CY007050, CY007058, CY007066, CY007074, CY007082, CY007090, CY007098, CY007106,
CY007114, CY007122, CY007130, CY007138, CY007146, CY007154, CY007162, CY007170,
CY007178, CY007186, CY007194, CY007202, CY007210, CY007218, CY007226, CY007234,
CY007242, CY007250, CY007258, CY007266, CY007274, CY007282, CY007290, CY007298,
CY007306, CY007314, CY007322, CY007330, CY007338, CY007346, CY007354, CY007362,
CY007370, CY007378, CY007386, CY007394, CY007402, CY007410, CY007418, CY007426,
CY007434, CY007442, CY007450, CY007458, CY007466, CY007474, CY007482, CY007490,
CY007498, CY007506, CY007514, CY007522, CY007530, CY007538, CY007546, CY007554,
CY007562, CY007570, CY007578, CY007586, CY007594, CY007602, CY007610, CY007618,
CY007626, CY007634, CY007642, CY007650, CY007658, CY007666, CY007674, CY007682,
CY007690, CY007698, CY007706, CY007714, CY007722, CY007730, CY007738, CY007746,
CY007754, CY007762, CY007770, CY007778, CY007786, CY007794, CY007802, CY007810,
CY007818, CY007826, CY007834, CY007842, CY007850, CY007858, CY007866, CY007874,
CY007882, CY007890, CY007898, CY007906, CY007914, CY007922, CY007930, CY007938,
CY007946, CY007954, CY007962, CY007970, CY007978, CY007986, CY007994, CY008002,
CY008010, CY008018, CY008026, CY008034, CY008042, CY008050, CY008058, CY008066,
CY008074, CY008082, CY008090, CY008098, CY008106, CY008114, CY008122, CY008130,
CY008138, CY008146, CY008155, CY008163, CY008171, CY008179, CY008187, CY008195,
CY008203, CY008211, CY008219, CY008227, CY008235, CY008243, CY008251, CY008259,
CY008267, CY008275, CY008283, CY008291, CY008299, CY008307, CY008315, CY008323,
CY008331, CY008339, CY008347, CY008355, CY008363, CY008371, CY008379, CY008387,
CY008395, CY008403, CY008411, CY008419, CY008427, CY008435, CY008443, CY008451,
CY008459, CY008467, CY008475, CY008483, CY008491, CY008499, CY008507, CY008515,
CY008523, CY008531, CY008539, CY008547, CY008555, CY008563, CY008571, CY008579,
CY008587, CY008595, CY008603, CY008611, CY008619, CY008627, CY008635, CY008643,
CY008651, CY008659, CY008667, CY008675, CY008683, CY008691, CY008699, CY008707,
CY008715, CY008723, CY008731, CY008739, CY008747, CY008755, CY008763, CY008771,
CY008779, CY008787, CY008795, CY008803, CY008811, CY008819, CY008827, CY008835,
CY008843, CY008851, CY008859, CY008867, CY008875, CY008883, CY008891, CY008899,
CY008907, CY008915, CY008923, CY008931, CY008939, CY008947, CY008955, CY008963,
CY008971, CY008979, CY008987, CY008995, CY009003, CY009011, CY009019, CY009027,
CY009035, CY009043, CY009051, CY009059, CY009067, CY009075, CY009083, CY009091,
CY009099, CY009107, CY009115, CY009123, CY009131, CY009139, CY009147, CY009155,
CY009163, CY009171, CY009179, CY009187, CY009195, CY009203, CY009211, CY009219,
CY009227, CY009235, CY009243, CY009251, CY009259, CY009267, CY009275, CY009283,
CY009291, CY009299, CY009323, CY009331, CY009339, CY009347, CY009355, CY009363,
CY009371, CY009395, CY009403, CY009411, CY009419, CY009427, CY009435, CY009443,
CY009451, CY009459, CY009467, CY009475, CY009483, CY009491, CY009499, CY009507,
CY009515, CY009523, CY009531, CY009539, CY009547, CY009555, CY009563, CY009571,
CY009579, CY009587, CY009595, CY009603, CY009611, CY009619, CY009627, CY009643,
CY009651, CY009659, CY009667, CY009675, CY009683, CY009691, CY009699, CY009707,
CY009715, CY009723, CY009731, CY009739, CY009747, CY009755, CY009763, CY009771,
CY009779, CY009787, CY009795, CY009803, CY009811, CY009819, CY009827, CY009835,
CY009843, CY009851, CY009859, CY009867, CY009875, CY009883, CY009891, CY009907,
CY009915, CY009931, CY009939, CY009947, CY009955, CY009963, CY009971, CY009979,
CY009987, CY009995, CY010003, CY010011, CY010019, CY010027, CY010035, CY010043,
CY010051, CY010059, CY010067, CY010075, CY010083, CY010091, CY010099, CY010107,
CY010115, CY010123, CY010131, CY010139, CY010147, CY010155, CY010163, CY010171,
CY010179, CY010187, CY010195, CY010203, CY010211, CY010219, CY010227, CY010235,
CY010243, CY010251, CY010259, CY010267, CY010275, CY010283, CY010291, CY010299,
CY010307, CY010315, CY010323, CY010331, CY010339, CY010347, CY010355, CY010363,
CY010371, CY010379, CY010387, CY010395, CY010403, CY010411, CY010419, CY010427,
CY010435, CY010443, CY010451, CY010459, CY010467, CY010475, CY010483, CY010491,
CY010499, CY010507, CY010515, CY010523, CY010531, CY010539, CY010547, CY010555,
CY010563, CY010595, CY010603, CY010611, CY010619, CY010627, CY010635, CY010643,
CY010651, CY010659, CY010667, CY010675, CY010683, CY010691, CY010699, CY010707,
CY010715, CY010723, CY010731, CY010739, CY010747, CY010755, CY010763, CY010771,
CY010779, CY010787, CY010795, CY010803, CY010811, CY010819, CY010827, CY010835,
CY010843, CY010851, CY010859, CY010867, CY010875, CY010883, CY010891, CY010899,
CY010907, CY010915, CY010923, CY010931, CY010939, CY010947, CY010955, CY010963,
CY010971, CY010979, CY010987, CY010995, CY011003, CY011011, CY011019, CY011027,
CY011071, CY011079, CY011087, CY011095, CY011127, CY011135, CY011143, CY011151,
CY011159, CY011167, CY011175, CY011183, CY011191, CY011199, CY011207, CY011215,
CY011223, CY011231, CY011239, CY011247, CY011263, CY011271, CY011279, CY011287,
CY011295, CY011303, CY011311, CY011319, CY011327, CY011335, CY011343, CY011351,
CY011359, CY011367, CY011375, CY011383, CY011391, CY011399, CY011407, CY011415,
CY011423, CY011431, CY011439, CY011447, CY011455, CY011463, CY011471, CY011479,
CY011487, CY011495, CY011503, CY011511, CY011519, CY011527, CY011535, CY011543,
CY011551, CY011559, CY011567, CY011575, CY011583, CY011591, CY011599, CY011607,
CY011615, CY011623, CY011631, CY011639, CY011647, CY011655, CY011663, CY011671,
CY011679, CY011687, CY011695, CY011703, CY011711, CY011719, CY011727, CY011735,
CY011743, CY011751, CY011759, CY011767, CY011775, CY011783, CY011791, CY011799,
CY011807, CY011815, CY011823, CY011831, CY011839, CY011847, CY011855, CY011863,
CY011871, CY011879, CY011887, CY011895, CY011903, CY011911, CY011919, CY011927,
CY011935, CY011943, CY011951, CY011959, CY011967, CY011975, CY011983, CY011991,
CY011999, CY012007, CY012015, CY012023, CY012031, CY012039, CY012047, CY012055,
CY012063, CY012071, CY012079, CY012087, CY012095, CY012103, CY012111, CY012119,
CY012127, CY012135, CY012143, CY012151, CY012159, CY012167, CY012175, CY012183,
CY012191, CY012199, CY012207, CY012215, CY012223, CY012231, CY012239, CY012247,
CY012255, CY012263, CY012271, CY012279, CY012287, CY012295, CY012303, CY012311,
CY012319, CY012327, CY012335, CY012343, CY012351, CY012359, CY012367, CY012375,
CY012383, CY012391, CY012399, CY012407, CY012415, CY012423, CY012431, CY012439,
CY012447, CY012455, CY012463, CY012471, CY012479, CY012487, CY012495, CY012503,
CY012511, CY012519, CY012527, CY012535, CY012543, CY012551, CY012559, CY012567,
CY012575, CY012583, CY012591, CY012599, CY012607, CY012615, CY012623, CY012631,
CY012639, CY012647, CY012655, CY012663, CY012671, CY012679, CY012687, CY012695,
CY012703, CY012711, CY012719, CY012727, CY012735, CY012743, CY012751, CY012759,
CY012767, CY012775, CY012783, CY012791, CY012799, CY012855, CY012863, CY012871,
CY012879, CY012887, CY012895, CY012903, CY012911, CY012919, CY012927, CY012935,
CY012943, CY012951, CY012959, CY012967, CY012975, CY012983, CY012991, CY012999,
CY013007, CY013015, CY013023, CY013031, CY013039, CY013047, CY013055, CY013063,
CY013071, CY013079, CY013087, CY013095, CY013103, CY013111, CY013119, CY013127,
CY013135, CY013143, CY013151, CY013159, CY013167, CY013175, CY013183, CY013191,
CY013199, CY013207, CY013215, CY013223, CY013231, CY013239, CY013247, CY013278,
CY013286, CY013294, CY013302, CY013310, CY013318, CY013326, CY013334, CY013342,
CY013350, CY013358, CY013366, CY013374, CY013382, CY013396, CY013404, CY013412,
CY013420, CY013428, CY013436, CY013444, CY013452, CY013460, CY013468, CY013476,
CY013484, CY013492, CY013500, CY013508, CY013516, CY013524, CY013532, CY013540,
CY013548, CY013556, CY013564, CY013572, CY013580, CY013588, CY013596, CY013604,
CY013612, CY013620, CY013628, CY013636, CY013644, CY013652, CY013660, CY013668,
CY013676, CY013684, CY013692, CY013700, CY013708, CY013716, CY013724, CY013732,
CY013740, CY013748, CY013756, CY013764, CY013772, CY013780, CY013788, CY013796,
CY013804, CY013812, CY013820, CY013828, CY013836, CY013844, CY013852, CY013860,
CY013862, CY013878, CY013886, CY013894, CY013902, CY013910, CY013918, CY013926,
CY013934, CY013942, CY013950, CY013958, CY013966, CY013974, CY013982, CY013990,
CY013998, CY014006, CY014014, CY014022, CY014030, CY014038, CY014046, CY014054,
CY014062, CY014070, CY014078, CY014086, CY014094, CY014102, CY014110, CY014118,
CY014126, CY014134, CY014142, CY014150, CY014158, CY014166, CY014167, CY014175,
CY014269, CY014277, CY014285, CY014293, CY014308, CY014320, CY014322, CY014325,
CY014329, CY014332, CY014333, CY014337, CY014341, CY014344, CY014346, CY014348,
CY014352, CY014356, CY014359, CY014360, CY014364, CY014367, CY014375, CY014383,
CY014391, CY014392, CY014396, CY014404, CY014412, CY014420, CY014428, CY014436,
CY014444, CY014452, CY014460, CY014468, CY014476, CY014484, CY014492, CY014500,
CY014512, CY014520, CY014526, CY014534, CY014983, CY015013, CY015515, CY015523,
CY015531, CY015539, CY015547, CY015555, CY015563, CY015571, CY015579, CY015587,
CY015595, CY015603, CY015611, CY015619, CY015627, CY015635, CY015643, CY015651,
CY015659, CY015667, CY015675, CY015683, CY015691, CY015699, CY015707, CY015715,
CY015723, CY015731, CY015739, CY015747, CY015755, CY015763, CY015771, CY015779,
CY015787, CY015795, CY015803, CY015811, CY015819, CY015827, CY015835, CY015843,
CY015851, CY015859, CY015867, CY015875, CY015883, CY015891, CY015899, CY015907,
CY015915, CY015923, CY015931, CY015939, CY015947, CY015955, CY015963, CY015971,
CY015979, CY015987, CY015995, CY016003, CY016011, CY016019, CY016027, CY016035,
CY016043, CY016051, CY016059, CY016067, CY016075, CY016083, CY016091, CY016099,
CY016107, CY016115, CY016123, CY016203, CY016211, CY016219, CY016235, CY016243,
CY016251, CY016259, CY016267, CY016275, CY016410, CY016434, CY016442, CY016450,
CY016458, CY016466, CY016474, CY016482, CY016490, CY016498, CY016506, CY016514,
CY016522, CY016530, CY016538, CY016546, CY016554, CY016562, CY016570, CY016578,
CY016586, CY016594, CY016602, CY016610, CY016634, CY016642, CY016650, CY016658,
CY016666, CY016674, CY016682, CY016690, CY016698, CY016706, CY016714, CY016722,
CY016730, CY016738, CY016746, CY016754, CY016762, CY016770, CY016778, CY016970,
CY016978, CY016986, CY016994, CY017002, CY017010, CY017018, CY017026, CY017090,
CY017098, CY017106, CY017114, CY017122, CY017130, CY017138, CY017146, CY017154,
CY017162, CY017170, CY017178, CY017202, CY017210, CY017218, CY017226, CY017234,
CY017242, CY017250, CY017258, CY017266, CY017274, CY017290, CY017298, CY017306,
CY017314, CY017322, CY017330, CY017338, CY017346, CY017354, CY017362, CY017370,
CY017378, CY017386, CY017394, CY017402, CY017426, CY017434, CY017442, CY017450,
CY017458, CY017466, CY017474, CY017482, CY017490, CY017498, CY017506, CY017514,
CY017522, CY017530, CY017538, CY017546, CY017554, CY017562, CY017570, CY017578,
CY017586, CY017594, CY017602, CY017610, CY017618, CY017626, CY017634, CY017635,
CY017643, CY017651, CY017659, CY017667, CY017675, CY017683, CY017685, DQ138181,
DQ208309, DQ226172, DQ249263, DQ360837, DQ372598, DQ415283, DQ415284, DQ415285,
DQ415286, DQ415287, DQ415288, DQ415289, DQ415290, DQ415291, DQ415292, DQ415293,
DQ469955, DQ486029, DQ487334, DQ492894, DQ492896, DQ492900, DQ492902, DQ508822,
DQ508830, DQ508838, DQ508846, DQ508854, DQ508862, DQ508870, DQ508878, DQ508886,
DQ508894, DQ508902, DQ508926, DQ535731, DQ643811, EF101747, EF101754,
ISDN125790, ISDN125870, ISDN128033, ISDN129529, ISDN129579, ISDN130372,
ISDN130380, ISDN132413, ISDN132421, ISDN133103, ISDN133322, ISDN133329,
ISDN133363, ISDN133371, ISDN133377, ISDN13419, ISDN136848, ISDN136856,
ISDN138755, ISDN138771, ISDN138779, ISDN140026, ISDN140027, ISDN140028,
ISDN140029, ISDN140030, ISDN140031, ISDN140817, ISDN140825, ISDN140832,
ISDN140840, ISDN140848, ISDN140856, ISDN140915, ISDN140916, ISDN140917,
ISDN140933, ISDN181362, ISDN183249, ISDN183251, ISDN183254, ISDN183258,
ISDN183261, ISDN183262, ISDN183266, ISDN183270, ISDN183273, ISDN183275,
ISDN183277, ISDN183281, ISDN183285, ISDN183288, ISDN183289, ISDN183293,
ISDN183296, ISDN183304, ISDN183312, ISDN183320, ISDN183321, ISDN183701,
ISDN183709, ISDN183717, ISDN183725, ISDN183733, ISDN183741, ISDN183749,
ISDN183757, ISDN183765, ISDN183773, ISDN183781, ISDN185505, ISDN187428,
ISDN187436, ISDN187444, ISDN187452, ISDN187460, ISDN204136, ISDN204144,
ISDN204152, ISDN207105, ISDN207113, ISDN40379, ISDN40380, ISDN40383, ISDN49457,
J02140, J02179, M23970, M38277, M73517, M73521, M73524, M81575, M81581, M81587,
M91712, M91713, NC_002023, U62543, U71132, U71133, U71134, U71135, X15283, X99035,
X99036
3: GenBank and LANL accession numbers for PA sequences (segment 3) used in this
analysis.
AB036780, AB126627, AB126633, AB212053, AB262462, AF037424, AF037425, AF037426,
AF037427, AF037428, AF037429, AF046095, AF084267, AF084268, AF084269, AF084270,
AF115294, AF115295, AF257191, AF257192, AF257193, AF257194, AF257195, AF257196,
AF257197, AF257198, AF257199, AF257200, AF257201, AF257202, AF258518, AF258519,
AF348174, AF348175, AF389117, AF398862, AF398863, AF398864, AF483603, AJ238020,
AJ289874, AJ291402, AJ293922, AJ404637, AJ605762, AJ605763, AY043028, AY209990,
AY209991, AY209992, AY209993, AY209994, AY209995, AY209996, AY209997, AY209998,
AY209999, AY210000, AY210001, AY210002, AY210003, AY210004, AY210005, AY210006,
AY210007, AY210193, AY210194, AY210195, AY210196, AY210197, AY210198, AY210199,
AY210200, AY210201, AY210202, AY210203, AY210204, AY210205, AY210206, AY576404,
AY576405, AY651610, AY651611, AY651612, AY651613, AY818132, CY000006, CY000014,
CY000022, CY000030, CY000038, CY000046, CY000054, CY000062, CY000070, CY000078,
CY000086, CY000094, CY000102, CY000110, CY000118, CY000126, CY000134, CY000142,
CY000150, CY000158, CY000166, CY000174, CY000182, CY000190, CY000198, CY000206,
CY000214, CY000222, CY000230, CY000238, CY000246, CY000254, CY000262, CY000270,
CY000278, CY000286, CY000294, CY000302, CY000310, CY000318, CY000326, CY000334,
CY000342, CY000350, CY000358, CY000366, CY000374, CY000382, CY000390, CY000398,
CY000406, CY000414, CY000422, CY000430, CY000438, CY000446, CY000454, CY000462,
CY000470, CY000478, CY000486, CY000494, CY000502, CY000510, CY000517, CY000526,
CY000534, CY000542, CY000550, CY000558, CY000566, CY000574, CY000581, CY000590,
CY000598, CY000606, CY000614, CY000622, CY000630, CY000638, CY000646, CY000654,
CY000662, CY000670, CY000678, CY000686, CY000694, CY000702, CY000710, CY000718,
CY000726, CY000734, CY000742, CY000750, CY000758, CY000766, CY000774, CY000782,
CY000790, CY000798, CY000806, CY000814, CY000822, CY000830, CY000838, CY000846,
CY000854, CY000862, CY000870, CY000878, CY000886, CY000894, CY000906, CY000914,
CY000922, CY000930, CY000938, CY000946, CY000954, CY000962, CY000970, CY000978,
CY000986, CY000994, CY001002, CY001010, CY001018, CY001026, CY001034, CY001042,
CY001050, CY001058, CY001069, CY001077, CY001085, CY001093, CY001101, CY001109,
CY001117, CY001125, CY001133, CY001141, CY001149, CY001157, CY001165, CY001173,
CY001181, CY001189, CY001194, CY001202, CY001210, CY001218, CY001226, CY001234,
CY001242, CY001250, CY001258, CY001266, CY001274, CY001282, CY001290, CY001298,
CY001306, CY001314, CY001322, CY001330, CY001338, CY001346, CY001354, CY001362,
CY001370, CY001378, CY001386, CY001402, CY001410, CY001418, CY001426, CY001434,
CY001442, CY001450, CY001458, CY001466, CY001474, CY001482, CY001490, CY001498,
CY001509, CY001517, CY001525, CY001533, CY001541, CY001549, CY001557, CY001565,
CY001573, CY001581, CY001589, CY001597, CY001605, CY001613, CY001621, CY001629,
CY001637, CY001645, CY001653, CY001661, CY001669, CY001677, CY001685, CY001693,
CY001701, CY001709, CY001717, CY001725, CY001733, CY001741, CY001749, CY001757,
CY001765, CY001773, CY001781, CY001789, CY001797, CY001805, CY001813, CY001821,
CY001829, CY001837, CY001845, CY001853, CY001861, CY001869, CY001877, CY001885,
CY001893, CY001901, CY001909, CY001917, CY001925, CY001933, CY001941, CY001949,
CY001957, CY001965, CY001973, CY001981, CY001989, CY001997, CY002005, CY002013,
CY002021, CY002029, CY002037, CY002045, CY002053, CY002061, CY002069, CY002077,
CY002085, CY002093, CY002101, CY002109, CY002117, CY002125, CY002133, CY002141,
CY002149, CY002157, CY002165, CY002173, CY002181, CY002189, CY002197, CY002205,
CY002213, CY002221, CY002229, CY002237, CY002245, CY002253, CY002261, CY002269,
CY002277, CY002285, CY002293, CY002301, CY002309, CY002317, CY002333, CY002341,
CY002349, CY002357, CY002365, CY002373, CY002381, CY002389, CY002397, CY002405,
CY002413, CY002421, CY002429, CY002437, CY002445, CY002453, CY002461, CY002469,
CY002477, CY002485, CY002493, CY002501, CY002509, CY002517, CY002525, CY002533,
CY002541, CY002549, CY002557, CY002565, CY002573, CY002581, CY002589, CY002597,
CY002605, CY002613, CY002621, CY002629, CY002637, CY002645, CY002653, CY002661,
CY002669, CY002677, CY002685, CY002693, CY002701, CY002709, CY002717, CY002725,
CY002733, CY002741, CY002749, CY002757, CY002765, CY002773, CY002781, CY002789,
CY002797, CY002805, CY002813, CY002821, CY002911, CY002919, CY002927, CY002935,
CY002943, CY002951, CY002959, CY002967, CY002974, CY002981, CY002989, CY002997,
CY003005, CY003013, CY003021, CY003029, CY003037, CY003045, CY003053, CY003061,
CY003069, CY003077, CY003085, CY003093, CY003101, CY003109, CY003117, CY003126,
CY003133, CY003141, CY003149, CY003157, CY003165, CY003173, CY003181, CY003189,
CY003197, CY003205, CY003213, CY003221, CY003229, CY003237, CY003245, CY003253,
CY003261, CY003269, CY003277, CY003285, CY003293, CY003301, CY003309, CY003317,
CY003325, CY003333, CY003341, CY003349, CY003357, CY003373, CY003381, CY003389,
CY003397, CY003405, CY003413, CY003421, CY003429, CY003437, CY003445, CY003453,
CY003461, CY003469, CY003477, CY003485, CY003493, CY003501, CY003509, CY003517,
CY003525, CY003533, CY003541, CY003549, CY003557, CY003565, CY003573, CY003581,
CY003589, CY003597, CY003605, CY003613, CY003621, CY003629, CY003637, CY003645,
CY003653, CY003661, CY003669, CY003677, CY003685, CY003693, CY003701, CY003709,
CY003717, CY003725, CY003733, CY003741, CY003749, CY003757, CY003766, CY003774,
CY003782, CY003790, CY003798, CY003806, CY003814, CY003822, CY003830, CY003838,
CY006049, CY006057, CY006065, CY006073, CY006081, CY006089, CY006097, CY006104,
CY006112, CY006120, CY006128, CY006136, CY006144, CY006152, CY006160, CY006168,
CY006176, CY006184, CY006192, CY006200, CY006208, CY006216, CY006224, CY006232,
CY006240, CY006248, CY006256, CY006264, CY006272, CY006280, CY006288, CY006296,
CY006304, CY006312, CY006320, CY006328, CY006336, CY006344, CY006352, CY006360,
CY006368, CY006376, CY006384, CY006392, CY006400, CY006408, CY006416, CY006424,
CY006432, CY006440, CY006448, CY006456, CY006464, CY006472, CY006480, CY006488,
CY006496, CY006504, CY006512, CY006520, CY006528, CY006536, CY006544, CY006552,
CY006560, CY006568, CY006576, CY006584, CY006592, CY006600, CY006608, CY006616,
CY006624, CY006632, CY006640, CY006664, CY006672, CY006680, CY006688, CY006696,
CY006704, CY006712, CY006720, CY006728, CY006736, CY006744, CY006752, CY006760,
CY006768, CY006776, CY006784, CY006792, CY006800, CY006808, CY006816, CY006824,
CY006832, CY006840, CY006848, CY006856, CY006864, CY006872, CY006880, CY006888,
CY006896, CY006904, CY006912, CY006920, CY006928, CY006936, CY006944, CY006952,
CY006960, CY006968, CY006976, CY006984, CY006992, CY007000, CY007008, CY007016,
CY007024, CY007032, CY007040, CY007048, CY007056, CY007064, CY007072, CY007080,
CY007088, CY007096, CY007104, CY007112, CY007120, CY007128, CY007136, CY007144,
CY007152, CY007160, CY007168, CY007176, CY007184, CY007192, CY007200, CY007208,
CY007216, CY007224, CY007232, CY007240, CY007248, CY007256, CY007264, CY007272,
CY007280, CY007288, CY007296, CY007304, CY007312, CY007320, CY007328, CY007336,
CY007344, CY007352, CY007360, CY007368, CY007376, CY007384, CY007392, CY007400,
CY007408, CY007416, CY007424, CY007432, CY007440, CY007448, CY007456, CY007464,
CY007472, CY007480, CY007488, CY007496, CY007504, CY007512, CY007520, CY007528,
CY007536, CY007544, CY007552, CY007560, CY007568, CY007576, CY007584, CY007592,
CY007600, CY007608, CY007616, CY007624, CY007632, CY007640, CY007648, CY007656,
CY007664, CY007672, CY007680, CY007688, CY007696, CY007704, CY007712, CY007720,
CY007728, CY007736, CY007744, CY007752, CY007760, CY007768, CY007776, CY007784,
CY007792, CY007800, CY007808, CY007816, CY007824, CY007832, CY007840, CY007848,
CY007856, CY007864, CY007872, CY007880, CY007888, CY007896, CY007904, CY007912,
CY007920, CY007928, CY007936, CY007944, CY007952, CY007960, CY007968, CY007976,
CY007984, CY007992, CY008000, CY008008, CY008016, CY008024, CY008032, CY008040,
CY008048, CY008056, CY008064, CY008072, CY008080, CY008088, CY008096, CY008104,
CY008112, CY008120, CY008128, CY008136, CY008144, CY008153, CY008161, CY008169,
CY008177, CY008185, CY008193, CY008201, CY008209, CY008217, CY008225, CY008233,
CY008241, CY008249, CY008257, CY008265, CY008273, CY008281, CY008289, CY008297,
CY008305, CY008313, CY008321, CY008329, CY008337, CY008345, CY008353, CY008361,
CY008369, CY008377, CY008385, CY008393, CY008401, CY008409, CY008417, CY008425,
CY008433, CY008441, CY008449, CY008457, CY008465, CY008473, CY008481, CY008489,
CY008497, CY008505, CY008513, CY008521, CY008529, CY008537, CY008545, CY008553,
CY008561, CY008569, CY008577, CY008585, CY008593, CY008601, CY008609, CY008617,
CY008625, CY008633, CY008641, CY008649, CY008657, CY008665, CY008673, CY008681,
CY008689, CY008697, CY008705, CY008713, CY008721, CY008729, CY008737, CY008745,
CY008753, CY008761, CY008769, CY008777, CY008785, CY008793, CY008801, CY008809,
CY008817, CY008825, CY008833, CY008841, CY008849, CY008857, CY008865, CY008873,
CY008881, CY008889, CY008897, CY008905, CY008913, CY008921, CY008929, CY008937,
CY008945, CY008953, CY008961, CY008969, CY008977, CY008985, CY008993, CY009001,
CY009009, CY009017, CY009025, CY009033, CY009041, CY009049, CY009057, CY009065,
CY009073, CY009081, CY009089, CY009097, CY009105, CY009113, CY009121, CY009129,
CY009137, CY009145, CY009153, CY009161, CY009169, CY009177, CY009185, CY009193,
CY009201, CY009209, CY009217, CY009225, CY009233, CY009241, CY009249, CY009257,
CY009265, CY009273, CY009281, CY009289, CY009297, CY009321, CY009329, CY009337,
CY009345, CY009353, CY009361, CY009369, CY009393, CY009401, CY009409, CY009417,
CY009425, CY009433, CY009441, CY009449, CY009457, CY009465, CY009473, CY009481,
CY009489, CY009497, CY009505, CY009513, CY009521, CY009529, CY009537, CY009545,
CY009553, CY009561, CY009569, CY009577, CY009585, CY009593, CY009601, CY009609,
CY009617, CY009625, CY009641, CY009649, CY009657, CY009665, CY009673, CY009681,
CY009689, CY009697, CY009705, CY009713, CY009721, CY009729, CY009737, CY009745,
CY009753, CY009761, CY009769, CY009777, CY009785, CY009793, CY009801, CY009809,
CY009817, CY009825, CY009833, CY009841, CY009849, CY009857, CY009865, CY009873,
CY009881, CY009889, CY009905, CY009913, CY009929, CY009937, CY009945, CY009953,
CY009961, CY009969, CY009977, CY009985, CY009993, CY010001, CY010009, CY010017,
CY010025, CY010033, CY010041, CY010049, CY010057, CY010065, CY010073, CY010081,
CY010089, CY010097, CY010105, CY010113, CY010121, CY010129, CY010137, CY010145,
CY010153, CY010161, CY010169, CY010177, CY010185, CY010193, CY010201, CY010209,
CY010217, CY010225, CY010233, CY010241, CY010249, CY010257, CY010265, CY010273,
CY010281, CY010289, CY010297, CY010305, CY010313, CY010321, CY010329, CY010337,
CY010345, CY010353, CY010361, CY010369, CY010377, CY010385, CY010393, CY010401,
CY010409, CY010417, CY010425, CY010433, CY010441, CY010449, CY010457, CY010465,
CY010473, CY010481, CY010489, CY010497, CY010505, CY010513, CY010521, CY010529,
CY010537, CY010545, CY010553, CY010561, CY010593, CY010601, CY010609, CY010617,
CY010625, CY010633, CY010641, CY010649, CY010657, CY010665, CY010673, CY010681,
CY010689, CY010697, CY010705, CY010713, CY010721, CY010729, CY010737, CY010745,
CY010753, CY010761, CY010769, CY010777, CY010785, CY010793, CY010801, CY010809,
CY010817, CY010825, CY010833, CY010841, CY010849, CY010857, CY010865, CY010873,
CY010881, CY010889, CY010897, CY010905, CY010913, CY010921, CY010929, CY010937,
CY010945, CY010953, CY010961, CY010969, CY010977, CY010985, CY010993, CY011001,
CY011009, CY011017, CY011025, CY011069, CY011077, CY011085, CY011093, CY011125,
CY011133, CY011141, CY011149, CY011157, CY011165, CY011173, CY011181, CY011189,
CY011197, CY011205, CY011213, CY011221, CY011229, CY011237, CY011245, CY011261,
CY011269, CY011277, CY011285, CY011293, CY011301, CY011309, CY011317, CY011325,
CY011333, CY011341, CY011349, CY011357, CY011365, CY011373, CY011381, CY011389,
CY011397, CY011405, CY011413, CY011421, CY011429, CY011437, CY011445, CY011453,
CY011461, CY011469, CY011477, CY011485, CY011493, CY011501, CY011509, CY011517,
CY011525, CY011533, CY011541, CY011549, CY011557, CY011565, CY011573, CY011581,
CY011589, CY011597, CY011605, CY011613, CY011621, CY011629, CY011637, CY011645,
CY011653, CY011661, CY011669, CY011677, CY011685, CY011693, CY011701, CY011709,
CY011717, CY011725, CY011733, CY011741, CY011749, CY011757, CY011765, CY011773,
CY011781, CY011789, CY011797, CY011805, CY011813, CY011821, CY011829, CY011837,
CY011845, CY011853, CY011861, CY011869, CY011877, CY011885, CY011893, CY011901,
CY011909, CY011917, CY011925, CY011933, CY011941, CY011949, CY011957, CY011965,
CY011973, CY011981, CY011989, CY011997, CY012005, CY012013, CY012021, CY012029,
CY012037, CY012045, CY012053, CY012061, CY012069, CY012077, CY012085, CY012093,
CY012101, CY012109, CY012117, CY012125, CY012133, CY012141, CY012149, CY012157,
CY012165, CY012173, CY012181, CY012189, CY012197, CY012205, CY012213, CY012221,
CY012229, CY012237, CY012245, CY012253, CY012261, CY012269, CY012277, CY012285,
CY012293, CY012301, CY012309, CY012317, CY012325, CY012333, CY012341, CY012349,
CY012357, CY012365, CY012373, CY012381, CY012389, CY012397, CY012405, CY012413,
CY012421, CY012429, CY012437, CY012445, CY012453, CY012461, CY012469, CY012477,
CY012485, CY012493, CY012501, CY012509, CY012517, CY012525, CY012533, CY012541,
CY012549, CY012557, CY012565, CY012573, CY012581, CY012589, CY012597, CY012605,
CY012613, CY012621, CY012629, CY012637, CY012645, CY012653, CY012661, CY012669,
CY012677, CY012685, CY012693, CY012701, CY012709, CY012717, CY012725, CY012733,
CY012741, CY012749, CY012757, CY012765, CY012773, CY012781, CY012789, CY012797,
CY012853, CY012861, CY012869, CY012877, CY012885, CY012893, CY012901, CY012909,
CY012917, CY012925, CY012933, CY012941, CY012949, CY012957, CY012965, CY012973,
CY012981, CY012989, CY012997, CY013005, CY013013, CY013021, CY013029, CY013037,
CY013045, CY013053, CY013061, CY013069, CY013077, CY013085, CY013093, CY013101,
CY013109, CY013117, CY013125, CY013133, CY013141, CY013149, CY013157, CY013165,
CY013173, CY013181, CY013189, CY013197, CY013205, CY013213, CY013221, CY013229,
CY013237, CY013245, CY013276, CY013284, CY013292, CY013300, CY013308, CY013316,
CY013324, CY013332, CY013340, CY013348, CY013356, CY013364, CY013372, CY013380,
CY013388, CY013394, CY013402, CY013410, CY013418, CY013426, CY013434, CY013442,
CY013450, CY013458, CY013466, CY013474, CY013482, CY013490, CY013498, CY013506,
CY013514, CY013522, CY013530, CY013538, CY013546, CY013554, CY013562, CY013570,
CY013578, CY013586, CY013594, CY013602, CY013610, CY013618, CY013626, CY013634,
CY013642, CY013650, CY013658, CY013666, CY013674, CY013682, CY013690, CY013698,
CY013706, CY013714, CY013722, CY013730, CY013738, CY013746, CY013754, CY013762,
CY013770, CY013778, CY013786, CY013794, CY013802, CY013810, CY013818, CY013826,
CY013834, CY013842, CY013850, CY013858, CY013876, CY013884, CY013892, CY013900,
CY013908, CY013916, CY013924, CY013932, CY013940, CY013948, CY013956, CY013964,
CY013972, CY013980, CY013988, CY013996, CY014004, CY014012, CY014020, CY014028,
CY014036, CY014044, CY014052, CY014060, CY014068, CY014076, CY014084, CY014092,
CY014100, CY014108, CY014116, CY014124, CY014132, CY014140, CY014148, CY014156,
CY014164, CY014169, CY014171, CY014271, CY014279, CY014287, CY014295, CY014302,
CY014310, CY014321, CY014326, CY014328, CY014330, CY014334, CY014338, CY014340,
CY014342, CY014347, CY014350, CY014353, CY014354, CY014357, CY014362, CY014366,
CY014373, CY014381, CY014389, CY014394, CY014402, CY014410, CY014418, CY014426,
CY014434, CY014442, CY014450, CY014458, CY014466, CY014474, CY014482, CY014490,
CY014498, CY014511, CY014519, CY014528, CY014536, CY014542, CY014981, CY015011,
CY015513, CY015521, CY015529, CY015537, CY015545, CY015553, CY015561, CY015569,
CY015577, CY015585, CY015593, CY015601, CY015609, CY015617, CY015625, CY015633,
CY015641, CY015649, CY015657, CY015665, CY015673, CY015681, CY015689, CY015697,
CY015705, CY015713, CY015721, CY015729, CY015737, CY015745, CY015753, CY015761,
CY015769, CY015777, CY015785, CY015793, CY015801, CY015809, CY015817, CY015825,
CY015833, CY015841, CY015849, CY015857, CY015865, CY015873, CY015881, CY015889,
CY015897, CY015905, CY015913, CY015921, CY015929, CY015937, CY015945, CY015953,
CY015961, CY015969, CY015977, CY015985, CY015993, CY016001, CY016009, CY016017,
CY016025, CY016033, CY016041, CY016049, CY016057, CY016065, CY016073, CY016081,
CY016089, CY016097, CY016105, CY016113, CY016121, CY016201, CY016209, CY016217,
CY016233, CY016241, CY016249, CY016257, CY016265, CY016273, CY016408, CY016432,
CY016440, CY016448, CY016456, CY016464, CY016472, CY016480, CY016488, CY016496,
CY016504, CY016512, CY016520, CY016528, CY016536, CY016544, CY016552, CY016560,
CY016568, CY016576, CY016584, CY016592, CY016600, CY016608, CY016632, CY016640,
CY016648, CY016656, CY016664, CY016672, CY016680, CY016688, CY016696, CY016704,
CY016712, CY016720, CY016728, CY016736, CY016744, CY016752, CY016760, CY016768,
CY016776, CY016968, CY016976, CY016984, CY016992, CY017000, CY017008, CY017016,
CY017024, CY017088, CY017096, CY017104, CY017112, CY017120, CY017128, CY017136,
CY017144, CY017152, CY017160, CY017168, CY017176, CY017200, CY017208, CY017216,
CY017224, CY017232, CY017240, CY017248, CY017256, CY017264, CY017272, CY017288,
CY017296, CY017304, CY017312, CY017320, CY017328, CY017336, CY017344, CY017352,
CY017360, CY017368, CY017376, CY017384, CY017392, CY017400, CY017424, CY017432,
CY017440, CY017448, CY017456, CY017464, CY017472, CY017480, CY017488, CY017496,
CY017504, CY017512, CY017520, CY017528, CY017536, CY017544, CY017552, CY017560,
CY017568, CY017576, CY017584, CY017592, CY017600, CY017608, CY017616, CY017624,
CY017632, CY017637, CY017645, CY017653, CY017661, CY017669, CY017677, CY017687,
DQ099791, DQ099792, DQ138184, DQ138185, DQ138186, DQ138187, DQ138188, DQ208311,
DQ360839, DQ372596, DQ381564, DQ415305, DQ415306, DQ415307, DQ415308, DQ415309,
DQ415310, DQ415311, DQ415312, DQ415313, DQ415314, DQ415315, DQ469957, DQ487327,
DQ487335, DQ493332, DQ493333, DQ493334, DQ493335, DQ493336, DQ493337, DQ493338,
DQ493339, DQ493340, DQ493341, DQ508824, DQ508832, DQ508840, DQ508848, DQ508856,
DQ508864, DQ508872, DQ508880, DQ508888, DQ508896, DQ508904, DQ508928, DQ535729,
EF100817, EF101746, EF101755, ISDN121932, ISDN121933, ISDN125788, ISDN125872,
ISDN128031, ISDN129532, ISDN129581, ISDN130370, ISDN130378, ISDN130917,
ISDN131200, ISDN132411, ISDN132419, ISDN133101, ISDN133321, ISDN133328,
ISDN133361, ISDN133369, ISDN133375, ISDN13421, ISDN136846, ISDN136854,
ISDN138753, ISDN138769, ISDN138777, ISDN140040, ISDN140041, ISDN140042,
ISDN140043, ISDN140044, ISDN140045, ISDN140046, ISDN140047, ISDN140815,
ISDN140823, ISDN140834, ISDN140842, ISDN140850, ISDN140858, ISDN140865,
ISDN140909, ISDN140910, ISDN140911, ISDN140931, ISDN181364, ISDN183250,
ISDN183255, ISDN183257, ISDN183259, ISDN183263, ISDN183267, ISDN183269,
ISDN183271, ISDN183276, ISDN183279, ISDN183282, ISDN183283, ISDN183286,
ISDN183291, ISDN183295, ISDN183302, ISDN183310, ISDN183318, ISDN183699,
ISDN183707, ISDN183715, ISDN183723, ISDN183731, ISDN183739, ISDN183747,
ISDN183755, ISDN183763, ISDN183771, ISDN183779, ISDN185502, ISDN187426,
ISDN187434, ISDN187442, ISDN187450, ISDN187458, ISDN204134, ISDN204142,
ISDN204150, ISDN207104, ISDN207112, ISDN40934, ISDN40940, J02139, M23974, M26078,
M26079, M81573, M81579, M81585, NC_002022, U71136, U71137, U71138, U71139, X17336,
X99039
4: GenBank and LANL accession numbers for HA sequences (segment 4) used in this
analysis.
AB019354, AB019355, AB019356, AB019357, AB056699, AB212054, AB239125, AB262463,
AB284320, AF017272, AF028020, AF028709, AF036356, AF046088, AF046096, AF046097,
AF046098, AF084279, AF084280, AF084281, AF084532, AF102671, AF102672, AF102673,
AF102674, AF102675, AF102676, AF102677, AF102678, AF102679, AF102680, AF102681,
AF102682, AF117241, AF348176, AF348177, AF348178, AF348179, AF363502, AF363503,
AF363504, AF382318, AF382319, AF382320, AF382321, AF382322, AF382323, AF382324,
AF382325, AF382326, AF382327, AF382328, AF386773, AF386774, AF386775, AF386776,
AF386777, AF386778, AF386780, AF386781, AF389118, AF398874, AF398875, AF398878,
AJ252129, AJ252131, AJ289702, AJ289703, AJ293926, AJ311466, AJ344014, AJ344022,
AJ404626, AJ404627, AJ867074, AY032978, AY035588, AY035589, AY035590, AY035591,
AY035592, AY271794, AY282756, AY282757, AY282758, AY282759, AY289927, AY289928,
AY289929, AY289930, AY531033, AY531035, AY531039, AY531040, AY531041, AY531042,
AY531043, AY531044, AY531045, AY531046, AY531047, AY531048, AY531049, AY531050,
AY531051, AY531052, AY531053, AY531054, AY531055, AY531056, AY531057, AY531058,
AY531059, AY531060, AY531061, AY555150, AY555153, AY575869, AY589647, AY589648,
AY589649, AY589650, AY589651, AY589652, AY589653, AY589654, AY589655, AY589656,
AY589657, AY589658, AY589659, AY589660, AY589661, AY643085, AY643086, AY643087,
AY651333, AY651334, AY651335, AY651336, AY661020, AY661041, AY661042, AY661043,
AY661053, AY661054, AY661055, AY661056, AY661059, AY661066, AY661073, AY679514,
AY682833, AY818135, AY884276, AY884277, AY884278, AY884279, AY884280, AY884281,
AY884282, AY884283, AY884284, CY000001, CY000009, CY000017, CY000025, CY000033,
CY000041, CY000049, CY000057, CY000065, CY000073, CY000081, CY000089, CY000097,
CY000105, CY000113, CY000121, CY000129, CY000137, CY000145, CY000153, CY000161,
CY000169, CY000177, CY000185, CY000193, CY000201, CY000209, CY000217, CY000225,
CY000233, CY000241, CY000249, CY000257, CY000265, CY000273, CY000281, CY000289,
CY000297, CY000305, CY000313, CY000321, CY000329, CY000337, CY000345, CY000353,
CY000361, CY000369, CY000377, CY000385, CY000393, CY000401, CY000409, CY000417,
CY000425, CY000433, CY000441, CY000449, CY000457, CY000465, CY000473, CY000481,
CY000489, CY000497, CY000505, CY000513, CY000521, CY000529, CY000537, CY000545,
CY000553, CY000561, CY000569, CY000584, CY000585, CY000593, CY000601, CY000609,
CY000617, CY000625, CY000633, CY000641, CY000649, CY000657, CY000665, CY000673,
CY000681, CY000689, CY000697, CY000705, CY000713, CY000721, CY000729, CY000737,
CY000745, CY000753, CY000761, CY000769, CY000777, CY000785, CY000793, CY000801,
CY000809, CY000817, CY000825, CY000833, CY000841, CY000849, CY000857, CY000865,
CY000873, CY000881, CY000889, CY000901, CY000909, CY000917, CY000925, CY000933,
CY000941, CY000949, CY000957, CY000965, CY000973, CY000981, CY000989, CY000997,
CY001005, CY001013, CY001021, CY001029, CY001037, CY001045, CY001053, CY001061,
CY001064, CY001072, CY001080, CY001088, CY001096, CY001104, CY001112, CY001120,
CY001128, CY001136, CY001144, CY001152, CY001160, CY001168, CY001176, CY001184,
CY001197, CY001205, CY001213, CY001221, CY001229, CY001237, CY001245, CY001253,
CY001261, CY001269, CY001277, CY001285, CY001293, CY001301, CY001309, CY001317,
CY001325, CY001333, CY001341, CY001349, CY001357, CY001365, CY001373, CY001381,
CY001397, CY001405, CY001413, CY001421, CY001429, CY001437, CY001445, CY001453,
CY001461, CY001469, CY001477, CY001485, CY001493, CY001504, CY001512, CY001520,
CY001528, CY001536, CY001544, CY001552, CY001560, CY001568, CY001576, CY001584,
CY001592, CY001600, CY001608, CY001616, CY001624, CY001632, CY001640, CY001648,
CY001656, CY001664, CY001672, CY001688, CY001696, CY001704, CY001712, CY001720,
CY001728, CY001736, CY001744, CY001752, CY001760, CY001768, CY001776, CY001784,
CY001792, CY001800, CY001808, CY001816, CY001824, CY001832, CY001840, CY001848,
CY001856, CY001864, CY001872, CY001880, CY001888, CY001896, CY001904, CY001912,
CY001920, CY001928, CY001936, CY001944, CY001952, CY001960, CY001968, CY001976,
CY001984, CY001992, CY002000, CY002008, CY002016, CY002024, CY002032, CY002040,
CY002048, CY002056, CY002064, CY002072, CY002080, CY002088, CY002096, CY002104,
CY002112, CY002120, CY002128, CY002136, CY002144, CY002152, CY002160, CY002168,
CY002176, CY002184, CY002192, CY002200, CY002208, CY002216, CY002224, CY002232,
CY002240, CY002248, CY002256, CY002264, CY002272, CY002280, CY002288, CY002296,
CY002304, CY002312, CY002328, CY002336, CY002344, CY002352, CY002360, CY002368,
CY002376, CY002384, CY002392, CY002400, CY002408, CY002416, CY002424, CY002432,
CY002440, CY002448, CY002456, CY002464, CY002472, CY002480, CY002488, CY002496,
CY002504, CY002512, CY002520, CY002528, CY002536, CY002544, CY002552, CY002560,
CY002568, CY002576, CY002584, CY002592, CY002600, CY002608, CY002616, CY002624,
CY002632, CY002640, CY002648, CY002656, CY002664, CY002672, CY002680, CY002688,
CY002696, CY002704, CY002712, CY002720, CY002728, CY002736, CY002744, CY002752,
CY002760, CY002768, CY002776, CY002784, CY002792, CY002800, CY002808, CY002816,
CY002904, CY002905, CY002906, CY002914, CY002922, CY002930, CY002938, CY002946,
CY002954, CY002962, CY002984, CY002992, CY003000, CY003008, CY003016, CY003024,
CY003032, CY003040, CY003048, CY003056, CY003064, CY003072, CY003080, CY003088,
CY003096, CY003104, CY003112, CY003120, CY003123, CY003136, CY003144, CY003152,
CY003160, CY003168, CY003176, CY003184, CY003192, CY003200, CY003208, CY003216,
CY003224, CY003232, CY003240, CY003248, CY003256, CY003264, CY003272, CY003280,
CY003288, CY003296, CY003304, CY003312, CY003320, CY003328, CY003336, CY003344,
CY003352, CY003368, CY003376, CY003384, CY003392, CY003400, CY003408, CY003416,
CY003424, CY003432, CY003440, CY003448, CY003456, CY003464, CY003472, CY003480,
CY003488, CY003496, CY003504, CY003512, CY003520, CY003528, CY003536, CY003544,
CY003552, CY003560, CY003568, CY003576, CY003584, CY003592, CY003600, CY003608,
CY003616, CY003624, CY003632, CY003640, CY003648, CY003656, CY003664, CY003672,
CY003680, CY003688, CY003696, CY003704, CY003712, CY003720, CY003728, CY003736,
CY003744, CY003752, CY003761, CY003769, CY003777, CY003785, CY003793, CY003801,
CY003809, CY003817, CY003825, CY003833, CY006043, CY006044, CY006052, CY006060,
CY006068, CY006076, CY006084, CY006092, CY006107, CY006115, CY006123, CY006131,
CY006139, CY006147, CY006155, CY006163, CY006171, CY006179, CY006187, CY006195,
CY006203, CY006211, CY006219, CY006227, CY006235, CY006243, CY006251, CY006259,
CY006267, CY006275, CY006283, CY006291, CY006299, CY006307, CY006315, CY006323,
CY006331, CY006339, CY006347, CY006355, CY006363, CY006371, CY006379, CY006387,
CY006395, CY006403, CY006411, CY006419, CY006427, CY006435, CY006443, CY006451,
CY006459, CY006467, CY006475, CY006483, CY006491, CY006499, CY006507, CY006515,
CY006523, CY006531, CY006539, CY006547, CY006555, CY006563, CY006571, CY006579,
CY006587, CY006595, CY006603, CY006611, CY006619, CY006627, CY006635, CY006659,
CY006667, CY006675, CY006683, CY006691, CY006699, CY006707, CY006715, CY006723,
CY006731, CY006739, CY006747, CY006755, CY006763, CY006771, CY006779, CY006787,
CY006795, CY006803, CY006811, CY006819, CY006827, CY006835, CY006843, CY006851,
CY006859, CY006867, CY006875, CY006883, CY006891, CY006899, CY006907, CY006915,
CY006923, CY006931, CY006939, CY006947, CY006955, CY006963, CY006971, CY006979,
CY006987, CY006995, CY007003, CY007011, CY007019, CY007027, CY007035, CY007043,
CY007051, CY007059, CY007067, CY007075, CY007083, CY007091, CY007099, CY007107,
CY007115, CY007123, CY007131, CY007139, CY007147, CY007155, CY007163, CY007171,
CY007179, CY007187, CY007195, CY007203, CY007211, CY007219, CY007227, CY007235,
CY007243, CY007251, CY007259, CY007267, CY007275, CY007283, CY007291, CY007299,
CY007307, CY007315, CY007323, CY007331, CY007339, CY007347, CY007355, CY007363,
CY007371, CY007379, CY007387, CY007395, CY007403, CY007411, CY007419, CY007427,
CY007435, CY007443, CY007451, CY007459, CY007467, CY007475, CY007483, CY007491,
CY007499, CY007507, CY007515, CY007523, CY007531, CY007539, CY007547, CY007555,
CY007563, CY007571, CY007579, CY007587, CY007595, CY007603, CY007611, CY007619,
CY007627, CY007635, CY007643, CY007651, CY007659, CY007667, CY007675, CY007683,
CY007691, CY007699, CY007707, CY007715, CY007723, CY007731, CY007739, CY007747,
CY007755, CY007763, CY007771, CY007779, CY007787, CY007795, CY007803, CY007811,
CY007819, CY007827, CY007835, CY007843, CY007851, CY007859, CY007867, CY007875,
CY007883, CY007891, CY007899, CY007907, CY007915, CY007923, CY007931, CY007939,
CY007947, CY007955, CY007963, CY007971, CY007979, CY007987, CY007995, CY008003,
CY008011, CY008019, CY008027, CY008035, CY008043, CY008051, CY008059, CY008067,
CY008075, CY008083, CY008091, CY008099, CY008107, CY008115, CY008123, CY008131,
CY008139, CY008148, CY008156, CY008164, CY008172, CY008180, CY008188, CY008196,
CY008204, CY008212, CY008220, CY008228, CY008236, CY008244, CY008252, CY008260,
CY008268, CY008276, CY008284, CY008292, CY008300, CY008308, CY008316, CY008324,
CY008332, CY008340, CY008348, CY008356, CY008364, CY008372, CY008380, CY008388,
CY008396, CY008404, CY008412, CY008420, CY008428, CY008436, CY008444, CY008452,
CY008460, CY008468, CY008476, CY008484, CY008492, CY008500, CY008508, CY008516,
CY008524, CY008532, CY008540, CY008548, CY008556, CY008564, CY008572, CY008580,
CY008588, CY008596, CY008604, CY008612, CY008620, CY008628, CY008636, CY008644,
CY008652, CY008660, CY008668, CY008676, CY008684, CY008692, CY008700, CY008708,
CY008716, CY008724, CY008732, CY008740, CY008748, CY008756, CY008764, CY008772,
CY008780, CY008788, CY008796, CY008804, CY008812, CY008820, CY008828, CY008836,
CY008844, CY008852, CY008860, CY008868, CY008876, CY008884, CY008892, CY008900,
CY008908, CY008916, CY008924, CY008932, CY008940, CY008948, CY008956, CY008964,
CY008972, CY008980, CY008988, CY008996, CY009004, CY009012, CY009020, CY009028,
CY009036, CY009044, CY009052, CY009060, CY009068, CY009076, CY009084, CY009092,
CY009100, CY009108, CY009116, CY009124, CY009132, CY009140, CY009148, CY009156,
CY009164, CY009172, CY009180, CY009188, CY009196, CY009204, CY009212, CY009220,
CY009228, CY009236, CY009244, CY009252, CY009260, CY009268, CY009276, CY009284,
CY009292, CY009316, CY009324, CY009332, CY009340, CY009348, CY009356, CY009364,
CY009388, CY009396, CY009404, CY009412, CY009420, CY009428, CY009436, CY009444,
CY009452, CY009460, CY009468, CY009476, CY009484, CY009492, CY009500, CY009508,
CY009516, CY009524, CY009532, CY009540, CY009548, CY009556, CY009564, CY009572,
CY009580, CY009588, CY009596, CY009604, CY009612, CY009620, CY009636, CY009644,
CY009652, CY009660, CY009668, CY009676, CY009684, CY009692, CY009700, CY009708,
CY009716, CY009724, CY009732, CY009740, CY009748, CY009756, CY009764, CY009772,
CY009780, CY009788, CY009796, CY009804, CY009812, CY009820, CY009828, CY009836,
CY009844, CY009852, CY009860, CY009868, CY009876, CY009884, CY009900, CY009908,
CY009924, CY009932, CY009940, CY009948, CY009956, CY009964, CY009972, CY009980,
CY009988, CY009996, CY010004, CY010012, CY010020, CY010028, CY010036, CY010044,
CY010052, CY010060, CY010068, CY010076, CY010084, CY010092, CY010100, CY010108,
CY010116, CY010124, CY010132, CY010140, CY010148, CY010156, CY010164, CY010172,
CY010180, CY010188, CY010196, CY010204, CY010212, CY010220, CY010228, CY010236,
CY010244, CY010252, CY010260, CY010268, CY010276, CY010284, CY010292, CY010300,
CY010308, CY010316, CY010324, CY010332, CY010340, CY010348, CY010356, CY010364,
CY010372, CY010380, CY010388, CY010396, CY010404, CY010412, CY010420, CY010428,
CY010436, CY010444, CY010452, CY010460, CY010468, CY010476, CY010484, CY010492,
CY010500, CY010508, CY010516, CY010524, CY010532, CY010540, CY010548, CY010556,
CY010588, CY010596, CY010604, CY010612, CY010620, CY010628, CY010636, CY010644,
CY010652, CY010660, CY010668, CY010676, CY010684, CY010692, CY010700, CY010708,
CY010716, CY010724, CY010732, CY010740, CY010748, CY010756, CY010764, CY010772,
CY010780, CY010788, CY010796, CY010804, CY010812, CY010820, CY010828, CY010836,
CY010844, CY010852, CY010860, CY010868, CY010876, CY010884, CY010892, CY010900,
CY010908, CY010916, CY010924, CY010932, CY010940, CY010948, CY010956, CY010964,
CY010972, CY010980, CY010988, CY010996, CY011004, CY011012, CY011020, CY011064,
CY011072, CY011080, CY011088, CY011120, CY011128, CY011136, CY011144, CY011152,
CY011160, CY011168, CY011176, CY011184, CY011192, CY011200, CY011208, CY011216,
CY011224, CY011232, CY011240, CY011256, CY011264, CY011272, CY011280, CY011288,
CY011296, CY011304, CY011312, CY011320, CY011328, CY011336, CY011344, CY011352,
CY011360, CY011368, CY011376, CY011384, CY011392, CY011400, CY011408, CY011416,
CY011424, CY011432, CY011440, CY011448, CY011456, CY011464, CY011472, CY011480,
CY011488, CY011496, CY011504, CY011512, CY011520, CY011528, CY011536, CY011544,
CY011552, CY011560, CY011568, CY011576, CY011584, CY011592, CY011600, CY011608,
CY011616, CY011624, CY011632, CY011640, CY011648, CY011656, CY011664, CY011672,
CY011680, CY011688, CY011696, CY011704, CY011712, CY011720, CY011728, CY011736,
CY011744, CY011752, CY011760, CY011768, CY011776, CY011784, CY011792, CY011800,
CY011808, CY011816, CY011824, CY011832, CY011840, CY011848, CY011856, CY011864,
CY011872, CY011880, CY011888, CY011896, CY011904, CY011912, CY011920, CY011928,
CY011936, CY011944, CY011952, CY011960, CY011968, CY011976, CY011984, CY011992,
CY012000, CY012008, CY012016, CY012024, CY012032, CY012040, CY012048, CY012056,
CY012064, CY012072, CY012080, CY012088, CY012096, CY012104, CY012112, CY012120,
CY012128, CY012136, CY012144, CY012152, CY012160, CY012168, CY012176, CY012184,
CY012192, CY012200, CY012208, CY012216, CY012224, CY012232, CY012240, CY012248,
CY012256, CY012264, CY012272, CY012280, CY012288, CY012296, CY012304, CY012312,
CY012320, CY012328, CY012336, CY012344, CY012352, CY012360, CY012368, CY012376,
CY012384, CY012392, CY012400, CY012408, CY012416, CY012424, CY012432, CY012440,
CY012448, CY012456, CY012464, CY012472, CY012480, CY012488, CY012496, CY012504,
CY012512, CY012520, CY012528, CY012536, CY012544, CY012552, CY012560, CY012568,
CY012576, CY012584, CY012592, CY012600, CY012608, CY012616, CY012624, CY012632,
CY012640, CY012648, CY012656, CY012664, CY012672, CY012680, CY012688, CY012696,
CY012704, CY012712, CY012720, CY012728, CY012736, CY012744, CY012752, CY012760,
CY012768, CY012776, CY012784, CY012792, CY012848, CY012856, CY012864, CY012872,
CY012880, CY012888, CY012896, CY012904, CY012912, CY012920, CY012928, CY012936,
CY012944, CY012952, CY012960, CY012968, CY012976, CY012984, CY012992, CY013000,
CY013008, CY013016, CY013024, CY013032, CY013040, CY013048, CY013056, CY013064,
CY013072, CY013080, CY013088, CY013096, CY013104, CY013112, CY013120, CY013128,
CY013136, CY013144, CY013152, CY013160, CY013168, CY013176, CY013184, CY013192,
CY013200, CY013208, CY013216, CY013224, CY013232, CY013240, CY013271, CY013279,
CY013287, CY013295, CY013303, CY013311, CY013319, CY013327, CY013335, CY013343,
CY013351, CY013359, CY013367, CY013375, CY013383, CY013389, CY013397, CY013405,
CY013413, CY013421, CY013429, CY013437, CY013445, CY013453, CY013461, CY013469,
CY013477, CY013485, CY013493, CY013501, CY013509, CY013517, CY013525, CY013533,
CY013541, CY013549, CY013557, CY013565, CY013573, CY013581, CY013589, CY013597,
CY013605, CY013613, CY013621, CY013629, CY013637, CY013645, CY013653, CY013661,
CY013669, CY013677, CY013685, CY013693, CY013701, CY013709, CY013717, CY013725,
CY013733, CY013741, CY013749, CY013757, CY013765, CY013773, CY013781, CY013789,
CY013797, CY013805, CY013813, CY013821, CY013829, CY013837, CY013845, CY013853,
CY013871, CY013879, CY013887, CY013895, CY013903, CY013911, CY013919, CY013927,
CY013935, CY013943, CY013951, CY013959, CY013967, CY013975, CY013983, CY013991,
CY013999, CY014007, CY014015, CY014023, CY014031, CY014039, CY014047, CY014055,
CY014063, CY014071, CY014079, CY014087, CY014095, CY014103, CY014111, CY014119,
CY014127, CY014135, CY014143, CY014151, CY014159, CY014168, CY014177, CY014197,
CY014198, CY014199, CY014200, CY014201, CY014202, CY014203, CY014204, CY014205,
CY014206, CY014207, CY014209, CY014210, CY014211, CY014212, CY014213, CY014272,
CY014280, CY014288, CY014296, CY014303, CY014311, CY014368, CY014376, CY014384,
CY014393, CY014401, CY014409, CY014417, CY014425, CY014433, CY014441, CY014449,
CY014457, CY014465, CY014477, CY014481, CY014489, CY014497, CY014510, CY014518,
CY014529, CY014537, CY014543, CY014976, CY015006, CY015508, CY015516, CY015524,
CY015532, CY015540, CY015548, CY015556, CY015564, CY015572, CY015580, CY015588,
CY015596, CY015604, CY015612, CY015620, CY015628, CY015636, CY015644, CY015652,
CY015660, CY015668, CY015676, CY015684, CY015692, CY015700, CY015708, CY015716,
CY015724, CY015732, CY015740, CY015748, CY015756, CY015764, CY015772, CY015780,
CY015788, CY015796, CY015804, CY015812, CY015820, CY015828, CY015836, CY015844,
CY015852, CY015860, CY015868, CY015876, CY015884, CY015892, CY015900, CY015908,
CY015916, CY015924, CY015932, CY015940, CY015948, CY015956, CY015964, CY015972,
CY015980, CY015988, CY015996, CY016004, CY016012, CY016020, CY016028, CY016036,
CY016044, CY016052, CY016060, CY016068, CY016076, CY016084, CY016092, CY016100,
CY016108, CY016116, CY016196, CY016204, CY016212, CY016228, CY016236, CY016244,
CY016252, CY016260, CY016268, CY016323, CY016324, CY016326, CY016327, CY016328,
CY016329, CY016403, CY016427, CY016435, CY016443, CY016451, CY016459, CY016467,
CY016475, CY016483, CY016491, CY016499, CY016507, CY016515, CY016523, CY016531,
CY016539, CY016547, CY016555, CY016563, CY016571, CY016579, CY016587, CY016595,
CY016603, CY016627, CY016635, CY016643, CY016651, CY016659, CY016667, CY016675,
CY016683, CY016691, CY016699, CY016707, CY016715, CY016723, CY016731, CY016739,
CY016747, CY016755, CY016763, CY016771, CY016963, CY016971, CY016979, CY016987,
CY016995, CY017003, CY017011, CY017019, CY017083, CY017091, CY017099, CY017107,
CY017115, CY017123, CY017131, CY017139, CY017147, CY017155, CY017163, CY017171,
CY017195, CY017203, CY017211, CY017219, CY017227, CY017235, CY017243, CY017251,
CY017259, CY017267, CY017283, CY017291, CY017299, CY017307, CY017315, CY017323,
CY017331, CY017339, CY017347, CY017355, CY017363, CY017371, CY017379, CY017387,
CY017395, CY017419, CY017427, CY017435, CY017443, CY017451, CY017459, CY017467,
CY017475, CY017483, CY017491, CY017499, CY017507, CY017515, CY017523, CY017531,
CY017539, CY017547, CY017555, CY017563, CY017571, CY017579, CY017587, CY017595,
CY017603, CY017611, CY017619, CY017627, CY017638, CY017646, CY017654, CY017662,
CY017670, CY017678, CY017688, D13573, D13574, D13579, D13580, DQ059385, DQ086157,
DQ086159, DQ086160, DQ086161, DQ089634, DQ089635, DQ089636, DQ089637, DQ089638,
DQ089639, DQ227423, DQ227424, DQ227425, DQ227426, DQ227427, DQ227428, DQ227429,
DQ227430, DQ227431, DQ249259, DQ249260, DQ249261, DQ249262, DQ371928, DQ371929,
DQ371930, DQ372591, DQ397950, DQ415316, DQ415317, DQ415318, DQ415319, DQ415320,
DQ415321, DQ415322, DQ415323, DQ415324, DQ415325, DQ415326, DQ435202, DQ464377,
DQ469962, DQ487340, DQ487341, DQ497719, DQ497720, DQ497722, DQ497723, DQ497724,
DQ497725, DQ497726, DQ497727, DQ497728, DQ497729, DQ508825, DQ508833, DQ508841,
DQ508849, DQ508857, DQ508865, DQ508873, DQ508881, DQ508889, DQ508897, DQ508905,
DQ508929, DQ649410, DQ842489, DQ883589, DQ883600, DQ883601, DQ883608, DQ885610,
DQ885612, DQ885614, DQ885616, DQ885618, EF061116, EF101741, EF101749, ISDN110940,
ISDN117777, ISDN117778, ISDN118371, ISDN119678, ISDN119760, ISDN121986,
ISDN125783, ISDN125873, ISDN128026, ISDN129400, ISDN129527, ISDN129582,
ISDN129921, ISDN129922, ISDN130365, ISDN130373, ISDN130912, ISDN132201,
ISDN132202, ISDN132406, ISDN132414, ISDN133098, ISDN133105, ISDN133316,
ISDN133323, ISDN133356, ISDN133364, ISDN13422, ISDN136806, ISDN136807,
ISDN136808, ISDN136809, ISDN136810, ISDN136811, ISDN136812, ISDN136813,
ISDN136814, ISDN136815, ISDN136816, ISDN136841, ISDN136849, ISDN136908,
ISDN136909, ISDN137412, ISDN137413, ISDN137414, ISDN138723, ISDN138724,
ISDN138748, ISDN138772, ISDN140050, ISDN140051, ISDN140052, ISDN140053,
ISDN140054, ISDN140055, ISDN140056, ISDN140057, ISDN140058, ISDN140059,
ISDN140060, ISDN140061, ISDN140810, ISDN140818, ISDN140835, ISDN140843,
ISDN140851, ISDN140859, ISDN140866, ISDN140894, ISDN140896, ISDN140926,
ISDN181365, ISDN183297, ISDN183305, ISDN183313, ISDN183698, ISDN183706,
ISDN183714, ISDN183722, ISDN183730, ISDN183738, ISDN183746, ISDN183754,
ISDN183762, ISDN183770, ISDN183782, ISDN185493, ISDN187421, ISDN187429,
ISDN187437, ISDN187445, ISDN187453, ISDN204133, ISDN204141, ISDN204149,
ISDN207103, ISDN207111, ISDN208026, ISDN208027, ISDN214632, ISDN214634,
ISDN214636, ISDN214638, ISDN214640, ISDN214642, ISDN214645, ISDN214648,
ISDN214651, ISDN214654, ISDN214657, ISDN214660, ISDN214663, ISDN214665,
ISDN214668, ISDN214671, ISDN214674, ISDN214677, ISDN214680, ISDN214683,
ISDN214686, ISDN214689, ISDN214692, ISDN219941, ISDN219944, ISDN219947,
ISDN219950, ISDN219953, ISDN38262, ISDN38686, ISDN38687, ISDN38688, ISDN40341,
ISDN40917, ISDN40918, ISDN48661, ISDN48662, ISDN48663, ISDN48664, ISDN48665,
ISDN48666, ISDN49460, ISDN69439, ISDN69608, ISDNASYD97, J02092, J02127, J02132,
J02135, J02176, K01330, L11125, L11126, L11133, L11134, L11142, L20406, L20407, L20408,
L20409, L20410, L24362, M21648, M38312, M38353, M54895, M55059, NC_002017, S62154,
U02085, U02464, U08858, U08859, U08903, U08904, U08905, U26830, U37727, U38242,
U53162, U53163, U97740, V01085, V01086, V01089, V01098, V01103, X05907, X17221,
Z54286, Z54287, Z54288, Z54289
5: GenBank and LANL accession numbers for NP sequences (segment 5) used in this
analysis.
AB019358, AB019359, AB019360, AB019361, AB036779, AB126624, AB126632, AB212055,
AB262464, AF028710, AF036359, AF038254, AF038255, AF038256, AF038257, AF038258,
AF038259, AF046092, AF072545, AF084276, AF084277, AF084278, AF115284, AF115285,
AF255742, AF255743, AF255744, AF255745, AF255746, AF255747, AF255748, AF255749,
AF255750, AF255751, AF255752, AF255753, AF258516, AF258517, AF342819, AF348180,
AF348181, AF348182, AF348183, AF389119, AF398867, AF483604, AJ238021, AJ289871,
AJ289872, AJ289873, AJ291400, AJ291401, AJ293924, AJ458276, AJ458277, AJ628066,
AJ867076, AY043026, AY210066, AY210067, AY210068, AY210069, AY210070, AY210071,
AY210072, AY210073, AY210074, AY210075, AY210076, AY210077, AY210078, AY210079,
AY210080, AY210081, AY210082, AY210083, AY210084, AY210085, AY210086, AY210087,
AY210088, AY210089, AY210090, AY210091, AY210092, AY210093, AY210094, AY210095,
AY210096, AY210097, AY210098, AY210099, AY210100, AY210101, AY210102, AY210103,
AY210104, AY210207, AY210208, AY210209, AY210210, AY210211, AY210212, AY210213,
AY210214, AY210215, AY210216, AY210217, AY210218, AY210219, AY210220, AY210221,
AY210222, AY210223, AY210224, AY210225, AY210226, AY210227, AY210228, AY210229,
AY210230, AY210231, AY210232, AY210233, AY210234, AY210235, AY210236, AY210237,
AY210238, AY575905, AY575906, AY651498, AY651499, AY651500, AY651501, AY744935,
AY818138, AY936880, AY936881, AY936882, CY000004, CY000012, CY000020, CY000028,
CY000036, CY000044, CY000052, CY000060, CY000068, CY000076, CY000084, CY000092,
CY000100, CY000108, CY000116, CY000124, CY000132, CY000140, CY000148, CY000156,
CY000164, CY000172, CY000180, CY000188, CY000196, CY000204, CY000212, CY000220,
CY000228, CY000236, CY000244, CY000252, CY000260, CY000268, CY000276, CY000284,
CY000292, CY000300, CY000308, CY000316, CY000324, CY000332, CY000340, CY000348,
CY000356, CY000364, CY000372, CY000380, CY000388, CY000396, CY000404, CY000412,
CY000420, CY000428, CY000436, CY000444, CY000452, CY000460, CY000468, CY000476,
CY000484, CY000492, CY000500, CY000508, CY000520, CY000524, CY000532, CY000540,
CY000548, CY000556, CY000564, CY000572, CY000579, CY000588, CY000596, CY000604,
CY000612, CY000620, CY000628, CY000636, CY000644, CY000652, CY000660, CY000668,
CY000676, CY000684, CY000692, CY000700, CY000708, CY000716, CY000724, CY000732,
CY000740, CY000748, CY000756, CY000764, CY000772, CY000780, CY000788, CY000796,
CY000804, CY000812, CY000820, CY000828, CY000836, CY000844, CY000852, CY000860,
CY000868, CY000876, CY000884, CY000892, CY000904, CY000912, CY000920, CY000928,
CY000936, CY000944, CY000952, CY000960, CY000968, CY000976, CY000984, CY000992,
CY001000, CY001008, CY001016, CY001024, CY001032, CY001040, CY001048, CY001056,
CY001067, CY001075, CY001083, CY001091, CY001099, CY001107, CY001115, CY001123,
CY001131, CY001139, CY001147, CY001155, CY001163, CY001171, CY001179, CY001187,
CY001192, CY001200, CY001208, CY001216, CY001224, CY001232, CY001240, CY001248,
CY001256, CY001264, CY001272, CY001280, CY001288, CY001296, CY001304, CY001312,
CY001320, CY001328, CY001336, CY001344, CY001352, CY001360, CY001368, CY001376,
CY001384, CY001400, CY001408, CY001416, CY001424, CY001432, CY001440, CY001448,
CY001456, CY001464, CY001472, CY001480, CY001488, CY001496, CY001507, CY001515,
CY001523, CY001531, CY001539, CY001547, CY001555, CY001563, CY001571, CY001579,
CY001587, CY001595, CY001603, CY001611, CY001619, CY001627, CY001635, CY001643,
CY001651, CY001659, CY001667, CY001675, CY001683, CY001691, CY001699, CY001707,
CY001715, CY001723, CY001731, CY001739, CY001747, CY001755, CY001763, CY001771,
CY001779, CY001787, CY001795, CY001803, CY001811, CY001819, CY001827, CY001835,
CY001843, CY001851, CY001859, CY001867, CY001875, CY001883, CY001891, CY001899,
CY001907, CY001915, CY001923, CY001931, CY001939, CY001947, CY001955, CY001963,
CY001971, CY001979, CY001987, CY001995, CY002003, CY002011, CY002019, CY002027,
CY002035, CY002043, CY002051, CY002059, CY002067, CY002075, CY002083, CY002091,
CY002099, CY002107, CY002115, CY002123, CY002131, CY002139, CY002147, CY002155,
CY002163, CY002171, CY002179, CY002187, CY002195, CY002203, CY002211, CY002219,
CY002227, CY002235, CY002243, CY002251, CY002259, CY002267, CY002275, CY002283,
CY002291, CY002299, CY002307, CY002315, CY002331, CY002339, CY002347, CY002355,
CY002363, CY002371, CY002379, CY002387, CY002395, CY002403, CY002411, CY002419,
CY002427, CY002435, CY002443, CY002451, CY002459, CY002467, CY002475, CY002483,
CY002491, CY002499, CY002507, CY002515, CY002523, CY002531, CY002539, CY002547,
CY002555, CY002563, CY002571, CY002579, CY002587, CY002595, CY002603, CY002611,
CY002619, CY002627, CY002635, CY002643, CY002651, CY002659, CY002667, CY002675,
CY002683, CY002691, CY002699, CY002707, CY002715, CY002723, CY002731, CY002739,
CY002747, CY002755, CY002763, CY002771, CY002779, CY002787, CY002795, CY002803,
CY002811, CY002819, CY002909, CY002917, CY002925, CY002933, CY002941, CY002949,
CY002957, CY002965, CY002972, CY002979, CY002987, CY002995, CY003003, CY003011,
CY003019, CY003027, CY003035, CY003043, CY003051, CY003059, CY003067, CY003075,
CY003083, CY003091, CY003099, CY003107, CY003115, CY003124, CY003130, CY003139,
CY003147, CY003155, CY003163, CY003171, CY003179, CY003187, CY003195, CY003203,
CY003211, CY003219, CY003227, CY003235, CY003243, CY003251, CY003259, CY003267,
CY003275, CY003283, CY003291, CY003299, CY003307, CY003315, CY003323, CY003331,
CY003339, CY003347, CY003355, CY003371, CY003379, CY003387, CY003395, CY003403,
CY003411, CY003419, CY003427, CY003435, CY003443, CY003451, CY003459, CY003467,
CY003475, CY003483, CY003491, CY003499, CY003507, CY003515, CY003523, CY003531,
CY003539, CY003547, CY003555, CY003563, CY003571, CY003579, CY003587, CY003595,
CY003603, CY003611, CY003619, CY003627, CY003635, CY003643, CY003651, CY003659,
CY003667, CY003675, CY003683, CY003691, CY003699, CY003707, CY003715, CY003723,
CY003731, CY003739, CY003747, CY003755, CY003764, CY003772, CY003780, CY003788,
CY003796, CY003804, CY003812, CY003820, CY003828, CY003836, CY006047, CY006055,
CY006063, CY006071, CY006079, CY006087, CY006095, CY006102, CY006110, CY006118,
CY006126, CY006134, CY006142, CY006150, CY006158, CY006166, CY006174, CY006182,
CY006190, CY006198, CY006206, CY006214, CY006222, CY006230, CY006238, CY006246,
CY006254, CY006262, CY006270, CY006278, CY006286, CY006294, CY006302, CY006310,
CY006318, CY006326, CY006334, CY006342, CY006350, CY006358, CY006366, CY006374,
CY006382, CY006390, CY006398, CY006406, CY006414, CY006422, CY006430, CY006438,
CY006446, CY006454, CY006462, CY006470, CY006478, CY006486, CY006494, CY006502,
CY006510, CY006518, CY006526, CY006534, CY006542, CY006550, CY006558, CY006566,
CY006574, CY006582, CY006590, CY006598, CY006606, CY006614, CY006622, CY006630,
CY006638, CY006662, CY006670, CY006678, CY006686, CY006694, CY006702, CY006710,
CY006718, CY006726, CY006734, CY006742, CY006750, CY006758, CY006766, CY006774,
CY006782, CY006790, CY006798, CY006806, CY006814, CY006822, CY006830, CY006838,
CY006846, CY006854, CY006862, CY006870, CY006878, CY006886, CY006894, CY006902,
CY006910, CY006918, CY006926, CY006934, CY006942, CY006950, CY006958, CY006966,
CY006974, CY006982, CY006990, CY006998, CY007006, CY007014, CY007022, CY007030,
CY007038, CY007046, CY007054, CY007062, CY007070, CY007078, CY007086, CY007094,
CY007102, CY007110, CY007118, CY007126, CY007134, CY007142, CY007150, CY007158,
CY007166, CY007174, CY007182, CY007190, CY007198, CY007206, CY007214, CY007222,
CY007230, CY007238, CY007246, CY007254, CY007262, CY007270, CY007278, CY007286,
CY007294, CY007302, CY007310, CY007318, CY007326, CY007334, CY007342, CY007350,
CY007358, CY007366, CY007374, CY007382, CY007390, CY007398, CY007406, CY007414,
CY007422, CY007430, CY007438, CY007446, CY007454, CY007462, CY007470, CY007478,
CY007486, CY007494, CY007502, CY007510, CY007518, CY007526, CY007534, CY007542,
CY007550, CY007558, CY007566, CY007574, CY007582, CY007590, CY007598, CY007606,
CY007614, CY007622, CY007630, CY007638, CY007646, CY007654, CY007662, CY007670,
CY007678, CY007686, CY007694, CY007702, CY007710, CY007718, CY007726, CY007734,
CY007742, CY007750, CY007758, CY007766, CY007774, CY007782, CY007790, CY007798,
CY007806, CY007814, CY007822, CY007830, CY007838, CY007846, CY007854, CY007862,
CY007870, CY007878, CY007886, CY007894, CY007902, CY007910, CY007918, CY007926,
CY007934, CY007942, CY007950, CY007958, CY007966, CY007974, CY007982, CY007990,
CY007998, CY008006, CY008014, CY008022, CY008030, CY008038, CY008046, CY008054,
CY008062, CY008070, CY008078, CY008086, CY008094, CY008102, CY008110, CY008118,
CY008126, CY008134, CY008142, CY008151, CY008159, CY008167, CY008175, CY008183,
CY008191, CY008199, CY008207, CY008215, CY008223, CY008231, CY008239, CY008247,
CY008255, CY008263, CY008271, CY008279, CY008287, CY008295, CY008303, CY008311,
CY008319, CY008327, CY008335, CY008343, CY008351, CY008359, CY008367, CY008375,
CY008383, CY008391, CY008399, CY008407, CY008415, CY008423, CY008431, CY008439,
CY008447, CY008455, CY008463, CY008471, CY008479, CY008487, CY008495, CY008503,
CY008511, CY008519, CY008527, CY008535, CY008543, CY008551, CY008559, CY008567,
CY008575, CY008583, CY008591, CY008599, CY008607, CY008615, CY008623, CY008631,
CY008639, CY008647, CY008655, CY008663, CY008671, CY008679, CY008687, CY008695,
CY008703, CY008711, CY008719, CY008727, CY008735, CY008743, CY008751, CY008759,
CY008767, CY008775, CY008783, CY008791, CY008799, CY008807, CY008815, CY008823,
CY008831, CY008839, CY008847, CY008855, CY008863, CY008871, CY008879, CY008887,
CY008895, CY008903, CY008911, CY008919, CY008927, CY008935, CY008943, CY008951,
CY008959, CY008967, CY008975, CY008983, CY008991, CY008999, CY009007, CY009015,
CY009023, CY009031, CY009039, CY009047, CY009055, CY009063, CY009071, CY009079,
CY009087, CY009095, CY009103, CY009111, CY009119, CY009127, CY009135, CY009143,
CY009151, CY009159, CY009167, CY009175, CY009183, CY009191, CY009199, CY009207,
CY009215, CY009223, CY009231, CY009239, CY009247, CY009255, CY009263, CY009271,
CY009279, CY009287, CY009295, CY009319, CY009327, CY009335, CY009343, CY009351,
CY009359, CY009367, CY009391, CY009399, CY009407, CY009415, CY009423, CY009431,
CY009439, CY009447, CY009455, CY009463, CY009471, CY009479, CY009487, CY009495,
CY009503, CY009511, CY009519, CY009527, CY009535, CY009543, CY009551, CY009559,
CY009567, CY009575, CY009583, CY009591, CY009599, CY009607, CY009615, CY009623,
CY009639, CY009647, CY009655, CY009663, CY009671, CY009679, CY009687, CY009695,
CY009703, CY009711, CY009719, CY009727, CY009735, CY009743, CY009751, CY009759,
CY009767, CY009775, CY009783, CY009791, CY009799, CY009807, CY009815, CY009823,
CY009831, CY009839, CY009847, CY009855, CY009863, CY009871, CY009879, CY009887,
CY009903, CY009911, CY009927, CY009935, CY009943, CY009951, CY009959, CY009967,
CY009975, CY009983, CY009991, CY009999, CY010007, CY010015, CY010023, CY010031,
CY010039, CY010047, CY010055, CY010063, CY010071, CY010079, CY010087, CY010095,
CY010103, CY010111, CY010119, CY010127, CY010135, CY010143, CY010151, CY010159,
CY010167, CY010175, CY010183, CY010191, CY010199, CY010207, CY010215, CY010223,
CY010231, CY010239, CY010247, CY010255, CY010263, CY010271, CY010279, CY010287,
CY010295, CY010303, CY010311, CY010319, CY010327, CY010335, CY010343, CY010351,
CY010359, CY010367, CY010375, CY010383, CY010391, CY010399, CY010407, CY010415,
CY010423, CY010431, CY010439, CY010447, CY010455, CY010463, CY010471, CY010479,
CY010487, CY010495, CY010503, CY010511, CY010519, CY010527, CY010535, CY010543,
CY010551, CY010559, CY010591, CY010599, CY010607, CY010615, CY010623, CY010631,
CY010639, CY010647, CY010655, CY010663, CY010671, CY010679, CY010687, CY010695,
CY010703, CY010711, CY010719, CY010727, CY010735, CY010743, CY010751, CY010759,
CY010767, CY010775, CY010783, CY010791, CY010799, CY010807, CY010815, CY010823,
CY010831, CY010839, CY010847, CY010855, CY010863, CY010871, CY010879, CY010887,
CY010895, CY010903, CY010911, CY010919, CY010927, CY010935, CY010943, CY010951,
CY010959, CY010967, CY010975, CY010983, CY010991, CY010999, CY011007, CY011015,
CY011023, CY011067, CY011075, CY011083, CY011091, CY011123, CY011131, CY011139,
CY011147, CY011155, CY011163, CY011171, CY011179, CY011187, CY011195, CY011203,
CY011211, CY011219, CY011227, CY011235, CY011243, CY011259, CY011267, CY011275,
CY011283, CY011291, CY011299, CY011307, CY011315, CY011323, CY011331, CY011339,
CY011347, CY011355, CY011363, CY011371, CY011379, CY011387, CY011395, CY011403,
CY011411, CY011419, CY011427, CY011435, CY011443, CY011451, CY011459, CY011467,
CY011475, CY011483, CY011491, CY011499, CY011507, CY011515, CY011523, CY011531,
CY011539, CY011547, CY011555, CY011563, CY011571, CY011579, CY011587, CY011595,
CY011603, CY011611, CY011619, CY011627, CY011635, CY011643, CY011651, CY011659,
CY011667, CY011675, CY011683, CY011691, CY011699, CY011707, CY011715, CY011723,
CY011731, CY011739, CY011747, CY011755, CY011763, CY011771, CY011779, CY011787,
CY011795, CY011803, CY011811, CY011819, CY011827, CY011835, CY011843, CY011851,
CY011859, CY011867, CY011875, CY011883, CY011891, CY011899, CY011907, CY011915,
CY011923, CY011931, CY011939, CY011947, CY011955, CY011963, CY011971, CY011979,
CY011987, CY011995, CY012003, CY012011, CY012019, CY012027, CY012035, CY012043,
CY012051, CY012059, CY012067, CY012075, CY012083, CY012091, CY012099, CY012107,
CY012115, CY012123, CY012131, CY012139, CY012147, CY012155, CY012163, CY012171,
CY012179, CY012187, CY012195, CY012203, CY012211, CY012219, CY012227, CY012235,
CY012243, CY012251, CY012259, CY012267, CY012275, CY012283, CY012291, CY012299,
CY012307, CY012315, CY012323, CY012331, CY012339, CY012347, CY012355, CY012363,
CY012371, CY012379, CY012387, CY012395, CY012403, CY012411, CY012419, CY012427,
CY012435, CY012443, CY012451, CY012459, CY012467, CY012475, CY012483, CY012491,
CY012499, CY012507, CY012515, CY012523, CY012531, CY012539, CY012547, CY012555,
CY012563, CY012571, CY012579, CY012587, CY012595, CY012603, CY012611, CY012619,
CY012627, CY012635, CY012643, CY012651, CY012659, CY012667, CY012675, CY012683,
CY012691, CY012699, CY012707, CY012715, CY012723, CY012731, CY012739, CY012747,
CY012755, CY012763, CY012771, CY012779, CY012787, CY012795, CY012851, CY012859,
CY012867, CY012875, CY012883, CY012891, CY012899, CY012907, CY012915, CY012923,
CY012931, CY012939, CY012947, CY012955, CY012963, CY012971, CY012979, CY012987,
CY012995, CY013003, CY013011, CY013019, CY013027, CY013035, CY013043, CY013051,
CY013059, CY013067, CY013075, CY013083, CY013091, CY013099, CY013107, CY013115,
CY013123, CY013131, CY013139, CY013147, CY013155, CY013163, CY013171, CY013179,
CY013187, CY013195, CY013203, CY013211, CY013219, CY013227, CY013235, CY013243,
CY013274, CY013282, CY013290, CY013298, CY013306, CY013314, CY013322, CY013330,
CY013338, CY013346, CY013354, CY013362, CY013370, CY013378, CY013386, CY013392,
CY013400, CY013408, CY013416, CY013424, CY013432, CY013440, CY013448, CY013456,
CY013464, CY013472, CY013480, CY013488, CY013496, CY013504, CY013512, CY013520,
CY013528, CY013536, CY013544, CY013552, CY013560, CY013568, CY013576, CY013584,
CY013592, CY013600, CY013608, CY013616, CY013624, CY013632, CY013640, CY013648,
CY013656, CY013664, CY013672, CY013680, CY013688, CY013696, CY013704, CY013712,
CY013720, CY013728, CY013736, CY013744, CY013752, CY013760, CY013768, CY013776,
CY013784, CY013792, CY013800, CY013808, CY013816, CY013824, CY013832, CY013840,
CY013848, CY013856, CY013874, CY013882, CY013890, CY013898, CY013906, CY013914,
CY013922, CY013930, CY013938, CY013946, CY013954, CY013962, CY013970, CY013978,
CY013986, CY013994, CY014002, CY014010, CY014018, CY014026, CY014034, CY014042,
CY014050, CY014058, CY014066, CY014074, CY014082, CY014090, CY014098, CY014106,
CY014114, CY014122, CY014130, CY014138, CY014146, CY014154, CY014162, CY014172,
CY014178, CY014241, CY014242, CY014243, CY014244, CY014245, CY014246, CY014247,
CY014248, CY014249, CY014250, CY014251, CY014252, CY014253, CY014273, CY014281,
CY014289, CY014297, CY014304, CY014312, CY014371, CY014379, CY014387, CY014399,
CY014407, CY014415, CY014423, CY014431, CY014439, CY014447, CY014455, CY014463,
CY014471, CY014480, CY014487, CY014495, CY014503, CY014508, CY014516, CY014530,
CY014538, CY014544, CY014979, CY015009, CY015511, CY015519, CY015527, CY015535,
CY015543, CY015551, CY015559, CY015567, CY015575, CY015583, CY015591, CY015599,
CY015607, CY015615, CY015623, CY015631, CY015639, CY015647, CY015655, CY015663,
CY015671, CY015679, CY015687, CY015695, CY015703, CY015711, CY015719, CY015727,
CY015735, CY015743, CY015751, CY015759, CY015767, CY015775, CY015783, CY015791,
CY015799, CY015807, CY015815, CY015823, CY015831, CY015839, CY015847, CY015855,
CY015863, CY015871, CY015879, CY015887, CY015895, CY015903, CY015911, CY015919,
CY015927, CY015935, CY015943, CY015951, CY015959, CY015967, CY015975, CY015983,
CY015991, CY015999, CY016007, CY016015, CY016023, CY016031, CY016039, CY016047,
CY016055, CY016063, CY016071, CY016079, CY016087, CY016095, CY016103, CY016111,
CY016119, CY016199, CY016207, CY016215, CY016231, CY016239, CY016247, CY016255,
CY016263, CY016271, CY016406, CY016430, CY016438, CY016446, CY016454, CY016462,
CY016470, CY016478, CY016486, CY016494, CY016502, CY016510, CY016518, CY016526,
CY016534, CY016542, CY016550, CY016558, CY016566, CY016574, CY016582, CY016590,
CY016598, CY016606, CY016630, CY016638, CY016646, CY016654, CY016662, CY016670,
CY016678, CY016686, CY016694, CY016702, CY016710, CY016718, CY016726, CY016734,
CY016742, CY016750, CY016758, CY016766, CY016774, CY016966, CY016974, CY016982,
CY016990, CY016998, CY017006, CY017014, CY017022, CY017086, CY017094, CY017102,
CY017110, CY017118, CY017126, CY017134, CY017142, CY017150, CY017158, CY017166,
CY017174, CY017198, CY017206, CY017214, CY017222, CY017230, CY017238, CY017246,
CY017254, CY017262, CY017270, CY017286, CY017294, CY017302, CY017310, CY017318,
CY017326, CY017334, CY017342, CY017350, CY017358, CY017366, CY017374, CY017382,
CY017390, CY017398, CY017422, CY017430, CY017438, CY017446, CY017454, CY017462,
CY017470, CY017478, CY017486, CY017494, CY017502, CY017510, CY017518, CY017526,
CY017534, CY017542, CY017550, CY017558, CY017566, CY017574, CY017582, CY017590,
CY017598, CY017606, CY017614, CY017622, CY017630, CY017639, CY017647, CY017655,
CY017663, CY017671, CY017679, CY017689, D00051, D00599, D00600, D00601, D00602,
D00603, DQ099775, DQ099776, DQ099779, DQ099780, DQ099781, DQ099782, DQ099783,
DQ226139, DQ249264, DQ360840, DQ372594, DQ415327, DQ415328, DQ415329, DQ415330,
DQ415331, DQ415332, DQ415333, DQ415334, DQ415335, DQ415336, DQ415337, DQ469959,
DQ487330, DQ487339, DQ493156, DQ493157, DQ493158, DQ493159, DQ493160, DQ493161,
DQ493162, DQ493163, DQ493164, DQ493165, DQ493166, DQ508826, DQ508834, DQ508842,
DQ508850, DQ508858, DQ508866, DQ508874, DQ508882, DQ508890, DQ508898, DQ508906,
DQ508930, DQ535727, DQ643812, EF101752, ISDN125786, ISDN125874, ISDN128029,
ISDN129533, ISDN129583, ISDN130368, ISDN130376, ISDN130915, ISDN132409,
ISDN132417, ISDN133319, ISDN133326, ISDN133359, ISDN133367, ISDN133373,
ISDN13423, ISDN13442, ISDN13443, ISDN13444, ISDN136844, ISDN136852, ISDN137814,
ISDN137815, ISDN137816, ISDN137817, ISDN137818, ISDN137819, ISDN137820,
ISDN137821, ISDN137822, ISDN137823, ISDN137824, ISDN137825, ISDN137826,
ISDN137827, ISDN138751, ISDN138767, ISDN138775, ISDN140077, ISDN140078,
ISDN140079, ISDN140080, ISDN140081, ISDN140084, ISDN140085, ISDN140813,
ISDN140821, ISDN140836, ISDN140844, ISDN140852, ISDN140860, ISDN140867,
ISDN140903, ISDN140904, ISDN140905, ISDN140929, ISDN181366, ISDN183300,
ISDN183308, ISDN183316, ISDN183704, ISDN183712, ISDN183720, ISDN183728,
ISDN183736, ISDN183744, ISDN183752, ISDN183760, ISDN183768, ISDN183776,
ISDN183785, ISDN185500, ISDN187424, ISDN187432, ISDN187440, ISDN187448,
ISDN187456, ISDN188294, ISDN188295, ISDN188296, ISDN188297, ISDN188298,
ISDN188299, ISDN188300, ISDN188301, ISDN188302, ISDN188304, ISDN188305,
ISDN188306, ISDN188307, ISDN188308, ISDN188309, ISDN188312, ISDN188313,
ISDN188315, ISDN188316, ISDN188317, ISDN188318, ISDN188319, ISDN188320,
ISDN188321, ISDN204139, ISDN204147, ISDN204155, ISDN207101, ISDN207109,
ISDN40086, ISDN41067, J02137, L07340, L07341, L07342, L07343, L07344, L07345, L07346,
L07347, L07349, L07350, L07351, L07352, L07353, L07354, L07355, L07356, L07357,
L07358, L07359, L07360, L07361, L07362, L07363, L07364, L07365, L07366, L07367,
L07368, L07369, L07370, L07371, L07372, L07373, L07374, L24394, M22577, M23976,
M30746, M38279, M59329, M59330, M59331, M59332, M59333, M59334, M63749, M63750,
M63751, M63752, M63753, M63754, M63755, M76602, M76604, M76605, M76606, M76610,
M81571, M81577, M81583, NC_002019, U02086, U71144, U71145, U71146, U71147, V01084,
X15890, X51972, Z54290, Z54291, Z54292
6: GenBank and LANL accession numbers for NA sequences (segment 6) used in this
analysis.
AB101671, AB101672, AB101673, AB101674, AB101675, AB124653, AB124654, AB124655,
AB124656, AB124657, AB124658, AB124659, AB124660, AB124661, AB124662, AB124663,
AB124664, AB126623, AB126631, AB212056, AB239126, AB262465, AF028708, AF036357,
AF038260, AF038261, AF038262, AF038263, AF038264, AF038265, AF046089, AF084271,
AF084272, AF084273, AF084274, AF084275, AF102656, AF102657, AF102658, AF102659,
AF102660, AF102661, AF102662, AF102663, AF102664, AF102665, AF102666, AF102667,
AF102668, AF102669, AF102670, AF250356, AF250357, AF250363, AF250365, AF296752,
AF316805, AF316806, AF316807, AF316808, AF316809, AF316810, AF316811, AF316812,
AF316814, AF316815, AF316816, AF330819, AF342820, AF348184, AF348185, AF348186,
AF348187, AF382329, AF382330, AF382331, AF382332, AF386761, AF386762, AF386763,
AF386764, AF389120, AF398868, AF398869, AF398872, AF494252, AF494253, AF494254,
AF503463, AF503464, AF503465, AF503466, AF503467, AF503468, AF503469, AF503470,
AF503471, AF503472, AF533730, AF533731, AF533732, AF533733, AF533734, AF533735,
AF533736, AF533737, AF533738, AF533739, AF533740, AF533741, AF533742, AF533743,
AF533744, AF533745, AF533746, AF533747, AF533748, AF533749, AF533750, AF533989,
AF533990, AF533991, AF533992, AF533993, AF533994, AF533995, AF533996, AF533997,
AF533998, AF533999, AF534000, AF534001, AJ006953, AJ006954, AJ291403, AJ293923,
AJ307599, AJ307600, AJ307601, AJ307602, AJ307603, AJ307604, AJ307605, AJ307606,
AJ307607, AJ307608, AJ307609, AJ307610, AJ307611, AJ307612, AJ307613, AJ307614,
AJ307615, AJ307616, AJ307617, AJ307618, AJ307619, AJ307620, AJ307621, AJ307622,
AJ307623, AJ307624, AJ307625, AJ307626, AJ307627, AJ307628, AJ307629, AJ316063,
AJ404628, AJ404629, AJ457931, AJ457932, AJ457933, AJ457934, AJ457935, AJ457936,
AJ457937, AJ457938, AJ457939, AJ457940, AJ457941, AJ457942, AJ457943, AJ457944,
AJ457945, AJ457946, AJ457956, AJ457957, AJ457958, AJ457959, AJ457960, AJ457961,
AJ457962, AJ457963, AJ457964, AJ457965, AJ457966, AJ489846, AJ489847, AJ489848,
AJ489849, AJ489850, AJ489851, AJ518091, AJ518092, AJ518093, AJ518094, AJ518095,
AJ518096, AJ518097, AJ518098, AJ518099, AJ518100, AJ518101, AJ518102, AJ518104,
AJ867075, AY043022, AY043024, AY122326, AY122327, AY209895, AY209896, AY209897,
AY209898, AY209899, AY209900, AY209901, AY209902, AY209903, AY209904, AY209905,
AY209906, AY209907, AY209908, AY209909, AY209910, AY209911, AY209912, AY209913,
AY209914, AY209915, AY209916, AY209917, AY209918, AY209919, AY209920, AY209921,
AY209922, AY209923, AY209924, AY209925, AY209926, AY209927, AY209928, AY209929,
AY209930, AY209931, AY209932, AY209933, AY210105, AY210106, AY210107, AY210108,
AY210109, AY210110, AY210111, AY210112, AY210113, AY210114, AY210115, AY210116,
AY210117, AY210118, AY210119, AY210120, AY210121, AY210122, AY210123, AY210124,
AY210125, AY210126, AY210127, AY210128, AY210129, AY210130, AY210131, AY210132,
AY210133, AY210134, AY210135, AY210136, AY271795, AY297140, AY297141, AY297142,
AY297143, AY297144, AY297145, AY297146, AY297147, AY297148, AY297149, AY297150,
AY297151, AY297152, AY297153, AY310407, AY310408, AY310409, AY310410, AY310411,
AY310412, AY531006, AY531007, AY531008, AY531009, AY531010, AY531011, AY531012,
AY531013, AY531014, AY531015, AY531016, AY531017, AY531018, AY531019, AY531020,
AY531021, AY531022, AY531023, AY531024, AY531025, AY531026, AY531027, AY531028,
AY531034, AY531036, AY555151, AY555152, AY575881, AY575882, AY589662, AY589663,
AY589664, AY589665, AY589666, AY589667, AY589668, AY589669, AY589670, AY589671,
AY589672, AY589673, AY589674, AY589675, AY589676, AY643088, AY643089, AY651445,
AY651446, AY651447, AY651448, AY679513, AY818141, AY884285, AY884286, AY904335,
AY947477, CY000003, CY000011, CY000019, CY000027, CY000035, CY000043, CY000051,
CY000059, CY000067, CY000075, CY000083, CY000091, CY000099, CY000107, CY000115,
CY000123, CY000131, CY000139, CY000147, CY000155, CY000163, CY000171, CY000179,
CY000187, CY000195, CY000203, CY000211, CY000219, CY000227, CY000235, CY000243,
CY000251, CY000259, CY000267, CY000275, CY000283, CY000291, CY000299, CY000307,
CY000315, CY000323, CY000331, CY000339, CY000347, CY000355, CY000363, CY000371,
CY000379, CY000387, CY000395, CY000403, CY000411, CY000419, CY000427, CY000435,
CY000443, CY000451, CY000459, CY000467, CY000475, CY000483, CY000491, CY000499,
CY000507, CY000515, CY000523, CY000531, CY000539, CY000547, CY000555, CY000563,
CY000571, CY000578, CY000587, CY000595, CY000603, CY000611, CY000619, CY000627,
CY000635, CY000643, CY000651, CY000659, CY000667, CY000675, CY000683, CY000691,
CY000699, CY000707, CY000715, CY000723, CY000731, CY000739, CY000747, CY000755,
CY000763, CY000771, CY000779, CY000787, CY000795, CY000803, CY000811, CY000819,
CY000827, CY000835, CY000843, CY000851, CY000859, CY000867, CY000875, CY000883,
CY000891, CY000903, CY000911, CY000919, CY000927, CY000935, CY000943, CY000951,
CY000959, CY000967, CY000975, CY000983, CY000991, CY000999, CY001007, CY001015,
CY001023, CY001031, CY001039, CY001047, CY001055, CY001063, CY001066, CY001074,
CY001082, CY001090, CY001098, CY001106, CY001114, CY001122, CY001130, CY001138,
CY001146, CY001154, CY001162, CY001170, CY001178, CY001186, CY001199, CY001207,
CY001215, CY001223, CY001231, CY001239, CY001247, CY001255, CY001263, CY001271,
CY001279, CY001287, CY001295, CY001303, CY001311, CY001319, CY001327, CY001335,
CY001343, CY001351, CY001359, CY001367, CY001375, CY001383, CY001399, CY001407,
CY001415, CY001423, CY001431, CY001439, CY001447, CY001455, CY001463, CY001471,
CY001479, CY001487, CY001495, CY001506, CY001514, CY001522, CY001530, CY001538,
CY001546, CY001554, CY001562, CY001570, CY001578, CY001586, CY001594, CY001602,
CY001610, CY001618, CY001626, CY001634, CY001642, CY001650, CY001658, CY001666,
CY001674, CY001682, CY001690, CY001698, CY001706, CY001714, CY001722, CY001730,
CY001738, CY001746, CY001754, CY001762, CY001770, CY001778, CY001786, CY001794,
CY001802, CY001810, CY001818, CY001826, CY001834, CY001842, CY001850, CY001858,
CY001866, CY001874, CY001882, CY001890, CY001898, CY001906, CY001914, CY001922,
CY001930, CY001938, CY001946, CY001954, CY001962, CY001970, CY001978, CY001986,
CY001994, CY002002, CY002010, CY002018, CY002026, CY002034, CY002042, CY002050,
CY002058, CY002066, CY002074, CY002082, CY002090, CY002098, CY002106, CY002114,
CY002122, CY002130, CY002138, CY002146, CY002154, CY002162, CY002170, CY002178,
CY002186, CY002194, CY002202, CY002210, CY002218, CY002226, CY002234, CY002242,
CY002250, CY002258, CY002266, CY002274, CY002282, CY002290, CY002298, CY002306,
CY002314, CY002330, CY002338, CY002346, CY002354, CY002362, CY002370, CY002378,
CY002386, CY002394, CY002402, CY002410, CY002418, CY002426, CY002434, CY002442,
CY002450, CY002458, CY002466, CY002474, CY002482, CY002490, CY002498, CY002506,
CY002514, CY002522, CY002530, CY002538, CY002546, CY002554, CY002562, CY002570,
CY002578, CY002586, CY002594, CY002602, CY002610, CY002618, CY002626, CY002634,
CY002642, CY002650, CY002658, CY002666, CY002674, CY002682, CY002690, CY002698,
CY002706, CY002714, CY002722, CY002730, CY002738, CY002746, CY002754, CY002762,
CY002770, CY002778, CY002786, CY002794, CY002802, CY002810, CY002818, CY002908,
CY002916, CY002924, CY002932, CY002940, CY002948, CY002956, CY002964, CY002971,
CY002978, CY002986, CY002994, CY003002, CY003010, CY003018, CY003026, CY003034,
CY003042, CY003050, CY003058, CY003066, CY003074, CY003082, CY003090, CY003098,
CY003106, CY003114, CY003122, CY003129, CY003138, CY003146, CY003154, CY003162,
CY003170, CY003178, CY003186, CY003194, CY003202, CY003210, CY003218, CY003226,
CY003234, CY003242, CY003250, CY003258, CY003266, CY003274, CY003282, CY003290,
CY003298, CY003306, CY003314, CY003322, CY003330, CY003338, CY003346, CY003354,
CY003370, CY003378, CY003386, CY003394, CY003402, CY003410, CY003418, CY003426,
CY003434, CY003442, CY003450, CY003458, CY003466, CY003474, CY003482, CY003490,
CY003498, CY003506, CY003514, CY003522, CY003530, CY003538, CY003546, CY003554,
CY003562, CY003570, CY003578, CY003586, CY003594, CY003602, CY003610, CY003618,
CY003626, CY003634, CY003642, CY003650, CY003658, CY003666, CY003674, CY003682,
CY003690, CY003698, CY003706, CY003714, CY003722, CY003730, CY003738, CY003746,
CY003754, CY003763, CY003771, CY003779, CY003787, CY003795, CY003803, CY003811,
CY003819, CY003827, CY003835, CY006046, CY006054, CY006062, CY006070, CY006078,
CY006086, CY006094, CY006101, CY006109, CY006117, CY006125, CY006133, CY006141,
CY006149, CY006157, CY006165, CY006173, CY006181, CY006189, CY006197, CY006205,
CY006213, CY006221, CY006229, CY006237, CY006245, CY006253, CY006261, CY006269,
CY006277, CY006285, CY006293, CY006301, CY006309, CY006317, CY006325, CY006333,
CY006341, CY006349, CY006357, CY006365, CY006373, CY006381, CY006389, CY006397,
CY006405, CY006413, CY006421, CY006429, CY006437, CY006445, CY006453, CY006461,
CY006469, CY006477, CY006485, CY006493, CY006501, CY006509, CY006517, CY006525,
CY006533, CY006541, CY006549, CY006557, CY006565, CY006573, CY006581, CY006589,
CY006597, CY006605, CY006613, CY006621, CY006629, CY006637, CY006661, CY006669,
CY006677, CY006685, CY006693, CY006701, CY006709, CY006717, CY006725, CY006733,
CY006741, CY006749, CY006757, CY006765, CY006773, CY006781, CY006789, CY006797,
CY006805, CY006813, CY006821, CY006829, CY006837, CY006845, CY006853, CY006861,
CY006869, CY006877, CY006885, CY006893, CY006901, CY006909, CY006917, CY006925,
CY006933, CY006941, CY006949, CY006957, CY006965, CY006973, CY006981, CY006989,
CY006997, CY007005, CY007013, CY007021, CY007029, CY007037, CY007045, CY007053,
CY007061, CY007069, CY007077, CY007085, CY007093, CY007101, CY007109, CY007117,
CY007125, CY007133, CY007141, CY007149, CY007157, CY007165, CY007173, CY007181,
CY007189, CY007197, CY007205, CY007213, CY007221, CY007229, CY007237, CY007245,
CY007253, CY007261, CY007269, CY007277, CY007285, CY007293, CY007301, CY007309,
CY007317, CY007325, CY007333, CY007341, CY007349, CY007357, CY007365, CY007373,
CY007381, CY007389, CY007397, CY007405, CY007413, CY007421, CY007429, CY007437,
CY007445, CY007453, CY007461, CY007469, CY007477, CY007485, CY007493, CY007501,
CY007509, CY007517, CY007525, CY007533, CY007541, CY007549, CY007557, CY007565,
CY007573, CY007581, CY007589, CY007597, CY007605, CY007613, CY007621, CY007629,
CY007637, CY007645, CY007653, CY007661, CY007669, CY007677, CY007685, CY007693,
CY007701, CY007709, CY007717, CY007725, CY007733, CY007741, CY007749, CY007757,
CY007765, CY007773, CY007781, CY007789, CY007797, CY007805, CY007813, CY007821,
CY007829, CY007837, CY007845, CY007853, CY007861, CY007869, CY007877, CY007885,
CY007893, CY007901, CY007909, CY007917, CY007925, CY007933, CY007941, CY007949,
CY007957, CY007965, CY007973, CY007981, CY007989, CY007997, CY008005, CY008013,
CY008021, CY008029, CY008037, CY008045, CY008053, CY008061, CY008069, CY008077,
CY008085, CY008093, CY008101, CY008109, CY008117, CY008125, CY008133, CY008141,
CY008150, CY008158, CY008166, CY008174, CY008182, CY008190, CY008198, CY008206,
CY008214, CY008222, CY008230, CY008238, CY008246, CY008254, CY008262, CY008270,
CY008278, CY008286, CY008294, CY008302, CY008310, CY008318, CY008326, CY008334,
CY008342, CY008350, CY008358, CY008366, CY008374, CY008382, CY008390, CY008398,
CY008406, CY008414, CY008422, CY008430, CY008438, CY008446, CY008454, CY008462,
CY008470, CY008478, CY008486, CY008494, CY008502, CY008510, CY008518, CY008526,
CY008534, CY008542, CY008550, CY008558, CY008566, CY008574, CY008582, CY008590,
CY008598, CY008606, CY008614, CY008622, CY008630, CY008638, CY008646, CY008654,
CY008662, CY008670, CY008678, CY008686, CY008694, CY008702, CY008710, CY008718,
CY008726, CY008734, CY008742, CY008750, CY008758, CY008766, CY008774, CY008782,
CY008790, CY008798, CY008806, CY008814, CY008822, CY008830, CY008838, CY008846,
CY008854, CY008862, CY008870, CY008878, CY008886, CY008894, CY008902, CY008910,
CY008918, CY008926, CY008934, CY008942, CY008950, CY008958, CY008966, CY008974,
CY008982, CY008990, CY008998, CY009006, CY009014, CY009022, CY009030, CY009038,
CY009046, CY009054, CY009062, CY009070, CY009078, CY009086, CY009094, CY009102,
CY009110, CY009118, CY009126, CY009134, CY009142, CY009150, CY009158, CY009166,
CY009174, CY009182, CY009190, CY009198, CY009206, CY009214, CY009222, CY009230,
CY009238, CY009246, CY009254, CY009262, CY009270, CY009278, CY009286, CY009294,
CY009318, CY009326, CY009334, CY009342, CY009350, CY009358, CY009366, CY009390,
CY009398, CY009406, CY009414, CY009422, CY009430, CY009438, CY009446, CY009454,
CY009462, CY009470, CY009478, CY009486, CY009494, CY009502, CY009510, CY009518,
CY009526, CY009534, CY009542, CY009550, CY009558, CY009566, CY009574, CY009582,
CY009590, CY009598, CY009606, CY009614, CY009622, CY009638, CY009646, CY009654,
CY009662, CY009670, CY009678, CY009686, CY009694, CY009702, CY009710, CY009718,
CY009726, CY009734, CY009742, CY009750, CY009758, CY009766, CY009774, CY009782,
CY009790, CY009798, CY009806, CY009814, CY009822, CY009830, CY009838, CY009846,
CY009854, CY009862, CY009870, CY009878, CY009886, CY009902, CY009910, CY009926,
CY009934, CY009942, CY009950, CY009958, CY009966, CY009974, CY009982, CY009990,
CY009998, CY010006, CY010014, CY010022, CY010030, CY010038, CY010046, CY010054,
CY010062, CY010070, CY010078, CY010086, CY010094, CY010102, CY010110, CY010118,
CY010126, CY010134, CY010142, CY010150, CY010158, CY010166, CY010174, CY010182,
CY010190, CY010198, CY010206, CY010214, CY010222, CY010230, CY010238, CY010246,
CY010254, CY010262, CY010270, CY010278, CY010286, CY010294, CY010302, CY010310,
CY010318, CY010326, CY010334, CY010342, CY010350, CY010358, CY010366, CY010374,
CY010382, CY010390, CY010398, CY010406, CY010414, CY010422, CY010430, CY010438,
CY010446, CY010454, CY010462, CY010470, CY010478, CY010486, CY010494, CY010502,
CY010510, CY010518, CY010526, CY010534, CY010542, CY010550, CY010558, CY010590,
CY010598, CY010606, CY010614, CY010622, CY010630, CY010638, CY010646, CY010654,
CY010662, CY010670, CY010678, CY010686, CY010694, CY010702, CY010710, CY010718,
CY010726, CY010734, CY010742, CY010750, CY010758, CY010766, CY010774, CY010782,
CY010790, CY010798, CY010806, CY010814, CY010822, CY010830, CY010838, CY010846,
CY010854, CY010862, CY010870, CY010878, CY010886, CY010894, CY010902, CY010910,
CY010918, CY010926, CY010934, CY010942, CY010950, CY010958, CY010966, CY010974,
CY010982, CY010990, CY010998, CY011006, CY011014, CY011022, CY011066, CY011074,
CY011082, CY011090, CY011122, CY011130, CY011138, CY011146, CY011154, CY011162,
CY011170, CY011178, CY011186, CY011194, CY011202, CY011210, CY011218, CY011226,
CY011234, CY011242, CY011258, CY011266, CY011274, CY011282, CY011290, CY011298,
CY011306, CY011314, CY011322, CY011330, CY011338, CY011346, CY011354, CY011362,
CY011370, CY011378, CY011386, CY011394, CY011402, CY011410, CY011418, CY011426,
CY011434, CY011442, CY011450, CY011458, CY011466, CY011474, CY011482, CY011490,
CY011498, CY011506, CY011514, CY011522, CY011530, CY011538, CY011546, CY011554,
CY011562, CY011570, CY011578, CY011586, CY011594, CY011602, CY011610, CY011618,
CY011626, CY011634, CY011642, CY011650, CY011658, CY011666, CY011674, CY011682,
CY011690, CY011698, CY011706, CY011714, CY011722, CY011730, CY011738, CY011746,
CY011754, CY011762, CY011770, CY011778, CY011786, CY011794, CY011802, CY011810,
CY011818, CY011826, CY011834, CY011842, CY011850, CY011858, CY011866, CY011874,
CY011882, CY011890, CY011898, CY011906, CY011914, CY011922, CY011930, CY011938,
CY011946, CY011954, CY011962, CY011970, CY011978, CY011986, CY011994, CY012002,
CY012010, CY012018, CY012026, CY012034, CY012042, CY012050, CY012058, CY012066,
CY012074, CY012082, CY012090, CY012098, CY012106, CY012114, CY012122, CY012130,
CY012138, CY012146, CY012154, CY012162, CY012170, CY012178, CY012186, CY012194,
CY012202, CY012210, CY012218, CY012226, CY012234, CY012242, CY012250, CY012258,
CY012266, CY012274, CY012282, CY012290, CY012298, CY012306, CY012314, CY012322,
CY012330, CY012338, CY012346, CY012354, CY012362, CY012370, CY012378, CY012386,
CY012394, CY012402, CY012410, CY012418, CY012426, CY012434, CY012442, CY012450,
CY012458, CY012466, CY012474, CY012482, CY012490, CY012498, CY012506, CY012514,
CY012522, CY012530, CY012538, CY012546, CY012554, CY012562, CY012570, CY012578,
CY012586, CY012594, CY012602, CY012610, CY012618, CY012626, CY012634, CY012642,
CY012650, CY012658, CY012666, CY012674, CY012682, CY012690, CY012698, CY012706,
CY012714, CY012722, CY012730, CY012738, CY012746, CY012754, CY012762, CY012770,
CY012778, CY012786, CY012794, CY012850, CY012858, CY012866, CY012874, CY012882,
CY012890, CY012898, CY012906, CY012914, CY012922, CY012930, CY012938, CY012946,
CY012954, CY012962, CY012970, CY012978, CY012986, CY012994, CY013002, CY013010,
CY013018, CY013026, CY013034, CY013042, CY013050, CY013058, CY013066, CY013074,
CY013082, CY013090, CY013098, CY013106, CY013114, CY013122, CY013130, CY013138,
CY013146, CY013154, CY013162, CY013170, CY013178, CY013186, CY013194, CY013202,
CY013210, CY013218, CY013226, CY013234, CY013242, CY013273, CY013281, CY013289,
CY013297, CY013305, CY013313, CY013321, CY013329, CY013337, CY013345, CY013353,
CY013361, CY013369, CY013377, CY013385, CY013391, CY013399, CY013407, CY013415,
CY013423, CY013431, CY013439, CY013447, CY013455, CY013463, CY013471, CY013479,
CY013487, CY013495, CY013503, CY013511, CY013519, CY013527, CY013535, CY013543,
CY013551, CY013559, CY013567, CY013575, CY013583, CY013591, CY013599, CY013607,
CY013615, CY013623, CY013631, CY013639, CY013647, CY013655, CY013663, CY013671,
CY013679, CY013687, CY013695, CY013703, CY013711, CY013719, CY013727, CY013735,
CY013743, CY013751, CY013759, CY013767, CY013775, CY013783, CY013791, CY013799,
CY013807, CY013815, CY013823, CY013831, CY013839, CY013847, CY013855, CY013873,
CY013881, CY013889, CY013897, CY013905, CY013913, CY013921, CY013929, CY013937,
CY013945, CY013953, CY013961, CY013969, CY013977, CY013985, CY013993, CY014001,
CY014009, CY014017, CY014025, CY014033, CY014041, CY014049, CY014057, CY014065,
CY014073, CY014081, CY014089, CY014097, CY014105, CY014113, CY014121, CY014129,
CY014137, CY014145, CY014153, CY014161, CY014179, CY014227, CY014228, CY014229,
CY014230, CY014231, CY014232, CY014233, CY014235, CY014236, CY014237, CY014238,
CY014239, CY014240, CY014274, CY014282, CY014290, CY014298, CY014305, CY014313,
CY014370, CY014378, CY014386, CY014398, CY014406, CY014414, CY014422, CY014430,
CY014438, CY014446, CY014454, CY014462, CY014470, CY014486, CY014494, CY014502,
CY014507, CY014515, CY014531, CY014539, CY014545, CY014978, CY015008, CY015510,
CY015518, CY015526, CY015534, CY015542, CY015550, CY015558, CY015566, CY015574,
CY015582, CY015590, CY015598, CY015606, CY015614, CY015622, CY015630, CY015638,
CY015646, CY015654, CY015662, CY015670, CY015678, CY015686, CY015694, CY015702,
CY015710, CY015718, CY015726, CY015734, CY015742, CY015750, CY015758, CY015766,
CY015774, CY015782, CY015790, CY015798, CY015806, CY015814, CY015822, CY015830,
CY015838, CY015846, CY015854, CY015862, CY015870, CY015878, CY015886, CY015894,
CY015902, CY015910, CY015918, CY015926, CY015934, CY015942, CY015950, CY015958,
CY015966, CY015974, CY015982, CY015990, CY015998, CY016006, CY016014, CY016022,
CY016030, CY016038, CY016046, CY016054, CY016062, CY016070, CY016078, CY016086,
CY016094, CY016102, CY016110, CY016118, CY016198, CY016206, CY016214, CY016230,
CY016238, CY016246, CY016254, CY016262, CY016270, CY016351, CY016353, CY016356,
CY016405, CY016429, CY016437, CY016445, CY016453, CY016461, CY016469, CY016477,
CY016485, CY016493, CY016501, CY016509, CY016517, CY016525, CY016533, CY016541,
CY016549, CY016557, CY016565, CY016573, CY016581, CY016589, CY016597, CY016605,
CY016629, CY016637, CY016645, CY016653, CY016661, CY016669, CY016677, CY016685,
CY016693, CY016701, CY016709, CY016717, CY016725, CY016733, CY016741, CY016749,
CY016757, CY016765, CY016773, CY016965, CY016973, CY016981, CY016989, CY016997,
CY017005, CY017013, CY017021, CY017085, CY017093, CY017101, CY017109, CY017117,
CY017125, CY017133, CY017141, CY017149, CY017157, CY017165, CY017173, CY017197,
CY017205, CY017213, CY017221, CY017229, CY017237, CY017245, CY017253, CY017261,
CY017269, CY017285, CY017293, CY017301, CY017309, CY017317, CY017325, CY017333,
CY017341, CY017349, CY017357, CY017365, CY017373, CY017381, CY017389, CY017397,
CY017421, CY017429, CY017437, CY017445, CY017453, CY017461, CY017469, CY017477,
CY017485, CY017493, CY017501, CY017509, CY017517, CY017525, CY017533, CY017541,
CY017549, CY017557, CY017565, CY017573, CY017581, CY017589, CY017597, CY017605,
CY017613, CY017621, CY017629, CY017640, CY017648, CY017656, CY017664, CY017672,
CY017680, CY017690, D10164, D21186, D31944, D31945, D31948, D31950, DQ059384,
DQ085795, DQ085796, DQ085797, DQ085798, DQ085799, DQ085800, DQ090706, DQ090707,
DQ090708, DQ090709, DQ090710, DQ091199, DQ094286, DQ094290, DQ094291, DQ094292,
DQ226128, DQ227437, DQ227439, DQ227440, DQ227441, DQ227442, DQ227443, DQ227444,
DQ227445, DQ227446, DQ227447, DQ227448, DQ249252, DQ249253, DQ249254, DQ249255,
DQ249256, DQ249257, DQ249258, DQ250159, DQ250160, DQ250161, DQ250162, DQ250163,
DQ250164, DQ250165, DQ345546, DQ360836, DQ372593, DQ415338, DQ415339, DQ415340,
DQ415341, DQ415342, DQ415343, DQ415344, DQ415345, DQ415346, DQ415347, DQ415348,
DQ469960, DQ487331, DQ487337, DQ493068, DQ493069, DQ493071, DQ493072, DQ493073,
DQ493074, DQ493075, DQ493076, DQ493077, DQ493078, DQ508827, DQ508835, DQ508843,
DQ508851, DQ508859, DQ508867, DQ508875, DQ508883, DQ508891, DQ508899, DQ508907,
DQ508931, DQ535726, DQ643810, DQ885611, DQ885613, DQ885615, DQ885617, DQ885620,
DQ986135, EF101743, EF101756, ISD3BF2A5CF, ISD3BF2A6C1, ISD3BF2A6F6,
ISD3BF2A72F, ISD3BF2A773, ISD3BF2A798, ISD3BF2A7C9, ISD3BF2A7F9,
ISD3BF2A81C, ISDN110641, ISDN110642, ISDN110643, ISDN110644, ISDN110661,
ISDN110662, ISDN110663, ISDN110664, ISDN110665, ISDN110666, ISDN110667,
ISDN110668, ISDN110669, ISDN110670, ISDN110893, ISDN110894, ISDN110920,
ISDN110939, ISDN117767, ISDN117782, ISDN117783, ISDN117792, ISDN119679,
ISDN119865, ISDN122818, ISDN12305, ISDN12306, ISDN12307, ISDN12308, ISDN12309,
ISDN12310, ISDN12311, ISDN12312, ISDN12313, ISDN12314, ISDN12315, ISDN12316,
ISDN12317, ISDN12318, ISDN12319, ISDN12320, ISDN12321, ISDN12324, ISDN12325,
ISDN12326, ISDN12327, ISDN12328, ISDN12329, ISDN12330, ISDN12332, ISDN12333,
ISDN12334, ISDN12335, ISDN12336, ISDN12337, ISDN125784, ISDN125875, ISDN126657,
ISDN126658, ISDN126665, ISDN126667, ISDN126669, ISDN127328, ISDN127338,
ISDN128027, ISDN12936, ISDN129528, ISDN129584, ISDN130367, ISDN130375,
ISDN130914, ISDN131234, ISDN131235, ISDN132196, ISDN132197, ISDN132198,
ISDN132199, ISDN13223, ISDN13224, ISDN13225, ISDN13226, ISDN132408, ISDN132416,
ISDN133097, ISDN133104, ISDN133317, ISDN133324, ISDN133358, ISDN133366,
ISDN13424, ISDN136490, ISDN137800, ISDN137801, ISDN137802, ISDN137803,
ISDN137804, ISDN137805, ISDN137806, ISDN137807, ISDN137808, ISDN137809,
ISDN137810, ISDN137811, ISDN137812, ISDN137813, ISDN138750, ISDN138766,
ISDN138774, ISDN140066, ISDN140067, ISDN140068, ISDN140069, ISDN140070,
ISDN140071, ISDN140072, ISDN140073, ISDN140812, ISDN140820, ISDN140837,
ISDN140845, ISDN140853, ISDN140861, ISDN140868, ISDN140900, ISDN140902,
ISDN140928, ISDN181367, ISDN183299, ISDN183307, ISDN183315, ISDN183703,
ISDN183711, ISDN183719, ISDN183727, ISDN183735, ISDN183743, ISDN183751,
ISDN183759, ISDN183767, ISDN183775, ISDN185497, ISDN186518, ISDN186519,
ISDN186520, ISDN186521, ISDN186523, ISDN186524, ISDN186525, ISDN186526,
ISDN186527, ISDN186528, ISDN186529, ISDN186530, ISDN186532, ISDN186533,
ISDN186534, ISDN186535, ISDN186537, ISDN186538, ISDN186539, ISDN186540,
ISDN186541, ISDN186545, ISDN186546, ISDN187423, ISDN187431, ISDN187447,
ISDN20056, ISDN204138, ISDN204146, ISDN204154, ISDN207100, ISDN207108,
ISDN214633, ISDN214635, ISDN214637, ISDN214639, ISDN214641, ISDN214643,
ISDN214646, ISDN214649, ISDN214652, ISDN214655, ISDN214658, ISDN214661,
ISDN214666, ISDN214669, ISDN214672, ISDN214675, ISDN214678, ISDN214681,
ISDN214684, ISDN214687, ISDN214690, ISDN214693, ISDN219942, ISDN219945,
ISDN219948, ISDN219951, ISDN219954, ISDN33627, ISDN38703, ISDN38704, ISDN48789,
ISDN48790, ISDN48791, ISDN48792, ISDN48793, ISDN68678, ISDN68692, ISDN68693,
ISDN68694, ISDN68695, ISDN68696, ISDN68697, ISDN68698, ISDN68699, ISDN69440,
ISDN69610, J02136, J02156, J02168, J02173, J02177, K01150, K01393, K02018, L25815,
L25816, L25817, L37329, L37330, L37331, M11205, M18647, M18648, M18649, M18650,
M18651, M27970, M38309, M38335, NC_002018, U42630, U42631, U42632, U42633, U42634,
U42635, U42636, U42637, U42770, U42771, U42772, U42773, U42774, U42775, U42776,
U42777, U42778, U42779, U42780, U43417, U43418, U43419, U43420, U43421, U43422,
U43423, U43424, U43425, U43426, U43427, U51245, US1246, U51247, U53166, U71140,
U71141, U71142, U71143, X15281, Y14193, Y14194
7: GenBank and LANL accession numbers for MP sequences (segment 7) used in this
analysis.
AB036778, AB126629, AB126637, AB212057, AB262466, AF036358, AF038271, AF038272,
AF038273, AF038274, AF046090, AF084282, AF084284, AF115286, AF231358, AF231359,
AF255363, AF255364, AF255365, AF255366, AF255367, AF255368, AF255369, AF255370,
AF255371, AF255372, AF255373, AF255374, AF258522, AF258523, AF342818, AF348188,
AF348189, AF348190, AF348191, AF348192, AF348193, AF348194, AF348195, AF348196,
AF348197, AF348912, AF348913, AF386765, AF386766, AF386767, AF386768, AF386769,
AF386770, AF386771, AF386772, AF389121, AF398876, AF401293, AJ278646, AJ278647,
AJ278648, AJ293925, AJ298947, AJ298948, AJ458305, AJ458306, AJ458307, AJ458308,
AJ458339, AY043025, AY130766, AY210026, AY210027, AY210028, AY210029, AY210030,
AY210031, AY210032, AY210033, AY210034, AY210035, AY210036, AY210037, AY210038,
AY210039, AY210040, AY210041, AY210042, AY210043, AY210044, AY210045, AY210046,
AY210047, AY210048, AY210049, AY210050, AY210051, AY210052, AY210053, AY210054,
AY210055, AY210056, AY210057, AY210058, AY210059, AY210060, AY210061, AY210062,
AY210063, AY210064, AY210065, AY210239, AY210240, AY210241, AY210242, AY210243,
AY210244, AY210245, AY210246, AY210247, AY210248, AY210249, AY210250, AY210251,
AY210252, AY210253, AY210254, AY210255, AY210256, AY210257, AY210258, AY210259,
AY210260, AY210261, AY210262, AY210263, AY210264, AY210265, AY210266, AY210267,
AY210268, AY210269, AY210270, AY575893, AY575894, AY651387, AY651388, AY651389,
AY651390, AY818144, AY947475, CY000002, CY000010, CY000018, CY000026, CY000034,
CY000042, CY000050, CY000058, CY000066, CY000074, CY000082, CY000090, CY000098,
CY000106, CY000114, CY000122, CY000130, CY000138, CY000146, CY000154, CY000162,
CY000170, CY000178, CY000186, CY000194, CY000202, CY000210, CY000218, CY000226,
CY000234, CY000242, CY000250, CY000258, CY000266, CY000274, CY000282, CY000290,
CY000298, CY000306, CY000314, CY000322, CY000330, CY000338, CY000346, CY000354,
CY000362, CY000370, CY000378, CY000386, CY000394, CY000402, CY000410, CY000418,
CY000426, CY000434, CY000442, CY000450, CY000458, CY000466, CY000474, CY000482,
CY000490, CY000498, CY000506, CY000514, CY000522, CY000530, CY000538, CY000546,
CY000554, CY000562, CY000570, CY000577, CY000586, CY000594, CY000602, CY000610,
CY000618, CY000626, CY000634, CY000642, CY000650, CY000658, CY000666, CY000674,
CY000682, CY000690, CY000698, CY000706, CY000714, CY000722, CY000730, CY000738,
CY000746, CY000754, CY000762, CY000770, CY000778, CY000786, CY000794, CY000802,
CY000810, CY000818, CY000826, CY000834, CY000842, CY000850, CY000858, CY000866,
CY000874, CY000882, CY000890, CY000902, CY000910, CY000918, CY000926, CY000934,
CY000942, CY000950, CY000958, CY000966, CY000974, CY000982, CY000990, CY000998,
CY001006, CY001014, CY001022, CY001030, CY001038, CY001046, CY001054, CY001062,
CY001065, CY001073, CY001081, CY001089, CY001097, CY001105, CY001113, CY001121,
CY001129, CY001137, CY001145, CY001153, CY001161, CY001169, CY001177, CY001185,
CY001198, CY001206, CY001214, CY001222, CY001230, CY001238, CY001246, CY001254,
CY001262, CY001270, CY001278, CY001286, CY001294, CY001302, CY001310, CY001318,
CY001326, CY001334, CY001342, CY001350, CY001358, CY001366, CY001374, CY001382,
CY001398, CY001406, CY001414, CY001422, CY001430, CY001438, CY001446, CY001454,
CY001462, CY001470, CY001478, CY001486, CY001494, CY001505, CY001513, CY001521,
CY001529, CY001537, CY001545, CY001553, CY001561, CY001569, CY001577, CY001585,
CY001593, CY001601, CY001609, CY001617, CY001625, CY001633, CY001641, CY001649,
CY001657, CY001665, CY001673, CY001681, CY001689, CY001697, CY001705, CY001713,
CY001721, CY001729, CY001737, CY001745, CY001753, CY001761, CY001769, CY001777,
CY001785, CY001793, CY001801, CY001809, CY001817, CY001825, CY001833, CY001841,
CY001849, CY001857, CY001865, CY001873, CY001881, CY001889, CY001897, CY001905,
CY001913, CY001921, CY001929, CY001937, CY001945, CY001953, CY001961, CY001969,
CY001977, CY001985, CY001993, CY002001, CY002009, CY002017, CY002025, CY002033,
CY002041, CY002049, CY002057, CY002065, CY002073, CY002081, CY002089, CY002097,
CY002105, CY002113, CY002121, CY002129, CY002137, CY002145, CY002153, CY002161,
CY002169, CY002177, CY002185, CY002193, CY002201, CY002209, CY002217, CY002225,
CY002233, CY002241, CY002249, CY002257, CY002265, CY002273, CY002281, CY002289,
CY002297, CY002305, CY002313, CY002329, CY002337, CY002345, CY002353, CY002361,
CY002369, CY002377, CY002385, CY002393, CY002401, CY002409, CY002417, CY002425,
CY002433, CY002441, CY002449, CY002457, CY002465, CY002473, CY002481, CY002489,
CY002497, CY002505, CY002513, CY002521, CY002529, CY002537, CY002545, CY002553,
CY002561, CY002569, CY002577, CY002585, CY002593, CY002601, CY002609, CY002617,
CY002625, CY002633, CY002641, CY002649, CY002657, CY002665, CY002673, CY002681,
CY002689, CY002697, CY002705, CY002713, CY002721, CY002729, CY002737, CY002745,
CY002753, CY002761, CY002769, CY002777, CY002785, CY002793, CY002801, CY002809,
CY002817, CY002907, CY002915, CY002923, CY002931, CY002939, CY002947, CY002955,
CY002963, CY002970, CY002977, CY002985, CY002993, CY003001, CY003009, CY003017,
CY003025, CY003033, CY003041, CY003049, CY003057, CY003065, CY003073, CY003081,
CY003089, CY003097, CY003105, CY003113, CY003121, CY003127, CY003137, CY003145,
CY003153, CY003161, CY003169, CY003177, CY003185, CY003193, CY003201, CY003209,
CY003217, CY003225, CY003233, CY003241, CY003249, CY003257, CY003265, CY003273,
CY003281, CY003289, CY003297, CY003305, CY003313, CY003321, CY003329, CY003337,
CY003345, CY003353, CY003369, CY003377, CY003385, CY003393, CY003401, CY003409,
CY003417, CY003425, CY003433, CY003441, CY003449, CY003457, CY003465, CY003473,
CY003481, CY003489, CY003497, CY003505, CY003513, CY003521, CY003529, CY003537,
CY003545, CY003553, CY003561, CY003569, CY003577, CY003585, CY003593, CY003601,
CY003609, CY003617, CY003625, CY003633, CY003641, CY003649, CY003657, CY003665,
CY003673, CY003681, CY003689, CY003697, CY003705, CY003713, CY003721, CY003729,
CY003737, CY003745, CY003753, CY003762, CY003770, CY003778, CY003786, CY003794,
CY003802, CY003810, CY003818, CY003826, CY003834, CY006045, CY006053, CY006061,
CY006069, CY006077, CY006085, CY006093, CY006100, CY006108, CY006116, CY006124,
CY006132, CY006140, CY006148, CY006156, CY006164, CY006172, CY006180, CY006188,
CY006196, CY006204, CY006212, CY006220, CY006228, CY006236, CY006244, CY006252,
CY006260, CY006268, CY006276, CY006284, CY006292, CY006300, CY006308, CY006316,
CY006324, CY006332, CY006340, CY006348, CY006356, CY006364, CY006372, CY006380,
CY006388, CY006396, CY006404, CY006412, CY006420, CY006428, CY006436, CY006444,
CY006452, CY006460, CY006468, CY006476, CY006484, CY006492, CY006500, CY006508,
CY006516, CY006524, CY006532, CY006540, CY006548, CY006556, CY006564, CY006572,
CY006580, CY006588, CY006596, CY006604, CY006612, CY006620, CY006628, CY006636,
CY006660, CY006668, CY006676, CY006684, CY006692, CY006700, CY006708, CY006716,
CY006724, CY006732, CY006740, CY006748, CY006756, CY006764, CY006772, CY006780,
CY006788, CY006796, CY006804, CY006812, CY006820, CY006828, CY006836, CY006844,
CY006852, CY006860, CY006868, CY006876, CY006884, CY006892, CY006900, CY006908,
CY006916, CY006924, CY006932, CY006940, CY006948, CY006956, CY006964, CY006972,
CY006980, CY006988, CY006996, CY007004, CY007012, CY007020, CY007028, CY007036,
CY007044, CY007052, CY007060, CY007068, CY007076, CY007084, CY007092, CY007100,
CY007108, CY007116, CY007124, CY007132, CY007140, CY007148, CY007156, CY007164,
CY007172, CY007180, CY007188, CY007196, CY007204, CY007212, CY007220, CY007228,
CY007236, CY007244, CY007252, CY007260, CY007268, CY007276, CY007284, CY007292,
CY007300, CY007308, CY007316, CY007324, CY007332, CY007340, CY007348, CY007356,
CY007364, CY007372, CY007380, CY007388, CY007396, CY007404, CY007412, CY007420,
CY007428, CY007436, CY007444, CY007452, CY007460, CY007468, CY007476, CY007484,
CY007492, CY007500, CY007508, CY007516, CY007524, CY007532, CY007540, CY007548,
CY007556, CY007564, CY007572, CY007580, CY007588, CY007596, CY007604, CY007612,
CY007620, CY007628, CY007636, CY007644, CY007652, CY007660, CY007668, CY007676,
CY007684, CY007692, CY007700, CY007708, CY007716, CY007724, CY007732, CY007740,
CY007748, CY007756, CY007764, CY007772, CY007780, CY007788, CY007796, CY007804,
CY007812, CY007820, CY007828, CY007836, CY007844, CY007852, CY007860, CY007868,
CY007876, CY007884, CY007892, CY007900, CY007908, CY007916, CY007924, CY007932,
CY007940, CY007948, CY007956, CY007964, CY007972, CY007980, CY007988, CY007996,
CY008004, CY008012, CY008020, CY008028, CY008036, CY008044, CY008052, CY008060,
CY008068, CY008076, CY008084, CY008092, CY008100, CY008108, CY008116, CY008124,
CY008132, CY008140, CY008149, CY008157, CY008165, CY008173, CY008181, CY008189,
CY008197, CY008205, CY008213, CY008221, CY008229, CY008237, CY008245, CY008253,
CY008261, CY008269, CY008277, CY008285, CY008293, CY008301, CY008309, CY008317,
CY008325, CY008333, CY008341, CY008349, CY008357, CY008365, CY008373, CY008381,
CY008389, CY008397, CY008405, CY008413, CY008421, CY008429, CY008437, CY008445,
CY008453, CY008461, CY008469, CY008477, CY008485, CY008493, CY008501, CY008509,
CY008517, CY008525, CY008533, CY008541, CY008549, CY008557, CY008565, CY008573,
CY008581, CY008589, CY008597, CY008605, CY008613, CY008621, CY008629, CY008637,
CY008645, CY008653, CY008661, CY008669, CY008677, CY008685, CY008693, CY008701,
CY008709, CY008717, CY008725, CY008733, CY008741, CY008749, CY008757, CY008765,
CY008773, CY008781, CY008789, CY008797, CY008805, CY008813, CY008821, CY008829,
CY008837, CY008845, CY008853, CY008861, CY008869, CY008877, CY008885, CY008893,
CY008901, CY008909, CY008917, CY008925, CY008933, CY008941, CY008949, CY008957,
CY008965, CY008973, CY008981, CY008989, CY008997, CY009005, CY009013, CY009021,
CY009029, CY009037, CY009045, CY009053, CY009061, CY009069, CY009077, CY009085,
CY009093, CY009101, CY009109, CY009117, CY009125, CY009133, CY009141, CY009149,
CY009157, CY009165, CY009173, CY009181, CY009189, CY009197, CY009205, CY009213,
CY009221, CY009229, CY009237, CY009245, CY009253, CY009261, CY009269, CY009277,
CY009285, CY009293, CY009317, CY009325, CY009333, CY009341, CY009349, CY009357,
CY009365, CY009389, CY009397, CY009405, CY009413, CY009421, CY009429, CY009437,
CY009445, CY009453, CY009461, CY009469, CY009477, CY009485, CY009493, CY009501,
CY009509, CY009517, CY009525, CY009533, CY009541, CY009549, CY009557, CY009565,
CY009573, CY009581, CY009589, CY009597, CY009605, CY009613, CY009621, CY009637,
CY009645, CY009653, CY009661, CY009669, CY009677, CY009685, CY009693, CY009701,
CY009709, CY009717, CY009725, CY009733, CY009741, CY009749, CY009757, CY009765,
CY009773, CY009781, CY009789, CY009797, CY009805, CY009813, CY009821, CY009829,
CY009837, CY009845, CY009853, CY009861, CY009869, CY009877, CY009885, CY009901,
CY009909, CY009925, CY009933, CY009941, CY009949, CY009957, CY009965, CY009973,
CY009981, CY009989, CY009997, CY010005, CY010013, CY010021, CY010029, CY010037,
CY010045, CY010053, CY010061, CY010069, CY010077, CY010085, CY010093, CY010101,
CY010109, CY010117, CY010125, CY010133, CY010141, CY010149, CY010157, CY010165,
CY010173, CY010181, CY010189, CY010197, CY010205, CY010213, CY010221, CY010229,
CY010237, CY010245, CY010253, CY010261, CY010269, CY010277, CY010285, CY010293,
CY010301, CY010309, CY010317, CY010325, CY010333, CY010341, CY010349, CY010357,
CY010365, CY010373, CY010381, CY010389, CY010397, CY010405, CY010413, CY010421,
CY010429, CY010437, CY010445, CY010453, CY010461, CY010469, CY010477, CY010485,
CY010493, CY010501, CY010509, CY010517, CY010525, CY010533, CY010541, CY010549,
CY010557, CY010589, CY010597, CY010605, CY010613, CY010621, CY010629, CY010637,
CY010645, CY010653, CY010661, CY010669, CY010677, CY010685, CY010693, CY010701,
CY010709, CY010717, CY010725, CY010733, CY010741, CY010749, CY010757, CY010765,
CY010773, CY010781, CY010789, CY010797, CY010805, CY010813, CY010821, CY010829,
CY010837, CY010845, CY010853, CY010861, CY010869, CY010877, CY010885, CY010893,
CY010901, CY010909, CY010917, CY010925, CY010933, CY010941, CY010949, CY010957,
CY010965, CY010973, CY010981, CY010989, CY010997, CY011005, CY011013, CY011021,
CY011065, CY011073, CY011081, CY011089, CY011121, CY011129, CY011137, CY011145,
CY011153, CY011161, CY011169, CY011177, CY011185, CY011193, CY011201, CY011209,
CY011217, CY011225, CY011233, CY011241, CY011257, CY011265, CY011273, CY011281,
CY011289, CY011297, CY011305, CY011313, CY011321, CY011329, CY011337, CY011345,
CY011353, CY011361, CY011369, CY011377, CY011385, CY011393, CY011401, CY011409,
CY011417, CY011425, CY011433, CY011441, CY011449, CY011457, CY011465, CY011473,
CY011481, CY011489, CY011497, CY011505, CY011513, CY011521, CY011529, CY011537,
CY011545, CY011553, CY011561, CY011569, CY011577, CY011585, CY011593, CY011601,
CY011609, CY011617, CY011625, CY011633, CY011641, CY011649, CY011657, CY011665,
CY011673, CY011681, CY011689, CY011697, CY011705, CY011713, CY011721, CY011729,
CY011737, CY011745, CY011753, CY011761, CY011769, CY011777, CY011785, CY011793,
CY011801, CY011809, CY011817, CY011825, CY011833, CY011841, CY011849, CY011857,
CY011865, CY011873, CY011881, CY011889, CY011897, CY011905, CY011913, CY011921,
CY011929, CY011937, CY011945, CY011953, CY011961, CY011969, CY011977, CY011985,
CY011993, CY012001, CY012009, CY012017, CY012025, CY012033, CY012041, CY012049,
CY012057, CY012065, CY012073, CY012081, CY012089, CY012097, CY012105, CY012113,
CY012121, CY012129, CY012137, CY012145, CY012153, CY012161, CY012169, CY012177,
CY012185, CY012193, CY012201, CY012209, CY012217, CY012225, CY012233, CY012241,
CY012249, CY012257, CY012265, CY012273, CY012281, CY012289, CY012297, CY012305,
CY012313, CY012321, CY012329, CY012337, CY012345, CY012353, CY012361, CY012369,
CY012377, CY012385, CY012393, CY012401, CY012409, CY012417, CY012425, CY012433,
CY012441, CY012449, CY012457, CY012465, CY012473, CY012481, CY012489, CY012497,
CY012505, CY012513, CY012521, CY012529, CY012537, CY012545, CY012553, CY012561,
CY012569, CY012577, CY012585, CY012593, CY012601, CY012609, CY012617, CY012625,
CY012633, CY012641, CY012649, CY012657, CY012665, CY012673, CY012681, CY012689,
CY012697, CY012705, CY012713, CY012721, CY012729, CY012737, CY012745, CY012753,
CY012761, CY012769, CY012777, CY012785, CY012793, CY012849, CY012857, CY012865,
CY012873, CY012881, CY012889, CY012897, CY012905, CY012913, CY012921, CY012929,
CY012937, CY012945, CY012953, CY012961, CY012969, CY012977, CY012985, CY012993,
CY013001, CY013009, CY013017, CY013025, CY013033, CY013041, CY013049, CY013057,
CY013065, CY013073, CY013081, CY013089, CY013097, CY013105, CY013113, CY013121,
CY013129, CY013137, CY013145, CY013153, CY013161, CY013169, CY013177, CY013185,
CY013193, CY013201, CY013209, CY013217, CY013225, CY013233, CY013241, CY013272,
CY013280, CY013288, CY013296, CY013304, CY013312, CY013320, CY013328, CY013336,
CY013344, CY013352, CY013360, CY013368, CY013376, CY013384, CY013390, CY013398,
CY013406, CY013414, CY013422, CY013430, CY013438, CY013446, CY013454, CY013462,
CY013470, CY013478, CY013486, CY013494, CY013502, CY013510, CY013518, CY013526,
CY013534, CY013542, CY013550, CY013558, CY013566, CY013574, CY013582, CY013590,
CY013598, CY013606, CY013614, CY013622, CY013630, CY013638, CY013646, CY013654,
CY013662, CY013670, CY013678, CY013686, CY013694, CY013702, CY013710, CY013718,
CY013726, CY013734, CY013742, CY013750, CY013758, CY013766, CY013774, CY013782,
CY013790, CY013798, CY013806, CY013814, CY013822, CY013830, CY013838, CY013846,
CY013854, CY013872, CY013880, CY013888, CY013896, CY013904, CY013912, CY013920,
CY013928, CY013936, CY013944, CY013952, CY013960, CY013968, CY013976, CY013984,
CY013992, CY014000, CY014008, CY014016, CY014024, CY014032, CY014040, CY014048,
CY014056, CY014064, CY014072, CY014080, CY014088, CY014096, CY014104, CY014112,
CY014120, CY014128, CY014136, CY014144, CY014152, CY014160, CY014173, CY014180,
CY014214, CY014215, CY014216, CY014217, CY014218, CY014219, CY014220, CY014222,
CY014223, CY014224, CY014225, CY014226, CY014275, CY014283, CY014291, CY014299,
CY014306, CY014314, CY014369, CY014377, CY014385, CY014397, CY014405, CY014413,
CY014421, CY014429, CY014437, CY014445, CY014453, CY014461, CY014469, CY014478,
CY014485, CY014493, CY014501, CY014506, CY014514, CY014532, CY014540, CY014546,
CY014977, CY015007, CY015509, CY015517, CY015525, CY015533, CY015541, CY015549,
CY015557, CY015565, CY015573, CY015581, CY015589, CY015597, CY015605, CY015613,
CY015621, CY015629, CY015637, CY015645, CY015653, CY015661, CY015669, CY015677,
CY015685, CY015693, CY015701, CY015709, CY015717, CY015725, CY015733, CY015741,
CY015749, CY015757, CY015765, CY015773, CY015781, CY015789, CY015797, CY015805,
CY015813, CY015821, CY015829, CY015837, CY015845, CY015853, CY015861, CY015869,
CY015877, CY015885, CY015893, CY015901, CY015909, CY015917, CY015925, CY015933,
CY015941, CY015949, CY015957, CY015965, CY015973, CY015981, CY015989, CY015997,
CY016005, CY016013, CY016021, CY016029, CY016037, CY016045, CY016053, CY016061,
CY016069, CY016077, CY016085, CY016093, CY016101, CY016109, CY016117, CY016197,
CY016205, CY016213, CY016229, CY016237, CY016245, CY016253, CY016261, CY016269,
CY016404, CY016428, CY016436, CY016444, CY016452, CY016460, CY016468, CY016476,
CY016484, CY016492, CY016500, CY016508, CY016516, CY016524, CY016532, CY016540,
CY016548, CY016556, CY016564, CY016572, CY016580, CY016588, CY016596, CY016604,
CY016628, CY016636, CY016644, CY016652, CY016660, CY016668, CY016676, CY016684,
CY016692, CY016700, CY016708, CY016716, CY016724, CY016732, CY016740, CY016748,
CY016756, CY016764, CY016772, CY016964, CY016972, CY016980, CY016988, CY016996,
CY017004, CY017012, CY017020, CY017084, CY017092, CY017100, CY017108, CY017116,
CY017124, CY017132, CY017140, CY017148, CY017156, CY017164, CY017172, CY017196,
CY017204, CY017212, CY017220, CY017228, CY017236, CY017244, CY017252, CY017260,
CY017268, CY017284, CY017292, CY017300, CY017308, CY017316, CY017324, CY017332,
CY017340, CY017348, CY017356, CY017364, CY017372, CY017380, CY017388, CY017396,
CY017420, CY017428, CY017436, CY017444, CY017452, CY017460, CY017468, CY017476,
CY017484, CY017492, CY017500, CY017508, CY017516, CY017524, CY017532, CY017540,
CY017548, CY017556, CY017564, CY017572, CY017580, CY017588, CY017596, CY017604,
CY017612, CY017620, CY017628, CY017641, CY017649, CY017657, CY017665, CY017673,
CY017681, CY017691, DQ094266, DQ094267, DQ094271, DQ094272, DQ094273, DQ094274,
DQ094275, DQ098266, DQ098267, DQ098269, DQ100422, DQ100423, DQ100424, DQ226095,
DQ249265, DQ249266, DQ249267, DQ249268, DQ299489, DQ360841, DQ372592, DQ415349,
DQ415350, DQ415351, DQ415352, DQ415353, DQ415354, DQ415355, DQ415356, DQ415357,
DQ415358, DQ415359, DQ469961, DQ487329, DQ487338, DQ492980, DQ492981, DQ492982,
DQ492983, DQ492984, DQ492985, DQ492986, DQ492987, DQ492989, DQ492990, DQ508828,
DQ508836, DQ508844, DQ508852, DQ508860, DQ508868, DQ508876, DQ508884, DQ508892,
DQ508900, DQ508908, DQ508932, DQ535725, DQ643814, DQ849002, DQ849003, DQ849004,
DQ849005, DQ849006, DQ849007, DQ849008, DQ849009, DQ849010, DQ849011, DQ849012,
DQ849013, DQ849014, DQ849015, DQ849016, DQ849017, DQ849018, DQ849019, DQ849020,
DQ849021, DQ849022, DQ849023, DQ849024, DQ986133, EF101742, EF101750,
ISDN111182, ISDN111183, ISDN111184, ISDN111185, ISDN125785, ISDN125876,
ISDN128028, ISDN129534, ISDN129585, ISDN130366, ISDN130374, ISDN132407,
ISDN132415, ISDN133183, ISDN133318, ISDN133325, ISDN133357, ISDN133365,
ISDN133372, ISDN13425, ISDN136480, ISDN136842, ISDN136850, ISDN137446,
ISDN137449, ISDN137450, ISDN137451, ISDN137452, ISDN137453, ISDN137454,
ISDN137455, ISDN137456, ISDN137457, ISDN137458, ISDN137459, ISDN137460,
ISDN138749, ISDN138765, ISDN138773, ISDN139827, ISDN139828, ISDN140087,
ISDN140088, ISDN140089, ISDN140090, ISDN140091, ISDN140092, ISDN140811,
ISDN140819, ISDN140838, ISDN140846, ISDN140854, ISDN140862, ISDN140869,
ISDN140897, ISDN140898, ISDN140899, ISDN140927, ISDN141892, ISDN141893,
ISDN181368, ISDN183298, ISDN183306, ISDN183314, ISDN183702, ISDN183710,
ISDN183718, ISDN183726, ISDN183734, ISDN183742, ISDN183750, ISDN183758,
ISDN183766, ISDN183774, ISDN183783, ISDN185499, ISDN186572, ISDN186573,
ISDN186574, ISDN186575, ISDN186586, ISDN186589, ISDN186590, ISDN187422,
ISDN187430, ISDN187438, ISDN187446, ISDN187454, ISDN204137, ISDN204145,
ISDN204153, ISDN207099, ISDN207107, ISDN209856, ISDN39957, ISDN39958, ISDN45755,
J02167, K01140, L18995, L18999, L25814, L25818, M19374, M23920, M23978, M54941,
M63515, M63516, M63521, M63531, M81570, M81576, M81582, NC_002016, U02084,
U02463, U08863, U53168, U53169, U65561, U65562, U65563, U65564, U65565, U65566,
U65567, U65568, U65569, U65570, U65571, U65572, U65573, U65574, U65575, U65576,
U65577, U65578, X08088, X08089, X08090, X08091, X08092, X08093, X53029, X59240
8: GenBank and LANL accession numbers for NS sequences (segment 8) used in this
analysis.
AB036777, AB126628, AB126636, AB212058, AB212059, AB262467, AF036360, AF038275,
AF038276, AF038277, AF038278, AF038279, AF046091, AF055422, AF055423, AF055424,
AF055425, AF084285, AF084286, AF084287, AF115288, AF256177, AF256178, AF256179,
AF256180, AF256181, AF256182, AF256183, AF256184, AF256185, AF256186, AF256187,
AF256188, AF258521, AF333238, AF342817, AF348198, AF348199, AF348200, AF348201,
AF348202, AF348203, AF348204, AF348205, AF348206, AF389122, AF398877, AJ238022,
AJ278649, AJ293941, AJ298949, AJ298950, AJ404735, AJ404736, AY043027, AY210151,
AY210152, AY210153, AY210154, AY210155, AY210156, AY210157, AY210158, AY210159,
AY210160, AY210161, AY210162, AY210163, AY210164, AY210165, AY210166, AY210167,
AY210168, AY210169, AY210170, AY210171, AY210172, AY210173, AY210174, AY210175,
AY210176, AY210177, AY210178, AY210179, AY210180, AY210181, AY210182, AY210183,
AY210184, AY210185, AY210186, AY210187, AY210188, AY210189, AY210190, AY210191,
AY210192, AY210285, AY210286, AY210287, AY210288, AY210289, AY210290, AY210291,
AY210292, AY210293, AY210294, AY210295, AY210296, AY210297, AY210298, AY210299,
AY210300, AY210301, AY210302, AY210303, AY210304, AY210305, AY210306, AY210307,
AY210308, AY210309, AY210310, AY210311, AY210312, AY210313, AY210314, AY210315,
AY210316, AY526747, AY576368, AY576369, AY651552, AY651553, AY651554, AY651555,
AY818147, CY000005, CY000013, CY000021, CY000029, CY000037, CY000045, CY000053,
CY000061, CY000069, CY000077, CY000085, CY000093, CY000101, CY000109, CY000117,
CY000125, CY000133, CY000141, CY000149, CY000157, CY000165, CY000173, CY000181,
CY000189, CY000197, CY000205, CY000213, CY000221, CY000229, CY000237, CY000245,
CY000253, CY000261, CY000269, CY000277, CY000285, CY000293, CY000301, CY000309,
CY000317, CY000325, CY000333, CY000341, CY000349, CY000357, CY000365, CY000373,
CY000381, CY000389, CY000397, CY000405, CY000413, CY000421, CY000429, CY000437,
CY000445, CY000453, CY000461, CY000469, CY000477, CY000485, CY000493, CY000501,
CY000509, CY000516, CY000525, CY000533, CY000541, CY000549, CY000557, CY000565,
CY000573, CY000580, CY000589, CY000597, CY000605, CY000613, CY000621, CY000629,
CY000637, CY000645, CY000653, CY000661, CY000669, CY000677, CY000685, CY000693,
CY000701, CY000709, CY000717, CY000725, CY000733, CY000741, CY000749, CY000757,
CY000765, CY000773, CY000781, CY000789, CY000797, CY000805, CY000813, CY000821,
CY000829, CY000837, CY000845, CY000853, CY000861, CY000869, CY000877, CY000885,
CY000893, CY000905, CY000913, CY000921, CY000929, CY000937, CY000945, CY000953,
CY000961, CY000969, CY000977, CY000985, CY000993, CY001001, CY001009, CY001017,
CY001025, CY001033, CY001041, CY001049, CY001057, CY001068, CY001076, CY001084,
CY001092, CY001100, CY001108, CY001116, CY001124, CY001132, CY001140, CY001148,
CY001156, CY001164, CY001172, CY001180, CY001188, CY001193, CY001201, CY001209,
CY001217, CY001225, CY001233, CY001241, CY001249, CY001257, CY001265, CY001273,
CY001281, CY001289, CY001297, CY001305, CY001313, CY001321, CY001329, CY001337,
CY001345, CY001353, CY001361, CY001369, CY001377, CY001385, CY001401, CY001409,
CY001417, CY001425, CY001433, CY001441, CY001449, CY001457, CY001465, CY001473,
CY001481, CY001489, CY001497, CY001508, CY001516, CY001524, CY001532, CY001540,
CY001548, CY001556, CY001564, CY001572, CY001580, CY001588, CY001596, CY001604,
CY001612, CY001620, CY001628, CY001636, CY001644, CY001652, CY001660, CY001668,
CY001676, CY001684, CY001692, CY001700, CY001708, CY001716, CY001724, CY001732,
CY001740, CY001748, CY001756, CY001764, CY001772, CY001780, CY001788, CY001796,
CY001804, CY001812, CY001820, CY001828, CY001836, CY001844, CY001852, CY001860,
CY001868, CY001876, CY001884, CY001892, CY001900, CY001908, CY001916, CY001924,
CY001932, CY001940, CY001948, CY001956, CY001964, CY001972, CY001980, CY001988,
CY001996, CY002004, CY002012, CY002020, CY002028, CY002036, CY002044, CY002052,
CY002060, CY002068, CY002076, CY002084, CY002092, CY002100, CY002108, CY002116,
CY002124, CY002132, CY002140, CY002148, CY002156, CY002164, CY002172, CY002180,
CY002188, CY002196, CY002204, CY002212, CY002220, CY002228, CY002236, CY002244,
CY002252, CY002260, CY002268, CY002276, CY002284, CY002292, CY002300, CY002308,
CY002316, CY002332, CY002340, CY002348, CY002356, CY002364, CY002372, CY002380,
CY002388, CY002396, CY002404, CY002412, CY002420, CY002428, CY002436, CY002444,
CY002452, CY002460, CY002468, CY002476, CY002484, CY002492, CY002500, CY002508,
CY002516, CY002524, CY002532, CY002540, CY002548, CY002556, CY002564, CY002572,
CY002580, CY002588, CY002596, CY002604, CY002612, CY002620, CY002628, CY002636,
CY002644, CY002652, CY002660, CY002668, CY002676, CY002684, CY002692, CY002700,
CY002708, CY002716, CY002724, CY002732, CY002740, CY002748, CY002756, CY002764,
CY002772, CY002780, CY002788, CY002796, CY002804, CY002812, CY002820, CY002910,
CY002918, CY002926, CY002934, CY002942, CY002950, CY002958, CY002966, CY002973,
CY002980, CY002988, CY002996, CY003004, CY003012, CY003020, CY003028, CY003036,
CY003044, CY003052, CY003060, CY003068, CY003076, CY003084, CY003092, CY003100,
CY003108, CY003116, CY003125, CY003131, CY003140, CY003148, CY003156, CY003164,
CY003172, CY003180, CY003188, CY003196, CY003204, CY003212, CY003220, CY003228,
CY003236, CY003244, CY003252, CY003260, CY003268, CY003276, CY003284, CY003292,
CY003300, CY003308, CY003316, CY003324, CY003332, CY003340, CY003348, CY003356,
CY003372, CY003380, CY003388, CY003396, CY003404, CY003412, CY003420, CY003428,
CY003436, CY003444, CY003452, CY003460, CY003468, CY003476, CY003484, CY003492,
CY003500, CY003508, CY003516, CY003524, CY003532, CY003540, CY003548, CY003556,
CY003564, CY003572, CY003580, CY003588, CY003596, CY003604, CY003612, CY003620,
CY003628, CY003636, CY003644, CY003652, CY003660, CY003668, CY003676, CY003684,
CY003692, CY003700, CY003708, CY003716, CY003724, CY003732, CY003740, CY003748,
CY003756, CY003765, CY003773, CY003781, CY003789, CY003797, CY003805, CY003813,
CY003821, CY003829, CY003837, CY006048, CY006056, CY006064, CY006072, CY006080,
CY006088, CY006096, CY006103, CY006111, CY006119, CY006127, CY006135, CY006143,
CY006151, CY006159, CY006167, CY006175, CY006183, CY006191, CY006199, CY006207,
CY006215, CY006223, CY006231, CY006239, CY006247, CY006255, CY006263, CY006271,
CY006279, CY006287, CY006295, CY006303, CY006311, CY006319, CY006327, CY006335,
CY006343, CY006351, CY006359, CY006367, CY006375, CY006383, CY006391, CY006399,
CY006407, CY006415, CY006423, CY006431, CY006439, CY006447, CY006455, CY006463,
CY006471, CY006479, CY006487, CY006495, CY006503, CY006511, CY006519, CY006527,
CY006535, CY006543, CY006551, CY006559, CY006567, CY006575, CY006583, CY006591,
CY006599, CY006607, CY006615, CY006623, CY006631, CY006639, CY006663, CY006671,
CY006679, CY006687, CY006695, CY006703, CY006711, CY006719, CY006727, CY006735,
CY006743, CY006751, CY006759, CY006767, CY006775, CY006783, CY006791, CY006799,
CY006807, CY006815, CY006823, CY006831, CY006839, CY006847, CY006855, CY006863,
CY006871, CY006879, CY006887, CY006895, CY006903, CY006911, CY006919, CY006927,
CY006935, CY006943, CY006951, CY006959, CY006967, CY006975, CY006983, CY006991,
CY006999, CY007007, CY007015, CY007023, CY007031, CY007039, CY007047, CY007055,
CY007063, CY007071, CY007079, CY007087, CY007095, CY007103, CY007111, CY007119,
CY007127, CY007135, CY007143, CY007151, CY007159, CY007167, CY007175, CY007183,
CY007191, CY007199, CY007207, CY007215, CY007223, CY007231, CY007239, CY007247,
CY007255, CY007263, CY007271, CY007279, CY007287, CY007295, CY007303, CY007311,
CY007319, CY007327, CY007335, CY007343, CY007351, CY007359, CY007367, CY007375,
CY007383, CY007391, CY007399, CY007407, CY007415, CY007423, CY007431, CY007439,
CY007447, CY007455, CY007463, CY007471, CY007479, CY007487, CY007495, CY007503,
CY007511, CY007519, CY007527, CY007535, CY007543, CY007551, CY007559, CY007567,
CY007575, CY007583, CY007591, CY007599, CY007607, CY007615, CY007623, CY007631,
CY007639, CY007647, CY007655, CY007663, CY007671, CY007679, CY007687, CY007695,
CY007703, CY007711, CY007719, CY007727, CY007735, CY007743, CY007751, CY007759,
CY007767, CY007775, CY007783, CY007791, CY007799, CY007807, CY007815, CY007823,
CY007831, CY007839, CY007847, CY007855, CY007863, CY007871, CY007879, CY007887,
CY007895, CY007903, CY007911, CY007919, CY007927, CY007935, CY007943, CY007951,
CY007959, CY007967, CY007975, CY007983, CY007991, CY007999, CY008007, CY008015,
CY008023, CY008031, CY008039, CY008047, CY008055, CY008063, CY008071, CY008079,
CY008087, CY008095, CY008103, CY008111, CY008119, CY008127, CY008135, CY008143,
CY008152, CY008160, CY008168, CY008176, CY008184, CY008192, CY008200, CY008208,
CY008216, CY008224, CY008232, CY008240, CY008248, CY008256, CY008264, CY008272,
CY008280, CY008288, CY008296, CY008304, CY008312, CY008320, CY008328, CY008336,
CY008344, CY008352, CY008360, CY008368, CY008376, CY008384, CY008392, CY008400,
CY008408, CY008416, CY008424, CY008432, CY008440, CY008448, CY008456, CY008464,
CY008472, CY008480, CY008488, CY008496, CY008504, CY008512, CY008520, CY008528,
CY008536, CY008544, CY008552, CY008560, CY008568, CY008576, CY008584, CY008592,
CY008600, CY008608, CY008616, CY008624, CY008632, CY008640, CY008648, CY008656,
CY008664, CY008672, CY008680, CY008688, CY008696, CY008704, CY008712, CY008720,
CY008728, CY008736, CY008744, CY008752, CY008760, CY008768, CY008776, CY008784,
CY008792, CY008800, CY008808, CY008816, CY008824, CY008832, CY008840, CY008848,
CY008856, CY008864, CY008872, CY008880, CY008888, CY008896, CY008904, CY008912,
CY008920, CY008928, CY008936, CY008944, CY008952, CY008960, CY008968, CY008976,
CY008984, CY008992, CY009000, CY009008, CY009016, CY009024, CY009032, CY009040,
CY009048, CY009056, CY009064, CY009072, CY009080, CY009088, CY009096, CY009104,
CY009112, CY009120, CY009128, CY009136, CY009144, CY009152, CY009160, CY009168,
CY009176, CY009184, CY009192, CY009200, CY009208, CY009216, CY009224, CY009232,
CY009240, CY009248, CY009256, CY009264, CY009272, CY009280, CY009288, CY009296,
CY009320, CY009328, CY009336, CY009344, CY009352, CY009360, CY009368, CY009392,
CY009400, CY009408, CY009416, CY009424, CY009432, CY009440, CY009448, CY009456,
CY009464, CY009472, CY009480, CY009488, CY009496, CY009504, CY009512, CY009520,
CY009528, CY009536, CY009544, CY009552, CY009560, CY009568, CY009576, CY009584,
CY009592, CY009600, CY009608, CY009616, CY009624, CY009640, CY009648, CY009656,
CY009664, CY009672, CY009680, CY009688, CY009696, CY009704, CY009712, CY009720,
CY009728, CY009736, CY009744, CY009752, CY009760, CY009768, CY009776, CY009784,
CY009792, CY009800, CY009808, CY009816, CY009824, CY009832, CY009840, CY009848,
CY009856, CY009864, CY009872, CY009880, CY009888, CY009904, CY009912, CY009928,
CY009936, CY009944, CY009952, CY009960, CY009968, CY009976, CY009984, CY009992,
CY010000, CY010008, CY010016, CY010024, CY010032, CY010040, CY010048, CY010056,
CY010064, CY010072, CY010080, CY010088, CY010096, CY010104, CY010112, CY010120,
CY010128, CY010136, CY010144, CY010152, CY010160, CY010168, CY010176, CY010184,
CY010192, CY010200, CY010208, CY010216, CY010224, CY010232, CY010240, CY010248,
CY010256, CY010264, CY010272, CY010280, CY010288, CY010296, CY010304, CY010312,
CY010320, CY010328, CY010336, CY010344, CY010352, CY010360, CY010368, CY010376,
CY010384, CY010392, CY010400, CY010408, CY010416, CY010424, CY010432, CY010440,
CY010448, CY010456, CY010464, CY010472, CY010480, CY010488, CY010496, CY010504,
CY010512, CY010520, CY010528, CY010536, CY010544, CY010552, CY010560, CY010592,
CY010600, CY010608, CY010616, CY010624, CY010632, CY010640, CY010648, CY010656,
CY010664, CY010672, CY010680, CY010688, CY010696, CY010704, CY010712, CY010720,
CY010728, CY010736, CY010744, CY010752, CY010760, CY010768, CY010776, CY010784,
CY010792, CY010800, CY010808, CY010816, CY010824, CY010832, CY010840, CY010848,
CY010856, CY010864, CY010872, CY010880, CY010888, CY010896, CY010904, CY010912,
CY010920, CY010928, CY010936, CY010944, CY010952, CY010960, CY010968, CY010976,
CY010984, CY010992, CY011000, CY011008, CY011016, CY011024, CY011068, CY011076,
CY011084, CY011092, CY011124, CY011132, CY011140, CY011148, CY011156, CY011164,
CY011172, CY011180, CY011188, CY011196, CY011204, CY011212, CY011220, CY011228,
CY011236, CY011244, CY011260, CY011268, CY011276, CY011284, CY011292, CY011300,
CY011308, CY011316, CY011324, CY011332, CY011340, CY011348, CY011356, CY011364,
CY011372, CY011380, CY011388, CY011396, CY011404, CY011412, CY011420, CY011428,
CY011436, CY011444, CY011452, CY011460, CY011468, CY011476, CY011484, CY011492,
CY011500, CY011508, CY011516, CY011524, CY011532, CY011540, CY011548, CY011556,
CY011564, CY011572, CY011580, CY011588, CY011596, CY011604, CY011612, CY011620,
CY011628, CY011636, CY011644, CY011652, CY011660, CY011668, CY011676, CY011684,
CY011692, CY011700, CY011708, CY011716, CY011724, CY011732, CY011740, CY011748,
CY011756, CY011764, CY011772, CY011780, CY011788, CY011796, CY011804, CY011812,
CY011820, CY011828, CY011836, CY011844, CY011852, CY011860, CY011868, CY011876,
CY011884, CY011892, CY011900, CY011908, CY011916, CY011924, CY011932, CY011940,
CY011948, CY011956, CY011964, CY011972, CY011980, CY011988, CY011996, CY012004,
CY012012, CY012020, CY012028, CY012036, CY012044, CY012052, CY012060, CY012068,
CY012076, CY012084, CY012092, CY012100, CY012108, CY012116, CY012124, CY012132,
CY012140, CY012148, CY012156, CY012164, CY012172, CY012180, CY012188, CY012196,
CY012204, CY012212, CY012220, CY012228, CY012236, CY012244, CY012252, CY012260,
CY012268, CY012276, CY012284, CY012292, CY012300, CY012308, CY012316, CY012324,
CY012332, CY012340, CY012348, CY012356, CY012364, CY012372, CY012380, CY012388,
CY012396, CY012404, CY012412, CY012420, CY012428, CY012436, CY012444, CY012452,
CY012460, CY012468, CY012476, CY012484, CY012492, CY012500, CY012508, CY012516,
CY012524, CY012532, CY012540, CY012548, CY012556, CY012564, CY012572, CY012580,
CY012588, CY012596, CY012604, CY012612, CY012620, CY012628, CY012636, CY012644,
CY012652, CY012660, CY012668, CY012676, CY012684, CY012692, CY012700, CY012708,
CY012716, CY012724, CY012732, CY012740, CY012748, CY012756, CY012764, CY012772,
CY012780, CY012788, CY012796, CY012852, CY012860, CY012868, CY012876, CY012884,
CY012892, CY012900, CY012908, CY012916, CY012924, CY012932, CY012940, CY012948,
CY012956, CY012964, CY012972, CY012980, CY012988, CY012996, CY013004, CY013012,
CY013020, CY013028, CY013036, CY013044, CY013052, CY013060, CY013068, CY013076,
CY013084, CY013092, CY013100, CY013108, CY013116, CY013124, CY013132, CY013140,
CY013148, CY013156, CY013164, CY013172, CY013180, CY013188, CY013196, CY013204,
CY013212, CY013220, CY013228, CY013236, CY013244, CY013275, CY013283, CY013291,
CY013299, CY013307, CY013315, CY013323, CY013331, CY013339, CY013347, CY013355,
CY013363, CY013371, CY013379, CY013387, CY013393, CY013401, CY013409, CY013417,
CY013425, CY013433, CY013441, CY013449, CY013457, CY013465, CY013473, CY013481,
CY013489, CY013497, CY013505, CY013513, CY013521, CY013529, CY013537, CY013545,
CY013553, CY013561, CY013569, CY013577, CY013585, CY013593, CY013601, CY013609,
CY013617, CY013625, CY013633, CY013641, CY013649, CY013657, CY013665, CY013673,
CY013681, CY013689, CY013697, CY013705, CY013713, CY013721, CY013729, CY013737,
CY013745, CY013753, CY013761, CY013769, CY013777, CY013785, CY013793, CY013801,
CY013809, CY013817, CY013825, CY013833, CY013841, CY013849, CY013857, CY013875,
CY013883, CY013891, CY013899, CY013907, CY013915, CY013923, CY013931, CY013939,
CY013947, CY013955, CY013963, CY013971, CY013979, CY013987, CY013995, CY014003,
CY014011, CY014019, CY014027, CY014035, CY014043, CY014051, CY014059, CY014067,
CY014075, CY014083, CY014091, CY014099, CY014107, CY014115, CY014123, CY014131,
CY014139, CY014147, CY014155, CY014163, CY014174, CY014181, CY014255, CY014256,
CY014257, CY014258, CY014259, CY014261, CY014262, CY014263, CY014264, CY014265,
CY014266, CY014267, CY014268, CY014276, CY014284, CY014292, CY014300, CY014307,
CY014315, CY014372, CY014380, CY014388, CY014400, CY014408, CY014416, CY014424,
CY014432, CY014440, CY014448, CY014456, CY014464, CY014472, CY014473, CY014488,
CY014496, CY014504, CY014509, CY014517, CY014533, CY014541, CY014547, CY014980,
CY015010, CY015512, CY015520, CY015528, CY015536, CY015544, CY015552, CY015560,
CY015568, CY015576, CY015584, CY015592, CY015600, CY015608, CY015616, CY015624,
CY015632, CY015640, CY015648, CY015656, CY015664, CY015672, CY015680, CY015688,
CY015696, CY015704, CY015712, CY015720, CY015728, CY015736, CY015744, CY015752,
CY015760, CY015768, CY015776, CY015784, CY015792, CY015800, CY015808, CY015816,
CY015824, CY015832, CY015840, CY015848, CY015856, CY015864, CY015872, CY015880,
CY015888, CY015896, CY015904, CY015912, CY015920, CY015928, CY015936, CY015944,
CY015952, CY015960, CY015968, CY015976, CY015984, CY015992, CY016000, CY016008,
CY016016, CY016024, CY016032, CY016040, CY016048, CY016056, CY016064, CY016072,
CY016080, CY016088, CY016096, CY016104, CY016112, CY016120, CY016200, CY016208,
CY016216, CY016232, CY016240, CY016248, CY016256, CY016264, CY016272, CY016407,
CY016431, CY016439, CY016447, CY016455, CY016463, CY016471, CY016479, CY016487,
CY016495, CY016503, CY016511, CY016519, CY016527, CY016535, CY016543, CY016551,
CY016559, CY016567, CY016575, CY016583, CY016591, CY016599, CY016607, CY016631,
CY016639, CY016647, CY016655, CY016663, CY016671, CY016679, CY016687, CY016695,
CY016703, CY016711, CY016719, CY016727, CY016735, CY016743, CY016751, CY016759,
CY016767, CY016775, CY016967, CY016975, CY016983, CY016991, CY016999, CY017007,
CY017015, CY017023, CY017087, CY017095, CY017103, CY017111, CY017119, CY017127,
CY017135, CY017143, CY017151, CY017159, CY017167, CY017175, CY017199, CY017207,
CY017215, CY017223, CY017231, CY017239, CY017247, CY017255, CY017263, CY017271,
CY017287, CY017295, CY017303, CY017311, CY017319, CY017327, CY017335, CY017343,
CY017351, CY017359, CY017367, CY017375, CY017383, CY017391, CY017399, CY017423,
CY017431, CY017439, CY017447, CY017455, CY017463, CY017471, CY017479, CY017487,
CY017495, CY017503, CY017511, CY017519, CY017527, CY017535, CY017543, CY017551,
CY017559, CY017567, CY017575, CY017583, CY017591, CY017599, CY017607, CY017615,
CY017623, CY017631, CY017642, CY017650, CY017658, CY017666, CY017674, CY017682,
CY017692, D10571, D30667, D30674, D30676, DQ096580, DQ098261, DQ098262, DQ098263,
DQ098264, DQ098265, DQ226117, DQ249269, DQ249270, DQ360842, DQ372595, DQ415360,
DQ415361, DQ415362, DQ415363, DQ415364, DQ415365, DQ415366, DQ415367, DQ415368,
DQ415369, DQ415370, DQ469958, DQ487332, DQ487336, DQ493244, DQ493245, DQ493246,
DQ493247, DQ493248, DQ493249, DQ493250, DQ493251, DQ493252, DQ493253, DQ493254,
DQ508829, DQ508837, DQ508845, DQ508853, DQ508861, DQ508869, DQ508877, DQ508885,
DQ508893, DQ508901, DQ508909, DQ508933, DQ535728, DQ643813, EF101744, EF101751,
ISDN125787, ISDN125877, ISDN128030, ISDN129535, ISDN129586, ISDN130369,
ISDN130377, ISDN132410, ISDN132418, ISDN133100, ISDN133320, ISDN13426,
ISDN136845, ISDN136853, ISDN137828, ISDN137829, ISDN137830, ISDN137831,
ISDN137832, ISDN137833, ISDN137834, ISDN137835, ISDN137836, ISDN137837,
ISDN137838, ISDN137839, ISDN137840, ISDN137841, ISDN138752, ISDN138768,
ISDN138776, ISDN140094, ISDN140096, ISDN140097, ISDN140098, ISDN140099,
ISDN140100, ISDN140101, ISDN140102, ISDN140814, ISDN140822, ISDN140839,
ISDN140847, ISDN140855, ISDN140863, ISDN140870, ISDN140906, ISDN140907,
ISDN140908, ISDN181369, ISDN183301, ISDN183309, ISDN183317, ISDN183705,
ISDN183713, ISDN183721, ISDN183729, ISDN183737, ISDN183745, ISDN183753,
ISDN183761, ISDN183769, ISDN183777, ISDN183778, ISDN185501, ISDN187433,
ISDN187441, ISDN187449, ISDN187457, ISDN188323, ISDN188325, ISDN188326,
ISDN188327, ISDN188329, ISDN188330, ISDN188331, ISDN188333, ISDN188334,
ISDN188335, ISDN188336, ISDN188337, ISDN188338, ISDN188341, ISDN188342,
ISDN188345, ISDN188346, ISDN188347, ISDN188348, ISDN188349, ISDN188351,
ISDN204140, ISDN204148, ISDN204156, ISDN207102, ISDN207110, ISDN40016,
ISDN40017, ISDN40040, ISDN41028, K00576, K00577, K00578, K01332, L25720, M12590,
M12592, M12593, M12594, M12595, M12596, M12597, M17699, M23968, M34829, M35094,
M57640, M57641, M57642, M57643, M80974, M80975, M81572, M81578, M81584,
NC_002020, U02087, U08862, U13682, U13683, U53170, U53171, U65670, U65671, U65672,
U65673, U65674, V01102, V01104, X15282, X52146, Z21498

Highly conserved 19-mer and 25-mer sequence fragments were identified by extracting all 19-mer and 25-mer sequence fragments from each of the influenza A viral sequences under study, and then tabulating whether or not each sequence fragment was present as an exact match within each of the influenza A viral sequences. Thus, a first viral sequence contains a 19-mer and 25-mer sequence fragment that extends from position 1 through 19, another from position 2 through 20, another from position 3 through 21, etc. Likewise the second, third, and fourth viral sequences were extracted in the same way, all the way down to the last viral sequence in the list (Table 1). The sequence fragments were then added to a growing table of sequence fragments and a count was maintained of the number of influenza A viral sequences that contain each 19-mer and 25-mer fragment. Finally, the fragment frequency was expressed as the percent of influenza A viral sequences that contained each specific 19-mer and 25-mer fragment relative to all sequences examined. Table 2 lists the most conserved 19-mer sequence fragments, down to 70%, and their frequency of occurrence. Table 3 lists the most conserved 25-mer sequence fragments, also down to 70%, and their frequency of occurrence.

Table 2 and the entire contents of the text file named “Table 2.txt,” created Mar. 8, 2007, size about 77 KB, which was filed electronically with this specification are hereby incorporated by reference.

The sequences in Table 2-1 are numbered SEQ ID NOS: 1-464, respectively in order of appearance.

The sequences in Table 2-2 are numbered SEQ ID NOS: 465-844, respectively in order of appearance.

The sequences in Table 2-3 are numbered SEQ ID NOS: 845-1278, respectively in order of appearance.

The sequences in Table 2-4 are numbered SEQ ID NOS: 1279-1282, respectively in order of appearance.

The sequences in Table 2-5 are numbered SEQ ID NOS: 1283-1550, respectively in order of appearance.

The sequences in Table 2-6 are numbered SEQ ID NOS: 1551-1693, respectively in order of appearance.

The sequences in Table 2-7 are numbered SEQ ID NOS: 1694-2091, respectively in order of appearance.

The sequences in Table 2-8 are numbered SEQ ID NOS: 2092-2314, respectively in order of appearance.

Table 3 and the entire contents of the text file named “Table 3.txt,” created Mar. 8, 2007, size about 51 KB, which was filed electronically with this specification are hereby incorporated by reference.

The sequences in Table 3-1 are numbered SEQ ID NOS: 2315-2568, respectively in order of appearance.

The sequences in Table 3-2 are numbered SEQ ID NOS: 2569-2781, respectively in order of appearance.

The sequences in Table 3-3 are numbered SEQ ID NOS: 2782-3011, respectively in order of appearance.

The sequences in Table 3-5 are numbered SEQ ID NOS: 3012-3130, respectively in order of appearance.

The sequences in Table 3-6 are numbered SEQ ID NOS: 3131-3184, respectively in order of appearance.

The sequences in Table 3-7 are numbered SEQ ID NOS: 3185-3452, respectively in order of appearance.

The sequences in Table 3-8 are numbered SEQ ID NOS: 3453-3584, respectively in order of appearance.

In Table 4, some of the highly conserved sequences from Table 2 are shown along with their close variants. The close variants were obtained from the same influenza viral sequences of Table 1. The close variants of the highly conserved sequences from Table 2 were obtained by using the highly conserved 19-mer sequences in Table 2 as reference sequences. For each 19-mer reference sequence in Table 2, a target fragment or fragments (variant or variants) were sought in each of the influenza A viral sequences described in Table 1 that most closely matched the 19-mer reference sequence. The most closely matching target fragments were those having the fewest number of nucleotide differences from the reference sequence.

If two target fragments had the same number of nucleotide differences from the reference sequence, then preference was given to the target fragment that can form more GU wobble base pairs between the sense strand of the target fragment and the antisense strand of the reference sequence.

The highly conserved reference sequences in Table 2 which were used to obtain the variants had an identical sequence match to a sequence in at least 85% of the influenza A viral sequences described in Table 1. The variants contained 3 (three) or fewer nucleotide differences (changes) from the reference sequence.

Table 4 and the entire contents of the text file named “Table 4.txt,” created Mar. 8, 2007, size 268 KB, which was filed electronically with this specification are hereby incorporated by reference.

The sequences in Table 4-1 are numbered SEQ ID NOS: 3585-4631, respectively in order of appearance.

The sequences in Table 4-2 are numbered SEQ ID NOS: 4632-4639, respectively in order of appearance.

The sequences in Table 4-3 are numbered SEQ ID NOS: 4640-6312, respectively in order of appearance.

The sequences in Table 4-5 are numbered SEQ ID NOS: 6313-6981, respectively in order of appearance.

The sequences in Table 4-7 are numbered SEQ ID NOS: 6982-8614, respectively in order of appearance.

The sequences in Table 4-8 are numbered SEQ ID NOS: 8615-10151, respectively in order of appearance.

Table 5 shows sequence variants for the highly conserved 25-mer sequences from Table 3. This analysis was performed as described for Table 4, above, with the following changes. First, the conserved reference sequences were chosen to match at least 80% of the influenza viral sequences of Table 1, rather than 85%. Second, the variants were chosen to contain 3 (three) or fewer nucleotide differences (changes) from the reference sequence in the left (5′) 19 nucleotides of the 25-mer sequence, rather than restricting it to 3 or fewer mismatches in the entire 25 nucleotide sequence. These changes were made because the 25-mer dicer sequence is cleaved by the enzyme dicer to leave a 19-mer duplex at the 5′ end of the coding strand. Thus, mismatches in the remaining 6 (six) nucleotides of the dicer sequence are not expected to significantly affect the activity of the siRNA.

A small number of sequences in Tables 4 and 5 contain the letter N (or n). The N indicates that any of the four nucleotides are possible at that position. These variants are obtained directly from the submitted GenBank and ISD sequences. The N may represent an uncertainty in determining the correct nucleotide at the time of sequence analysis, or it may indicate that a mixed population of nucleotides was observed at that position.

Table 5 and the entire contents of the text file named “Table 5.txt,” created Mar. 8, 2007, size 324 KB, which was filed electronically with this specification are hereby incorporated by reference.

The sequences in Table 5-1 are numbered SEQ ID NOS: 10152-11244, respectively in order of appearance.

The sequences in Table 5-2 are numbered SEQ ID NOS: 11245-11393, respectively in order of appearance.

The sequences in Table 5-3 are numbered SEQ ID NOS: 11394-13235, respectively in order of appearance.

The sequences in Table 5-5 are numbered SEQ ID NOS: 13236-13824, respectively in order of appearance.

The sequences in Table 5-7 are numbered SEQ ID NOS: 13825-15393, respectively in order of appearance.

The sequences in Table 5-8 are numbered SEQ ID NOS: 15394-16949, respectively in order of appearance.

### EXAMPLE 2

### Synthesized siRNAs

Table 6 and 7 lists the influenza siRNAs (DX) that were synthesized. Table 6 lists the sequences of the sense strands, while Table 7 lists the corresponding sequences of the antisense strands, in order of appearance.

The sequences listed in Table 6 are numbered SEQ ID NOS: 16950-17007, respectively in order of appearance.

TABLE 6

Sense strands of influenza siRNAs

| | | |
|---|---|---|
| Compound | Sense Sequence | No. |

| | | |
|---|---|---|
| DX2844 | Cy5;rG;rG;rA;rU;rC;rU;rU;rA;rU;rU;rU;rC;rU;rU;rC;rG;rG;rA;rG;dT;dT | 16950 |
| DX3003 | Cy5;rG;rG;rA;rU;rC;rU;rU;rA;rU;rU;rU;rC;rU;rU;rC;rG;rG;rA;rG;dT;dT | 16951 |
| DX2852 | rA;rG;rA;rC;rA;rG;rC;rG;rA;rC;rC;rA;rA;rA;rA;rG;rA;rA;rU;rU;rC;rG;rG;dA;dT | 16952 |
| DX3044 | rA;rG;rA;rC;rA;rG;rC;rG;rA;rC;rC;rA;rA;rA;rA;rG;rA;rA;rU;rU;rC;rG;rG;dA;dT | 16953 |
| DX2855 | rA;rU;rG;rA;rA;rG;rA;rU;rC;rU;rG;rU;rU;rC;rC;rA;rC;rC;rA;rU;rU;rG;rA;dA;dG | 16954 |
| DX3046 | rA;rU;rG;rA;rA;rG;rA;rU;rC;rU;rG;rU;rU;rC;rC;rA;rC;rC;rA;rU;rU;rG;rA;dA;dG | 16955 |
| DX2858 | rG;rA;rU;rC;rU;rG;rU;rU;rC;rC;rA;rC;rC;rA;rU;rU;rG;rA;rA;rG;rA;rA;rC;dT;dC | 16956 |
| DX3048 | rG;rA;rU;rC;rU;rG;rU;rU;rC;rC;rA;rC;rC;rA;rU;rU;rG;rA;rA;rG;rA;rA;rC;dT;dC | 16957 |
| DX2861 | rU;rU;rG;rA;rG;rG;rA;rG;rU;rG;rC;rC;rU;rG;rA;rU;rU;rA;rA;rU;rG;rA;rU;dC;dC | 16958 |
| DX3050 | rU;rU;rG;rA;rG;rG;rA;rG;rU;rG;rC;rC;rU;rG;rA;rU;rU;rA;rA;rU;rG;rA;rU;dC;dC | 16959 |
| DX2871 | rG;rG;rC;rU;rC;rU;rU;rA;rU;rU;rU;rC;rU;rU;rC;rG;rG;rA;rG;dT;dT | 16960 |
| DX2874 | rG;rG;rA;rU;rC;rC;rU;rA;rU;rU;rU;rC;rU;rU;rC;rG;rG;rA;rG;dT;dT | 16961 |
| DX2877 | rG;rG;rA;rU;rC;rU;rU;rA;rC;rU;rU;rC;rU;rU;rC;rG;rG;rA;rG;dT;dT | 16962 |
| DX2744 | rG;rG;rA;rU;rC;rU;rU;rA;rU;rU;rU;rC;rU;rU;rC;rG;rG;rA;rG;dT;dT | 16963 |
| DX2880 | rG;rG;rA;rU;rC;rU;rU;rA;rU;rU;rU;rC;rU;rU;rC;rG;rG;rA;rG;dT;dT | 16964 |
| DX2882 | rG;rG;rA;rU;rC;rU;rU;rA;rU;rU;rU;rC;rU;rU;rC;rG;rG;rA;rG;dT;dT | 16965 |
| DX2889 | rG;rG;rA;rU;rC;rU;rU;rA;rU;rU;rU;rC;rU;rU;rC;rG;rG;rA;rG;dT;dT | 16966 |
| DX2890 | rG;rG;rA;rU;rC;rU;rU;rA;rU;rU;rU;rC;rU;rU;rC;rG;rG;rA;rG;dT;dT | 16967 |
| DX2891 | rG;rG;rA;rU;rC;rU;rU;rA;rU;rU;rU;rC;rU;rU;rC;rG;rG;rA;rG;dT;dT | 16968 |
| DX2892 | rG;rG;rA;rU;rC;rU;rU;rA;rU;rU;rU;rC;rU;rU;rC;rG;rG;rA;rG;dT;dT | 16969 |
| DX2888 | rG;rG;rA;rU;rC;rU;rU;rA;rU;rU;rU;rC;rU;rU;rC;rG;rG;rC;rG;dT;dT | 16970 |
| DX2895 | rG;rA;rU;rC;rU;rU;rA;rU;rU;rU;rC;rU;rU;rC;rG;rG;rA;rG;rA;dT;dT | 16971 |
| DX2906 | rG;rA;rU;rC;rU;rU;rA;rU;rU;rU;rC;rU;rU;rC;rG;rG;rA;rG;rA;dT;dT | 16972 |
| DX2908 | rG;rA;rU;rC;rU;rU;rA;rU;rU;rU;rC;rU;rU;rC;rG;rG;rA;rG;rA;dT;dT | 16973 |
| DX2912 | rG;rA;rU;rC;rU;rU;rA;rU;rU;rU;rC;rU;rU;rC;rG;rG;rA;rG;rA;dT;dT | 16974 |
| DX2913 | rG;rA;rU;rC;rU;rU;rA;rU;rU;rU;rC;rU;rU;rC;rG;rG;rA;rG;rA;dT;dT | 16975 |
| DX2914 | rG;rA;rU;rC;rU;rU;rA;rU;rU;rU;rC;rU;rU;rC;rG;rG;rA;rG;rA;dT;dT | 16976 |
| DX2915 | rG;rA;rU;rC;rU;rU;rA;rU;rU;rU;rC;rU;rU;rC;rG;rG;rA;rG;rA;dT;dT | 16977 |
| DX2898 | rG;rC;rU;rC;rU;rU;rA;rU;rU;rU;rC;rU;rU;rC;rG;rG;rA;rG;rA;dT;dT | 16978 |
| DX2901 | rG;rA;rU;rC;rC;rU;rA;rU;rU;rU;rC;rU;rU;rC;rG;rG;rA;rG;rA;dT;dT | 16979 |
| DX2904 | rG;rA;rU;rC;rU;rU;rA;rC;rU;rU;rC;rU;rU;rC;rG;rG;rA;rG;rA;dT;dT | 16980 |
| DX2911 | rG;rA;rU;rC;rU;rU;rA;rU;rU;rU;rC;rU;rU;rC;rG;rG;rC;rG;rA;dT;dT | 16981 |
| DX3054 | rG;rA;rU;rC;rU;rU;rA;rU;rU;rU;rC;rU;rU;rC;rG;rG;rA;rG;rA;rC;rA;rA;dT;dG | 16982 |
| DX3056 | rA;rU;rC;rU;rU;rA;rU;rU;rU;rC;rU;rU;rC;rG;rG;rA;rG;rA;rC;rA;rA;dT;dG | 16983 |
| DX2956 | rG;rG;rA;rU;rC;rU;rU;rA;rU;rU;rU;rC;rU;rU;rC;rG;rG;rA;rG;rA;rC;rA;rA;dT;dG | 16984 |
| DX3030 | rG;rG;rA;rU;rC;rU;rU;rA;rU;rU;rU;rC;rU;rU;rC;rG;rG;rA;rG;rA;rC;rA;rA;dT;dG | 16985 |
| DX3052 | rG;rG;rA;rU;rC;rU;rU;rA;rU;rU;rU;rC;rU;rU;rC;rG;rG;rA;rG;rA;rC;rA;rA;dT;dG | 16986 |
| DX3058 | rG;rG;rA;rU;rC;rU;rU;rA;rU;rU;rU;rC;rU;rU;rC;rG;rG;rA;rG;rA;rC;rA;rA;dT;dG | 16987 |
| DX3060 | rG;rG;rA;rU;rC;rU;rU;rA;rU;rU;rU;rC;rU;rU;rC;rG;rG;rA;rG;rA;rC;rA;rA;dT;dG | 16988 |
| DX3161 | rG;rG;rA;rU;rC;rU;rU;rA;rU;rU;rU;rC;rU;rU;rC;rG;rG;rA;rG;rA;rC;rA;rA;dT;dG | 16989 |
| DX2819 | rG;rA;rU;rC;rU;rG;rU;rU;rC;rC;rA;rC;rC;rA;rU;rU;rG;rA;rA;dT;dT | 16990 |
| DX2820 | rA;rU;rG;rA;rA;rG;rA;rU;rC;rU;rG;rU;rU;rC;rC;rA;rC;rC;rA;dT;dT | 16991 |
| DX2821 | rG;rC;rA;rA;rU;rU;rG;rA;rG;rG;rA;rG;rU;rG;rC;rC;rU;rG;rA;dT;dT | 16992 |
| DX2822 | rU;rU;rG;rA;rG;rG;rA;rG;rU;rG;rC;rC;rU;rG;rA;rU;rU;rA;rA;dT;dT | 16993 |
| DX2823 | rC;rG;rG;rG;rA;rC;rU;rC;rU;rA;rG;rC;rA;rU;rA;rC;rU;rU;rA;dT;dT | 16994 |
| DX2824 | rA;rC;rU;rG;rA;rC;rA;rG;rC;rC;rA;rG;rA;rC;rA;rG;rC;rG;rA;dT;dT | 16995 |
| DX2825 | rA;rG;rA;rC;rA;rG;rC;rG;rA;rC;rC;rA;rA;rA;rA;rG;rA;rA;rU;dT;dT | 16996 |
| DX2962 | rG;rG;rA;rT;rC;rT;rT;rA;rT;rT;rT;rC;rT;rT;rC;rG;rG;rA;rG;dT;dT | 16997 |
| DX3078 | Cy5;rG;rG;rA;rU;rC;rU;rU;rA;rU;rU;rU;rC;rU;rU;rC;rG;rG;rA;rG;rA;rC;rA;rA;dT;dG | 16998 |
| DX3151 | rG;rG;rA;rU;rC;rU;rU;rA;rU;rU;rU;rC;rU;rU;rC;rG;rG;rA;rG;rA;rC;rA;rA;rT;dG | 16999 |
| DX3154 | rG;rG;rA;rU;rC;rU;rU;rA;rU;rU;rU;rC;rU;rU;rC;rG;rG;rA;rG;rA;rC;rA;rA;rT;rT | 17000 |
| DX3156 | rG;rG;rA;rT;rC;rT;rT;rA;rT;rT;rT;rC;rT;rT;rC;rG;rG;rA;rG;rA;rC;rA;rA;rT;dG | 17001 |
| DX3159 | rG;omeG;omeA;rU;rC;rU;rU;rA;rU;rU;rU;rC;rU;rU;rC;rG;rG;rA;rG;rA;rC;rA;rA;dT;dG | 17002 |
| DX3160 | rG;omeG;omeA;rU;rC;rU;rU;rA;rU;rU;rU;rC;rU;rU;rC;rG;rG;rA;rG;rA;rC;rA;rA;dT;dG | 17003 |
| DX3163 | rG;rG;rA;rU;rC;rU;rU;rA;rU;rU;rU;rC;rU;rU;rC;rG;rG;rA;rG;rA;rC;rA;rA;omeU;omeG | 17004 |
| DX3165 | rG;rG;rA;rU;rC;rU;rU;rA;rU;rU;rU;rC;rU;rU;rC;rG;rG;rA;rG;rA;rC;omeA;omeA;dT;dG | 17005 |
| DX3029 | rG;rG;rA;rT;rC;rT;rT;rA;rT;rT;rT;rC;rT;rT;rC;rG;rG;rA;rG;rA;rC;rA;rA;dT;dG | 17006 |
| DX3076 | rG;rG;rA;rT;rC;rT;rT;rA;rT;rT;rT;rC;rT;rT;rC;rG;rG;rA;rG;rA;rC;rA;rA;dT;dG | 17007 |

The sequences listed in Table 7 are numbered SEQ ID NOS: 17008-17065, respectively in order of appearance.

TABLE 7

Antisense strands of influenza siRNAs

| | |
|---|---|
| Antisense sequence | No. |

| | |
|---|---|
| p;rC;rU;rC;rC;rG;rA;rA;rG;rA;rA;rA;rU;rA;rA;rG;rA;rC;rC;rU;dT;dT | 17008 |
| rC;rU;rC;rC;rG;rA;rA;rG;rA;rA;rA;rU;rA;rA;rG;rA;rU;rC;rC;dT;dT | 17009 |
| rA;rU;rC;rC;rG;rA;rA;rU;rU;rC;rU;rU;rU;rU;rG;rG;rU;rC;rG;rC;rU;rG;rU;rC;rU;dT;dT | 17010 |
| rA;rU;rC;rC;rG;rA;rA;rU;rU;rC;rU;rU;rU;rU;rG;rG;rU;rC;rG;rC;rU;rG;rU;rC;rU;rU;rU | 17011 |
| rC;rU;rU;rC;rA;rA;rU;rG;rG;rU;rG;rG;rA;rA;rC;rA;rG;rA;rU;rC;rU;rU;rC;rA;rU;dT;dT | 17012 |
| rC;rU;rU;rC;rA;rA;rU;rG;rG;rU;rG;rG;rA;rA;rC;rA;rG;rA;rU;rC;rU;rU;rC;rA;rU;rU;rU | 17013 |
| rG;rA;rG;rU;rU;rC;rU;rU;rC;rA;rA;rU;rG;rG;rU;rG;rG;rA;rA;rC;rA;rG;rA;rU;rC;dT;dT | 17014 |
| rG;rA;rG;rU;rU;rC;rU;rU;rC;rA;rA;rU;rG;rG;rU;rG;rG;rA;rA;rC;rA;rG;rA;rU;rC;rU;rU | 17015 |
| rG;rG;rA;rU;rC;rA;rU;rU;rA;rA;rU;rC;rA;rG;rG;rC;rA;rC;rU;rC;rC;rU;rC;rA;rA;dT;dT | 17016 |
| rG;rG;rA;rU;rC;rA;rU;rU;rA;rA;rU;rC;rA;rG;rG;rC;rA;rC;rU;rC;rC;rU;rC;rA;rA;rU;rU | 17017 |
| rC;rU;rC;rC;rG;rA;rA;rG;rA;rA;rA;rU;rA;rA;rG;rA;rI;rC;rC;dT;dT | 17018 |
| rC;rU;rC;rC;rG;rA;rA;rG;rA;rA;rA;rU;rA;rI;rG;rA;rU;rC;rC;dT;dT | 17019 |
| rC;rU;rC;rC;rG;rA;rA;rG;rA;rA;rI;rU;rA;rA;rG;rA;rU;rC;rC;dT;dT | 17020 |
| rC;rU;rC;rC;rG;rA;rA;rG;rA;rA;rA;rU;rA;rA;rG;rA;rU;rC;rC;dT;dT | 17021 |
| rC;rU;rC;rC;rG;rA;rA;rI;rA;rA;rA;rU;rA;rA;rG;rA;rU;rC;rC;dT;dT | 17022 |
| rC;rU;rC;rC;rI;rA;rA;rG;rA;rA;rA;rU;rA;rA;rG;rA;rU;rC;rC;dT;dT | 17023 |
| rC;rU;rC;rC;rG;rA;rA;rG;rA;rA;rA;rU;rA;rA;rG;rA;rI;rC;rC;dT;dT | 17024 |
| rC;rU;rC;rC;rG;rA;rA;rG;rA;rA;rA;rU;rA;rI;rG;rA;rU;rC;rC;dT;dT | 17025 |
| rC;rU;rC;rC;rG;rA;rA;rG;rA;rA;rI;rU;rA;rA;rG;rA;rU;rC;rC;dT;dT | 17026 |
| rC;rI;rC;rC;rG;rA;rA;rG;rA;rA;rA;rU;rA;rA;rG;rA;rU;rC;rC;dT;dT | 17027 |
| rC;rI;rC;rC;rG;rA;rA;rG;rA;rA;rA;rU;rA;rA;rG;rA;rU;rC;rC;dT;dT | 17028 |
| rU;rC;rU;rC;rC;rG;rA;rA;rG;rA;rA;rA;rU;rA;rA;rG;rA;rU;rC;dT;dT | 17029 |
| rU;rC;rU;rC;rC;rG;rA;rA;rI;rA;rA;rA;rU;rA;rA;rG;rA;rU;rC;dT;dT | 17030 |
| rU;rC;rU;rC;rC;rI;rA;rA;rG;rA;rA;rA;rU;rA;rA;rG;rA;rU;rC;dT;dT | 17031 |
| rU;rC;rU;rC;rC;rG;rA;rA;rG;rA;rA;rA;rU;rA;rA;rG;rA;rI;rC;dT;dT | 17032 |
| rU;rC;rU;rC;rC;rG;rA;rA;rG;rA;rA;rA;rU;rA;rI;rG;rA;rU;rC;dT;dT | 17033 |
| rU;rC;rU;rC;rC;rG;rA;rA;rG;rA;rA;rI;rU;rA;rA;rG;rA;rU;rC;dT;dT | 17034 |
| rU;rC;rI;rC;rC;rG;rA;rA;rG;rA;rA;rA;rU;rA;rA;rG;rA;rU;rC;dT;dT | 17035 |
| rU;rC;rU;rC;rC;rG;rA;rA;rG;rA;rA;rA;rU;rA;rA;rG;rA;rI;rC;dT;dT | 17036 |
| rU;rC;rU;rC;rC;rG;rA;rA;rG;rA;rA;rA;rU;rA;rI;rG;rA;rU;rC;dT;dT | 17037 |
| rU;rC;rU;rC;rC;rG;rA;rA;rG;rA;rA;rI;rU;rA;rA;rG;rA;rU;rC;dT;dT | 17038 |
| rU;rC;rI;rC;rC;rG;rA;rA;rG;rA;rA;rA;rU;rA;rA;rG;rA;rU;rC;dT;dT | 17039 |
| rC;rA;rU;rU;rG;rU;rC;rU;rC;rC;rG;rA;rA;rG;rA;rA;rA;rU;rA;rA;rG;rA;rU;rC;rC;rU;rU | 17040 |
| rC;rA;rU;rU;rG;rU;rC;rU;rC;rC;rG;rA;rA;rG;rA;rA;rA;rU;rA;rA;rG;rA;rU;rC;rC;rU;rU | 17041 |
| rC;rA;rU;rU;rG;rU;rC;rU;rC;rC;rG;rA;rA;rG;rA;rA;rA;rU;rA;rA;rG;rA;rU;rC;rC;dT;dT | 17042 |
| rC;rA;rU;rU;rG;rU;rC;rU;rC;rC;rG;rA;rA;rG;rA;rA;rA;rU;rA;rA;rG;rA;rU;rC;rC;rU;rU | 17043 |
| rC;rA;rU;rU;rG;rU;rC;rU;rC;rC;rG;rA;rA;rG;rA;rA;rA;rU;rA;rA;rG;rA;rU;rC;rC;rT;rT | 17044 |
| rA;rU;rU;rG;rU;rC;rU;rC;rC;rG;rA;rA;rG;rA;rA;rA;rU;rA;rA;rG;rA;rU;rC;rC;rU;rU | 17045 |
| rU;rU;rG;rU;rC;rU;rC;rC;rG;rA;rA;rG;rA;rA;rA;rU;rA;rA;rG;rA;rU;rC;rC;rU;rU | 17046 |
| rC;rA;rU;rU;rG;rU;rC;rU;rC;rC;rG;rA;rA;rG;rA;rA;rA;rU;rA;rA;rG;rA;rU;omeC;omeC;rU;rU | 17047 |
| rU;rU;rC;rA;rA;rU;rG;rG;rU;rG;rG;rA;rA;rC;rA;rG;rA;rU;rC;dT;dT | 17048 |
| rU;rG;rG;rU;rG;rG;rA;rA;rC;rA;rG;rA;rU;rC;rU;rU;rC;rA;rU;dT;dT | 17049 |
| rU;rC;rA;rG;rG;rC;rA;rC;rU;rC;rC;rU;rC;rA;rA;rU;rU;rG;rC;dT;dT | 17050 |
| rU;rU;rA;rA;rU;rC;rA;rG;rG;rC;rA;rC;rU;rC;rC;rU;rC;rA;rA;dT;dT | 17051 |
| rU;rA;rA;rG;rU;rA;rU;rG;rC;rU;rA;rG;rA;rG;rU;rC;rC;rC;rG;dT;dT | 17052 |
| rU;rC;rG;rC;rU;rG;rU;rC;rU;rG;rG;rC;rU;rG;rU;rC;rA;rG;rU;dT;dT | 17053 |
| rA;rU;rU;rC;rU;rU;rU;rU;rG;rG;rU;rC;rG;rC;rU;rG;rU;rC;rU;dT;dT | 17054 |
| rC;rT;rC;rC;rG;rA;rA;rG;rA;rA;rA;rT;rA;rA;rG;rA;rT;rC;rC;dT;dT | 17055 |
| rC;rA;rU;rU;rG;rU;rC;rU;rC;rC;rG;rA;rA;rG;rA;rA;rA;rU;rA;rA;rG;rA;rU;rC;rC;rU;rU | 17056 |
| rC;rA;rU;rU;rG;rU;rC;rU;rC;rC;rG;rA;rA;rG;rA;rA;rA;rU;rA;rA;rG;rA;rU;rC;rC;rU;rU | 17057 |
| rA;rA;rU;rU;rG;rU;rC;rU;rC;rC;rG;rA;rA;rG;rA;rA;rA;rU;rA;rA;rG;rA;rU;rC;rC;rU;rU | 17058 |
| rC;rA;rT;rT;rG;rT;rC;rT;rC;rC;rG;rA;rA;rG;rA;rA;rA;rT;rA;rA;rG;rA;rT;rC;rC;rU;rU | 17059 |
| rC;rA;rU;rU;rG;rU;rC;rU;rC;rC;rG;rA;rA;rG;rA;rA;rA;rU;rA;rA;rG;rA;rU;omeC;omeC;rU;rU | 17060 |
| rC;rA;rU;rU;rG;rU;rC;rU;rC;rC;rG;rA;rA;rG;rA;rA;rA;rU;rA;rA;rG;rA;rU;rC;rC;rU;rU | 17061 |
| rC;rA;rU;rU;rG;rU;rC;rU;rC;rC;rG;rA;rA;rG;rA;rA;rA;rU;rA;rA;rG;rA;rU;rC;rC;rU;rU | 17062 |
| rC;rA;rU;rU;rG;rU;rC;rU;rC;rC;rG;rA;rA;rG;rA;rA;rA;rU;rA;rA;rG;rA;rU;rC;rC;rU;rU | 17063 |
| rC;rA;rT;rT;rG;rT;rC;rT;rC;rC;rG;rA;rA;rG;rA;rA;rA;rT;rA;rA;rG;rA;rT;rC;rC;rT;rT | 17064 |
| rC;rA;rT;rT;rG;rT;rC;rT;rC;rC;rG;rA;rA;rG;rA;rA;rA;rT;rA;rA;rG;rA;rT;rC;rC;rU;rU | 17065 |

A multi-letter code shown in Table 8 has been used in describing the siRNA structures in Tables 6 and 7 to unambiguously represent each sequence. In this code, the prefix “ribo” or “r” signifies an RNA nucleoside. The prefixes “d,” “ome,” and “r” refer only to the letter immediately following the prefix.

TABLE 8
siRNA Structure Code

| Code | Represents |
|---|---|
| Cy5 | Cy5 fluorescent dye |
| dA | deoxyadenosine |
| dC | deoxycytidine |
| dG | deoxyguanosine |
| dT | deoxythymidine |
| omeA | 2′-O-methyl adenosine |
| omeC | 2′-O-methyl cytidine |
| omeG | 2′-O-methyl guanosine |
| omeU | 2′-O-methyl uridine |
| p | 5′ phosphate |
| rA | (ribo)adenosine |
| rC | (ribo)cytidine |
| rG | (ribo)guanosine |
| rI | (ribo)inosine |
| rT | (ribo)thymidine |
| | (5-methyluridine) |
| rU | (ribo)uridine |

In Table 9, the top nine siRNA sites identified from laboratory screening studies have been identified.

TABLE 9

Top nine siRNA sites as identified from lab-
oratory screening studies, showing conserved
and minor variant 19-mer sequences from the
Influenza A

| | | | | |
|---|---|---|---|---|
| Seq | Ref | | | % |
| ID | ID | Segment | Match Seq | Total |

| | | | | |
|---|---|---|---|---|
| 17066 | 7 | PB2 | AGACAGCGACCAAAAGAAU | 99.1% |
| 17067 | 7 | | AGACAGCGACCAAAAGgAU | 0.3% |
| 17068 | 7 | | AGACAGCGACCAAAgGAAU | 0.1% |
| 17069 | 7 | | AGACAGCGACCAAAAGAuU | 0.1% |
| 17070 | 7 | | AGACgaCGAuCAAAAGAAU | 0.1% |
| 17071 | 17 | PB2 | ACUGACAGCCAGACAGCGA | 99.0% |
| 17072 | 17 | | ACUGACAGuCAGACAGCGA | 0.2% |
| 17073 | 17 | | ACUGAuAGCCAGACAGCGA | 0.3% |
| 17074 | 17 | | ACcGACAGCCAGACAGCGA | 0.2% |
| 17075 | 17 | | ACUGACAGCCAGACgaCGA | 0.1% |
| 17076 | 48 | PB2 | CGGGACUCUAGCAUACUUA | 98.0% |
| 17077 | 48 | | CGGGACUCUAGCAUgCUUA | 0.1% |
| 17078 | 48 | | CGGGACUuUAGCAUACUUA | 0.2% |
| 17079 | 48 | | aGGGACUCUAGCAUACUUA | 0.1% |
| 17080 | 48 | | CGaGACUCUAGCAUACUUA | 0.4% |
| 17081 | 48 | | CGGaACUCUAGCAUACUUA | 0.6% |
| 17082 | 48 | | CGGGACUaUAGCAUACUUA | 0.1% |
| 17083 | 48 | | CGGGACUCcAGCAUACUUA | 0.3% |
| 17084 | 48 | | CGGGACUCUAaCAUACUUA | 0.1% |
| 17085 | 1187 | PB1 | GAUCUGUUCCACCAUUGAA | 90.9% |
| 17086 | 1187 | | GAUCUGUUuCACCAUUGAA | 0.7% |
| 17087 | 1187 | | aAUCUGUUCCACCAUUGAA | 0.1% |
| 17088 | 1187 | | GAcCUGUUCCACCAUUGAA | 6.9% |
| 17089 | 1187 | | GAUCUGcUCCACCAUUGAA | 0.2% |
| 17090 | 1187 | | GAUCUGUUaCACCAUUGAA | 0.1% |
| 17091 | 1187 | | GAUCUGUUCCACCAUUaAA | 0.1% |
| 17092 | 1187 | | GAcCUGUUCuACCAUUGAA | 0.1% |
| 17093 | 1187 | | GAcCUGcUCCACCAUUGAA | 0.3% |
| 17094 | 1206 | PB1 | AUGAAGAUCUGUUCCACCA | 88.6% |
| 17095 | 1206 | | AUGAAGAUCUGUUuCACCA | 0.7% |
| 17096 | 1206 | | AUGAgGAUCUGUUCCACCA | 0.2% |
| 17097 | 1206 | | AcGAAGAUCUGUUCCACCA | 0.1% |
| 17098 | 1206 | | AUGAAaAUCUGUUCCACCA | 0.1% |
| 17099 | 1206 | | AUGAAGAcCUGUUCCACCA | 6.8% |
| 17100 | 1206 | | AUGAAGAUCUGcUCCACCA | 0.2% |
| 17101 | 1206 | | AUGAAGAUCUGUUaCACCA | 0.1% |
| 17102 | 1206 | | cUGAAGAUCUGUUCCACCA | 0.1% |
| 17103 | 1206 | | uUGAAGAUCUGUUCCACCA | 2.0% |
| 17104 | 1206 | | AUGAAGAcCUGUUCuACCA | 0.1% |
| 17105 | 1206 | | AUaAAGAcCUGUUCCACCA | 0.1% |
| 17106 | 1206 | | AUGAAGAcCUGcUCCACCA | 0.2% |
| 17107 | 2393 | PA | UUGAGGAGUGCCUGAUUAA | 98.7% |
| 17108 | 2393 | | UUGAGGAGUGCCUGgUUAA | 0.1% |
| 17109 | 2393 | | UUGAGGAaUGCCUGAUUAA | 1.0% |
| 17110 | 2393 | | UUGAGGAGUGCCUaAUUAA | 0.2% |
| 17111 | 2394 | PA | GCAAUUGAGGAGUGCCUGA | 98.6% |
| 17112 | 2394 | | GCAAUUGAGGAGUGCCUGg | 0.1% |
| 17113 | 2394 | | GCAgUUGAGGAGUGCCUGA | 0.1% |
| 17114 | 2394 | | GCAAUUGAGGAaUGCCUGA | 1.0% |
| 17115 | 2394 | | GCAAUUGAGGAGUGCCUaA | 0.2% |
| 17116 | 3560 | NP | GAUCUUAUUUCUUCGGAGA | 96.0% |
| 17117 | 3560 | | GAUCUUAUUUCUUCGGgGA | 1.7% |
| 17118 | 3560 | | GAUCUUAUUUCUUuGGAGA | 0.2% |
| 17119 | 3560 | | GgUCUUAUUUCUUCGGAGA | 0.9% |
| 17120 | 3560 | | GuUCUUAUUUCUUCGGAGA | 1.1% |
| 17121 | 3561 | NP | GGAUCUUAUUUCUUCGGAG | 96.0% |
| 17122 | 3561 | | GGAUCUUAUUUCUUCGGgG | 1.7% |
| 17123 | 3561 | | GGAUCUUAUUUCUUuGGAG | 0.2% |
| 17124 | 3561 | | GGgUCUUAUUUCUUCGGAG | 0.9% |
| 17125 | 3561 | | GGuUCUUAUUUCUUCGGAG | 1.1% |

Madin-Darby canine kidney cells (MDCK) were used. For electroporation, the cells were kept in serum-free RPMI 1640 medium. Virus infections were done in infection medium. Influenza viruses A/PR/8/34 (PR8) and A/WSN/33 (WSN), subtypes H1N1 were used. Sense and antisense sequences that were tested are listed in Table 10.

TABLE 10

siRNA Sequences

| | |
|---|---|
| Name | siRNA sequence (5′–3′) |

| | | |
|---|---|---|
| PB2-2210/2230 (sense) | ggagacgugguguugguaadTdT | (SEQ ID NO: 17126) |
| PB2-2210/2230 (antisense) | uuaccaacaccacgucuccdTdT | (SEQ ID NO: 17127) |
| PB2-2240/2260 (sense) | cgggacucuagcauacuuadTdT | (SEQ ID NO: 17128) |
| PB2-2240/2260 (antisense) | uaaguaugcuagagucccgdTdT | (SEQ ID NO: 17129) |
| PB1-6/26 (sense) | gcaggcaaaccauuugaaudTdT | (SEQ ID NO: 17130) |
| PB1-6/26 (antisense) | auucaaaugguuugccugcdTdT | (SEQ ID NO: 17131) |
| PB1-129/149 (sense) | caggauacaccauggauacdTdT | (SEQ ID NO: 17132) |
| PB1-129/149 (antisense) | guauccaugguguauccugdTdT | (SEQ ID NO: 17133) |
| PB1-2257/2277 (sense) | gaucuguuccaccauugaadTdT | (SEQ ID NO: 17134) |
| PB1-2257/2277 (antisense) | uucaaugguggaacagaucdTdT | (SEQ ID NO: 17135) |
| PA-44/64 (sense) | ugcuucaauccgaugauugdTdT | (SEQ ID NO: 17136) |
| PA-44/64 (antisense) | caaucaucggauugaagcadTdT | (SEQ ID NO: 17137) |
| PA-739/759 (sense) | cggcuacauugagggcaagdTdT | (SEQ ID NO: 17138) |
| PA-739/759 (antisense) | cuugcccucaauguagccgdTdT | (SEQ ID NO: 17139) |
| PA-2087/2107 (G) (sense) | gcaauugaggagugccugadTdT | (SEQ ID NO: 17140) |
| PA-2087/2107 (G) (antisense) | ucaggcacuccucaauugcdTdT | (SEQ ID NO: 17141) |
| PA-2110/2130 (sense) | ugaucccuggguuuugcuudTdT | (SEQ ID NO: 17142) |
| PA-2110/2130 (antisense) | aagcaaaacccagggaucadTdT | (SEQ ID NO: 17143) |
| PA-2131/2151 (sense) | ugcuucuugguucaacuccdTdT | (SEQ ID NO: 17144) |
| PA-2131/2151 (antisense) | ggaguugaaccaagaagcadTdT | (SEQ ID NO: 17145) |
| NP-231/251 (sense) | uagagagaauggugcucucdTdT | (SEQ ID NO: 17146) |
| NP-231/251 (antisense) | gagagcaccauucucucuadTdT | (SEQ ID NO: 17147) |
| NP-390/410 (sense) | uaaggcgaaucuggcgccadTdT | (SEQ ID NO: 17148) |
| NP-390/410 (antisense) | uggcgccagauucgccuuadTdT | (SEQ ID NO: 17149) |
| NP-1496/1516 (sense) | ggaucuuauuucuucggagdTdT | (SEQ ID NO: 17150) |
| NP-1496/1516 (antisense) | cuccgaagaaauaagauccdTdT | (SEQ ID NO: 17151) |
| NP-1496/1516a (sense) | ggaucuuauuucuucggagadTdT | (SEQ ID NO: 17152) |
| NP-1496/1516a (antisense) | ucuccgaagaaauaagauccdTdT | (SEQ ID NO: 17153) |
| M-37/57 (sense) | ccgaggucgaaacguacgudTdT | (SEQ ID NO: 17154) |
| M-37/57 (antisense) | acguacguuucgaccucggdTdT | (SEQ ID NO: 17155) |
| M-480/500 (sense) | cagauugcugacucccagcdTdT | (SEQ ID NO: 17156) |
| M-480/500 (antisense) | gcugggagucagcaaucugdTdT | (SEQ ID NO: 17157) |
| M-598/618 (sense) | uggcuggaucgagugagcadTdT | (SEQ ID NO: 17158) |
| M-598/618 (antisense) | ugcucacucgauccagccadTdT | (SEQ ID NO: 17159) |
| M-934/954 (sense) | gaauaucgaaaggaacagcdTdT | (SEQ ID NO: 17160) |
| M-934/954 (antisense) | gcuguuccuuucgauauucdTdT | (SEQ ID NO: 17161) |
| NS-128/148 (sense) | cggcuucgccgagaucagadAdT | (SEQ ID NO: 17162) |
| NS-128/148 (antisense) | ucugaucucggcgaagccgdAdT | (SEQ ID NO: 17163) |
| NS-562/582 (R) (sense) | guccuccgaugaggacuccdTdT | (SEQ ID NO: 17164) |
| NS-562/582 (R) (antisense) | ggaguccucaucggaggacdTdT | (SEQ ID NO: 17165) |
| NS-589/609 (sense) | ugauaacacaguucgagucdTdT | (SEQ ID NO: 17166) |
| NS-589/609 (antisense) | gacucgaacuguguuaucadTdT | (SEQ ID NO: 17167) |

All siRNAs were synthesized by Dharmacon Research (Lafayette, Colo.) using 2′ACE protection chemistry and transfected into the cells by electroporation. Six to eight h following electroporation, the serum-containing medium was washed away and PR8 or WSN virus at the appropriate multiplicity of infection was inoculated into the wells. Cells were infected with either 1,000 PFU (one virus per 1,000 cells; MOI=0.001) or 10,000 PFU (one virus per 100 cells; MOI=0.01) of virus. After 1 h incubation at room temperature, 2 ml of infection medium with 4 μg/ml of trypsin was added to each well and the cells were incubated and, at indicated times, supernatants were harvested from infected cultures and the titer of virus was determined by hemagglutination of chicken erythrocytes.

Supernatants were harvested at 24, 36, 48, and 60 hours after infection. Viral titer was measured using a standard hemagglutinin assay as described in Knipe, D. M. and P. M. Howley, Fundamental Virology, 4th ed., pp. 34-35. The hemagglutination assay was done in V-bottomed 96-well plates. Serial 2-fold dilutions of each sample were incubated for 1 h on ice with an equal volume of a 0.5% suspension of chicken erythrocytes (Charles River Laboratories). Wells containing an adherent, homogeneous layer of erythrocytes were scored as positive. For plaque assays, serial 10-fold dilutions of each sample were titered for virus as described in Fundamental Virology, 4th ed., p. 32, as well known in the art.

To investigate the feasibility of using siRNA to suppress influenza virus replication, various influenza virus A RNAs were targeted. Specifically, the MDCK cell line, which is readily infected and widely used to study influenza virus, was utilized.

Each siRNA was individually introduced into populations of MDCK cells by electroporation. siRNA targeted to GFP (sense: 5′-GGCUACGUCCAGGAGCGCAUU-3′ (SEQ ID NO: 17168); antisense: 5′-UGCGCUCCUGGACGUAGCCUU-3′ (SEQ ID NO: 17169)) was used as control. This siRNA is referred to as GFP-949. In subsequent experiments (described in examples below) the UU overhang at the 3′ end of both strands was replaced by dTdT with no effect on results. A mock electroporation was also performed as a control. Eight hours after electroporation cells were infected with either influenza A virus PR8 or WSN at an MOI of either 0.1 or 0.01 and were analyzed for virus production at various time points (24, 36, 48, 60 hours) thereafter using a standard hemagglutination assay. GFP expression was assayed by flow cytometry using standard methods.

The ability of individual siRNAs to inhibit replication of influenza virus A strain A/Puerto Rico/8/34 (H1N1) or influenza virus A strain A/WSN/33 (H1N1) was determined by measuring HA titer. Thus a high HA titer indicates a lack of inhibition while a low HA titer indicates effective inhibition. MDCK cells were infected at an MOI of 0.01. For these experiments one siRNA that targets the PB1 segment (PB1-2257/2277), one siRNA that targets the PB2 segment (PB2-2240/2260), one siRNA that targets the PA segment (PA-2087/2107 (G)), and three different siRNAs that target the NP genome and transcript (NP-231/251, NP-390/410, and NP-1496/1516) were tested.

In the absence of siRNA (mock TF) or the presence of control (GFP) siRNA, the titer of virus increased over time, reaching a peak at approximately 48-60 hours after infection. In contrast, at 60 hours the viral titer was significantly lower in the presence of any of the siRNAs. For example, in strain WSN the HA titer (which reflects the level of virus) was approximately half as great in the presence of siRNAs PB2-2240 or NP-231 than in the controls. In particular, the level of virus was below the detection limit (10,000 PFU/ml) in the presence of siRNA NP-1496 in both strains. This represents a decrease by a factor of more than 60-fold in the PR8 strain and more than 120-fold in the WSN strain. The level of virus was also below the detection limit (10,000 PFU/ml) in the presence of siRNA PA-2087(G) in strain WSN and was extremely low in strain PR8. Suppression of virus production by siRNA was evident even from the earliest time point measured. Effective suppression, including suppression of virus production to undetectable levels (as determined by HA titer) has been observed at time points as great as 72 hours post-infection.

A total of twenty siRNAs, targeted to 6 segments of the influenza virus genome (PB2, PB1, PA, NP, M and NS), were tested in the MDCK cell line system. About 15% of the siRNA (PB1-2257, PA-2087G and NP-1496) tested displayed a strong effect, inhibiting viral production by more than 100 fold in most cases at MOI=0.001 and by 16 to 64 fold at MOI=0.01, regardless of whether PR8 or WSN virus was used. In particular, when siRNA NP-1496 or PA-2087 was used, inhibition was so pronounced that culture supernatants lacked detectable hemagglutinin activity. These potent siRNAs target 3 different viral gene segments: PB1 and PA, which are involved in the RNA transcriptase complex, and NP which is a single-stranded RNA binding nucleoprotein. Consistent with findings in other systems, the sequences targeted by these siRNAs are all positioned relatively close to the 3-prime end of the coding region.

Approximately 40% of the siRNAs significantly inhibited virus production, but the extent of inhibition varied depending on certain parameters. Approximately 15% of siRNAs potently inhibited virus production regardless of whether PR8 or WSN virus was used. However, in the case of certain siRNAs, the extent of inhibition varied somewhat depending on whether PR8 or WSN was used. Some siRNAs significantly inhibited virus production only at early time points (24 to 36 hours after infection) or only at lower dosage of infection (MOI=0.001), such as PB2-2240, PB1-129, NP-231 and M-37. These siRNAs target different viral gene segments, and the corresponding sequences are positioned either close to 3-prime end or 5-prime end of the coding region. Approximately 45% of the siRNAs had no discernible effect on the virus titer, indicating that they were not effective in interfering with influenza virus production in MDCK cells. In particular, none of the four siRNAs which target the NS gene segment showed any inhibitory effect.

To estimate virus titers more precisely, plaque assays with culture supernatants were performed (at 60 hrs) from culture supernatants obtained from virus-infected cells that had undergone mock transfection or transfection with NP-1496. Approximately 6×10⁵ pfu/ml was detected in mock supernatant, whereas no plaques were detected in undiluted NP-1496 supernatant. As the detection limit of the plaque assay is about 20 pfu (plaque forming unit)/ml, the inhibition of virus production by NP-1496 is at least about 30,000 fold. Even at an MOI of 0.1, NP-1496 inhibited virus production about 200-fold.

To determine the potency of siRNA, a graded amount of NP-1496 was transfected into MDCK cells followed by infection with PR8 virus. Virus titers in the culture supernatants were measured by hemagglutinin assay. As the amount of siRNA decreased, virus titer increased in the culture supernatants. However, even when as little as 25 pmol of siRNA was used for transfection, approximately 4-fold inhibition of virus production was detected as compared to mock transfection, indicating the potency of NP-1496 siRNA in inhibiting influenza virus production.

For therapy, it is desirable for siRNA to be able to effectively inhibit an existing virus infection. In a typical influenza virus infection, new virions are released beginning at about 4 hours after infection. To determine whether siRNA could reduce or eliminate infection by newly released virus in the face of an existing infection, MDCK cells were infected with PR8 virus and then transfected with NP-1496 siRNA. Virus titer increased steadily over time following mock transfection, whereas virus titer increased only slightly in NP-1496 transfected cells. Thus administration of siRNA after virus infection is effective.

Together, these results show that (i) certain siRNAs can potently inhibit influenza virus production; (ii) influenza virus production can be inhibited by siRNAs specific for different viral genes, including those encoding NP, PA, and PB1 proteins; and (iii) siRNA inhibition occurs in cells that were infected previously in addition to cells infected simultaneously with or following administration of siRNAs.

### EXAMPLE 3

### siRNAs That Target Viral RNA Polymerase or Nucleoprotein Inhibit Influenza A Virus Production in Chicken Embryos

siRNA-oligofectamine complex formation and chicken embryo inoculation. SiRNAs were prepared as described above. Chicken eggs were maintained under standard conditions. 30 μl of Oligofectamine (product number: 12252011 from Life Technologies, now Invitrogen) was mixed with 30 μl of Opti-MEM I (Gibco) and incubated at RT for 5 min. 2.5 nmol (10 μl) of siRNA was mixed with 30 μl of Opti-MEM I and added into diluted oligofectamine. The siRNA and oligofectamine was incubated at RT for 30 min. 10-day old chicken eggs were inoculated with siRNA-oligofectamine complex together with 100 μl of PR8 virus (5000 pfu/ml). The eggs were incubated at 37° C. for indicated time and allantoic fluid was harvested. Viral titer in allantoic fluid was tested by HA assay as described above.

To confirm the results in MDCK cells, the ability of siRNA to inhibit influenza virus production in fertilized chicken eggs was also assayed. Because electroporation cannot be used on eggs, Oligofectamine, a lipid-based agent that has been shown to facilitate intracellular uptake of DNA oligonucleotides as well as siRNAs in vitro was used (25). Briefly, PR8 virus alone (500 pfu) or virus plus siRNA-oligofectamine complex was injected into the allantoic cavity of 10-day old chicken eggs. Allantoic fluids were collected 17 hours later for measuring virus titers by hemagglutinin assay. When virus was injected alone (in the presence of Oligofectamine), high virus titers were readily detected. Co-injection of GFP-949 did not significantly affect the virus titer. (No significant reduction in virus titer was observed when Oligofectamine was omitted.) The injection of siRNAs specific for influenza virus showed results consistent with those observed in MDCK cells: The same siRNAs (NP-1496, PA2087 and PB1-2257) that inhibited influenza virus production in MDCK cells also inhibited virus production in chicken eggs, whereas the siRNAs (NP-231, M-37 and PB1-129) that were less effective in MDCK cells were ineffective in fertilized chicken eggs. Thus, siRNAs are also effective in interfering with influenza virus production in fertilized chicken eggs.

### EXAMPLE 4

### siRNA Inhibits Influenza Virus Production at the mRNA Level

siRNA preparation was performed as described above.

RNA extraction, reverse transcription and real time PCR. 1×10⁷ MDCK cells were electroporated with 2.5 nmol of NP-1496 or mock electroporated (no siRNA). Eight hours later, influenza A PR8 virus was inoculated into the cells at MOI=0.1. At times 1, 2, and 3-hour post-infection, the supernatant was removed, and the cells were lysed with Trizol reagent (Gibco). RNA was purified according to the manufacturer's instructions. Reverse transcription (RT) was carried out at 37° C. for 1 hr, using 200 ng of total RNA, specific primers (see below), and Omniscript Reverse transcriptase kit (Qiagen) in a 20-μl reaction mixture according to the manufacturer's instructions. Primers specific for either mRNA, NP vRNA, NP cRNA, NS vRNA, or NS cRNA were as follows:

(SEQ ID NO: 17170)

mRNA, dT₁₈ =
5′-TTTTTTTTTTTTTTTTTT-3′

(SEQ ID NO: 17171)

NP vRNA, NP-367:
5′-CTCGTCGCTTATGACAAAGAAG-3′.

(SEQ ID NO: 17172)

NP cRNA, NP-1565R:
5′-ATATCGTCTCGTATTAGTAGAAACAAGGGTATTTTT-3′.

(SEQ ID NO: 17173)

NS vRNA, NS-527:
5′-CAGGACATACTGATGAGGATG-3′.

(SEQ ID NO: 17174)

NS cRNA, NS-890R:
5′-ATATCGTCTCGTATTAGTAGAAACAAGGGTGTTTT-3′.

1 μl of RT reaction mixture (i.e., the sample obtained by performing reverse transcription) and sequence-specific primers were used for real-time PCR using SYBR Green PCR master mix (AB Applied Biosystems) including SYBR Green I double-stranded DNA binding dye. PCRs were cycled in an ABI PRISM 7000 sequence detection system (AB applied Biosystem) and analyzed with ABI PRISM 7000 SDS software (AB Applied Biosystems). The PCR reaction was carried out at 50° C., 2 min, 95° C., 10 min, then 95° C., 15 sec and 60° C., 1 min for 50 cycles. Cycle times were analyzed at a reading of 0.2 fluorescence units. All reactions were done in duplicate. Cycle times that varied by more than 1.0 between the duplicates were discarded. The duplicate cycle times were then averaged and the cycle time of β-actin was subtracted from them for a normalized value. PCR primers were as follows.

| | |
|---|---|
| For NP RNAs | |
| NP-367: | |
| 5′-CTCGTCGCTTATGACAAAGAAG-3′. | (SEQ ID NO: 17171) |
| NP-460R: | |
| 5′-AGATCATCATGTGAGTCAGAC-3′. | (SEQ ID NO: 17175) |
| For NS RNAs: | |
| NS-527: | |
| 5′-CAGGACATACTGATGAGGATG-3′. | (SEQ ID NO: 17173) |
| NS-617R: | |
| 5′-GTTTCAGAGACTCGAACTGTG-3′. | (SEQ ID NO: 17176) |

As described above, during replication of influenza virus, vRNA is transcribed to produce cRNA, which serves as a template for more vRNA synthesis, and mRNA, which serves as a template for protein synthesis (1). Although RNAi is known to target the degradation of mRNA in a sequence-specific manner (16-18), there is a possibility that vRNA and cRNA are also targets for siRNA since vRNA of influenza A virus is sensitive to nuclease (1). To investigate the effect of siRNA on the degradation of various RNA species, reverse transcription using sequence-specific primers followed by real time PCR was used to quantify the levels of vRNA, cRNA and mRNA. The cRNA is the exact complement of vRNA, but mRNA contains a polyA sequence at the 3′ end, beginning at a site complementary to a site 15-22 nucleotides downstream from the 5′ end of the vRNA segment. Thus compared to vRNA and cRNA, mRNA lacks 15 to 22 nucleotides at the 3′ end. To distinguish among the three viral RNA species, primers specific for vRNA, cRNA and mRNA were used in the first reverse transcription reaction. For mRNA, poly dT18 was used as primer. For cRNA, a primer complementary to the 3′ end of the RNA that is missing from mRNA was used. For vRNA, the primer can be almost anywhere along the RNA as long as it is complementary to vRNA and not too close to the 5′ end. The resulting cDNA transcribed from only one of the RNAs was amplified by real time PCR.

Following influenza virus infection, new virions are starting to be packaged and released by about 4 hrs. To determine the effect of siRNA on the first wave of mRNA and cRNA transcription, RNA was isolated early after infection. Briefly, NP-1496 was electroporated into MDCK cells. A mock electroporation (no siRNA) was also performed). Six to eight hours later, cells were infected with PR8 virus at MOI=0.1. The cells were then lysed at 1, 2 and 3 hours post-infection and RNA was isolated. The levels of mRNA, vRNA and cRNA were assayed by reverse transcription using primers for each RNA species, followed by real time PCR.

One hour after infection, there was no significant difference in the amount of NP mRNA between samples with or without NP siRNA transfection. As early as 2 hours post-infection, NP mRNA increased by 38 fold in the mock transfection group, whereas the levels of NP mRNA did not increase (or even slightly decreased) in cells transfected with siRNA. Three hours post-infection, mRNA transcript levels continued to increase in the mock transfection whereas a continuous decrease in the amount of NP mRNA was observed in the cells that received siRNA treatment. NP vRNA and cRNA displayed a similar pattern except that the increase in the amount of vRNA and cRNA in the mock transfection was significant only at 3 hrs post-infection.

These results indicate that, consistent with the results of measuring intact, live virus by hemagglutinin assay or plaque assay, the amounts of all NP RNA species were also significantly reduced by the treatment with NP siRNA. Although it is known that siRNA mainly mediates degradation of mRNA, the data from this experiment does not exclude the possibility of siRNA-mediated degradation of NP cRNA and vRNA although the results described below suggest that reduction in NP protein levels as a result of reduction in NP mRNA results in decreased stability of NP cRNA and/or vRNA.

### EXAMPLE 5

### Identification of the Target of RNA Interference

siRNA preparation of unmodified siRNAs was performed as described above. Modified RNA oligonucleotides, in which the 2′-hydroxyl group was substituted with a 2′-O-methyl group at every nucleotide residue of either the sense or antisense strand, or both, were also synthesized by Dharmacon. Modified oligonucleotides were deprotected and annealed to the complementary strand.as described for unmodified oligonucleotides. siRNA duplexes were analyzed for completion of duplex formation by gel electrophoresis.

Cell culture, transfection with siRNAs, and infection with virus. These were performed essentially as described above. Briefly, for the experiment involving modified NP-1496 siRNA, MDCK cells were first transfected with NP-1496 siRNAs (2.5 nmol) formed from wild type (wt) and modified (m) strands and infected 8 hours later with PR8 virus at a MOI of 0.1. Virus titers in the culture supernatants were assayed 24 hours after infection. For the experiment involving M-37 siRNA, MDCK cells were transfected with M-37 siRNAs (2.5 nmol), infected with PR8 virus at an MOI of 0.01, and harvested for RNA isolation 1, 2, and 3 hours after infection. See Table 2 for M-37 sense and antisense sequences.

RNA extraction, reverse transcription and real time PCR were performed essentially as described above. Primers specific for either mRNA, M-specific vRNA, and M-specific cRNA, used for reverse transcription, were as follows:

(SEQ ID NO: 17170)

mRNA, dT₁₈ =
5′-TTTTTTTTTTTTTTTTTT-3′

(SEQ ID NO: 17177)

M vRNA:
5′-CGCTCAGACATGAGAACAGAATGG-3′

(SEQ ID NO: 17178)

M cRNA:
5′-ATATCGTCTCGTATTAGTAGAAACAAGGTAGTTTTT-3′.

PCR primers for M RNAs were as follows:

| | |
|---|---|
| M forward: | |
| 5′-CGCTCAGACATGAGAACAGAATGG-3′ | (SEQ ID NO: 17177) |
| M reverse: | |
| 5′-TAACTAGCCTGACTAGCAACCTC-3′ | (SEQ ID NO: 17179) |

To investigate the possibility that siRNA might interfere with vRNA and/or cRNA in addition to mRNA, NP-1496 siRNAs in which either the sense (S or +) or antisense (AS or −) strand was modified were synthesized. The modification, which substitutes a 2′-O-methyl group for the 2′-hydroxyl group in every nucleotide residue, does not affect base-pairing for duplex formation, but the modified RNA strand no longer supports RNA interference. In other words, an siRNA in which the sense strand is modified but the antisense strand is wild type will support degradation of RNAs having a sequence complementary to the antisense strand but not a sequence complementary to the sense strand. Conversely, an siRNA in which the sense strand is wild type but the antisense strand is modified will support degradation of RNAs having a sequence complementary to the sense strand but will not support degradation of RNAs having a sequence complementary to the sense strand.

MDCK cells were either mock transfected or transfected with NP-1496 siRNAs in which either the sense strand or the antisense strand, was modified while the other strand was wild type. Cells were also transfected with NP-1496 siRNA in which both strands were modified. Cells were then infected with PR8 virus, and virus titer in supernatants was measured. High virus titers were detected in cultures subjected to mock transfection. As expected, very low virus titers were detected in cultures transfected with wild type siRNA, but high virus titers were detected in cultures transfected with siRNA in which both strands were modified. Virus titers were high in cultures transfected with siRNA in which the antisense strand was modified, whereas the virus titers were low in cultures transfected with siRNA in which the sense strand only was modified. While not wishing to be bound by any theory, the inventors suggest that the requirement for a wild type antisense (−) strand of siRNA duplex to inhibit influenza virus production suggests that the target of RNA interference is either mRNA (+) or cRNA (+) or both.

To further distinguish these possibilities, the effect of siRNA on the accumulation of corresponding mRNA, vRNA, and cRNA was examined. To follow transcription in a cohort of simultaneously infected cells, siRNA-transfected MDCK cells were harvested for RNA isolation 1, 2, and 3 hours after infection (before the release and re-infection of new virions). The viral mRNA, vRNA, and cRNA were first independently converted to cDNA by reverse transcription using specific primers. Then, the level of each cDNA was quantified by real time PCR. When M-specific siRNA M-37 was used, little M-specific mRNA was detected one or two hours after infection. Three hours after infection, M-specific mRNA was readily detected in the absence of M-37. In cells transfected with M-37, the level of M-specific mRNA was reduced by approximately 50%. In contrast, the levels of M-specific vRNA and cRNA were not inhibited by the presence of M-37. While not wishing to be bound by any theory, these results indicate that viral mRNA is probably the target of siRNA-mediated interference.

### EXAMPLE 6

### Effects of Certain siRNAs on Viral RNA Accumulation

siRNA preparation was performed as described above. Primers were specific for either mRNA, NP vRNA, NP cRNA, NS vRNA, NS cRNA, M vRNA, or M cRNA. Primers specific for PB1 vRNA, PB1 cRNA, PB2 vRNA, PB2 cRNA, PA vRNA, or PA cRNA, used for reverse transcription, were as follows:

(SEQ ID NO: 17180)

| | |
|---|---|
| PB1 vRNA: | 5′-GTGCAGAAATCAGCCCGAATGGTTC-3′ |

(SEQ ID NO: 17181)

| | |
|---|---|
| PB1 cRNA: | 5′-ATATCGTCTCGTATTAGTAGAAACAAGGCATTT-3′ |

(SEQ ID NO: 17182)

| | |
|---|---|
| PB2 vRNA: | 5′-GCGAAAGGAGAGAAGGCTAATGTG-3′ |

(SEQ ID NO: 17183)

| | |
|---|---|
| PB2 cRNA: | 5′-ATATGGTCTCGTATTAGTAGAAACAAGGTCGTTT-3′ |

(SEQ ID NO: 17184)

| | |
|---|---|
| PA vRNA: | 5′-GCTTCTTATCGTTCAGGCTCTTAGG-3′ |

(SEQ ID NO: 17185)

| | |
|---|---|
| PA cRNA: | 5′-ATATCGTCTCGTATTAGTAGAAACAAGGTACTT-3′ |

PCR primers for PB1, PB2, and PA RNAs were as follows:

(SEQ ID NO: 17186)

| | |
|---|---|
| PB1 forward: | 5′-CGGATTGATGCACGGATTGATTTC-3′ |

(SEQ ID NO: 17187)

| | |
|---|---|
| PB1 reverse: | 5′-GACGTCTGAGCTCTTCAATGGTGGAAC-3′ |

(SEQ ID NO: 17182)

| | |
|---|---|
| PB2 forward: | 5′-GCGAAAGGAGAGAAGGCTAATGTG-3′ |

(SEQ ID NO: 17188)

| | |
|---|---|
| PB2 reverse: | 5′-AATCGCTGTCTGGCTGTCAGTAAG-3′ |

(SEQ ID NO: 17184)

| | |
|---|---|
| PA forward: | 5′-GCTTCTTATCGTTCAGGCTCTTAGG-3′ |

(SEQ ID NO: 17189)

| | |
|---|---|
| PA reverse: | 5′-CCGAGAAGCATTAAGCAAAACCCAG-3′ |

To determine whether NP-1496 targets the degradation of the NP gene segment specifically or whether the levels of viral RNAs other than NP are also affected, primers specific for NS were used for RT and real time PCR to measure the amount of different NS RNA species (mRNA, vRNA, cRNA) as described above. The changes in NS mRNA, vRNA and cRNA showed the same pattern as that observed for NP RNAs. At 3 hours post-infection, a significant increase in all NS RNA species could be seen in mock transfected cells, whereas no significant changes in NS RNA levels were seen in the cells that received NP-1496 siRNA. This result indicates that the transcription and replication of different viral RNAs are coordinately regulated, at least with respect to NP RNAs. By coordinately regulated is meant that levels of one transcript affect levels of another transcript, either directly or indirectly. No particular mechanism is implied. When NP transcripts are degraded by siRNA treatment the levels of other viral RNAs are also reduced.

To further explore the effect of NP siRNAs on other viral RNAs, accumulation of mRNA, vRNA, and cRNA of all viral genes was measured in cells that had been treated with NP-1496. NP-specific mRNA was low one or two hours after infection. Three hours after infection, NP mRNA was readily detected in the absence of NP-1496, whereas in the presence of NP-1496, the level of NP mRNA remained at the background level, indicating that siRNA inhibited the accumulation of specific mRNA. Levels of NP-specific and NS-specific vRNA and cRNA were greatly inhibited by the presence of NP-1496. In addition, in the NP-1496-treated cells, the accumulation of mRNA, vRNA, and cRNA of the M, NS, PB1, PB2, and PA genes was also inhibited. Furthermore, the broad inhibitory effect was also observed for PA-2087. At three hours after infection PA, M, and NS mRNA were readily detected in the absence of PA-2087, whereas the presence of PA-2087 inhibited transcription of PA, M, and NS mRNA. At three hours after infection PA, M, and NS vRNA were readily detected in the absence of PA-2087, whereas the presence of PA-2087 inhibited accumulation of PA, M, and NS vRNA. At three hours after infection PA, M, and NS cRNA were readily detected in the absence of PA-2087, whereas the presence of PA-2087 inhibited accumulation of PA, M, and NS cRNA. In addition, NP-specific siRNA inhibits the accumulation of PB1-, PB2- and PA-specific mRNA.

The NP gene segment in influenza virus encodes a single-stranded RNA-binding nucleoprotein, which can bind to both vRNA and cRNA. During the viral life cycle, NP mRNA is first transcribed and translated. The primary function of the NP protein is to encapsidate the virus genome for the purpose of RNA transcription, replication and packaging. In the absence of NP protein, the full-length synthesis of both vRNA and cRNA is strongly impaired. When NP siRNA induces the degradation of NP RNA, NP protein synthesis is impaired and the resulting lack of sufficient NP protein subsequently affects the replication of other viral gene segments. In this way, NP siRNA could potently inhibit virus production at a very early stage.

The number of NP protein molecules in infected cells has been hypothesized to regulate the levels of mRNA synthesis versus genome RNA (vRNA and cRNA) replication (1). Using a temperature-sensitive mutation in the NP protein, previous studies have shown that cRNA, but not mRNA, synthesis was temperature sensitive both in vitro and in vivo. NP protein was shown to be required for elongation and antitermination of the nascent cRNA and vRNA transcripts. The results presented above show that NP-specific siRNA inhibited the accumulation of all viral RNAs in infected cells. While not wishing to be bound by any theory, it appears probable that in the presence of NP-specific siRNA, the newly transcribed NP mRNA is degraded, resulting in the inhibition of NP protein synthesis following virus infection. Without newly synthesized NP, further viral transcription and replication, and therefore new virion production is inhibited.

Similarly, in the presence of PA-specific, the newly transcribed PA mRNA is degraded, resulting in the inhibition of PA protein synthesis. Despite the presence of 30-60 copies of RNA transcriptase per influenza virion (1), without newly synthesized RNA transcriptase, further viral transcription and replication are likely inhibited. Similar results were obtained using siRNA specific for PB1. In contrast, the matrix (M) protein is not required until the late phase of virus infection (1). Thus, M-specific siRNA inhibits the accumulation of M-specific mRNA but not vRNA, cRNA, or other viral RNAs. Taken together, these findings demonstrate a critical requirement for newly synthesized nucleoprotein and polymerase proteins in influenza viral RNA transcription and replication. Both mRNA- and virus-specific mechanisms by which NP-, PA-, and PB1-specific siRNAs interfere with mRNA accumulation and other viral RNA transcription suggest that these siRNAs may be especially potent inhibitors of influenza virus infection.

### EXAMPLE 7

### Broad Inhibition of Influenza Virus RNA Accumulation by Certain siRNAs is Not Due to the Interferon Response or to Virus-Induced RNA Degradation

RNA levels were measured using PCR under standard conditions. The following PCR primers were used for measurement of γ-actin RNA.

(SEQ ID NO: 17190)

γ-actin forward:
5′-TCTGTCAGGGTTGGAAAGTC-3′

(SEQ ID NO: 17191)

γ-actin reverse:
5′-AAATGCAAACCGCTTCCAAC-3′

One possible cause for the broad inhibition of viral RNA accumulation described above is an interferon response of the infected cells in the presence of siRNA. Thus, the above experiments were repeated in Vero cells in which the entire IFN locus, including all α, β, and ω genes, are deleted. Just as in MDCK cells, the accumulation of NP-, M-, and NS-specific mRNAs were all inhibited by NP-1496. In addition, the effect of siRNA on the levels of transcripts from cellular genes, including β-actin, γ-actin, and GAPDH, was assayed using PCR. No significant difference in the transcript levels was detected in the absence or presence of siRNA indicating that the inhibitory effect of siRNA is specific for viral RNAs. These results suggest that the broad inhibition of viral RNA accumulation by certain siRNAs is not a result of a cellular interferon response.

Following influenza virus infection, the presence of dsRNA also activates a cellular pathway that targets RNA for degradation. To examine the effect of siRNA on the activation of this pathway, we assayed the levels of phosphorylated protein kinase R (PKR), the most critical component of the pathway. Transfection of MDCK cells with NP-1496 in the absence of virus infection did not affect the levels of activated PKR. Infection by influenza virus resulted in an increased level of phosphorylated PKR, consistent with previous studies. However, the increase was the same in the presence or absence of NP-1496. Thus, the broad inhibition of viral RNA accumulation is not a result of enhanced virus-induced degradation in the presence of siRNA.

### EXAMPLE 8

### Inhibition of Influenza Virus Production in Mice by siRNAs

This example describes experiments showing that administration of siRNAs targeted to influenza virus NP or PA transcripts inhibit production of influenza virus in mice when administered either prior to or following infection with influenza virus. The inhibition is dose-dependent and shows additive effects when two siRNAs each targeted to a transcript expressed from a different influenza virus gene were administered together. Materials and Methods:

siRNA preparation. This was performed as described above.

siRNA delivery. siRNAs (30 or 60 μg of GFP-949, NP-1496, or PA-2087) were incubated with jetPEI™ for oligonucleotides cationic polymer transfection reagent, N/P ratio=5 (Qbiogene, Inc., Carlsbad, Calif.; Cat. No. GDSP20130; N/P refers to the number of nitrogens per nucleotide phosphate in the jetPEI/siRNA mixture) or with poly-L-lysine (MW (vis) 52,000; MW (LALLS) 41,800, Sigma Cat. No. P2636) for 20 min at room temperature in 5% glucose. The mixture was injected into mice intravenously, into the retro-orbital vein, 200 μl per mouse, 4 mice per group. 200 μl 5% glucose was injected into control (no treatment) mice. The mice were anesthetized with 2.5% Avertin before siRNA injection or intranasal infection.

Viral infection. B6 mice (maintained under standard laboratory conditions) were intranasally infected with PR8 virus by dropping virus-containing buffer into the mouse's nose with a pipette, 30 ul (12,000 pfu) per mouse.

Determination of viral titer. Mice were sacrificed at various times following infection, and lungs were harvested. Lungs were homogenized, and the homogenate was frozen and thawed twice to release virus. PR8 virus present in infected lungs was titered by infection of MDCK cells. Flat-bottom 96-well plates were seeded with 3×10⁴ MDCK cells per well, and 24 hrs later the serum-containing medium was removed. 25 μl of lung homogenate, either undiluted or diluted from 1×10⁻¹ to 1×10⁻⁷, was inoculated into triplicate wells. After 1 h incubation, 175 μl of infection medium with 4 μg/ml of trypsin was added to each well. Following a 48 h incubation at 37° C., the presence or absence of virus was determined by hemagglutination of chicken RBC by supernatant from infected cells. The hemagglutination assay was carried out in V-bottom 96-well plates. Serial 2-fold dilutions of supernatant were mixed with an equal volume of a 0.5% suspension (vol/vol) of chicken erythrocytes (Charles River Laboratories) and incubated on ice for 1 h. Wells containing an adherent, homogeneous layer of erythrocytes were scored as positive. The virus titers were determined by interpolation of the dilution end point that infected 50% of wells by the method of Reed and Muench (TCID₅₀), thus a lower TCID₅₀ reflects a lower virus titer. The data from any two groups were compared by Student t test, which was used throughout the experiments described herein to evaluate significance.

siRNA targeted to viral NP transcripts inhibits influenza virus production in mice when administered prior to infection. 30 or 60 μg of GFP-949 or NP-1496 siRNAs were incubated with jetPEI and injected intravenously into mice as described above in Materials and Methods. Three hours later mice were intranasally infected with PR8 virus, 12000 pfu per mouse. Lungs were harvested 24 hours after infection. The average log₁₀ TCID₅₀ of the lung homogenate for mice that received no siRNA treatment or received an siRNA targeted to GFP was 4.2. In mice that were pretreated with 30 μg siRNA targeted to NP and jetPEI, the average log₁₀ TCID₅₀ of the lung homogenate was 3.9. In mice that were pretreated with 60 μg siRNA targeted to NP and jetPEI, the average log₁₀ TCID₅₀ of the lung homogenate was 3.2. The difference in virus titer in the lung homogenate between the group that received no treatment and the group that received 60 μg NP siRNA was significant with P=0.0002. Data for individual mice are presented in Table 11A.

siRNA targeted to viral NP transcripts inhibits influenza virus production in mice when administered intravenously prior to infection in a composition containing the cationic polymer PLL. 30 or 60 μg of GFP-949 or NP-1496 siRNAs were incubated with PLL and injected intravenously into mice as described above in Materials and Methods. Three hours later mice were intranasally infected with PR8 virus, 12000 pfu per mouse. Lungs were harvested 24 hours after infection. The average log₁₀ TCID₅₀ of the lung homogenate for mice that received no siRNA treatment (NT) or received an siRNA targeted to GFP (GFP 60 μg) was 4.1. In mice that were pretreated with 60 μg siRNA targeted to NP (NP 60 μg) and PLL, the average log₁₀ TCID₅₀ of the lung homogenate was 3.0. The difference in virus titer in the lung homogenate between the group that received 60 μg GFP and the group that received 60 μg NP siRNA was significant with P=0.001. Data for individual mice are presented in Table 11A. These data indicate that siRNA targeted to the influenza NP transcript reduced the virus titer in the lung when administered prior to virus infection. They also indicate that a mixtures of an siRNA with a cationic polymer effectively inhibits influenza virus in the lung when administered by intravenous injection, not requiring techniques such as hydrodynamic transfection.

TABLE 11A
Inhibition of influenza virus production in mice by siRNA with
cationic polymers

| | |
|---|---|
| Treatment | log₁₀TCID50 |

| | | | | |
|---|---|---|---|---|
| NT (jetPEI experiment) | 4.3 | 4.3 | 4.0 | 4.0 |
| GFP (60 μg) + jetPEI | 4.3 | 4.3 | 4.3 | 4.0 |
| NP (30 μg) + jetPEI | 4.0 | 4.0 | 3.7 | 3.7 |
| NP (60 μg) + jetPEI | 3.3 | 3.3 | 3.0 | 3.0 |
| NT (PLL experiment) | 4.0 | 4.3 | 4.0 | 4.0 |
| GFP (60 μg) + PLL | 4.3 | 4.0 | 4.0 | (not done) |
| NP (60 μg) + PLL | 3.3 | 3.0 | 3.0 | 2.7 |

siRNA targeted to viral NP transcripts inhibits influenza virus production in mice when administered prior to infection and demonstrates that the presence of a cationic polymer significantly increases the inhibitory efficacy of siRNA. 60 μg of GFP-949 or NP-1496 siRNAs were incubated with phosphate buffered saline (PBS) or jetPEI and injected intravenously into mice as described above in Materials and Methods. Three hours later mice were intranasally infected with PR8 virus, 12000 pfu per mouse. Lungs were harvested 24 hours after infection. The average log₁₀ TCID₅₀ of the lung homogenate for mice that received no siRNA treatment was 4.1, while the average log₁₀ TCID₅₀ of the lung homogenate for mice that received an siRNA targeted to GFP in PBS was 4.4. In mice that were pretreated with 60 μg siRNA targeted to NP in PBS the average log₁₀ TCID₅₀ of the lung homogenate was 4.2, showing only a modest increase in efficacy relative to no treatment or treatment with an siRNA targeted to GFP. In mice that were pretreated with 60 μg siRNA targeted to GFP in jetPEI, the average log₁₀ TCID₅₀ of the lung homogenate was 4.2. However, in mice that received 60 μg siRNA targeted to NP in jetPEI, the average log₁₀ TCID₅₀ of the lung homogenate was 3.2. The difference in virus titer in the lung homogenate between the group that received GFP siRNA in PBS and the group that received NP siRNA in PBS was significant with P=0.04, while the difference in virus titer in the lung homogenate between the group that received GFP siRNA with jetPEI and the group that received NP siRNA with jetPEI was highly significant with P=0.003. Data for individual mice are presented in Table 11B.

TABLE 11B
Inhibition of influenza virus production in mice by siRNA
showing increased efficacy with cationic polymer

| | |
|---|---|
| Treatment | log₁₀TCID50 |

| | | | | |
|---|---|---|---|---|
| NT | 4.3 | 4.3 | 4.0 | 3.7 |
| GFP (60 μg) + PBS | 4.3 | 4.3 | 4.7 | 4.3 |
| NP (60 μg) + PBS | 3.7 | 4.3 | 4.0 | 4.0 |
| GFP (60 μg) + jetPEI | 4.3 | 4.3 | 4.0 | 3.0 |
| NT (60 μg) + jetPEI | 3.3 | 3.0 | 3.7 | 3.0 |

### Additional experiments were performed to assess the ability of siRNA to inhibit influenza virus production at various times after infection, when administered at various time points prior to or following infection.

siRNA was administered as described above except that 120 ug siRNA was administered 12 hours before virus infection. Table 11C shows the results expressed as log₁₀ TCID₅₀. The P value comparing NP-treated with control group was 0.049

TABLE 11C

| | Mouse 1 | Mouse 2 | Mouse 3 | Mouse 4 |
|---|---|---|---|---|
| NT | 4.3 | 4 | 4 | 4 |
| GFP-949 | 4.3 | 4 | 4 | 4 |
| NP-1496 | 4 | 3.7 | 3.7 | 3.3 |

In another experiment, siRNA (60 ug) was administered 3 hours before infection. 1500 pfu of PR8 virus was administered intranasally. The infected lung was harvested 48 h after infection. Table 11D shows the results expressed as log₁₀ TCID₅₀. The P value comparing NP-treated with control group was 0.03.

TABLE 11D

| | Mouse 1 | Mouse 2 | Mouse 3 | Mouse 4 |
|---|---|---|---|---|
| NT | 4 | 4 | 4 | 4 |
| GFP-949 | 4.3 | 4 | 4 | 3.7 |
| NP-1496 | 3 | 3.7 | 3.7 | 3.3 |

In another experiment, siRNA (120 ug) was administered 24 hours after PR8 (1500 pfu) infection. 52 hours post-infection, the lung was harvested and virus titer was measured. Table 11E shows the results expressed as log₁₀ TCID₅₀. The P value comparing NP-treated with control group was 0.03.

TABLE 11E

| | Mouse 1 | Mouse 2 | Mouse 3 | Mouse 4 |
|---|---|---|---|---|
| GFP-949 | 2.3 | 2.7 | 2 | 2.7 |
| NP-1496 | 2 | 2 | 1.7 | 2 |

Other polymers were also shown to be effective siRNA delivery agents. FIG. 19D is a plot showing that siRNA targeted to NP (NP-1496) inhibits influenza virus production in mice when administered intravenously together with a poly(beta amino ester) (J28). siRNA targeted to NP (NP-1496) inhibits influenza virus production in mice when administered intraperitoneally together with a poly(beta amino ester) while a control RNA has no significant effect. The experiments were performed essentially as described above except that the ratio of polymer to siRNA was a weight/weight ratio (for instance, 60:1 w/w). Polymers and siRNA were mixed and administered to mice either intravenously or intraperitoneally 3 hours prior to intranasal infection with 12,000 pfu of PR8 virus. Lungs were harvested 24 hours later and HA assays were performed. The amine and bis(acrylate ester)monomers present in J28 and C32 are described and depicted in U.S. Ser. No. 10/446,444.

siRNAs targeted to different influenza virus transcripts exhibit an additive effect. Sixty μg of NP-1496 siRNA, 60 μg PA-2087 siRNA, or 60 μg NP-1496 siRNA+60 μg PA-2087 siRNA were incubated with jetPEI and injected intravenously into mice as described above. Three hours later mice were intranasally infected with PR8 virus, 12000 pfu per mouse. Lungs were harvested 24 hours after infection. The average log₁₀ TCID₅₀ of the lung homogenate for mice that received no siRNA treatment was 4.2. In mice that received 60 μg siRNA targeted to NP, the average log₁₀ TCID₅₀ of the lung homogenate was 3.2. In mice that received 60 μg siRNA targeted to PA, the average log₁₀ TCID₅₀ of the lung homogenate was 3.4. In mice that received 60 μg siRNA targeted to NP+60 μg siRNA targeted to PA, the average log₁₀ TCID₅₀ of the lung homogenate was 2.4. The differences in virus titer in the lung homogenate between the group that received no treatment and the groups that received 60 μg NP siRNA, 60 μg PA siRNA, or 60 μg NP siRNA+60 μg PA siRNA were significant with P=0.003, 0.01, and 0.0001, respectively. The differences in lung homogenate between the groups that received 60 μg NP siRNA or 60 μg NP siRNA and the group that received 60 μg NP siRNA+60 μg PA siRNA were significant with p=0.01. Data for individual mice are presented in Table 12. These data indicate that pretreatment with siRNA targeted to the influenza NP or PA transcript reduced the virus titer in the lungs of mice subsequently infected with influenza virus. The data further indicate that a combination of siRNA targeted to different viral transcripts exhibit an additive effect, suggesting that therapy with a combination of siRNAs targeted to different transcripts may allow a reduction in dose of each siRNA, relative to the amount of a single siRNA that would be needed to achieve equal efficacy.

TABLE 12
Additive effect of siRNA against influenza virus in mice

| | |
|---|---|
| Treatment | log₁₀TCID50 |

| | | | | |
|---|---|---|---|---|
| NT | 4.3 | 4.3 | 4.0 | 4.0 |
| NP (60 μg) | 3.7 | 3.3 | 3.0 | 3.0 |
| PA (60 μg) | 3.7 | 3.7 | 3.0 | 3.0 |
| NP + PA (60 μg each) | 2.7 | 2.7 | 2.3 | 2.0 |

siRNA targeted to viral NP transcripts inhibits influenza virus production in mice when administered following infection. Mice were intranasally infected with PR8 virus, 500 pfu. Sixty μg of GFP-949 siRNA, 60 μg PA-2087 siRNA, 60 μg NP-1496 siRNA, or 60 μg NP siRNA+60 μg PA siRNA were incubated with jetPEI and injected intravenously into mice 5 hours later as described above. Lungs were harvested 28 hours after administration of siRNA. The average log₁₀ TCID₅₀ of the lung homogenate for mice that received no siRNA treatment or received the GFP-specific siRNA GFP-949 was 3.0. In mice that received 60 μg siRNA targeted to PA, the average log₁₀ TCID₅₀ of the lung homogenate was 2.2. In mice that received 60 μg siRNA targeted to NP (NP 60 μg), the average log₁₀ TCID₅₀ of the lung homogenate was 2.2. In mice that received 60 μg NP siRNA+60 μg PA siRNA, the average log₁₀ TCID₅₀ of the lung homogenate was 1.8. The differences in virus titer in the lung homogenate between the group that received no treatment and the groups that received 60 μg PA, NP siRNA, or 60 μg NP siRNA+60 μg PA siRNA were significant with P=0.09, 0.02, and 0.003, respectively. The difference in virus titer in the lung homogenate between the group that received NP siRNA and PA+NP siRNAs had a P value of 0.2. Data for individual mice are presented in Table 13. These data indicate that siRNA targeted to the influenza NP and/or PA transcripts reduced the virus titer in the lung when administered following virus infection.

TABLE 13
Inhibition of influenza virus production in infected mice by siRNA

| | |
|---|---|
| Treatment | log₁₀TCID50 |

| | | | | |
|---|---|---|---|---|
| NT | 3.0 | 3.0 | 3.0 | 3.0 |
| GFP (60 μg) | 3.0 | 3.0 | 3.0 | 2.7 |
| PA (60 μg) | 2.7 | 2.7 | 2.3 | 1.3 |
| NP (60 μg) | 2.7 | 2.3 | 2.3 | 1.7 |
| NP + PA (60 μg each) | 2.3 | 2.0 | 1.7 | 1.3 |

### EXAMPLE 9

### Inhibition of Influenza Virus Production in Cells by Administration of a Lentivirus that Provides a Template for Production of shRNA

An oligonucleotide that serves as a template for synthesis of an NP-1496a shRNA was cloned between the U6 promoter and termination sequence of lentiviral vector pLL3.7 (Rubinson, D., et al., Nature Genetics 33:401-406, 2003). The oligonucleotide was inserted between the HpaI and XhoI restriction sites within the multiple cloning site of pLL3.7. This lentiviral vector also expresses EGFP for easy monitoring of transfected/infected cells. Lentivirus was produced by co-transfecting the DNA vector comprising a template for production of NP-1496a shRNA and packaging vectors into 293T cells. Forty-eight h later, culture supernatant containing lentivirus was collected, spun at 2000 rpm for 7 min at 4° C. and then filtered through a 0.45 um filter. Vero cells were seeded at 1×10⁵ per well in 24-well plates. After overnight culture, culture supernatants containing that contained the insert (either 0.25 ml or 1.0 ml) were added to wells in the presence of 8 ug/ml polybrene. The plates were then centrifuged at 2500 rpm, room temperature for 1 h and returned to culture. Twenty-four h after infection, the resulting Vero cell lines (Vero-NP-0.25, and Vero-NP-1.0) were analyzed for GFP expression by flow cytometry along with parental (non-infected) Vero cells. It is noted that NP-1496a differs from NP-1496 due to the inadvertent inclusion of an additional nucleotide (A) at the 3′ end of the sense portion and a complementary nucleotide (U) at the 5′ end of the antisense portion, resulting in a duplex portion that is 20 nt in length rather than 19 as in NP-1496. (See Table 2). According to other embodiments of the invention NP-1496 sequences rather than NP-1496a sequences are used. In addition, the loop portion of NP-1496a shRNA differs from that of NP-1496 shRNA.

Control Vero cells and Vero cells infected with lentivirus containing the insert (Vero-NP-0.25 and Vero-NP-1.0) were infected with PR8 virus at MOI of 0.04, 0.2 and 1. Influenza virus titers in the supernatants were determined by HA assay 48 hrs after infection as described above.

Lentivirus containing templates for production of NP-1496a shRNA were tested for ability to inhibit influenza virus production in Vero cells. The NP-1496a shRNA includes two complementary regions capable of forming a stem-loop structure containing a double-stranded portion that has the same sequence as the NP-1496a siRNA described above. Incubation of lentivirus-containing supernatants with Vero cells overnight resulted in expression of EGFP, indicating infection of Vero cells by lentivirus. The shaded curve represents mean fluorescence intensity in control cells (uninfected). When 1 ml of supernatant was used, almost all cells became EGFP positive and the mean fluorescence intensity was high (1818) (Vero-NP-1.0). When 0.25 ml of supernatant was used, most cells (˜95%) were EGFP positive and the mean fluorescence intensity was lower (503) (Vero-NP-0.25).

Parental Vero cells and lentivirus-infected Vero cells were then infected with influenza virus at MOI of 0.04, 0.2, and 0.1, and virus titers were assayed 48 hrs after influenza virus infection. With increasing MOI, the virus titers increased in the supernatants of parental Vero cell cultures. In contrast, the virus titers remained very low in supernatants of Vero-NP-1.0 cell cultures, indicating influenza virus production was inhibited in these cells. Similarly, influenza virus production in Vero-NP-0.25 cell cultures was also partially inhibited. The viral titers are presented in Table 14. These results suggest that NP-1496 shRNA expressed from lentivirus vectors can be processed into siRNA to inhibit influenza virus production in Vero cells. The extent of inhibition appears to be proportional to the extent of virus infection per cell (indicated by EGFP level).

TABLE 14
Inhibition of influenza virus production by siRNA
expressed in cells in tissue culture

| Cell Line | Viral Titer | | |
|---|---|---|---|
| Vero | 16 | 64 | 128 |
| Vero-NP-0.25 | 8 | 32 | 64 |
| Vero-NP-1.0 | 1 | 4 | 8 |

### EXAMPLE 10

### Inhibition of Influenza Production in Mice by Intranasal Administration of a DNA Vector from Which siRNA Precursors are Transcribed

Construction of a plasmid from which NP-1496a shRNA is expressed is described above. Oligonucleotides that serve as templates for synthesis of PB1-2257 shRNA or RSV-specific shRNA were cloned between the U6 promoter and termination sequence of lentiviral vector pLL3.7 as described above for NP-1496a shRNA. The sequences of the oligonucleotides were as follows:

(SEQ ID NO: 17192)

NP-1496a sense:
5′-
TGGATCTTATTTCTTCGGAGATTCAAGAGATCTCCGAAGAAATAAGATCC
TTTTTTC-3′

(SEQ ID NO: 17193)

NP-1496a antisense:
5′-
TCGAGAAAAAAGGATCTTATTTCTTCGGAGATCTCTTGAATCTCCGAAGA
AATAAGATCCA-3′

(SEQ ID NO: 17194)

PB1-2257 sense:
5′-
TGATCTGTTCCACCATTGAATTCAAGAGATTCAATGGTGGAACAGATCTT
TTTTC-3′

(SEQ ID NO: 17195)

PB1-2257 antisense:
5′-
TCGAGAAAAAAGATCTGTTCCACCATTGAATCTCTTGAATTCAATGGTGG
AACAGATCA-3′

(SEQ ID NO: 17196)

RSV sense:
5′-
TGCGATAATATAACTGCAAGATTCAAGAGATCTTGCAGTTATATTATCGT
TTTTTC-3′

(SEQ ID NO: 17197)

RSV antisense:
5′-
TCGAGAAAAAACGATAATATAACTGCAAGATCTCTTGAATCTTGCAGTTA
TATTATCGCA-3′

The RSV shRNA expressed from the vector comprising the above oligonucleotide is processed in vivo to generate an siRNA having sense and antisense strands with the following sequences:

(SEQ ID NO: 17198)

Sense:
5′-CGATAATATAACTGCAAGA-3′

(SEQ ID NO: 17199)

Antisense:
5′-TCTTGCAGTTATATTATCG-3′
A PA-specific hairpin may be similarly constructed
using the following oligonucleotides:

(SEQ ID NO: 17200)

PA-2087 sense:
5′-
TGCAATTGAGGAGTGCCTGATTCAAGAGATCAGGCACTCCTCAATTGCTT
TTTTC-3′

(SEQ ID NO: 17201)

PA-2087 antisense:
5′-
TCGAGAAAAAAGCAATTGAGGAGTGCCTGATCTCTTGAATCAGGCACTCC
TCAATTGCA-3′

Plasmid DNAs capable of serving as templates for expression of NP-1496a shRNA, PB1-2257 shRNA, or RSV-specific shRNA (60 μg each) were individually mixed with 40 μl Infasurf ® (ONY, Inc., Amherst N.Y.) and 20 μl of 5% glucose and were administered intranasally to groups of mice, 4 mice each group, as described above. A mixture of 40 μl Infasurf and 20 μl of 5% glucose was administered to mice in the no treatment (NT) group. The mice were intranasally infected with PR8 virus, 12000 pfu per mouse, 13 hours later, as described above. Lungs were harvested and viral titer determined 24 hours after infection.

The ability of shRNAs expressed from DNA vectors to inhibit influenza virus infection in mice was tested. For these experiments, plasmid DNA was mixed with Infasurf, a natural surfactant extract from calf lung similar to vehicles previously shown to promote gene transfer in the lung. The DNA/Infasurf mixtures were instilled into mice by dropping the mixture into the nose using a pipette. Mice were infected with PR8 virus, 12000 pfu per mouse, 13 hours later. Twenty-four hrs after influenza virus infection, lungs were harvested and virus titers were measured by MDCK/hemagglutinin assay.

Virus titers were high in mice that were not given any plasmid DNA or were given a DNA vector expressing a respiratory syncytial virus (RSV)-specific shRNA. Lower virus titers were observed when mice were given plasmid DNA that expresses either NP-1496a shRNA or PB1-2257 shRNA. The virus titers were more significantly decreased when mice were given both influenza-specific plasmid DNAs together, one expressing NP-1496a shRNA and the other expressing PB1-2257 shRNA.

The average log₁₀ TCID₅₀ of the lung homogenate for mice that received no treatment or received a plasmid encoding an RSV-specific shRNA was 4.0 or 4.1, respectively. In mice that received plasmid capable of serving as a template for NP-1496a shRNA, the average log₁₀ TCID₅₀ of the lung homogenate was 3.4. In mice that received plasmid capable of serving as a template for PB1-2257 shRNA, the average log₁₀ TCID₅₀ of the lung homogenate was 3.8. In mice that received plasmids capable of serving as templates for NP and PB shRNAs, the average log₁₀ TCID₅₀ of the lung homogenate was 3.2. The differences in virus titer in the lung homogenate between the group that received no treatment or RSV-specific shRNA plasmid and the groups that received NP shRNA plasmid, PB1 shRNA plasmid, or NP and PB1 shRNA plasmids had P values of 0.049, 0.124, and 0.004 respectively. Data for individual mice are presented in Table 15. These results show that shRNA expressed from DNA vectors can be processed into siRNA to inhibit influenza virus production in mice and demonstrate that Infasurf is a suitable vehicle for the delivery of plasmids from which shRNA can be expressed. In particular, these data indicate that shRNA targeted to the influenza NP and/or PB1 transcripts reduced the virus titer in the lung when administered following virus infection.

TABLE 15
Inhibition of influenza virus production by shRNA expressed in mice

| | |
|---|---|
| Treatment | log₁₀TCID50 |

| | | | | |
|---|---|---|---|---|
| NT | 4.3 | 4.0 | 4.0 | 4.3 |
| RSV (60 μg) | 4.3 | 4.0 | 4.0 | 4.0 |
| NP (60 μg) | 4.0 | 3.7 | 3.0 | 3.0 |
| PB1 (60 μg) | 4.0 | 4.0 | 3.7 | 3.3 |
| NP + PB1 (60 μg each) | 3.7 | 3.3 | 3.0 | 3.0 |

### EXAMPLE 11A

### Inhibition of Luciferase Activity in the Lung by Delivery of siRNA to the Vascular System or the Respiratory Tract

siRNAs were obtained from Dharmacon and were deprotected and annealed as described above. siRNA sequences for NP (NP-1496), PA (PA-2087), PB1 (PB1-2257), and GFP were as given above. Luc-specific siRNA was as described in (McCaffrey, A P, et al., Nature 418:38-39)

pCMV-luc DNA (Promega) was mixed with PEI (Qbiogene, Carlsbad, Calif.) at a nitrogen/phosphorus molar ratio (N/P ratio) of 10 at room temperature for 20 min. For i.v. administration, 200 μl of the mixture containing 60 μg of DNA was injected retroorbitally into 8 week old male C57BL/6 mice (Taconic Farms). For intratracheal (i.t.) administration, 50 μl of the mixture containing 30 μg or 60 μg of DNA was administered into the lungs of anesthetized mice using a Penn Century Model 1A-1C insufflator.

siRNA-PEI compositions were formed by mixing 60 μg of luc-specific or GFP-specific siRNA with jetPEI at an N/P ratio of 5 at room temperature for 20 min. For i.v. administration, 200 μl of the mixture containing the indicated amounts of siRNA was injected retroorbitally. For pulmonary administration, 50 μl was delivered intratracheally.

At various times after pCMV-luc DNA administration, lungs, spleen, liver, heart, and kidney were harvested and homogenized in Cell Lysis Buffer (Marker Gene Technologies, Eugene, Oreg.). Luminescence was analyzed with the Luciferase Assay System (Promega) and measured with an Optocomp®I luminometer (MGM Instruments, Hamden, Conn.). The protein concentrations in homogenates were measured by the BCA assay (Pierce).

To determine the tissue distribution of PEI-mediated nucleic acid delivery in mice, pCMV-luc DNA-PEI complexes were injected i.v., and 24 hr later, Luc activity was measured in various organs. Activity was highest in the lungs, where Luc activity was detected for at least 4 days, whereas in heart, liver, spleen, and kidney, levels were 100-1,000 times lower and were detected for a shorter time after injection. When DNA-PEI complexes were instilled i.t., significant Luc activity was also detected in the lungs, although at a lower level than after i.v. administration.

To test the ability of PEI to promote uptake of siRNAs by the lungs following i.v. administration, mice were first given pCMV-luc DNA-PEI complexes i.t., followed by i.v. injection of Luc-specific siRNA complexed with PEI, control GFP-specific siRNA complexed with PEI, or the same volume of 5% glucose. Twenty-four hours later, Luc activity in the lungs was 17-fold lower in mice that received Luc siRNA than in those given GFP siRNA or no treatment. Because Luc siRNA can inhibit Luc expression only in the same lung cells that were transfected with the DNA vector, these results indicate that i.v. injection of a siRNA-PEI mixture achieves effective inhibition of a target transcript in the lung.

To test the ability of PEI to promote uptake of siRNAs by the lungs following pulmonary administration, mice were first given pCMVDNA-PEI complexes i.v., followed immediately by i.t. administration of Luc-specific siRNA mixed with PEI, control GFP-specific siRNA mixed with PEI, or the same volume of 5% glucose. Twenty-four hours later, luciferase activities were assayed in lung homogenates. Luciferase activity was 6.8-fold lower in mice that were treated with luciferase siRNA than those treated with GFP siRNA. These results indicate that pulmonary administration of an siRNA-PEI mixture achieves effective inhibition of a target transcript in lung cells.

### EXAMPLE 11B

### Inhibition of Cyclophilin B in the Lung by Delivery of siRNA to the Respiratory System

Cyclophilin B is an endogenous gene that is widely expressed in mammals. To assess the ability of siRNA delivered directly to the respiratory system to inhibit expression of an endogenous gene, outbred Blackswiss mice (around 30 g or more body weight) were anesthetized by isofluorane/oxygen, and siRNA targeted to cyclophilin B (Dharmacon, D-001136-01-20 siCONTROL Cyclophilin B siRNA (Human/Mouse/Rat) or control GFP-949 siRNA (2 mg/kg) was administered intranasally to groups of 2 mice for each siRNA. Lungs were harvested 24 hours after administration. RNA was extracted from the lung and reverse transcription was done using a random primer. Real time PCR was then performed using cyclophilin B and GAPDH Taqman gene expression assay (Applied Biosystems). Results (Table 16-1) showed 70% silencing of cyclophilin B by siRNA targeted to cyclophilin B.

TABLE 16-1
Inhibition of Cyclophilin B in the Lung

| Average | Normalized | Ave normal | silencing % |
|---|---|---|---|
| PBS-1 | 5.395406 | 4.288984 | |
| PBS-2 | 3.182562 | | |
| GFP-1 | 2.547352 | 3.752446 | 12.50968 |
| GFP-2 | 4.957539 | | |
| Cyclo-1 | 1.173444 | 1.256672 | 70.7 |
| Cyclo-2 | 1.339901 | | |

TABLE 16-2

Target Portions in NP Gene

| | | |
|---|---|---|
| ID | | Nucleo- |
| Num- | | tide |
| ber | Sequence | Position |

| | | | |
|---|---|---|---|
| 1 | agcaaaagcaggguagaua | (SEQ ID NO: 17202) | 1-19 |
| 2 | gcaaaagcaggguagauaa | (SEQ ID NO: 17203) | 2-20 |
| 3 | caaaagcaggguagauaau | (SEQ ID NO: 17204) | 3-21 |
| 4 | aaaagcaggguagauaauc | (SEQ ID NO: 17205) | 4-22 |
| 5 | aaagcaggguagauaauca | (SEQ ID NO: 17206) | 5-23 |
| 6 | aagcaggguagauaaucac | (SEQ ID NO: 17207) | 6-24 |

### EXAMPLE 12

### Inhibition of Influenza Virus by Direct Delivery of Naked siRNA to the Respiratory System

siRNA preparation, viral infection, lung harvests, and influenza virus titer assays were performed as described above. Mice were anesthetized using isofluorane (administered by inhalation). siRNA was delivered in a volume of 50 μl by intranasal drip. p values were computed using Student's T test.

siRNA (NP-1496) in phosphate buffered saline (PBS) was administered to groups of mice (5 mice per group). Mice were infected with influenza virus (2000 PFU) 3 hours after siRNA administration. Lungs were harvested 24 hours post-infection and virus titer measured. In a preliminary experiment mice were anesthetized with avertin and 2 mg/kg siRNA was administered by intranasal drip. A reduction in virus titer relative to controls was observed, although it did not reach statistical significance (data not shown). In a second experiment, Black Swiss mice were anesthetized using isofluorane/O₂. Various amounts of siRNA in PBS was intranasally administered into the mice., 50 ul each mouse. Three different groups (5 mice per group) received doses of 2 mg/kg, 4 mg/kg, or 10 mg/kg siRNA in PBS by intranasal drip. A fourth group that received PBS alone served as a control. Three hours later, the mice were anesthetized again using isofluorane/O₂, 30 ul of PR8 virus (2000 pfu=4× lethal dose) was intranasally administered into the mice. 24 h after infection, the mouse lungs were harvested, homogenized and virus titer was measured by evaluation of the TCID₅₀ as described above. Serial 5-fold dilutions of the lung homogenate were performed rather than 10-fold dilutions.

A significant and dose-dependent difference in virus titer was seen between mice in each of the three treated groups and the controls (Table 17). The reduction in virus titer relative to controls was 3.45-fold (p=0.0125), 4.16-fold (p=0.0063), and 4.62-fold (p=0.0057) in the groups that received doses of 2 mg/kg, 4 mg/kg, and 10 mg/kg respectively.

In summary, these results demonstrate the efficacy of siRNA delivered to the respiratory system in an aqueous medium in the absence of specific agents to enhance delivery.

TABLE 17
Intranasal Delivery of Naked siRNA Inhibits Influenza Virus Production

| Treatment | log₁₀TCID50 | | | | | Average | P value |
|---|---|---|---|---|---|---|---|
| PBS | 26718.37 | 45687.78 | 45687.78 | 15625 | 26718.37 | 32087.46 | |
| NP (2 mg/kg) | 15625 | 15625 | 3125 | 3125 | 9137.56 | 9327.51 | 0.008 |
| NP (4 mg/kg) | 9137.56 | 9137.56 | 5343.68 | 9137.56 | 5343.68 | 7620 | 0.004 |
| NP (10 mg/kg) | 9137.56 | 9137.56 | 9137.56 | 3125 | 3125 | 6732.53 | 0.003 |

### EXAMPLE 13

### Inhibition of Influenza Virus Production in Mice by Direct Delivery of Naked siRNA to the Respiratory System

This example confirms results above and demonstrates inhibition of influenza virus production in the lung by administration of siRNA targeted to NP to the respiratory system in an aqueous medium in the absence of delivery-enhancing agents. Six μg, 15 ug, 30 μg, and 60 μg of NP-1496 siRNAs or 60 μg of GFP-949 siRNAs in PBS were intranasally instilled into mice essentially as described above, except that mice were intranasally infected with PR8 virus, 1000 pfu per mouse, two hours after siRNA delivery. Lungs were harvested 24 hours after infection. NP-specific siRNA was effective for the inhibition of influenza virus when administered by intranasal installation in an aqueous medium in the absence of delivery agents. A significant and dose-dependent difference in virus titer was seen between mice in each of the three treated groups and the controls (Table 18).

TABLE 18
Inhibition of Influenza Virus Production in the Lung Using Naked siRNA

| Treatment | TCID50 | | | | | Average | P value |
|---|---|---|---|---|---|---|---|
| PBS | 125 | 365.5 | 213.7 | 365.5 | 125 | 239.95 | |
| GFP (60 μg) | 125 | 213.7 | 213.7 | 213.7 | 365.5 | 226.32 | |
| NP (6 μg) | 213.7 | 213.7 | 125 | 213.7 | 42.7 | 161.8 | 0.263 |
| NP (15 μg) | 125 | 125 | 42.7 | 25 | 73.1 | 78.17 | 0.024 |
| NP (30 μg) | 8.5 | 125 | 42.7 | 125 | 14.6 | 63.18 | 0.019 |
| NP (60 μg) | 73.1 | 14.6 | 25 | 25 | 25 | 32.54 | 0.006 |

### EXAMPLE 14

### Modified siRNAs Mediate Effective Silencing

To explore the silencing potential of siRNAs containing modified nucleotides, NP-1496 siRNA containing sense and antisense strands with 2′-O-methyl modifications at alternate ribonucleotides in each strand were synthesized and tested in comparison with unmodified NP-1496 siRNA. The 2′-O-methyl modified NP1496 siRNA sequences were as follows: (2′-O-methyl shown as “m” in front of the modified nucleotide):

Sense: 5′-GmGA mUCmU UmAU mUUmC UmUC mGGmA G dTdT-3′ (SEQ ID NO: 17208)

Antisense: 5′-mCUmC CmGA mAGmA AmAU mAAmG AmUC mC dTdT-3′ (SEQ ID NO: 17209)

The 2′-O-methyl modified NP1496 siRNA and unmodified NP1496 siRNA were transfected into Vero cells in 24-well plate using lipofectamine 2000 (Invitrogen) following the manufacturer's instructions. 6 hours after transfection, the culture media was aspirated. The cells were inoculated with 200 μl of PR8 virus at MOI of 0.1. The culture supernatant was collected at 24, 36 and 48 hours after infection. Virus titer was determined as described above. The 2′-O-methyl modified NP1496 showed slightly more inhibition of virus growth than unmodified NP1496. Results are shown in Table 19.

TABLE 19
Effective Inhibition of Influenza Virus
Production Using Modified siRNA

| HA units | 24 h | 36 h | 48 h |
|---|---|---|---|
| No siRNA control | 4 | 8 | 16 |
| Unmodified NP1496 (400 uM) | 1 | 2 | 8 |
| Modified NP1496 (100 uM) | 1 | 2 | 8 |
| Modified NP1496 (200 uM) | 1 | 2 | 4 |
| Modified NP1496 (400 uM) | 1 | 1 | 4 |

### EXAMPLE 15

### Inhibition of Influenza Virus by Oraltracheal Delivery of Naked siRNA to the Respiratory System

siRNA preparation, viral infection, lung harvests, and influenza virus titer assays were performed as described above. Mice were anesthetized using avertin (administered by intraperitoneal injection). 1 mg/kg siRNA was delivered in a volume of 175 μl by oraltracheal injection.

siRNA (NP-1496), 1 mg/kg, and 30 ul Infasurf in 5% glucose was administered to groups of mice (5 mice per group). Mice were infected with influenza virus (2000 PFU) 3 hours after siRNA administration. Lungs were harvested 24 hours post-infection and virus titer measured.

In a second experiment, Black Swiss mice were anesthetized using intraperitoneally administered avertin. NP-1496 siRNA and GFP-949 siRNA in PBS was intratracheally administered into the mice, 50 μl each mouse. A third group that received PBS alone served as a control. Three hours later, the mice were anesthetized again using isofluorane/O₂, 30 ul of PR8 virus (2000 pfu=4× lethal dose) was intranasally administered into the mice. Twenty-four hours after infection, the mouse lungs were harvested, homogenized and virus titer was measured by evaluation of the TCID₅₀ as described above. Serial 5-fold dilutions of the lung homogenate were performed rather than 10-fold dilutions.

In summary, these results demonstrate the efficacy of siRNA delivered to the respiratory system in an aqueous medium in the absence of specific agents to enhance delivery.

### EXAMPLE 16

### Nucleotide Mutation Tolerance Studies to Select siRNAs That Target a Broad Spectrum of Influenza Viruses

This example demonstrates that siRNAs whose antisense strands are less than 100% complementary to the targeted transcript within the inhibitory region (e.g., within the 19 base pair region that is complementary to the target transcript) mediate effective silencing. The results demonstrate that the RNAi agents described herein will effectively inhibit a wide range of influenza strains whose sequences vary from that of PR8 within the target portion.

A dual luciferase assay was used to evaluate the ability of siRNAs to inhibit expression of influenza genes that are not 100% complementary to the antisense strand of the siRNA within the 19 nucleotide inhibitory region. Mismatches derived from the alignment of human and avian influenza virus strains (using PR8 as standards) were introduced into the DNA vector (psiCHECK) using a site-directed mutagenesis kit (Stratagene), i.e., the influenza target site was modified to include either 1 or 2 differences relative to the PR8 sequence, with the specific differences corresponding to differences found in one or more of the human or avian influenza strains.

Table 20 shows results of an experiment demonstrating that variations in the viral NP target (target for NP-1496) do not substantially reduce RNAi activity. (The data shown is the average of triplicates). Mismatches at positions near the 5′ or 3′ end of the antisense strand, or near the middle, were tested.

TABLE 20
Effect of mismatches between antisense strand and target
region on silencing by NP-1496

| | | A3 to | T9 to | C12 to | C15 to | A18 to |
|---|---|---|---|---|---|---|
| | Original | G3 | C9 | T12 | T15 | G18 |
| Renilla luciferase | 85.6 | 81.8 | 58.3 | 67.8 | 72.9 | 54.7 |
| silencing (%) | | | | | | |
| Remaining silencing | 100 | 91.3 | 65.1 | 75.7 | 81.4 | 61.1 |
| comparing with | | | | | | |
| original (%) | | | | | | |

Variations in the viral PA target (target for PA-2087 or PA-8282) do not substantially reduce RNAi activity. However, G18 to A18 mutations found in 7 among 157 human influenza strains did substantially affect the RNA interference activity. Mismatches at positions near the 5′ or 3′ end of the antisense strand, or near the middle, were tested. The presence of two mismatches between the antisense strand inhibitory region and the target reduced the silencing by about 70-75%, but a useful degree of silencing was still observed (Table 21).

TABLE 21
Effect of mismatches between antisense strand and target region
on silencing by PA-2087 or PA-8242

| | | | | | | | | T6 to C6 |
|---|---|---|---|---|---|---|---|---|
| | | A4 to | T6 to | T6 to | C15 to | G18 to | A19 to | and C15 to |
| | Original | G4 | A6 | C6 | T15 | A18 | G19 | T15 |
| Renilla luciferase | 91.7 | 80.8 | 75.9 | 88.8 | 87.5 | 7.0 | 89.3 | 26.8 |
| silencing (%) | | | | | | | | |
| Remaining | 100 | 88.1 | 82.8 | 96.9 | 95.5 | 7.6 | 97.4 | 29.3 |
| silencing | | | | | | | | |
| comparing with | | | | | | | | |
| original (%) | | | | | | | | |

Table 22 shows results of an experiment demonstrating that variations in the viral PB2 target (target for PB2-3817) do not substantially reduce RNAi activity. (The data shown is the average of triplicates).

TABLE 22
Effect of mismatches between antisense strand and target region
on silencing by PB2-3817

| | Original | A17 to G17 | A18 to T18 |
|---|---|---|---|
| Renilla luciferase | 86.7 | 73.4 | 75.8 |
| silencing (%) | | | |
| Remaining silencing | 100 | 100 | 87.4 |
| comparing with | | | |
| original (%) | | | |

Table 23 shows results of an experiment demonstrating that variations in the viral PB1 target (target for PB1-6124) do not substantially reduce RNAi activity. (The data shown is the average of triplicates). Mismatches at positions near the 5′ or 3′ end of the antisense strand, or near the middle, were tested. The presence of two mismatches between the antisense strand inhibitory region and the target reduced the silencing by about 70-75%, but a useful degree of silencing was still observed.

TABLE 23
Effect of mismatches between antisense strand and target
region on silencing by PB1-6124

| | | | | | | A1 to T1 |
|---|---|---|---|---|---|---|
| PB1 (Lab ID# | | C15 to | A1 to | T2 to | G3 to | and G3 to |
| 6124 siRNA) | Original | T15 | G1 | C2 | A3 | A3 |
| Ranilla luciferase | 84.1 | 71.8 | 82.8 | 84.5 | 74.9 | 73.9 |
| silencing (%) | | | | | | |
| Remaining silencing | 100 | 85.4 | 98.5 | 100 | 89.1 | 87.9 |
| comparing with | | | | | | |
| original (%) | | | | | | |

Effect of mismatches between antisense strand and target
region on silencing by PB1-6129

| | | | | | | T3 to C3 |
|---|---|---|---|---|---|---|
| PB1 (Lab ID# | | T3 to | T7 to | T2 to | C10 to | and C10 |
| 6129 siRNA) | Original | C3 | C7 | C2 | T10 | to T10 |
| Ranilla luciferase | 86.4 | 87.3 | 84.4 | 84.5 | 81.3 | 59.0 |
| silencing (%) | | | | | | |
| Remaining silencing | 100 | 100 | 97.8 | 100 | 94.2 | 68.3 |
| comparing with | | | | | | |
| original (%) | | | | | | |

In addition to the results shown in the tables above, the present example also demonstrates that ten exemplary siRNAs duplexes of the present invention tolerate mismatches between the nucleotide sequence of the anti-sense strand of the siRNA and the nucleotide sequence of the targeted regions of viral transcripts. In the instant example, the capacity of the 21 previously identified siRNA to tolerate target sequence mutations was determined. To accomplish this, the target sites of all human and avian influenza gene sequences (available at www.lan1.flu.gov) were aligned using the PR8 strain as a reference. Single nucleotide polymorphism (SNPs) were identified and introduced into the dual-luciferase reporter construct using site-directed mutagenesis. Expression vectors containing the control target sequence (PR8) or the variants were subsequently transfected into Vero cells along with the 50 nM of the appropriate targeting siRNAs to determine the sensitivity of each siRNA to tolerate nucleotide mismatches.

Of the 21 siRNAs, ten siRNAs exhibited high degrees of silencing (% silencing) and were highly tolerant of target site polymorphisms. Table 24 summarizes the percent silencing data for the ten siRNAs (INFsi-1 through INFsi-8 and G1499 and G4276). The nucleotide sequence for each siRNA is shown and the nucleotides that were the target for site-directed mutagenesis are bolded and underlined. The “Mismatch” column illustrates the original nucleotide and its position, shown in parenthesis, within the siRNA along with the nucleotide that was substituted for the original nucleotide (mutated nucleotide). The percent silencing is presented as percentage of silencing observed with the native (PR8) silencing. Therefore, 100% relative silencing indicates that the mismatch had no effect on the functionality of the siRNA compared to its ability to silence the exact match target sequence (PR8). Any decrease in the percent relative silencing represents the degree of sensitivity of the siRNA for that mismatch in the target sequence (i.e., a lower percentage equates to a decrease in the functionality of the siRNA; “functionality” defined in this context as the ability of the siRNA to degrade it target RNA).

TABLE 24

Sensitivity of siRNAs to Naturally Occurring
Target Site Point Mutations

| | |
|---|---|
| Mismatch | Rela- |

| | | Original | Mutated | tive |
|---|---|---|---|---|
| | | Nucleo- | Nucleo- | % Si- |
| | siRNA Nucleotide | tide | tide | lenc- |
| siRNA | Sequence* | (Position) | (Position) | ing |
| G3789 | CGGGACTCTAGCATACTTA | G(3) | A(3) | 100% |
| (INFsi- | (SEQ ID NO: 17210) | G(4) | A(4) | 100% |
| 1) | | C(8) | T(8) | 72.4% |
| | | T(9) | C(9) | 50.1% |
| | | A(15) | G(15) | 81.9% |
| | | C(16) | G(16) | 0 |
| | | T(18) | G(18) | 74.2% |
| G3807 | ACTGACAGCCAGACAGCGA | C(2) | A(2) | 100% |
| (INFsi- | (SEQ ID NO: 17211) | C(6) | T(6) | 91.8% |
| 2) | | C(9) | T(9) | 50.7% |
| | | A(15) + | G(15) + | 41.5% |
| | | G(16) | A(16) | |
| G3817 | AGACAGCGACCAAAAGAAT | A(17) | G(17) | 100% |
| (INFsi- | (SEQ ID NO: 17212) | A(18) | T(18) | 87% |
| 3) | | A(18) | C(18) | 80.5% |
| | | T(19) | C(19) | 95.7% |
| | | T(19) | A(19) | 100% |
| G6124 | ATGAAGATCTGTTCCACCA | A(1) | T(1) | 100% |
| (INFsi- | (SEQ ID NO: 17213) | A(1) | G(1) | 98.5% |
| 4) | | T(2) | C(2) | 100% |
| | | G(3) | A(3) | 89.1% |
| | | A(5) | G(5) | 94.7% |
| | | T(8) | C(8) | 77% |
| | | T(12) | C(12) | 100% |
| | | C(15) | T(15) | 85.4% |
| | | A(1) + | T(1) + | 87.9% |
| | | G(3) | A(3) | |
| | | T(8) + | C(8) + | 32.2% |
| | | C(15) | T(15) | |
| G6129 | GATCTGTTCCACCATTGAA | T(3) | C(3) | 100% |
| (INFsi- | (SEQ ID NO: 17214) | T(7) | C(7) | 97.8% |
| 5) | | C(10) | T(10) | 94.2% |
| | | T(3) + | C(3) + | 68.3% |
| | | C(10) | T(10) | |
| G8282 | GCAATTGAGGAGTGCCTGA | A(4) | G(4) | 88.1% |
| (INFsi- | (SEQ ID NO: 17215) | T(6) | A(6) | 82.8% |
| 6) | | T(6) | C(6) | 96.9% |
| | | C(15) | T(15) | 95.5% |
| | | G(18) | A(18) | 7.6% |
| | | A(19) | G(19) | 97.4% |
| | | T(6) + | C(6) + | 29.3% |
| | | C(15) | T(15) | |
| G8286 | TTGAGGAGTGCCTGATTAA | T(2) | C(2) | 100% |
| (INFsi- | (SEQ ID NO: 17216) | T(2) | A(2) | 100% |
| 7) | | G(8) | A(8) | 100% |
| | | C(11) | T(11) | 100% |
| | | G(14) | A(14) | 85.2% |
| | | A(15) | G(15) | 95.4% |
| G1498 | GGATCTTATTTCTTCGGAG | A(3) | G(3) | 95.7% |
| (INFsi- | (SEQ ID NO: 17217) | T(9) | C(9) | 86.5% |
| 8) | | C(12) | T(12) | 85.4% |
| | | C(15) | T(15) | 91.7% |
| | | A(18) | G(18) | 94.4% |
| G1499 | GATCTTATTTCTTCGGAGA | A(2) | G(2) | 63.6% |
| | (SEQ ID NO: 17218) | T(8) | C(8) | 27.5% |
| | | C(11) | T(11) | 38.3% |
| | | C(14) | T(14) | 36.8% |
| | | A(17) | G(17) | 33.2% |
| G4276 | ACCTATGACTGGACTCTAA | C(3) | A(3) | 100% |
| | (SEQ ID NO: 17819) | C(3) | T(3) | 99.5% |
| | | A(5) | T(5) | 95.1% |
| | | T(6) | C(6) | 99% |
| | | C(9) | T(9) | 89.5% |
| | | T(15) | C(15) | 88.1% |
| | | T(15) | G(15) | 57.4% |
| | | T(15) | A(15) | 40.8% |
| | | C(16) | T(16) | 47.5% |
| | | A(18) | G(18) | 82.2% |
| | | C(9) + | T(9) + | 17.4% |
| | | T(15) | A(15) | |
| | | C(16) + | T(16) + | 28.4% |
| | | A(18) | G(18) | |
| | | T(15) + | G(15) + | 4.9% |
| | | C(16) + | T(16) + | |
| | | A(18) | G(18) | |
| | | T(15) + | A(15) + | 10.3% |
| | | C(16) + | T(16) + | |
| | | A(18) | G(18) | |
| | | C(3) + | T(3) + | 7.6% |
| | | A(5) + | T(5) + | |
| | | T(6) + | C(6) + | |
| | | C(9) + | T(9) + | |
| | | A(18) | G(18) | |

As shown in Table 24, only 19 of the 70 avian/human targets containing SNPs disrupted siRNA functionality by more than 30%. Two of these mutants, INFsi-2 have changes at position 9 (Mutation-9 for siRNA INFsi-2 and Mutation-4 for siRNA INFsi-1), which is adjacent to the RISC mediated cleavage site and predicted to be particularly sensitive to base pair mismatches. Surprisingly, three of the four variants containing two target site polymorphisms showed significant reductions in the levels of target cleavage (e.g. siRNA INFsi-4). None of the variants carrying the individual polymorphisms significantly impeded siRNA functionality.

These data show that ten siRNAs exhibited broad targeting properties against the majority of human and avian influenza virus strains demonstrating that these ten siRNAs have great potential as a multi-gene targeting strategy for effective RNAi therapeutics.

### EXAMPLE 17

### Intravenous Delivery of siRNA Inhibits Influenza Virus Production In Vivo

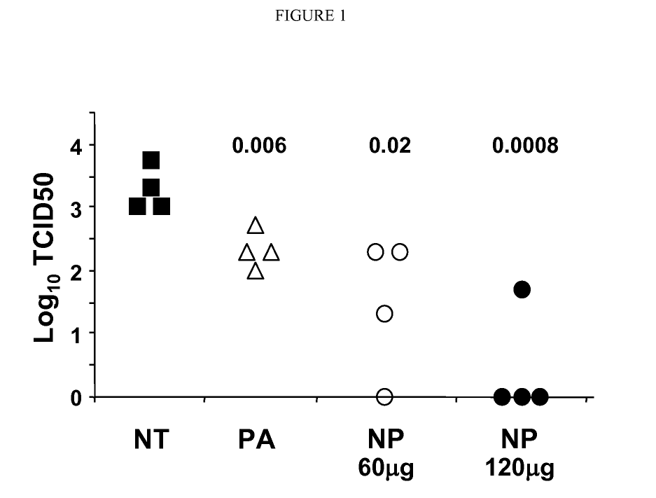

The present example demonstrates that both the prophylactic and post-infection intravenous administration of siRNA targeted to viral NP transcripts significantly inhibited influenza virus replication in the mouse (FIG. 1). The following is a list of exemplary human influenza virus conserved target sequence (derived from Accession No. AF389119):

| | |
|---|---|
| gccacugaaaucagagcau, | (SEQ ID NO: 17220) |
| ucagagcauccgucggaaa, | (SEQ ID NO: 17221) |
| ggacgauucuacauccaaa, | (SEQ ID NO: 17222) |
| cagcuuaacaauagagaga, | (SEQ ID NO: 17223) |
| gcuuaacaauagagagaau, | (SEQ ID NO: 17224) |
| aauagagagaauggugcuc, | (SEQ ID NO: 17225) |
| gggaaagauccuaagaaaa, | (SEQ ID NO: 17226) |
| ggaaagauccuaagaaaac, | (SEQ ID NO: 17227) |
| ugagagaacucauccuuua, | (SEQ ID NO: 17228) |
| uuaugacaaagaagaaaua, | (SEQ ID NO: 17229) |
| acaagaauugcuuaugaaa, | (SEQ ID NO: 17230) |
| gaauugcuuaugaaagaau, | (SEQ ID NO: 17231) |
| aagcaaugauggaucaagu, | (SEQ ID NO: 17232) |
| gcaaugauggaucaaguga, | (SEQ ID NO: 17233) |
| ugauggaucaagugagaga, | (SEQ ID NO: 17234) |
| ccacuagaggaguucaaau, | (SEQ ID NO: 17235) |
| cacuagaggaguucaaauu, | (SEQ ID NO: 17236) |
| gaggaaacaccaaucaaca, | (SEQ ID NO: 17237) |
| ggaaacaccaaucaacaga, | (SEQ ID NO: 17238) |
| gggccaaaucagcauacaa, | (SEQ ID NO: 17239) |
| caaccauuauggcagcauu, | (SEQ ID NO: 17240) |
| ccauuauggcagcauucaa, | (SEQ ID NO: 17241) |
| aggaugauggaaagugcaa, | (SEQ ID NO: 17242) |
| gaugauggaaagugcaaga, | (SEQ ID NO: 17243) |
| gaguaaugaaggaucuuau, | (SEQ ID NO: 17244) |
| ggaucuuauuucuucggag, | (SEQ ID NO: 17245) |
| gaucuuauuucuucggaga, | (SEQ ID NO: 17246) |
| ucuuauuucuucggagaca, | (SEQ ID NO: 17247) |
| ggaguacgacaauuaaaga, | (SEQ ID NO: 17248) |

The following is a list of sequences that represent a fragment of the influenza NP gene for multiple species. The bolded region is a conserved region shared among these sequences.

| | Nucleotide Sequence of | SEQ |
|---|---|---|
| Influenza Strain, | Conserved Region of Influenza NP Gene, | ID NO |
| PR8 (H1N1), | AATGAAGGATCTTATTTCTTCGGAGACAATGCAGAGGAGTACGACAATTA, | 17249 |
| WSN (H1N1), | AATGAAGGATCTTATTTCTTCGGAGACAATGCAGAGGAGTACGACAATTA, | 17250 |
| Lenn.(H2N2), | AATGAAGGATCTTATTTCTTCGGAGACAATGCAGAGGAGTACGACAATTA, | 17251 |
| HK (H3N2), | AATGAAGGATCTTATTTCTTCGGAGACAATGCAGAGGAGTACGACAATTA, | 17252 |
| Memphis (H3N2), | AATGAAGGATCTTATTTCTTCGGAGACAATGCAGAGGAGTACGACAATTA, | 17253 |
| HK (H5N1), | AATGAAGGATCTTATTTCTTCGGAGACAATGCAGAGGAATATGACAATTG, | 17254 |
| Duck (H10N7), | AACGAGGGATCTTATTTCTTCGGAGACAATGCAGAGGAATATGACAATTA, | 17255 |
| Equine (H7N7), | AATGAAGGGTCTTATTTCTTCGGAGACAATGCTGAGGAGTTTGACAGTTA, | 17256 |
| Whale (H13N2), | AATGAGGGATCTTATTTCTTCGGAGACAATGCTGAGGAGTATGACAATTG, | 17257 |
| Chicken(H9N2), | AATGAAGGATCTTATTTCTTCGGAGACAATGCATAGGAGTATGACAATTA, | 17258 |
| Swine (H4N6), | AACGAAGGGTCTTATTTCTTCGGAGACAATGCAGAGGAATATGACAATTA, | 17259 |

To test the prophylactic use of viral targeting siRNA, NP-1496 (INFsi-9) was mixed with the cationic delivery polymer jetPEI (Qbiogene) and administered (2 mg/Kg) to C57BL/6 mice intravenously (IV). Three hours later, mice were inoculated (intranasally) with 1×10⁴ PR8 viral particles to initiate infection and later sacrificed 24 hrs post-infection to assay lung homogenates for viral titers using the MDCK hemagglutinin assay.

The average log₁₀ TCID₅₀ of the lung homogenate for mice that received no siRNA treatment or received a siRNA targeted to GFP was 4.2. In mice that were pretreated with 30 μg siRNA targeted to NP and jetPEI, the average log₁₀ TCID₅₀ of the lung homogenate was 3.9. In mice that were pretreated with 60 μg siRNA targeted to NP and jetPEI, the average log₁₀ TCID₅₀ of the lung homogenate was 3.2. The difference in virus titer in the lung homogenate between the group that received no treatment and the group that received 60 μg NP siRNA was significant with P=0.0002.

In order to evaluate siRNA as a therapeutic treatment for an existing influenza infection, mice were infected with PR8 virus intranasally and five hours later were given NP-1496/jetPEI or PA-2087/jetPEI mixture intravenously. Viral titers in the lungs were assayed by MDCK-HA assay 28 hours post-infection. All treatments significantly reduced viral titer in comparison to untreated, infected mice; dose-responsive decreases in viral titers were observed in mice treated with NP-1496 (FIG. 1). A suppression effect of siRNA treatment at 24 hours post-infection was also seen in mice.

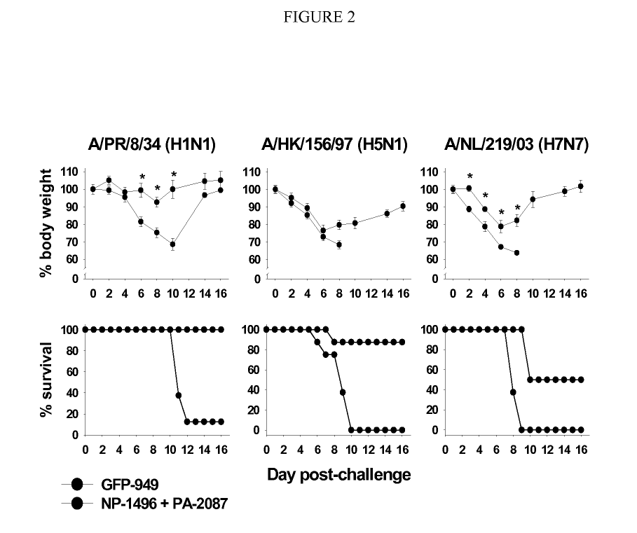

Further, the designed siRNAs can also protect mice from a lethal challenge of avian influenza virus. Mice injected (2×) with control (GFP-targeting) siRNAs and subsequently challenged with a lethal dose of H1N1 (PR8), H5N1 or H7N7 virus, continuously lost weight and succumbed to the infection between days 7-10, as shown in FIG. 2. However, infected mice that received the combined siRNAs NP-1496 and PA-2087 recovered from the initial weight loss. At least 50% of the mice survived the lethal H7N7 challenge, 87% survived the lethal H5N1 challenge and 100% survived the H1N1 challenge. Thus, siRNAs specific for the conserved regions of the influenza viral genome confers broad protection, including protection against the highly pathogenic avian influenza viruses (FIG. 2).

### EXAMPLE 18A

### Intranasal Delivery of siRNA Inhibits Influenza Production in Mice

The present example demonstrates that prophylactic intranasal administration of siRNA targeted to viral NP transcripts inhibited influenza virus replication and reduced viral RNA levels in a dose-dependent manner in the mouse.

Influenza normally infects and replicates in the upper respiratory tract and lungs. Therefore, due to accessibility, topical administration, i.e. intranasal and/or pulmonary delivery of drug should be ideal for influenza prophylaxis and therapy. Specifically, intranasal and/or pulmonary delivery of siRNAs is advantageous in treating influenza virus infection, because, 1) high local siRNA concentration are easily achieved when local delivery route is used and thus less siRNA is required compared to systemic delivery and 2) intranasal and/or pulmonary delivery methods are non-invasive. Thus, an intranasal delivery of siRNA in the influenza mouse model was pursued.

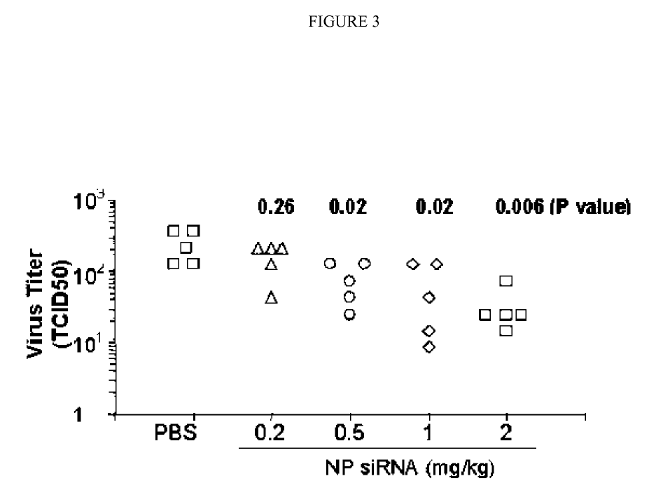

Unlike conventional intravenous delivery of naked siRNA which resulted in negligible silencing effect, intranasally administered siRNA (unmodified, in PBS or saline) can be detected in the lungs and is able to silence endogenous gene expression or inhibit virus production in lung tissue. To test the efficacy of non-invasive delivery of influenza targeting siRNA, the NP-1496 siRNA (in PBS) was delivered intranasally. BALB/c mice were treated intranasally with indicated amounts of NP specific siRNA in PBS or PBS control. Two hours later, all mice were infected intranasally (1000 pfu/mouse) with the PR8 serotype. The lungs were harvested 24 hours post-infection and viral titer was measured from lung homogenates by MDCK-HA assay. P values between PBS and siRNA groups indicated statistical significance with 0.5, 1 and 2 mg/kg siRNA treated groups. The data is shown in FIG. 3.

In the absence of a carrier, naked NP targeting siRNA was effective in suppressing viral production in the mouse lung (FIG. 3; 24 hours post-infection). Suppression was dose dependent, with a 7-fold reduction being observed when 2 mg/kg of siRNA was delivered two hours prior to infection.

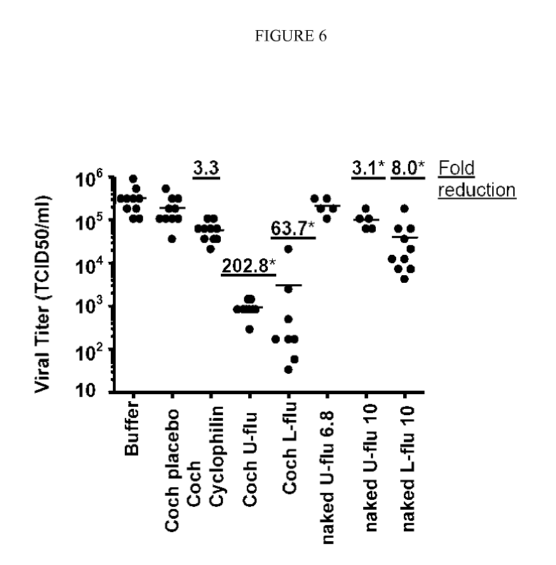

The effects of intranasal delivery of NP-targeting siRNA were also investigated at higher concentrations (10 mg/kg, delivered 3 hours prior to infection) using target mRNA expression (quantitative RT-PCR) and viral titer (MDCK-HA) to measure efficacy. BALB/c mice were administered control and NP-targeting siRNA intranasally (10 mg/kg, in PBS). Three hours later, all the mice were infected intranassally with PR8 virus (50 pfu/mouse). The lungs were harvested at 24 and 48 hours post-infection and total RNA was isolated from the left lung. Total mRNA was reverse transcribed to cDNA using dT18 primers. Real time PCR was carried out using PB1 specific primers to quantify viral mRNA levels. GAPDH was used as an internal control. The right and middle lungs were homogenized and the viral titer was measured by MDCK-HA assay. The virus titer in the samples at 48 hours post-infection is shown in the FIG. 6 statistic significance was found between PBS and NP siRNA treated group using student t test (p=0.01); the titer in the samples 24 hours post-infection was too low to detect, possibly due to siRNA directed suppression.

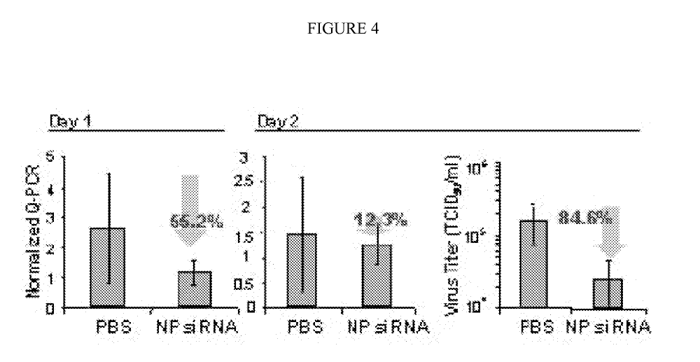

The results are shown in FIG. 4, which compares the normalized quantitative PCR results and the viral titer assay results. Viral mRNA level measured at 24 hours post-infection show a 55.2% inhibition but by 48 hours post-infection only minimal inhibition was observed. In contrast, the MDCK-HA assay of mouse lung samples indicated 84.6% viral titer suppression on day 2. Compared to the MDCK-HA assay that measures live virus particles, viral mRNA quantification is probably more sensitive in reflecting the early changes in viral replication. Thus, the decrease in viral mRNA suppression on day 2 is probably due to the decreased RNAi effect in the mouse lung by that time.

Also, the effect on influenza viral titer in mouse between naked siRNA targeting the NP transcript delivered intranasally and the influenza treatment, Tamiflu, was compared. Relative to the level of viral titer observed with the GPF control siRNA, both the intranasally delivered naked siRNA and Tamiflu treatments reduced influenza viral titers.

The effect on viral titer by NP-viral transcript targeting in mouse after intranasal delivery the siRNA G1498 (INFsi-8) was also addressed. The G1498 siRNA exhibited significant ability to reduce viral titers in vitro and thus was chosen for further characterization in vivo. The control for this study was an unmodified siRNA targeted against luciferase (Dharmacon; Luc). Ten week old female BALB/c (Taconic) mice with a weight range of 18-22 grams were used in the study. There were ten mice per study group. The mice were dosed with G1498 siRNA in PBS at 2 mg/kg, 5 mg/kg, 10 mg/kg, 20 mg/kg and 30 mg/kg. The control groups were dosed the same, except no controls received a 2 mg/kg dose. Both the G1498 and Luc siRNA control groups were infected with PR8 influcenze virus at 30 pfu in 30 μl in PBS four hours post-siRNA administration. Forty-eights hours post-infection, the mouse lungs were harvested and viral titers measured therefrom in MDCK cells with a TCID₅₀ assay.

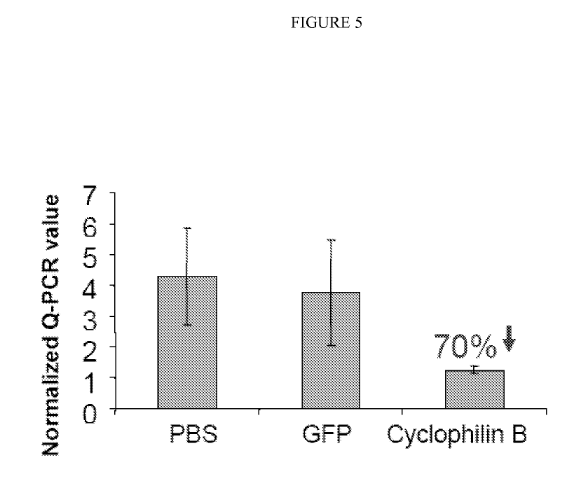

The results are shown in FIG. 5. The results of the TCID₅₀ assay show that the G1498 siRNA at 2 mg/kg suppressed influenza production in the mouse lung by 86%, at 5 mg/kg and 10 mg/kg by 90.6%, at 20 mg/kg by 96.6% and at 30 mg/kg by 95.2%. In relation to PBS alone or the Luc control siRNA study groups, the mice administered the G1498 siRNA intranasally, as a whole, showed significant differences (P<0.001). The mice that received PBS did not exhibit significant difference compared to the mice group that received the Luc siRNA, as a whole, (P>0.05). Each study group that received a dose of the G1498 siRNA significantly differed from the PBS group or Luc siRNA control study group at 30 mg/kg (P<0.05). Finally, no significant dose response was observed with mice that received the range of G1498 siRNA doses.

### EXAMPLE 18B

### Intranasal Delivery of siRNA Inhibits Cyclophilin B Expression in a Non-Influenza Mouse Model

The present example demonstrates that the airway cell uptake of naked siRNA via intranasal administration is not a specific phenomenon of influenza-infected cells. In addition, naked siRNA delivered intranasally also reduced endogenous gene, cyclophilin B, expression in the lungs of healthy mice. Balb/c mice were treated intranasally with 10 mg/kg cyclophilin B specific siRNA (Dharmacon) or GFP siRNA in PBS or PBS control. There were five mice per group. The mouse lungs were harvested 24 hours later. Total RNA was purified from the lung samples and reverse transcription was conducted using dT18 primer. Cyclophilin B-specific primers (Applied Biosystem) were used in real-time PCR to quantify the target mRNA level. GAPDH-specific primers (Applied Biosystem) were also used in the PCR as a control.

As shown in FIG. 6, the cyclophilin B mRNA in the lungs was inhibited by 70% 24 hours after the mice received 10 mg/kg cyclophilin B siRNA intranasally. These data indicate that the airway cell uptake of naked siRNA via intranasal administration is not a specific phenomenon in influenza-infected cells. Endogenous gene silencing in healthy cells in healthy animal can be also achieved by naked siRNA delivered intranasally. This finding is highly relevant for the utility of siRNA for prophylaxis, which would occur in the absence of infection.

### EXAMPLE 19

### Influenza Viral Titer Reduction Following Intranasal Administration of Cochleate siRNA Formulations in Mouse

The present demonstrates that intranasal administration of the G1498 siRNA in a cochleate delivery formulation (BDSI, North Carolina) enhances influenza viral suppression relative to naked siRNA in mouse. For the instant example, the formulations tested are shown below in Table 25.

TABLE 25
Formulations Administered Intranasally to Mice

| Study Group | Formulation |
|---|---|
| Buffer (Control) | TES + 2 mM Ca⁺² |
| Cochleate Placebo | 40 mg DOPS/ml |
| Cochleate Cyclophilin B siRNA | 4 mg/ml (10 mg/kg) |
| | siRNA by starting material |
| | (0.963 mg/ml by HPLC) |
| Cochleate Unlipidated G1498 | 4 mg/ml siRNA by staring |
| siRNA (U-flu) | material (2.752 mg/ml by HPCL) |
| Cochleate Lipidated G1498 | 4 mg/ml siRNA by |
| siRNA (L-flu) | staring material (1.546 mg/ml HPLC) |
| Naked Unlipidated G1498 siRNA | 6.8 mg/kg siRNA by |
| (U-flu) | staring material (2.752 mg/ml HPLC) |
| Naked Unlipidated G1498 siRNA | 10 mg/kg siRNA |
| (U-flu) | by staring material (4 mg/ml HPLC) |
| Naked Lipidated G1498 siRNA | 10 mg/kg siRNA |
| (U-flu) | by staring material (4 mg/ml HPLC) |

The formulations listed above were administered intranasally five hours post-infection with influenza. The viral titer for the whole lung was measured 48 hours post-infection. There were ten mice in each study group, except the naked unlipidated U-flu groups, which had five.

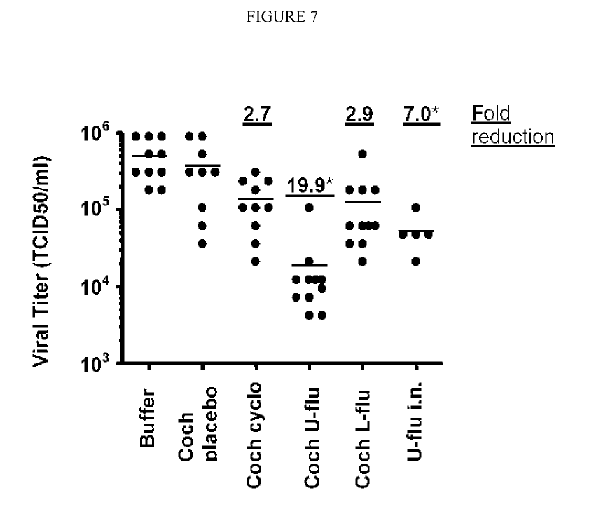

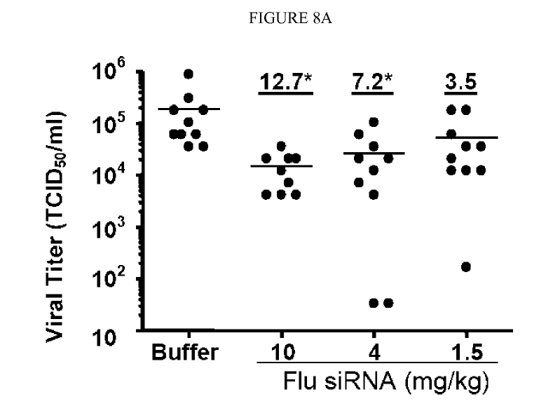

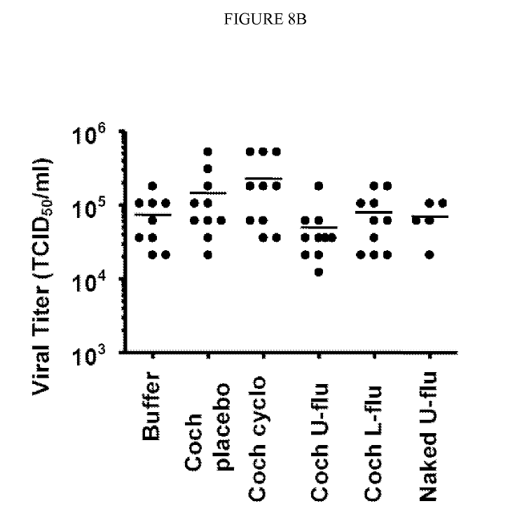

The lung viral titer results are shown in FIG. 7. Each dot on the graph represents one animal. Statistics was performed by One-Way-Anova. The numbers with an asteric indicates that the mean value has a statistical difference relative to the controls (placebo or buffer). The data in FIG. 8 show that the G1498 siRNA administered intranasally with the cochleate delivery formulation exhibit greater viral titer reduction relative to the naked G1498 siRNA and the controls (Buffer alone or the Cochleate Placebo). Some degree of toxicity was observed in all groups that receive the cochleate formulations or naked lapidated siRNA.

### EXAMPLE 20

### Influenza Viral Titer Reduction Following Intravenousl Administration of Cochleate siRNA Formulations in Mouse

The present demonstrates that intravenous administration of the G1498 siRNA in a cochleate delivery formulation enhances influenza viral suppression relative to naked siRNA in mouse. The formulations tested are shown below in Table 26.

TABLE 26
Formulations Administered Intravenouslly to Mice

| Study Group | Formulation |
|---|---|
| Buffer (Control) | TES + 2 mM Ca⁺² |
| Cochleate Placebo | 10 mg DOPS/ml |
| Cochleate Cyclophilin B siRNA | 1 mg/ml (10 mg/kg) |
| | siRNA by starting material |
| | (0.607 mg/ml by HPLC) |
| Cochleate Unlipidated G1498 | 1 mg/ml siRNA by staring |
| siRNA (U-flu) | material (1.196 mg/ml by HPCL) |
| Cochleate Lipidated G1498 | 1 mg/ml siRNA by |
| siRNA (L-flu) | staring material (0.381 mg/ml HPLC) |
| Naked Unlipidated G1498 | 10 mg/kg siRNA by |
| siRNA (U-flu) | staring material (4 mg/ml HPLC) |

The formulations listed above were administered intranasally five hours post-infection with influenza. The viral titer for the whole lung was measured 48 hours post-infection. There were ten mice in each study group, except the naked unlipidated U-flu groups, which had five.

The lung viral titer results are shown in FIG. 8A. Each dot on the graph represents one animal. Statistics was performed by One-Way-Anova. The numbers with an asteric indicates that the mean value has a statistical difference relative to the controls (placebo or buffer). The data in FIG. 7 show that the G1498 siRNA administered intravenously with the cochleate delivery formulation exhibited greater viral titer reduction relative to the controls (Buffer alone or the Cochleate Placebo). Moreover, the U-flu formulation administered intranasally also reduced lung viral titers.

A dose response profile was also generated for intravenously administered siRNA delivered in cochleate formulations. The formulations tested are shown below in Table 27.

TABLE 27
Formulations Administered Intravenouslly
to Mice for Dose-Response Study

| Study Group | Formulation |
|---|---|
| Buffer (Control) | TES + 2 mM Ca⁺² |
| Cochleate Unlipidated | 2.5 mg/ml (10 mg/kg) |
| Cyclophilin B siRNA | siRNA by starting material |
| Cochleate Unlipidated G1498 | 2.5 mg/ml |
| siRNA (U-flu) | siRNA by staring material |
| Cochleate Unlipidated G1498 | 1 mg/ml |
| siRNA (L-flu) | siRNA by staring material |
| Cochleate Unlipidated G1498 | 0.4 mg/kg |
| siRNA (U-flu) | siRNA by staring material |
| Cochleate Unlipidated G1498 | 0.15 mg/kg |
| siRNA (U-flu) | siRNA by staring material |

The formulations listed above were administered intranasally five hours post-infection with influenza. The viral titer for the whole lung was measured 48 hours post-infection. There were ten mice in each study group.

The lung viral titer results are shown in FIG. 8B. Each dot on the graph represents one animal. Statistics was performed by One-Way-Anova. The numbers with an asteric indicates that the mean value has a statistical difference relative to the controls (placebo or buffer). The data in FIG. 8 show that the G1498 siRNA administered intravenously with the cochleate delivery formulation exhibited greater viral titer reduction relative to the Buffer control. Moreover, a dose-response was observed. As the dose of the G1498 siRNA in the cochleate formulation increased a greater reduction in mouse lung viral titers was observed.

The same formulations listed in Table 27 above were administered via oral gavage to mice following the same protocols and procedures for measuring mouse lung viral titers. The lung viral titer results are shown in FIG. 8B. As stated previously, each dot represents an animal. No statistical differences among the test formulation were observed. Additionally, no toxicity was observed in any of the study groups.

### EXAMPLE 21

### Distribution of Rhodamine-Cochleate in the Mouse Lung Before and After Influenza Infection

The present example demonstrates that intranasal administration of the rhodamine-cochleate siRNA-free formulation showed a wide distribution of the rhodamine compared to intravenous or oral gavage administration. For the purpose of the instant example, rhodamine was encapsulated in the siRNA-free cochleate. The rhodamine-cochleate formulation was administered via intranasal (40 mg/ml, 50 μl/mouse), intravenous (10 mg/ml, 200 μl/mouse) or oral gavage (10 mg/ml, 200 μl/mouse) four hours either pre- or post-influenza infection. The mouse lung tissue was collected five hours after the final injection or infection and then frozen with dry ice and sectioned for analysis. The frozen sections was stained with DAPI for nuclei and imaged.

The results from pre- and post-influenza infection were similar for all groups. However, the group subject to the intranasal administration of the rhodamine-cochleate formulation showed a wide distribution of rhodamine in the lung section. The group subject to the intravenous administration showed some rhodamine aggregation but had limited distributed positive signal. The ones with oral gavage did not show positive signals. These data indicate that thorough distribution of a cochleate formulation in the lung for treatment of a respiratory virus infection is best achieved with intranasal administration. Thorough distribution within the lung is essential for effective reduction of viral titers.

What is claimed is:

1. A double-stranded ribonucleic acid (dsRNA) molecule that decreases expression of a respiratory virus gene by RNA interference, wherein the dsRNA molecule has a double-stranded region of from about 15 to about 60 base pairs, the dsRNA molecule comprising:
a. a first strand comprising from about 15 to about 60 nucleotides having a region of at least 15 contiguous nucleotides that are 100% complementary to a nucleic acid sequence of the respiratory virus gene, wherein the region of at least 15 contiguous nucleotides is present in a homologous gene of the virus in at least about 70% of the respiratory virus variants (strains) containing the homologous gene, except for no more than three (3) nucleotide mismatches among the region of at least 15 contiguous nucleotides; and
b. a second strand comprising from about 15 to about 60 nucleotides that is at least 70% complementary to the first strand.

2. The dsRNA molecule of claim 1, wherein the double-stranded region is from about 15 to about 40 base pairs, the dsRNA molecule comprising:
a. a first strand comprising from about 15 to about 40 nucleotides having a region of at least 19 contiguous nucleotides that are 100% complementary to a nucleic acid sequence of the respiratory virus gene, wherein the region of at least 19 contiguous nucleotides is present in a homologous gene of the virus in at least about 84% of the respiratory virus variants (strains) containing the homologous gene, except for no more than three (3) nucleotide mismatches among the region of at least 19 contiguous nucleotides; and
b. a second strand comprising from about 15 to about 40 nucleotides that is at least 70% complementary to the first strand.

3. The dsRNA molecule of claim 1, wherein the double-stranded region is from about 15 to about 40 base pairs, the dsRNA molecule comprising:
a. a first strand comprising from about 15 to about 40 nucleotides having a region of at least 25 contiguous nucleotides that are 100% complementary to a nucleic acid sequence of the respiratory virus gene, wherein the region of at least 25 contiguous nucleotides is present in a homologous gene of the virus in at least about 88% of the respiratory virus variants (strains) containing the homologous gene, except for no more than three (3) nucleotide mismatches among the region of at least 25 contiguous nucleotides; and
b. a second strand comprising from about 15 to about 40 nucleotides that is at least 70% complementary to the first strand.

4. The dsRNA molecule of claim 1, wherein the double-stranded region is from about 15 to about 60 base pairs, the dsRNA molecule comprising:
a. a first strand comprising from about 15 to about 60 nucleotides having a region of at least 15 contiguous nucleotides that are 100% complementary to a nucleic acid sequence of the respiratory virus gene, wherein the region of at least 15 contiguous nucleotides is present in a homologous gene of the virus in at least about 88% of the respiratory virus variants (strains) containing the homologous gene, except for no more than one (1) nucleotide mismatch among the region of at least 15 contiguous nucleotides; and
b. a second strand comprising from about 15 to about 60 nucleotides that is at least 70% complementary to the first strand.

5. The dsRNA molecule of claim 1, wherein the dsRNA molecule is an siRNA or an shRNA.

6. The dsRNA molecule of claim 1, wherein the dsRNA molecule has a 3′ overhang.

7. The dsRNA molecule of claim 1, wherein the dsRNA molecule has a 3′ overhang containing a deoxythymidine (dT).

8. The dsRNA molecule of claim 1, wherein the respiratory virus is influenza A virus.

9. The dsRNA molecule of claim 1, wherein the dsRNA molecule is an shRNA further comprising a hairpin loop structure.

10. The dsRNA molecule of claim 1, wherein the dsRNA molecule is an shRNA further comprising a hairpin loop structure having from 4 to 11 nucleotides.

11. The dsRNA molecule of claim 1, wherein the dsRNA molecule decreases expression of a respiratory virus gene by at least about 25% by RNA interference in a mammalian cell.

12. A double-stranded ribonucleic acid (dsRNA) molecule that decreases expression of a respiratory virus gene by RNA interference, wherein the dsRNA molecule has a double-stranded region of from about 15 to about 60 base pairs, the dsRNA molecule comprising:
a. a first strand comprising from about 15 to about 60 nucleotides having a region of at least 15 contiguous nucleotides that are 100% complementary to a nucleic acid sequence of the respiratory virus gene selected from the group consisting of SEQ ID NOS. 1-16949, wherein the region of at least 15 contiguous nucleotides is present in a homologous gene of the virus in at least about 70% of the respiratory virus variants (strains) containing the homologous gene, except for no more than three (3) nucleotide mismatches among the region of at least 15 contiguous nucleotides; and
b. a second strand comprising from about 15 to about 60 nucleotides that is at least 70% complementary to the first strand.

13. A double-stranded ribonucleic acid (dsRNA) molecule that decreases expression of a respiratory virus gene by RNA interference, wherein the dsRNA molecule has a double-stranded region of from about 15 to about 40 base pairs, the dsRNA molecule comprising:
a. a first strand comprising from about 15 to about 40 nucleotides having a region of at least 19 contiguous nucleotides that are 100% complementary to a nucleic acid sequence of the respiratory virus gene selected from the group consisting of SEQ ID NOS. 1-16949, wherein the region of at least 19 contiguous nucleotides is present in a homologous gene of the virus in at least about 84% of the respiratory virus variants (strains) containing the homologous gene, except for no more than three (3) nucleotide mismatches among the region of at least 19 contiguous nucleotides; and
b. a second strand comprising from about 15 to about 40 nucleotides that is at least 70% complementary to the first strand.

14. A double-stranded ribonucleic acid (dsRNA) molecule that decreases expression of a respiratory virus gene by RNA interference, wherein the dsRNA molecule has a double-stranded region of from about 15 to about 40 base pairs, the dsRNA molecule comprising:
a. a first strand comprising from about 15 to about 40 nucleotides having a region of at least 25 contiguous nucleotides that are 100% complementary to a nucleic acid sequence of the respiratory virus gene selected from the group consisting of SEQ ID NOS. 1-16949, wherein the region of at least 25 contiguous nucleotides is present in a homologous gene of the virus in at least about 88% of the respiratory virus variants (strains) containing the homologous gene, except for no more than three (3) nucleotide mismatches among the region of at least 25 contiguous nucleotides; and
b. a second strand comprising from about 15 to about 40 nucleotides that is at least 70% complementary to the first strand.

15. A double-stranded ribonucleic acid (dsRNA) molecule that decreases expression of a respiratory virus gene by RNA interference, wherein the dsRNA molecule has a double-stranded region of from about 15 to about 60 base pairs, the dsRNA molecule comprising:
a. a first strand comprising from about 15 to about 60 nucleotides having a region of at least 15 contiguous nucleotides that are 100% complementary to a nucleic acid sequence of the respiratory virus gene selected from the group consisting of SEQ ID NOS. 1-16949, wherein the region of at least 15 contiguous nucleotides is present in a homologous gene of the virus in at least about 70% of the respiratory virus variants (strains) containing the homologous gene, except for no more than one (1) nucleotide mismatch among the region of at least 15 contiguous nucleotides; and
b. a second strand comprising from about 15 to about 60 nucleotides that is at least 70% complementary to the first strand.

16. The dsRNA molecule of claim 12, wherein the dsRNA molecule is an siRNA or an shRNA.

17. The dsRNA molecule of claim 12, wherein the dsRNA molecule has a 3′ overhang.

18. The dsRNA molecule of claim 12, wherein the dsRNA molecule has a 3′ overhang containing a deoxythymidine (dT).

19. The dsRNA molecule of claim 12, wherein the respiratory virus is influenza A virus.

20. The dsRNA molecule of claim 12, wherein the dsRNA molecule is an shRNA further comprising a hairpin loop structure.

21. The dsRNA molecule of claim 12, wherein the dsRNA molecule is an shRNA further comprising a hairpin loop structure having from 4 to 11 nucleotides.

22. The dsRNA molecule of claim 12, wherein the dsRNA molecule decreases expression of a respiratory virus gene by at least about 25% by RNA interference in a mammalian cell.

23. A composition comprising a dsRNA according to claim 1 and a delivery agent.

24. A composition comprising two or more dsRNAs according to claim 1.

25. A composition comprising two or more dsRNAs according to claim 1, wherein the genes are from two or more strains of the same viral species.

26. A composition comprising two or more dsRNAs according to claim 1, wherein the genes are from two or more species of the same viral genus.

27. A composition comprising two or more dsRNAs according to claim 1, wherein the genes are from two viruses of the same viral family.

28. A composition comprising two or more dsRNAs according to claim 1, further comprising a delivery agent.

29. A method of preventing or treating a viral infection in a subject comprising administering an effective amount of a therapeutic dsRNA molecule according to claim 1.

30. The method of claim 29, wherein the virus is influenza A.

31. The method of claim 29, wherein the dsRNA is administered to the subject at a dosage of from about 0.1 mg/kg to about 20 mg/kg of body weight.

32. The method of claim 29, wherein the administration is intranasal, inhalation, oral, or intravenous.

33. A method of inhibiting production of a respiratory virus by RNA interference in a mammalian cell comprising providing a dsRNA according to claim 1 and contacting the cell with the dsRNA.

34. The method of claim 33, wherein the virus is influenza A.
